(19) United States
(12) Patent Application Publication
(10) Pub. No.: US 2011/0105509 A1
(43) Pub. Date: May 5, 2011

(54) INDOLE BASED RECEPTOR CRTH2 ANTAGONISTS

(75) Inventors: Neelu Kaila, Lexington, MA (US); Eddine Saiah, Brookline, MA (US); Bruce Christopher Follows, Littleton, MA (US); Adrian Huang, Lexington, MA (US); Kristin Marie Janz, Arlington, MA (US); Michael Dennis Lowe, Boston, MA (US); Alessandro Fabio Moretto, Somerville, MA (US); Jennifer Romanic Thomason, Bedford, MA (US); Tarek Suhayl Mansour, New City, NY (US); Cara Margaret May Williams, Methuen, MA (US)

(73) Assignee: Wyeth, LLC, Madison, NJ (US)

(21) Appl. No.: 12/938,479

(22) Filed: Nov. 3, 2010

Related U.S. Application Data
(60) Provisional application No. 61/257,975, filed on Nov. 4, 2009.

(51) Int. Cl. A61K 31/502; C07D 403/04; A61K 31/501; C07D 401/06; A61K 31/4439; C07D 403/14; C07D 403/06; A61K 31/4725; C07D 417/14; C07D 401/14; A61P 11/00; A61P 1/00; A61P 17/00; A61P 29/00; A61P 37/06; A61P 37/08; C12N 5/02

(52) U.S. Cl. 514/248; 544/237; 544/238; 514/252.06; 546/278.1; 514/339; 546/141; 514/309; 435/375

(57) ABSTRACT

Disclosed are compounds of Formula (I):

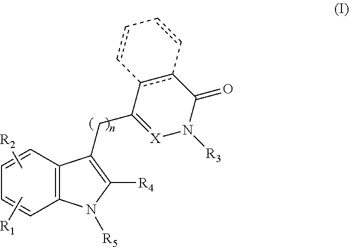

which are useful as antagonists of the CRTH2 receptors. Pharmaceutical compositions containing compounds of Formula (I) and the use of compounds of Formula (I) to treat diseases or disorders that are responsive to inhibition of the binding of endogenous ligands to the CRTH2 receptor are also disclosed. Methods for preparing and using these compounds are further described.

### FIELD OF THE INVENTION

The present invention relates to compounds that are effective as antagonists of the CRTH2 receptors. The present invention also relates to compositions comprising receptor CRTH2 antagonists, and to methods for preparing such compounds. The invention further relates to the use of these compounds to inhibit the binding of endogenous ligands to the CRTH2 receptor and to treat disorders responsive to such inhibition.

### BACKGROUND OF THE INVENTION

The Chemoattractant Receptor-homologous molecule expressed on T-Helper type 2 cells (CRTH2) receptor binds prostaglandin D₂ (PGD₂) and its metabolites. Efforts have been made to inhibit the binding of PGD₂ and other ligands to the CRTH2 receptor in order to treat disorders and diseases related to excess activation of CRTH2.

Elevated PGD₂ is thought to play a causative role in both asthma and atopic dermatitis. For example, PGD₂ is one of the major prostanoids released by mast cells in the asthmatic lung and this molecule is found at high levels in the bronchial fluid of asthmatics (Liu et al., Am. Rev. Respir. Dis. 142: 126 (1990)). Evidence of a role of PGD₂ in asthma is provided by a recent publication examining the effects of overexpression of prostaglandin D synthase on induction of allergic asthma in transgenic mice (Fujitani, J. Immunol. 168:443 (2002)). After allergen challenge, these animals had increased PGD₂ in the lungs, and the number of Th2 cells and eosinophils were greatly elevated relative to non-transgenic animals. These results are consistent with PGD₂ being a primary chemotactic agent in the recruitment of inflammatory cells during allergic asthma.

PGD₂ can bind to two G-protein coupled receptors, DP (Boie et al., J. Biol. Chem. 270:18910 (1995)) and CRTH2 (Nagata et al., J. Immunol. 162: 1278 (1999); Hirai et al., J. Exp. Med. 193:255 (2001)). The latter receptor might play a particularly important role in diseases such as asthma and atopic dermatitis that are characterized by Th2 cell involvement, since Th2 cell chemotaxis in response to PGD₂ appears to be mediated by CRTH2 (Hirai et al., above). Moreover, eosinophils, the major inflammatory cell type seen in asthmatic lungs, show a CRTH2-mediated chemotactic response to PGD₂ (Hirai et al.) and certain thromboxane metabolites (Bohm et al., J. Biol. Chem. 279:7663 (2004)).

Recently, indole derivatives showing moderate CRTH receptor antagonism have been reported, e.g., WO 2003/066046, WO 2003/066047, WO 2003/101961, WO 2003/101981, WO 2004/007451.However, there remains a need for novel and potent receptor CRTH2 antagonists that meet the demanding biological and pharmaceutical criteria required to justify the time and expense of human clinical trials. The present invention addresses this and other needs.

### SUMMARY OF THE INVENTION

The present invention provides compounds of Formula (I):

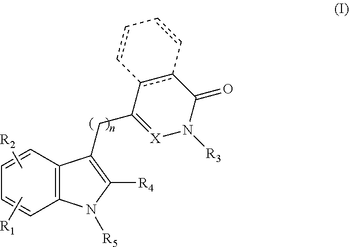

or a pharmaceutically acceptable salt thereof; wherein:

- - - - - - is a single bond or a double bond or is absent;

R₁ and R₂ are each independently H, halogen, OR₆, SO₂R₇, NR₈R₉, or alkyl; wherein

R₆ is H or alkyl;

R₇ is alkyl;

R₈ and R₉ are each independently H, COCH₃ or alkyl;

R₃ is hydroxy, alkyl, alkenyl, alkynyl, aryl, heteroaryl, cycloalkyl or heterocycloalkyl, wherein each alkyl, alkenyl, aryl, heteroaryl, cycloalkyl, heterocycloalkyl is optionally substituted with Rₐ; wherein

Rₐ is alkyl, aryl, heteroaryl, cycloalkyl, alkoxy, phenoxy, halogen, hydroxy, amino, mono- or di-alkylamino, nitro, haloalkyl, haloalkoxy, halophenoxy, CO, carboxamide, sulfonamide or SO₂Me, wherein each alkyl, aryl, heteroaryl is further optionally substituted with H, alkyl, aryl, alkoxy, phenoxy, halogen, hydroxy, haloalkyl, haloalkoxy, halophenoxy or SO₂Me;

R₄ is H or alkyl;

R₅ is CR₁₀R₁₁COOR₁₂, CR₁₀R₁₁CR₁₃NR₁₄R₁₅, COR₁₇, CR₁₀R₁₁CN, CR₁₀R₁₁CR₁₉; wherein

R₁₀ and R₁₁ are each independently H or alkyl;

R₁₂ is H or alkyl;

R₁₃ is O;

R₁₄ and R₁₅ are each independently H, COCH₃, SO₂R₁₆, alkyl, aryl, heteroaryl, cycloalkyl or heterocycloalkyl; wherein

R₁₆ is H, alkyl, aryl, heteroaryl, cycloalkyl or heterocycloalkyl;

R₁₇ is alkyl, aryl, heteroaryl, wherein each of which is optionally substituted with —OH or OR₁₈; wherein

R₁₈ is alkyl;

R₁₉ is alkyl, aryl, heteroaryl, or alkyl optionally substituted with —OH;

X is CH or N; and

n is 0 or 1.

The present invention also provides a pharmaceutical composition comprising an effective amount of one or more compounds of Formula (I) or a pharmaceutically acceptable salt thereof and a pharmaceutically acceptable carrier.

The present invention also provides a method for treating a disease or a disorder comprising administering to a patient in need thereof an effective amount of a compound of Formula (I) or a pharmaceutically acceptable salt thereof or a pharmaceutical composition comprising an effective amount of a compound of Formula (I).

In another embodiment, the disease or disorder is selected from the group consisting of asthma, chronic obstructive pulmonary disease (COPD), bronchitis, rhinitis, nasal polyposis, sarcoidosis, farmer's lung, fibroid lung, idiopathic intestinal pneumonia, cystic fibrosis, cough, psoriasis, dermatitis, urticaria, cutaneous eosinophilias, chronic sinusitis, eosinophilic esophagitis, eosinophilic gastroenteritis, eosinophilic colitis, eosinophilic fasciitis, lupus, rheumatoid arthritis, inflammatory Bowel disease, Celiac disease, scleroderma, ankylosing spondylitis, autoimmune diseases, allergic diseases and hyper IgE syndrome.

In other embodiment, the treatment of a disease or a disorder further comprises administering an additional therapeutic agent.

In another embodiment, the disease or disorder is characterized by elevated levels of a thromboxane metabolite, prostaglandin D₂ (PGD₂) or a metabolite thereof.

The present invention also provides a method of inhibiting the binding of endogenous ligands to the CRTH-2 receptor in a cell, comprising contacting the cell with a therapeutically effective amount of a compound of Formula (I) or a pharmaceutically acceptable salt thereof or a pharmaceutical composition of Formula (I).

In another embodiment, the endogenous ligand is a thromboxane metabolite, prostaglandin D₂ (PGD₂) or a metabolite thereof.

### DETAILED DESCRIPTION OF THE INVENTION

The present invention related to compounds of Formula (I), which are antagonists of the CRTH2 receptor and function as inhibitors of the binding of endogenous ligands to CRTH2 receptor. By inhibiting the binding of endogenous ligands such as PGD₂ and its metabolites, these compounds at least partially inhibit the effects of the endogenous ligands in a patient. Therefore, the invention related to methods of inhibiting the binding of endogenous ligands to the CRTH2 receptor on a cell, comprising contacting the cell with a compound of Formula (I). The invention further relates to methods of treating diseases or disorders in a patient that are responsive to inhibition of the CRTH2 receptor comprising administering to the patient a compound of Formula (I). Such diseases or disorders include those characterized by elevated levels of PGD2 or its metabolites or certain thromoxane metabolites.

### Definitions

Before describing the present invention in detail, it is to be understood that this invention is not limited to specific compositions or process steps, as such may vary. It should be noted that, as used in this specification and the appended claims, the singular form “a”, “an” and “the” include plural references unless the context clearly dictates otherwise. Thus, for example, reference to “a compound” includes a plurality of compounds.

Unless defined otherwise, all technical and scientific terms used herein have the same meaning as commonly understood by one of ordinary skill in the art to which this invention is related. The following terms are defined for purposes of the invention as described herein.

As used herein, unless otherwise noted, “alkyl” whether used alone or as part of a substituent group refers to a saturated straight and branched carbon chain having 1 to 20 carbon atoms or any number within this range, for example, 1 to 6 carbon atoms or 1 to 4 carbon atoms. Designated numbers of carbon atoms (e.g. C₁₋₆) shall refer independently to the number of carbon atoms in an alkyl moiety or to the alkyl portion of a larger alkyl-containing substituent. Non-limiting examples of alkyl groups include methyl, ethyl, n-propyl, iso-propyl, n-butyl, sec-butyl, iso-butyl, tert-butyl, and the like. Where so indicated, alkyl groups can be optionally substituted. In substituent groups with multiple alkyl groups such as N(C₁₋₆alkyl)₂, the alkyl groups may be the same or different.

As used herein, unless otherwise noted, “alkoxy” refers to groups of formula —Oalkyl. Designated numbers of carbon atoms (e.g. —OC₁₋₆) shall refer independently to the number of carbon atoms in the alkoxy group. Non-limiting examples of alkyl groups include methoxy, ethoxy, n-propoxy, iso-propoxy, n-butoxy, sec-butoxy, iso-butoxy, tert-butoxy, and the like. Where so indicated, alkoxy groups can be optionally substituted.

As used herein, the terms “alkenyl” and “alkynyl” groups, whether used alone or as part of a substituent group, refer to straight and branched carbon chains having 2 or more carbon atoms, preferably 2 to 20, having at least one carbon-carbon double bond (“alkenyl”) or at least one carbon-carbon triple bond (“alkynyl”). Where so indicated, alkenyl and alkynyl groups can be optionally substituted. Nonlimiting examples of alkenyl groups include ethenyl, 3-propenyl, 1-propenyl (also 2-methylethenyl), isopropenyl (also 2-methylethen-2-yl), buten-4-yl, and the like. Nonlimiting examples of alkynyl groups include ethynyl, prop-2-ynyl (also propargyl), propyn-1-yl, and 2-methyl-hex-4-yn-1-yl.

As used herein, “cycloalkyl” whether used alone or as part of another group, refers to a non-aromatic hydrocarbon ring including cyclized alkyl, alkenyl, or alkynyl groups, e.g., having from 3 to 14 ring carbon atoms, for example, from 3 to 7 or 3 to 6 ring carbon atoms, and optionally containing one or more (e.g., 1, 2, or 3) double or triple bonds. Cycloalkyl groups can be monocyclic (e.g., cyclohexyl) or polycyclic (e.g., containing fused, bridged, and/or spiro ring systems), wherein the carbon atoms are located inside or outside of the ring system. Any suitable ring position of the cycloalkyl group can be covalently linked to the defined chemical structure. Where so indicated, cycloalkyl rings can be optionally substituted. Nonlimiting examples of cycloalkyl groups include: cyclopropyl, cyclopropenyl, cyclobutyl, cyclobutenyl, cyclopentyl, cyclopentenyl, cyclopentadienyl, cyclohexyl, cyclohexenyl, cycloheptyl, cyclooctanyl, decalinyl, octahydropentalenyl, octahydro-1H-indenyl, 3a,4,5,6,7,7a-hexahydro-3H-inden-4-yl, decahydro-azulenyl; bicyclo[6.2.0]decanyl, decahydronaphthalenyl, and dodecahydro-1H-fluorenyl. The term “cycloalkyl” also includes carbocyclic rings which are bicyclic hydrocarbon rings, non-limiting examples of which include, bicyclo-[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3]undecanyl.

“Haloalkyl” is intended to include both branched and straight-chain saturated aliphatic hydrocarbon groups having the specified number of carbon atoms, substituted with 1 or more halogen atoms. As used herein, halogen refers to F, Cl, Br and I. Haloalkyl groups include perhaloalkyl groups, wherein all hydrogens of an alkyl group have been replaced with halogens (e.g., —CF₃, —CF₂CF₃). The halogens can be the same (e.g., CHF₂, —CF₃) or different (e.g., CF₂Cl). Where so indicated, haloalkyl groups can optionally be substituted with one or more substituents in addition to halogen. Examples of haloalkyl groups include, but are not limited to, fluoromethyl, dichloroethyl, trifluoromethyl, trichloromethyl, pentafluoroethyl, and pentachloroethyl groups.

The term “aryl” wherein used alone or as part of another group, is defined herein as an aromatic monocyclic ring of 6 carbons or an aromatic polycyclic ring of from 10 to 14 carbons. Aryl groups include but are not limited to, for example, phenyl or naphthyl (e.g., naphthylen-1-yl or naphthylen-2-yl). Where so indicated, aryl groups may be optionally substituted with one or more substituents. Aryl groups also include, but are not limited to for example, phenyl or naphthyl rings fused with one or more saturated or partially saturated carbon rings (e.g., bicyclo[4.2.0]octa-1,3,5-trienyl, indanyl), which can be substituted at one or more carbon atoms of the aromatic and/or saturated or partially saturated rings.

The term “heterocycloalkyl” whether used alone or as part of another group, is defined herein as a group having one or more rings (e.g., 1, 2 or 3 rings) and having from 3 to 20 atoms (e.g., 3 to 10 atoms, 3 to 6 atoms) wherein at least one atom in at least one ring is a heteroatom selected from nitrogen (N), oxygen (O), and sulfur (S), and wherein the ring that includes the heteroatom is non-aromatic. In heterocyclyl groups that include 2 or more fused rings, the non-heteroatom bearing ring may be aryl (e.g., indolinyl, tetrahydroquinolinyl, chromanyl). Exemplary heterocycloalkyl groups have from 3 to 14 ring atoms of which from 1 to 5 are heteroatoms independently selected from nitrogen (N), oxygen (O), or sulfur (S). One or more N or S atoms in a heterocycloalkyl group can be oxidized (e.g., N→O⁻, S(O), SO₂). Where so indicated, heterocycloalkyl groups can be optionally substituted.

Non-limiting examples of monocyclic heterocycloalkyl groups include, for example: diazirinyl, aziridinyl, urazolyl, azetidinyl, pyrazolidinyl, imidazolidinyl, oxazolidinyl, isoxazolinyl, isoxazolyl, thiazolidinyl, isothiazolyl, isothiazolinyl oxathiazolidinonyl, oxazolidinonyl, hydantoinyl, tetrahydropyranyl, pyrrolidinyl, morpholinyl, piperazinyl, piperidinyl, dihydropyranyl, tetrahydropyranyl, piperidin-2-onyl (valerolactam), 2,3,4,5-tetrahydro-1H-azepinyl, 2,3-dihydro-1H-indole, and 1,2,3,4-tetrahydro-quinoline. Non-limiting examples of heterocyclic groups having 2 or more rings include, for example: hexahydro-1H-pyrrolizinyl, 3a,4,5,6,7,7a-hexahydro-1H-benzo[d]imidazolyl, 3a,4,5,6,7,7a-hexahydro-1H-indolyl, 1,2,3,4-tetrahydroquinolinyl, chromanyl, isochromanyl, indolinyl, isoindolinyl, and decahydro-1H-cycloocta[b]pyrrolyl.

The term “heteroaryl” whether used alone or as part of another group, is defined herein as a single or fused ring system having from 5 to 20 atoms (e.g., 5 to 10 atoms, 5 to 6 atoms) wherein at least one atom in at least one ring is a heteroatom selected from nitrogen (N), oxygen (O), and sulfur (S), and wherein further at least one of the rings that includes a heteroatom is aromatic. In heteroaryl groups that include 2 or more fused rings, the non-heteroatom bearing ring may be a carbocycle (e.g., 6,7-Dihydro-5H-cyclopentapyrimidine) or aryl (e.g., benzofuranyl, benzo-thiophenyl, indolyl). Exemplary heteroaryl groups have from 5 to 14 ring atoms and contain from 1 to 5 ring heteroatoms independently selected from nitrogen (N), oxygen (O), and sulfur (S). One or more N or S atoms in a heteroaryl group can be oxidized (e.g., N→O⁻, S(O), SO₂). Where so indicated, heteroaryl groups can be substituted. Non-limiting examples of monocyclic heteroaryl rings include, for example: 1,2,3,4-tetrazolyl, [1,2,3]triazolyl, [1,2,4]triazolyl, triazinyl, thiazolyl, 1H-imidazolyl, oxazolyl, furanyl, thiopheneyl, pyrimidinyl, and pyridinyl. Non-limiting examples of heteroaryl rings containing 2 or more fused rings include: benzofuranyl, benzothiophenyl, benzoxazolyl, benzthiazolyl, benztriazolyl, cinnolinyl, naphthyridinyl, phenanthridinyl, 7H-purinyl, 9H-purinyl, 5H-pyrrolo[3,2-d]pyrimidinyl, 7H-pyrrolo[2,3-d]pyrimidinyl, pyrido[2,3-d]pyrimidinyl, 2-phenylbenzo[d]thiazolyl, 1H-indolyl, 4,5,6,7-tetrahydro-1-H-indolyl, quinoxalinyl, 5-methylquinoxalinyl, quinazolinyl, quinolinyl, and isoquinolinyl.

One non-limiting example of a heteroaryl group as described above is C₁-C₅ heteroaryl, which is a monocyclic aromatic ring having 1 to 5 carbon ring atoms and at least one additional ring atom that is a heteroatom (preferably 1 to 4 additional ring atoms that are heteroatoms) independently selected from nitrogen (N), oxygen (O), and sulfur (S). Examples of C₁-C₅ heteroaryl include, but are not limited to for example, triazinyl, thiazol-2-yl, thiazol-4-yl, imidazol-1-yl, 1H-imidazol-2-yl, 1H-imidazol-4-yl, isoxazolin-5-yl, furan-2-yl, furan-3-yl, thiophen-2-yl, thiophen-4-yl, pyrimidin-2-yl, pyrimidin-4-yl, pyrimidin-5-yl, pyridin-2-yl, pyridin-3-yl, and pyridin-4-yl.

For the purposes of the present invention, fused ring groups, spirocyclic rings, bicyclic rings and the like, which comprise a single heteroatom will be considered to belong to the cyclic family corresponding to the heteroatom containing ring. For example, 1,2,3,4-tetrahydroquinoline having the formula:

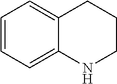

is, for the purposes of the present invention, considered a heterocycloalkyl group. 6,7-Dihydro-5H-cyclopentapyrimidine having the formula:

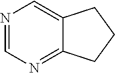

is, for the purposes of the present invention, considered a heteroaryl group. When a fused ring unit contains heteroatoms in both a saturated and an aryl ring, the aryl ring will predominate and determine the type of category to which the ring is assigned. For example, 1,2,3,4-tetrahydro-[1,8]naphthyridine having the formula:

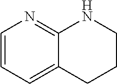

is, for the purposes of the present invention, considered a heteroaryl group.

The term “heteroarylene” whether used alone or as part of another group, is defined herein as a divalent single or fused ring system having from 5 to 20 atoms (e.g., 5 to 10 atoms, 5 to 6 atoms), wherein at least one atom in at least one ring is a heteroatom selected from nitrogen (N), oxygen (O), and sulfur (S), and wherein further at least one of the rings that includes a heteroatom is aromatic. In heteroarylene groups that include 2 or more fused rings, the non-heteroatom bearing ring may be a carbocycle (e.g., 6,7-Dihydro-5H-cyclopentapyrimidinylene) or aryl (e.g., benzofuranylene, benzothiophenylene, indolylene). Exemplary heteroarylene groups have from 5 to 14 ring atoms and contain from 1 to 5 ring heteroatoms independently selected from nitrogen (N), oxygen (O), and sulfur (S). One or more N or S atoms in a heteroarylene group can be oxidized (e.g., N→O⁻, S(O), SO₂). Where so indicated, heteroarylene groups can be substituted. Non-limiting examples of monocyclic heteroarylene rings include, for example: 1,2,3,4-tetrazolylene, [1,2,3]triazolylene, [1,2,4]triazolylene, triazinylene, thiazolylene, 1H-imidazolylene, oxazolylene, furanylene, thiopheneylene, pyrimidinylene, and pyridinylene. Non-limiting examples of heteroarylene rings containing 2 or more fused rings include: benzofuranylene, benzothiophenylene, benzoxazolylene, benzthiazolylene, benztriazolylene, cinnolinylene, naphthyridinylene, phenanthridinylene, 7H-purinylene, 9H-purinylene, 5H-pyrrolo[3,2-d]pyrimidinylene, 7H-pyrrolo[2,3-d]pyrimidinylene, pyrido[2,3-d]pyrimidinylene, 2-phenylbenzo [d]thiazolylene, 1H-indolylene, 4,5,6,7-tetrahydro-1-H-indolylene, quinoxalinylene, 5-methylquinoxalinylene, quinazolinylene, quinolinylene, and isoquinolinylene.

One non-limiting example of a heteroarylene group as described above is C₁-C₅ heteroarylene, which is a monocyclic aromatic ring having 1 to 5 carbon ring atoms and at least one additional ring atom that is a heteroatom (preferably 1 to 4 additional ring atoms that are heteroatoms) independently selected from nitrogen (N), oxygen (O), and sulfur (S). Examples of C₁-C₅ heteroarylene include, but are not limited to for example, triazinylene, thiazol-2-ylene, thiazol-4-ylene, imidazol-1-ylene, 1H-imidazol-2-ylene, 1H-imidazol-4-ylene, isoxazolin-5-ylene, furan-2-ylene, furan-3-ylene, thiophen-2-ylene, thiophen-4-ylene, pyrimidin-2-ylene, pyrimidin-4-ylene, pyrimidin-5-ylene, pyridin-2-ylene, pyridin-3-ylene, and pyridin-4-ylene.

The term “carbocyclic ring” refers to a saturated cyclic, partially saturated cyclic, or aromatic ring containing from 3 to 14 carbon ring atoms. A carbocyclic ring may be monocyclic, bicyclic or tricyclic. A carbocyclic ring typically contains from 3 to 10 carbon ring atoms and is monocyclic or bicyclic.

The term “heterocyclic ring” refers to a saturated cyclic, partially saturated cyclic, or aromatic ring containing from 3 to 14 ring atoms, in which at least one of the ring atoms is a heteroatom that is oxygen, nitrogen, or sulfur. A heterocyclic ring may be monocyclic, bicyclic or tricyclic. A heterocyclic ring typically contains from 3 to 10 ring atoms and is monocyclic or bicyclic.

The term “amino” refers to —NH₂.

The term “alkylamino” refers to —N(H)alkyl. Examples of alkylamino substituents include methylamino, ethylamino, and propylamino.

The term “dialkylamino” refers to —N(alkyl)₂ where the two alkyls may be the same or different. Examples of dialkylamino substituents include dimethylamino, diethylamino, ethylmethylamino, and dipropylamino.

The term “halogen” refers to fluorine (which may be depicted as —F), chlorine (which may be depicted as —Cl), bromine (which may be depicted as —Br), or iodine (which may be depicted as —I).

The term “azide” refers to —N₃.

The terms “treat” and “treating,” as used herein, refer to partially or completely alleviating, inhibiting, ameliorating and/or relieving a condition from which a patient is suspected to suffer.

As used herein, “therapeutically effective” refers to a substance or an amount that elicits a desirable biological activity or effect.

Except when noted, the terms “subject” or “patient” are used interchangeably and refer to mammals such as human patients and non-human primates, as well as experimental animals such as rabbits, rats, and mice, and other animals. Accordingly, the term “subject” or “patient” as used herein means any mammalian patient or subject to which the compounds of the invention can be administered. In an exemplary embodiment of the present invention, to identify subject patients for treatment according to the methods of the invention, accepted screening methods are employed to determine risk factors associated with a targeted or suspected disease or condition or to determine the status of an existing disease or condition in a subject. These screening methods include, but are not limited to for example, conventional work-ups to determine risk factors that may be associated with the targeted or suspected disease or condition. These and other routine methods allow the clinician to select patients in need of therapy using the methods and compounds of the present invention.

The term “substituted” is used throughout the specification. The term “substituted” is defined herein as a moiety, whether acyclic or cyclic, which has one or more (e.g. 1-10) hydrogen atoms replaced by a substituent as defined herein below. Substituents include those that are capable of replacing one or two hydrogen atoms of a single moiety at a time, and also those that can replace two hydrogen atoms on two adjacent carbons to form said substituent. For example, substituents that replace single hydrogen atoms includes, for example, halogen, hydroxyl, and the like. A two hydrogen atom replacement includes carbonyl, oximino, and the like. Substituents that replace two hydrogen atoms from adjacent carbon atoms include, for example, epoxy, and the like. When a moiety is described as “substituted” any number of its hydrogen atoms can be replaced, as described above. For example, difluoromethyl is a substituted C₁ alkyl; trifluoromethyl is a substituted C₁ alkyl; 4-hydroxyphenyl is a substituted aryl ring; (N,N-dimethyl-5-amino)octanyl is a substituted C₈ alkyl; 3-guanidinopropyl is a substituted C₃ alkyl; and 2-carboxypyridinyl is a substituted heteroaryl.

At various places in the present specification, substituents of compounds are disclosed in groups or in ranges. It is specifically intended that the description include each and every individual subcombination of the members of such groups and ranges. For example, the term “C₁₋₆ alkyl” is specifically intended to individually disclose C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

Compounds described herein can contain an asymmetric atom (also referred as a chiral center), and some of the compounds can contain one or more asymmetric atoms or centers, which can thus give rise to optical isomers (enantiomers) and diastereomers. The present teachings and compounds disclosed herein include such enantiomers and diastereomers, as well as the racemic and resolved, enantiomerically pure R and S stereoisomers, as well as other mixtures of the R and S stereoisomers and pharmaceutically acceptable salts thereof. Optical isomers can be obtained in pure form by standard procedures known to those skilled in the art, which include, but are not limited to for example, chiral chromatography, diastereomeric salt formation, kinetic resolution, and asymmetric synthesis. The present invention also includes cis and trans or E/Z isomers of compounds of Formula (I) containing alkenyl moieties (e.g., alkenes and imines). It is also understood that the present teachings encompass all possible regioisomers, and mixtures thereof, which can be obtained in pure form by standard separation procedures known to those skilled in the art, and include, but are not limited to, column chromatography, thin-layer chromatography, and high-performance liquid chromatography.

The term “CRTH2 receptor” as used herein, refers to any known member of the CRTH2 receptor family, including but not limited to, hCRTH2.

The term “elevated levels of PGD₂ or its metabolites or certain thromboxane metabolites” as used herein, refers to an elevated level (e.g., aberrant level) of these molecules in biological tissue or fluid as compared to similar corresponding non-pathological tissue or fluid containing basal levels of PGD₂ or its metabolites or thromboxanes and metabolites.

The term “other therapeutic agents” as used herein, refers to any therapeutic agent that has been used, is currently used or is known to be useful for treating a disease or a disorder encompassed by the present invention. For example, agents used to treat asthma and rhinitis include steroids, β-receptor agonists and leukotriene receptor antagonists.

The term “prodrug” as used herein, refers to a pharmacologically inactive derivative of a parent “drug” molecule that requires biotransformation (e.g., either spontaneous or enzymatic) within the target physiological system to release or convert the prodrug into the active drug. Prodrugs are designed to overcome problems associated with stability, toxicity, lack of specificity, or limited bioavailability. Exemplary prodrugs comprise an active drug molecule itself and a chemical masking group (e.g., a group that reversibly suppresses the activity of the drug). Some preferred prodrugs are variations or derivatives of compounds that have groups cleavable under metabolic conditions. Exemplary prodrugs become pharmaceutically active in vivo or in vitro when they undergo solvolysis under physiological conditions or undergo enzymatic degradation or other biochemical transformation (e.g., phosphorylation, hydrogenation, dehydrogenation, glycosylation). Prodrugs often offer advantages of solubility, tissue compatibility, or delayed release in the mammalian organism. (See e.g., Bundgard, Design of Prodrugs, pp. 7-9, 21-24, Elsevier, Amsterdam (1985); and Silverman, The Organic Chemistry of Drug Design and Drug Action, pp. 352-401, Academic Press, San Diego, Calif. (1992)). Common prodrugs include acid derivatives such as esters prepared by reaction of parent acids with a suitable alcohol (e.g., a lower alkanol), amides prepared by reaction of the parent acid compound with an amine, or basic groups reacted to form an acylated base derivative (e.g., a lower alkylamide).

The term “pharmaceutically acceptable salt” as used herein, refers to any salt (e.g., obtained by reaction with an acid or a base) of a compound of the present invention that is physiologically tolerated in the target animal (e.g., a mammal). Salts of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, sulfonic, naphthalene-2-sulfonic, benzenesulfonic acid, and the like.

Examples of bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, chloride, bromide, iodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁻, and NW₄⁺ (wherein W is a C₁₋₄ alkyl group), and the like. For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

The term “therapeutically effective amount” as used herein, refers to that amount of the therapeutic agent sufficient to result in amelioration of one or more symptoms of a disorder, or prevent advancement of a disorder, or cause regression of the disorder. For example, with respect to the treatment of asthma, a therapeutically effective amount preferably refers to the amount of a therapeutic agent that increases peak air flow by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

The compounds described herein may be administered to humans and other animals orally, parenterally, sublingually, by aerosolization or inhalation spray, rectally, intracisternally, intravaginally, intraperitoneally, bucally, intrathecally or topically in dosage unit formulations containing conventional nontoxic pharmaceutically acceptable carriers, adjuvants, and vehicles as desired. The term parenteral as used herein includes subcutaneous injection, intravenous injection, intramuscular injection, intrasternal injection, or infusion techniques. Topical administration may also involve the use of transdermal administration such as transdermal patches or ionophoresis devices.

Methods of formulation are well known in the art and are disclosed, for example, in Remington: The Science and Practice of Pharmacy, Mack Publishing Company, Easton, Pa., 21st Edition (2005), incorporated herein by reference.

Pharmaceutical compositions for use in the present invention can be in the form of sterile, non-pyrogenic liquid solutions or suspensions, coated capsules, suppositories, lyophilized powders, transdermal patches or other forms known in the art.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent.

In addition, sterile, fixed oils are conventionally employed as a solvent or suspending medium. For this purpose any bland fixed oil may be employed including synthetic mono- or di-glycerides. In addition, fatty acids such as oleic acid find use in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

Formulations comprising crystalline forms of the compositions described herein for slow absorption from subcutaneous or intramuscular injection are provided herein. Additionally, delayed absorption of a parenterally administered drug form may be accomplished by dissolving or suspending the compounds in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations may also be prepared by entrapping the drug in liposomes or microemulsions, which are compatible with body tissues.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, acetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

The compounds described herein can also be in micro-encapsulated form with one or more excipients as noted above. The solid dosage forms of tablets, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, EtOAc, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulations, ear drops, and the like are also contemplated as being within the scope of this invention.

Compositions of the invention may also be formulated for delivery as a liquid aerosol or inhalable dry powder. Liquid aerosol formulations may be nebulized predominantly into particle sizes that can be delivered to the terminal and respiratory bronchioles.

The amount of active ingredient that may be combined with the carrier materials to produce a single dosage form will vary depending upon the host treated and the particular mode of administration. It will be understood, however, that the specific dose level for any particular subject will depend upon a variety of factors including the activity of the specific compound employed, the age, body weight, general health, sex, diet, time of administration, route of administration, rate of excretion, drug combination, and the severity of the particular disease undergoing therapy. The therapeutically effective amount for a given situation can be readily determined by routine experimentation and is within the skill and judgment of the ordinary clinician.

In another aspect of the invention, kits that include one or more compounds of the invention are provided. Representative kits include a compound described herein (e.g., a compound of Formula I) and a package insert or other labeling including directions for treating a disease or a disorder by administering an effective amount of a compound of the present invention.

In another aspect of the invention, kits that include one or more compounds of the invention are provided. Representative kits include a compound described herein (e.g., a compound of Formula I) and a package insert or other labeling including directions for inhibiting the binding of endogenous ligands to the CRTH-2 receptor in a cell by administering an effective amount of a compound of the present invention.

The phrase “pharmaceutically acceptable carrier” as used herein means a pharmaceutically-acceptable material, composition or vehicle, such as a liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically- acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. A physiologically acceptable carrier should not cause significant irritation to an organism and does not abrogate the biological activity and properties of the administered compound.

An “excipient” refers to an inert substance added to a pharmacological composition to further facilitate administration of a compound. Examples of excipients include but are not limited to calcium carbonate, calcium phosphate, various sugars and types of starch, cellulose derivatives, gelatin, vegetable oils and polyethylene glycols.

A “pharmaceutically effective amount” means an amount which is capable of providing a therapeutic and/or prophylactic effect. The specific dose of compound administered according to this invention to obtain therapeutic and/or prophylactic effect will, of course, be determined by the particular circumstances surrounding the case, including, for example, the specific compound administered, the route of administration, the condition being treated, and the individual being treated. A typical daily dose (administered in single or divided doses) will contain a dosage level of from about 0.01 mg/kg to about 50-100 mg/kg of body weight of an active compound of the invention. Preferred daily doses generally will be from about 0.05 mg/kg to about 20 mg/kg and ideally from about 0.1 mg/kg to about 10 mg/kg. Factors such as clearance rate, half-life and maximum tolerated dose (MTD) have yet to be determined but one of ordinary skill in the art can determine these using standard procedures.

As used herein, the term “IC₅₀” refers to an amount, concentration or dosage of a particular test compound that achieves a 50% inhibition of a maximal response in an assay that measures such response. The value depends on the assay used.

In one aspect, the present invention provides compounds of Formula (I):

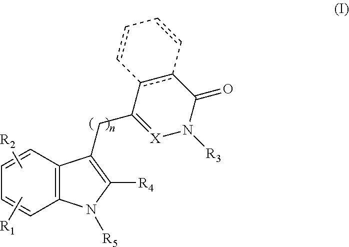

or a pharmaceutically acceptable salt thereof; wherein:

- - - - - - is a single bond or a double bond or is absent;

R₁ and R₂ are each independently H, halogen, OR₆, SO₂R₇, NR₈R₉, or alkyl; wherein

R₆ is H or alkyl;

R₇ is alkyl;

R₈ and R₉ are each independently H, COCH₃ or alkyl;

R₃ is hydroxy, alkyl, alkenyl, alkynyl, aryl, heteroaryl, cycloalkyl or heterocycloalkyl, wherein each alkyl, alkenyl, aryl, heteroaryl, cycloalkyl, heterocycloalkyl is optionally substituted with Rₐ; wherein

Rₐ is alkyl, aryl, heteroaryl, cycloalkyl, alkoxy, phenoxy, halogen, hydroxy, amino, mono- or di-alkylamino, nitro, haloalkyl, haloalkoxy, halophenoxy, CO, carboxamide, sulfonamide or SO₂Me, wherein each alkyl, aryl, heteroaryl is further optionally substituted with H, alkyl, aryl, alkoxy, phenoxy, halogen, hydroxy, haloalkyl, haloalkoxy, halophenoxy or SO₂Me;

R₄ is H or alkyl;

R₅ is CR₁₀R₁₁COOR₁₂, CR₁R₁₁CR₁₃NR₁₄R₁₅, COR₁₇, CR₁₀R₁₁CN, CR₁₀R₁₁CR₁₉; wherein

R₁₀ and R₁₁ are each independently H or alkyl;

R₁₂ is H or alkyl;

R₁₃ is O;

R₁₄ and R₁₅ are each independently H, COCH₃, SO₂R₁₆, alkyl, aryl, heteroaryl, cycloalkyl or heterocycloalkyl; wherein

R₁₆ is H, alkyl, aryl, heteroaryl, cycloalkyl or heterocycloalkyl;

R₁₇ is alkyl, aryl, heteroaryl, wherein each of which is optionally substituted with —OH or OR₁₈; wherein

R₁₈ is alkyl;

R₁₉ is alkyl, aryl, heteroaryl, or alkyl optionally substituted with —OH;

X is CH or N; and

n is 0 or 1.

In some embodiments, R₁ is halogen.

In some embodiments, R₁ is alkyl.

In some embodiments, R₁ is SO₂Me.

In some embodiments, R₂ is halogen.

In some embodiments, R₂ is alkyl.

In some embodiments, R₂ is SO₂Me.

In some embodiments, R₃ is alkyl optionally substituted with alkyl, aryl, heteroaryl, cycloalkyl, alkoxy, halogen, hydroxy, amino, mono- or di-alkylamino, nitro, haloalkyl, haloalkoxy, carboxamide, sulfonamide or SO₂Me.

In some embodiments, R₃ is aryl optionally substituted with alkyl, aryl, heteroaryl, cycloalkyl, alkoxy, halogen, hydroxy, amino, mono- or di-alkylamino, nitro, haloalkyl, haloalkoxy, carboxamide, sulfonamide or SO₂Me.

In some embodiments, R₃ is heteroaryl optionally substituted with alkyl, aryl, heteroaryl, cycloalkyl, alkoxy, halogen, hydroxy, amino, mono- or di-alkylamino, nitro, haloalkyl, haloalkoxy, carboxamide, sulfonamide or SO₂Me.

In some embodiments, R₃ is cycloalkyl optionally substituted with alkyl, aryl, heteroaryl, cycloalkyl, alkoxy, halogen, hydroxy, amino, mono- or di-alkylamino, nitro, haloalkyl, haloalkoxy, carboxamide, sulfonamide or SO₂Me.

In some embodiments, R₄ is alkyl.

In some embodiments, R₅ is CH₂COOH.

In some embodiments, R₅ is CH₂CONHSO₂Me.

In some embodiments, X is CH.

In some embodiments, X is N.

In some embodiments, n is O.

In some embodiments, n is 1.

In some embodiments, the compounds include:

2-(5-Chloro-3-(3-(4-chlorobenzyl)-4-oxo-3,4-dihydrophthalazin-1-yl)-2-methyl-1H-indol-1-yl)acetic acid;

2-(5-Chloro-3-(3-(4-chloro-3-fluorobenzyl)-4-oxo-3,4-dihydrophthalazin-1-yl)-2-methyl-1H-indol-1-yl)acetic acid;

2-(5-Chloro-3-(3-(3-fluoro-4-(trifluoromethyl)benzyl)-4-oxo-3,4-dihydrophthalazin-1-yl)-2-methyl-1H-indol-1-yl)acetic acid;

2-(3-(3-(4-Chlorobenzyl)-4-oxo-3,4-dihydropthalazin-1-yl)-5-fluoro-2-methyl-1H-indol-1-yl)acetic acid;

2-(3-(3-(4-Chloro-3-fluorobenzyl)-4-oxo-3,4-dihydrophthalazin-1-yl)-5-fluoro-2-methyl-1H-indol-1-yl)acetic acid;

2-(5-Fluoro-3-(3-(3-fluoro-4-(trifluoromethyl)benzyl)-4-oxo-3,4-dihydrophthalazin-1-yl)-2-methyl-1H-indol-1-yl)acetic acid;

2-(3-(3-(2,4-dichlorobenzyl)-4-oxo-3,4-dihydrophthalazin-1-yl)-5-fluoro-2-methyl-1H-indol-1-yl)acetic acid;

2-(5-Chloro-3-(1-(4-chloro-3-fluorobenzyl)-6-oxo-1,6-dihydropyridazin-3-yl)-2-methyl-1H-indol-1-yl)acetic acid;

2-(5-Chloro-3-(1-(4-chlorobenzyl)-6-oxo-1,6-dihydropyridazin-3-yl)-2-methyl-1H-indol-1-yl)acetic acid;

2-(3-(1-Benzyl-6-oxo-1,6-dihydropyridazin-3-yl)-5-chloro-7-fluoro-2-methyl-1H-indol-1-yl)acetic acid;

2-(3-(3-Benzyl-4-oxo-3,4-dihydrophthalazin-1-yl)-5-chloro-7-fluoro-2-methyl-1H-indol-1-yl)acetic acid;

2-(3-(1-(4-Chloro-3-fluorobenzyl)-6-oxo-1,6-dihydropyridazin-3-yl)-5-fluoro-2-methyl-1H-indol-1-yl)acetic acid;

2-(3-(1-(4-Chlorobenzyl)-6-oxo-1,6-dihydropyridazin-3-yl)-5-fluoro-2-methyl-1H-indol-1-yl)acetic acid;

2-(3-(3-Benzyl-4-oxo-3,4-dihydrophthalazin-1-yl)-7-chloro-2-methyl-5-(methylsulfonyl)-1H-indol-1-yl)acetic acid;

2-(3-(3-Benzyl-4-oxo-3,4-dihydrophthalazin-1-yl)-7-fluoro-2-methyl-5-(methylsulfonyl)-1H-indol-1-yl)acetic acid;

2-(3-(3-Benzyl-4-oxo-3,4-dihydrophthalazin-1-yl)-5-methoxy-2-methyl-1H-indol-1-yl)acetic acid;

2-(3-(1-Benzyl-6-oxo-1,6-dihydropyridazin-3-yl)-7-fluoro-2-methyl-5-(methylsulfonyl)-1H-indol-1-yl)acetic acid;

2-(3-(1-Benzyl-6-oxo-1,6-dihydropyridazin-3-yl)-5-methoxy-2-methyl-1H-indol-1-yl)acetic acid;

2-(3-(3-Isopropyl-4-oxo-3,4-dihydrophthalazin-1-yl)-5-methoxy-2-methyl-1H-indol-1-yl)-acetic acid;

2-(3-((1-(2,4-Difluorobenzyl)-6-oxo-1,6-dihydropyridin-3-yl)methyl)-5-fluoro-2-methyl-1H-indol-1-yl)acetic acid;

2-(3-((1-(2,4-Difluorobenzyl)-6-oxo-1,6-dihydropyridin-3-yl)methyl)-2-methyl-1H-indol-1-yl)acetic acid;

2-(3-((1-Isopropyl-6-oxo-1,6-dihydropyridin-3-yl)methyl-2-methyl-1H-indol-1-yl)acetic acid;

2-(5-Fluoro-2-methyl-3-((6-oxo-1-(2,4,5-trifluorobenzyl)-1,6-dihydropyridin-3-yl)methyl)-1H-indol-1-yl)acetic acid;

2-(2-Methyl-3-((6-oxo-1-(2,4,5-trifluorobenzyl)-1,6-dihydropyridin-3-yl)methyl-1H-indol-1-yl)acetic acid;

2-(3-((1-(2,5-Difluorobenzyl)-6-oxo-1,6-dihydropyridin-3-yl)methyl)-2-methyl-1H-indol-1-yl)acetic acid;

2-(3-((1-(2,5-Difluorobenzyl)-6-oxo-1,6-dihydropyridin-3-yl)methyl)-5-fluoro-2-methyl-1H-indol-1-yl)acetic acid;

2-(5-fluoro-3-((1-isopropyl-6-oxo-1,6-dihydropyridin-3-yl)methyl)-2-methyl-1H-indol-1-yl)acetic acid;

2-(3-((1-(2,3-Difluorobenzyl)-6-oxo-1,6-dihydropyridin-3-yl)methyl)-5-fluoro-2-methyl-1H-indol-1-yl)acetic acid;

2-(3-(1-(2,3-Difluorobenzyl)-6-oxo-1,6-dihydropyridin-3-yl)methyl)-2-methyl-1H-indol-1-yl)acetic acid;

2-(3-((1-(3,4-Difluorobenzyl)-6-oxo-1,6-dihydropyridin-3-yl)methyl)-2-methyl-1H-indol-1-yl)acetic acid;

2-(3-((1-(2-Fluorobenzyl)-6-oxo-1,6-dihydropyridin-3-yl)methyl)-2-methyl-1H-indol-1-yl)-acetic acid;

2-(5-Fluoro-3-((1-(2-fluorobenzyl)-6-oxo-1,6-dihydropyridin-3-yl)methyl)-2-methyl-1H-indol-1-yl)acetic acid;

2-(3-((1-(3,4-Difluorobenzyl)-6-oxo-1,6-dihydropyridin-3-yl)methyl)-5-fluoro-2-methyl-1H-indol-1-yl)acetic acid;

2-(3-((1-(3-Fluorobenzyl)-6-oxo-1,6-dihydropyridin-3-yl)methyl)-2-methyl-1H-indol-1-yl)-acetic acid;

2-(3-((1-(3,5-Difluorobenzyl)-6-oxo-1,6-dihydropyridin-3-yl)methyl)-2-methyl-1H-indol-1-yl)acetic acid;

2-(3-((1-(3,5-Difluorobenzyl)-6-oxo-1,6-dihydropyridin-3-yl)methyl)-5-fluoro-2-methyl-1H-indol-1-yl)acetic acid;

2-(3-((1-(2,6-Difluorobenzyl)-6-oxo-1,6-dihydropyridin-3-yl)methyl)-2-methyl-1H-indol-1-yl)acetic acid;

2-(3-((1-(2,6-Difluorobenzyl)-6-oxo-1,6-dihydropyridin-3-yl)methyl)-5-fluoro-2-methyl-1H-indol-1-yl)acetic acid;

2-(5-Fluoro-3-((1-(3-fluorobenzyl)-6-oxo-1,6-dihydropyridin-3-yl)methyl)-2-methyl-1H-indol-1-yl)acetic acid;

2-(3-((1-(4-Fluorobenzyl)-6-oxo-1,6-dihydropyridin-3-yl)methyl)-2-methyl-1H-indol-1-yl)-acetic acid;

2-(5-Fluoro-3-((1-(4-fluorobenzyl)-6-oxo-1,6-dihydropyridin-3-yl)methyl)-2-methyl-1H-indol-1-yl)acetic acid;

2-(5-Chloro-3-((1-(2,4-difluorobenzyl)-6-oxo-1,6-dihydropyridin-3-yl)methyl)-2-methyl-1H-indol-1-yl)acetic acid;

2-(5-Chloro-3-((1-(2,3-difluorobenzyl)-6-oxo-1,6-dihydropyridin-3-yl)methyl)-2-methyl-1H-indol-1-yl)acetic acid;

2-(5-Chloro-2-methyl-3-((6-oxo-1-(2,4,5-trifluorobenzyl)-1,6-dihydropyridin-3-yl)methyl)-1H-indol-1-yl)acetic acid;

2-(5-Chloro-3-((1-(3-fluorobenzyl)-6-oxo-1,6-dihydropyridin-3-yl)methyl)-2-methyl-1H-indol-1-yl)acetic acid;

2-(5-Fluoro-2-methyl-3-((6-oxo-1-(2,2,2-trifluoroethyl)-1,6-dihydropyridin-3-yl)methyl-1H-indol-1-yl)acetic acid;

2-(3-(3-Benzyl-4-oxo-3,4-dihydrophthalazin-1-yl)-2-methyl-1H-indol-1-yl)acetamide;

2-(3-(3-Benzyl-4-oxo-3,4-dihydrophthalazin-1-yl)-2-methyl-1H-indol-1-yl)-N,N-dimethylacetamide;

2-Benzyl-4-(2-methyl-1-(2-oxo-2-(pyrrolidin-1-yl)ethyl)-1H-indol-3-yl)phthalazin-1(2H)-one;

2-(3-(3-Benzyl-4-oxo-3,4-dihydrophthalazin-1-yl)-2-methyl-1H-indol-1-yl)-N-(methyl-sulfonyl)acetamide;

4-(1-((2H-Tetrazol-5-yl)methyl)-2-methyl-1H-indol-3-yl)-2-benzylphthalazin-1(2H)-one;

2-Benzyl-4-(1-(2-hydroxyethyl)-2-methyl-1H-indol-3-yl)phthalazin-1(2H)-one;

2-(5-Chloro-2-methyl-3-((6-oxo-1-(2,2,2-trifluoroethyl)-1,6-dihydropyridin-3-yl)methyl)-1H-indol-1-yl)acetic acid;

2-(5-Fluoro-2-methyl-3-((6-oxo-1-(4,4,4-trifluorobutyl)-1,6-dihydropyridin-3-yl)methyl)-1H-indol-1-yl)acetic acid;

2-(4-Acetamido-3-((1-(2,4-difluorobenzyl)-6-oxo-1,6-dihydropyridazin-3-yl)methyl)-2-methyl-1H-indol-1-yl)acetic acid;

2-(3-((1-(2,4-Difluorobenzyl)-6-oxo-1,4,5,6-tetrahydropyridazin-3-yl)methyl)-5-fluoro-2-methyl-1H-indol-1-yl)acetic acid;

2-(5-Fluoro-2-methyl-3-((6-oxo-1-(2,2,2-trifluoroethyl)-1,4,5,6-tetrahydropyridazin-3-yl)-methyl)-1H-indol-1-yl)acetic acid;

2-(5-Fluoro-2-methyl-3-((6-oxo-1-(4,4,4-trifluorobutyl)-1,4,5,6-tetrahydropyridazin-3-yl)-methyl)-1H-indol-1-yl)acetic acid;

2-(5-Chloro-3-(3-(2,5-difluorobenzyl)-4-oxo-3,4-dihydrophthalazin-1-yl)-2-methyl-1H-indol-1-yl)acetic acid;

2-(5-Chloro-2-methyl-3-(4-oxo-3-(2,4,5-trifluorobenzyl)-3,4-dihydrophthalazin-1-yl)-1H-indol-1-yl)acetic acid;

2-(5-Chloro-3-(3-(2,4-difluorobenzyl)-4-oxo-3,4-dihydrophthalazin-1-yl)-2-methyl-1H-indol-1-yl)acetic acid;

2-(3-(3-(2,5-Difluorobenzyl)-4-oxo-3,4-dihydrophthalazin-1-yl)-5-fluoro-2-methyl-1H-indol-1-yl)acetic acid;

2-(5-Fluoro-2-methyl-3-(4-oxo-3-(2,4,5-trifluorobenzyl)-3,4-dihydrophthalazin-1-yl)-1H-indol-1-yl)acetic acid;

2-(3-(3-(2,4-Difluorobenzyl)-4-oxo-3,4-dihydrophthalazin-1-yl)-5-fluoro-2-methyl-1H-indol-1-yl)acetic acid;

2-(5-Fluoro-2-methyl-3-(3-(4-(methylsulfonyl)benzyl)-4-oxo-3,4-dihydrophthalazin-1-yl)-1H-indol-1-yl)acetic acid;

2-(5-Chloro-2-methyl-3-(1-(4-(methylsulfonyl)benzyl)-6-oxo-1,6-dihydropyridazin-3-yl)-1H-indol-1-yl)acetic acid;

2-(5-Chloro-3-(1-(2,5-difluorobenzyl)-6-oxo-1,6-dihydropyridazin-3-yl)-2-methyl-1H-indol-1-yl)acetic acid;

2-(5-Chloro-3-(1-(2,4-difluorobenzyl)-6-oxo-1,6-dihydropyridazin-3-yl)-2-methyl-1H-indol-1-yl)acetic acid;

2-(5-Chloro-2-methyl-3-(6-oxo-1-(2,4,5-trifluorobenzyl)-1,6-dihydropyridazin-3-yl)-1H-indol-1-yl)acetic acid;

2-(5-Fluoro-2-methyl-3-(6-oxo-1-(2,4,5-trifluorobenzyl)-1,6-dihydropyridazin-3-yl)-1H-indol-1-yl)acetic acid;

2-(3-(1-(2,4-Difluorobenzyl)-6-oxo-1,6-dihydropyridazin-3-yl)-5-fluoro-2-methyl-1H-indol-1-yl)acetic acid;

2-(3-(1-(2,5-Difluorobenzyl)-6-oxo-1,6-dihydropyridazin-3-yl)-5-fluoro-2-methyl-1H-indol-1-yl)acetic acid;

2-(5-Fluoro-2-methyl-3-(1-(4-(methylsulfonyl)benzyl)-6-oxo-1,6-dihydropyridazin-3-yl)-1H-indol-1-yl)acetic acid;

2-(3-((1-(2-Fluorobenzyl)-6-oxo-1,6-dihydropyridazin-3-yl)methyl)-2-methyl-1H-indol-1-yl)acetic acid;

2-(3-((1-(3-Fluorobenzyl)-6-oxo-1,6-dihydropyridazin-3-yl)methyl)-2-methyl-1H-indol-1-yl)acetic acid;

2-(3-((1-(4-Fluorobenzyl)-6-oxo-1,6-dihydropyridazin-3-yl)methyl)-2-methyl-1H-indol-1-yl)acetic acid;

2-(3-((1-Benzyl-6-oxo-1,6-dihydropyridazin-3-yl)methyl)-2-methyl-1H-indol-1-yl)acetic acid;

2-(3-((1-Benzyl-6-oxo-1,4,5,6-tetrahydropyridazin-3-yl)methyl)-5-fluoro-2-methyl-1H-indol-1-yl)acetic acid;

2-(3-((1-(2,3-Difluorobenzyl)-6-oxo-1,6-dihydropyridazin-3-yl)methyl)-2-methyl-1H-indol-1-yl)acetic acid;

2-(3-((1-Benzyl-6-oxo-1,6-dihydropyridazin-3-yl)methyl)-5-chloro-2-methyl-1H-indol-1-yl)acetic acid;

2-(5-Chloro-2-methyl-3-((6-oxo-1-(4-(trifluoromethyl)benzyl)-1,6-dihydropyridazin-3-yl)-methyl)-1H-indol-1-yl)acetic acid;

2-(5-Fluoro-2-methyl-3-((6-oxo-1-(4-(trifluoromethyl)benzyl)-1,6-dihydropyridazin-3-yl)-methyl)-1H-indol-1-yl)acetic acid;

2-(3-((1-(2,4-Difluorobenzyl)-6-oxo-1,6-dihydropyridazin-3-yl)methyl)-5-fluoro-2-methyl-1H-indol-1-yl)acetic acid;

2-(3-((3-Benzyl-4-oxo-3,4-dihydrophthalazin-1-yl)methyl)-5-chloro-2-methyl-1H-indol-1-yl)acetic acid;

2-(3-((2-Benzyl-1-oxo-1,2-dihydroisoquinolin-4-yl)methyl)-5-fluoro-2-methyl-1H-indol-1-yl)acetic acid;

2-(3-((2-Benzyl-1-oxo-1,2-dihydroisoquinolin-4-yl)methyl)-2-methyl-1H-indol-1-yl)acetic acid;

2-(3-((2-Benzyl-1-oxo-1,2-dihydroisoquinolin-4-yl)methyl)-5-chloro-2-methyl-1H-indol-1-yl)acetic acid;

2-(3-(2-Benzyl-1-oxo-1,2,5,6,7,8-hexahydroisoquinolin-4-yl)-5-fluoro-2-methyl-1H-indol-1-yl)acetic acid;

2-(5-Fluoro-2-methyl-3-(1-oxo-2-(4,4,4-trifluorobutyl)-1,2,5,6,7,8-hexahydroisoquinolin-4-yl)-1H-indol-1-yl)acetic acid;

2-(5-Fluoro-2-methyl-3-(1-oxo-2-(2,2,2-trifluoroethyl)-1,2,5,6,7,8-hexahydroisoquinolin-4-yl)-1H-indol-1-yl)acetic acid;

2-(5-Fluoro-3-(2-isopropyl-1-oxo-1,2,5,6,7,8-hexahydroisoquinolin-4-yl)-2-methyl-1H-indol-1-yl)acetic acid;

2-(5-Fluoro-3-(2-(2-hydroxy-2-methylpropyl)-1-oxo-1,2,5,6,7,8-hexahydroisoquinolin-4-yl)-2-methyl-1H-indol-1-yl)acetic acid;

2-(5-Fluoro-2-methyl-3-(1-oxo-2-phenethyl-1,2,5,6,7,8-hexahydroisoquinolin-4-yl)-1H-indol-1-yl)acetic acid;

2-(3-(2-(2,4-Difluorobenzyl)-1-oxo-1,2,5,6,7,8-hexahydroisoquinolin-4-yl)-5-fluoro-2-methyl-1H-indol-1-yl)acetic acid;

2-(5-Fluoro-2-methyl-3-(1-oxo-2-(pyridin-2-ylmethyl)-1,2-dihydroisoquinolin-4-yl)-1H-indol-1-yl)acetic acid;

2-(5-Fluoro-2-methyl-3-(1-oxo-2-(4,4,4-trifluoro-3-(trifluoromethyl)butyl)-1,2-dihydro-isoquinolin-4-yl)-1H-indol-1-yl)acetic acid;

2-(5-Chloro-3-(3-(2,3-difluorobenzyl)-4-oxo-3,4-dihydrophthalazin-1-yl)-2-methyl-1H-indol-1-yl)acetic acid;

2-(5-Chloro-3-(3-(2-fluorobenzyl)-4-oxo-3,4-dihydrophthalazin-1-yl)-2-methyl-1H-indol-1-yl)acetic acid;

2-(5-Chloro-3-(3-((5-fluorobenzo[d]thiazol-2-yl)methyl)-4-oxo-3,4-dihydrophthalazin-1-yl)-2-methyl-1H-indol-1-yl)acetic acid;

2-(5-Chloro-2-methyl-3-(4-oxo-3-((5-(trifluoromethyl)benzo[d]thiazol-2-yl)methyl)-3,4-dihydrophthalazin-1-yl)-1H-indol-1-yl)acetic acid;

2-(5-Chloro-3-(3-(2,6-difluorobenzyl)-4-oxo-3,4-dihydrophthalazin-1-yl)-2-methyl-1H-indol-1-yl)acetic acid;

2-(5-Chloro-2-methyl-3-(4-oxo-3-(4-(trifluoromethoxy)benzyl)-3,4-dihydrophthalazin-1-yl)-1H-indol-1-yl)acetic acid;

2-(5-Chloro-2-methyl-3-(4-oxo-3-(quinolin-2-ylmethyl)-3,4-dihydrophthalazin-1-yl)-1H-indol-1-yl)acetic acid;

2-(5-Chloro-2-methyl-3-(3-((2-methylquinolin-4-yl)methyl)-4-oxo-3,4-dihydrophthalazin-1-yl)-1H-indol-1-yl)acetic acid;

2-(5-Chloro-2-methyl-3-(3-methyl-4-oxo-3,4-dihydrophthalazin-1-yl)-1H-indol-1-yl)acetic acid;

2-(5-Chloro-3-(3-ethyl-4-oxo-3,4-dihydrophthalazin-1-yl)-2-methyl-1H-indol-1-yl)acetic acid;

2-(5-Chloro-3-(3-isopropyl-4-oxo-3,4-dihydrophthalazin-1-yl)-2-methyl-1H-indol-1-yl)acetic acid;

2-(5-Chloro-3-(3-(cyclopropylmethyl)-4-oxo-3,4-dihydrophthalazin-1-yl)-2-methyl-1H-indol-1-yl)acetic acid;

2-(5-Chloro-2-methyl-3-(4-oxo-3-(2,2,2-trifluoroethyl)-3,4-dihydrophthalazin-1-yl)-1H-indol-1-yl)acetic acid;

2-(3-(3-(Benzo[d]thiazol-2-ylmethyl)-4-oxo-3,4-dihydrophthalazin-1-yl)-5-fluoro-2-methyl-1H-indol-1-yl)acetic acid;

2-(5-Fluoro-3-(3-(4-fluorobenzyl)-4-oxo-3,4-dihydrophthalazin-1-yl)-2-methyl-1H-indol-1-yl)acetic acid;

2-(3-(3-(2,3-Difluorobenzyl)-4-oxo-3,4-dihydrophthalazin-1-yl)-5-fluoro-2-methyl-1H-indol-1-yl)acetic acid;

2-(5-Fluoro-3-(3-(2-fluorobenzyl)-4-oxo-3,4-dihydrophthalazin-1-yl)-2-methyl-1H-indol-1-yl)acetic acid;

2-(5-Fluoro-3-(3-((5-fluorobenzo[d]thiazol-2-yl)methyl)-4-oxo-3,4-dihydrophthalazin-1-yl)-2-methyl-1H-indol-1-yl)acetic acid;

2-(5-Fluoro-2-methyl-3-(4-oxo-3-((5-(trifluoromethyl)benzo[d]thiazol-2-yl)methyl)-3,4-dihydrophthalazin-1-yl)-1H-indol-1-yl)acetic acid;

2-(3-(3-(2,6-Difluorobenzyl)-4-oxo-3,4-dihydrophthalazin-1-yl)-5-fluoro-2-methyl-1H-indol-1-yl)acetic acid;

2-(5-Fluoro-2-methyl-3-(4-oxo-3-(4-(trifluoromethoxy)benzyl)-3,4-dihydrophthalazin-1-yl)-1H-indol-1-yl)acetic acid;

2-(5-Fluoro-2-methyl-3-(4-oxo-3-(quinolin-2-ylmethyl)-3,4-dihydrophthalazin-1-yl)-1H-indol-1-yl)acetic acid;

2-(5-Fluoro-2-methyl-3-(3-((2-methylquinolin-4-yl)methyl)-4-oxo-3,4-dihydrophthalazin-1-yl)-1H-indol-1-yl)acetic acid;

2-(5-Fluoro-2-methyl-3-(3-methyl-4-oxo-3,4-dihydrophthalazin-1-yl)-1H-indol-1-yl)acetic acid;

2-(3-(3-Ethyl-4-oxo-3,4-dihydrophthalazin-1-yl)-5-fluoro-2-methyl-1H-indol-1-yl)acetic acid;

2-(3-(3-(Cyclopropylmethyl)-4-oxo-3,4-dihydrophthalazin-1-yl)-5-fluoro-2-methyl-1H-indol-1-yl)acetic acid;

2-(3-(3-Cyclopropyl-4-oxo-3,4-dihydrophthalazin-1-yl)-5-fluoro-2-methyl-1H-indol-1-yl)acetic acid;

2-(5-Fluoro-2-methyl-3-(4-oxo-3-(2,2,2-trifluoroethyl)-3,4-dihydrophthalazin-1-yl)-1H-indol-1-yl)acetic acid;

2-(5-Fluoro-2-methyl-3-(4-oxo-3-(4,4,4-trifluorobutyl)-3,4-dihydrophthalazin-1-yl)-1H-indol-1-yl)acetic acid;

2-(5-Fluoro-2-methyl-3-(3-neopentyl-4-oxo-3,4-dihydrophthalazin-1-yl)-1H-indol-1-yl)-acetic acid;

2-(5-Fluoro-2-methyl-3-(4-oxo-3-(3,3,3-trifluoropropyl)-3,4-dihydrophthalazin-1-yl)-1H-indol-1-yl)acetic acid;

2-(3-(3-(2-Ethyl-2-hydroxybutyl)-4-oxo-3,4-dihydrophthalazin-1-yl)-5-fluoro-2-methyl-1H-indol-1-yl)acetic acid;

2-(5-Fluoro-3-(3-(2-hydroxy-2-methylpropyl)-4-oxo-3,4-dihydrophthalazin-1-yl)-2-methyl-1H-indol-1-yl)acetic acid;

2-(5-Fluoro-2-methyl-3-(3-(3-methylbut-2-enyl)-4-oxo-3,4-dihydrophthalazin-1-yl)-1H-indol-1-yl)acetic acid;

2-(5-Fluoro-2-methyl-3-(4-oxo-3-(3,3,3-trifluoro-2-hydroxy-2-(trifluoromethyl)propyl)-3,4-dihydrophthalazin-1-yl)-1H-indol-1-yl)acetic acid;

2-(5-Fluoro-3-(3-(3-hydroxy-3-methylbutyl)-4-oxo-3,4-dihydrophthalazin-1-yl)-2-methyl-1H-indol-1-yl)acetic acid;

2-(5-Fluoro-2-methyl-3-(4-oxo-3-(2-oxobutyl)-3,4-dihydrophthalazin-1-yl)-1H-indol-1-yl)-acetic acid;

2-(5-Fluoro-2-methyl-3-(4-oxo-3-(pyridin-4-ylmethyl)-3,4-dihydrophthalazin-1-yl)-1H-indol-1-yl)acetic acid;

2-(5-Fluoro-2-methyl-3-(4-oxo-3-(pyridin-3-ylmethyl)-3,4-dihydrophthalazin-1-yl)-1H-indol-1-yl)acetic acid;

2-(5-Fluoro-2-methyl-3-(4-oxo-3-(pyridin-2-ylmethyl)-3,4-dihydrophthalazin-1-yl)-1H-indol-1-yl)acetic acid;

2-(5-Fluoro-2-methyl-3-(4-oxo-3-(4,4,4-trifluoro-3-(trifluoromethyl)butyl)-3,4-dihydrophthalazin-1-yl)-1H-indol-1-yl)acetic acid;

2-(5-Fluoro-3-(3-((3-fluoropyridin-4-yl)methyl)-4-oxo-3,4-dihydrophthalazin-1-yl)-2-methyl-1H-indol-1-yl)acetic acid;

2-(5-Chloro-3-(1-(4-fluorobenzyl)-6-oxo-1,6-dihydropyridazin-3-yl)-2-methyl-1H-indol-1-yl)acetic acid;

2-(5-Chloro-3-(1-(2-(4-chlorophenoxy)ethyl)-6-oxo-1,6-dihydropyridazin-3-yl)-2-methyl-1H-indol-1-yl)acetic acid;

2-(3-(1-(Benzo[d]thiazol-2-ylmethyl)-6-oxo-1,6-dihydropyridazin-3-yl)-5-chloro-2-methyl-1H-indol-1-yl)acetic acid;

2-(5-Chloro-3-(1-(4-fluoro-2-(trifluoromethyl)benzyl)-6-oxo-1,6-dihydropyridazin-3-yl)-2-methyl-1H-indol-1-yl)acetic acid; p 2-(5-Chloro-3-(1-(3-fluoro-2-(trifluoromethyl)benzyl)-6-oxo-1,6-dihydropyridazin-3-yl)-2-methyl-1H-indol-1-yl)acetic acid; p 2-(5-Chloro-2-methyl-3-(6-oxo-1-(quinolin-2-ylmethyl)-1,6-dihydropyridazin-3-yl)-1H-indol-1-yl)acetic acid;

2-(5-chloro-2-methyl-3-(1-((2-methylquinolin-4-yl)methyl)-6-oxo-1,6-dihydropyridazin-3-yl)-1H-indol-1-yl)acetic acid;

2-(5-Chloro-2-methyl-3-(1-(4-methylbenzyl)-6-oxo-1,6-dihydropyridazin-3-yl)-1H-indol-1-yl)acetic acid;

2-(5-Chloro-3-(1-(4-isopropylbenzyl)-6-oxo-1,6-dihydropyridazin-3-yl)-2-methyl-1H-indol-1-yl)acetic acid;

2-(5-chloro-3-(1-(4-methoxybenzyl)-6-oxo-1,6-dihydropyridazin-3-yl)-2-methyl-1H-indol-1-yl)acetic acid;

2-(5-Chloro-2-methyl-3-(1-methyl-6-oxo-1,6-dihydropyridazin-3-yl)-1H-indol-1-yl)acetic acid;

2-(5-Chloro-3-(1-ethyl-6-oxo-1,6-dihydropyridazin-3-yl)-2-methyl-1H-indol-1-yl)acetic acid;

2-(5-Fluoro-3-(1-(4-fluorobenzyl)-6-oxo-1,6-dihydropyridazin-3-yl)-2-methyl-1H-indol-1-yl)acetic acid;

2-(3-(1-(2-(4-Chlorophenoxy)ethyl)-6-oxo-1,6-dihydropyridazin-3-yl)-5-fluoro-2-methyl-1H-indol-1-yl)acetic acid;

2-(3-(1-(Benzo[d]thiazol-2-ylmethyl)-6-oxo-1,6-dihydropyridazin-3-yl)-5-fluoro-2-methyl-1H-indol-1-yl)acetic acid;

2-(5-Fluoro-3-(1-(3-fluorobenzyl)-6-oxo-1,6-dihydropyridazin-3-yl)-2-methyl-1H-indol-1-yl)acetic acid;

2-(5-Fluoro-3-(1-(4-fluoro-2-(trifluoromethyl)benzyl)-6-oxo-1,6-dihydropyridazin-3-yl)-2-methyl-1H-indol-1-yl)acetic acid;

2-(5-Fluoro-3-(1-(3-fluoro-2-(trifluoromethyl)benzyl)-6-oxo-1,6-dihydropyridazin-3-yl)-2-methyl-1H-indol-1-yl)acetic acid;

2-(5-fluoro-2-methyl-3-(6-oxo-1-(quinolin-2-ylmethyl)-1,6-dihydropyridazin-3-yl)-1H-indol-1-yl)acetic acid;

2-(5-Fluoro-2-methyl-3-(1-((2-methylquinolin-4-yl)methyl)-6-oxo-1,6-dihydropyridazin-3-yl)-1H-indol-1-yl)acetic acid;

2-(5-Fluoro-2-methyl-3-(1-methyl-6-oxo-1,6-dihydropyridazin-3-yl)-1H-indol-1-yl)acetic acid;

2-(3-(1-Ethyl-6-oxo-1,6-dihydropyridazin-3-yl)-5-fluoro-2-methyl-1H-indol-1-yl)acetic acid;

2-(3-(1-(Cyclopropylmethyl)-6-oxo-1,6-dihydropyridazin-3-yl)-5-fluoro-2-methyl-1H-indol-1-yl)acetic acid;

2-(5-Fluoro-2-methyl-3-(6-oxo-1-(2,2,2-trifluoroethyl)-1,6-dihydropyridazin-3-yl)-1H-indol-1-yl)acetic acid;

2-(5-Fluoro-2-methyl-3-(6-oxo-1-(4,4,4-trifluorobutyl)-1,6-dihydropyridazin-3-yl)-1H-indol-1-yl)acetic acid;

2-(5-Fluoro-2-methyl-3-(6-oxo-1-(3,3,3-trifluoropropyl)-1,6-dihydropyridazin-3-yl)-1H-indol-1-yl)acetic acid;

2-(5-Fluoro-2-methyl-3-(1-neopentyl-6-oxo-1,6-dihydropyridazin-3-yl)-1H-indol-1-yl)acetic acid;

2-(3-(3-(4-Fluorobenzyl)-4-oxo-3,4-dihydrophthalazin-1-yl)-2-methyl-1H-indol-1-yl)acetic acid;

2-(3-(3-(Benzo[d]thiazol-2-ylmethyl)-4-oxo-3,4-dihydrophthalazin-1-yl)-2-methyl-1H-indol-1-yl)acetic acid;

2-(2-Methyl-3-(3-(4-(methylsulfonyl)benzyl)-4-oxo-3,4-dihydrophthalazin-1-yl)-1H-indol-1-yl)acetic acid;

2-(2-Methyl-3-(4-oxo-3-(quinolin-2-ylmethyl)-3,4-dihydrophthalazin-1-yl)-1H-indol-1-yl)-acetic acid;

2-(2-methyl-3-(4-oxo-3-(4-(trifluoromethoxy)benzyl)-3,4-dihydrophthalazin-1-yl)-1H-indol-1-yl)acetic acid;

2-(2-Methyl-3-(4-oxo-3-(4-(trifluoromethyl)benzyl)-3,4-dihydrophthalazin-1-yl)-1H-indol-1-yl)acetic acid;

2-(3-(3-(2,6-Difluorobenzyl)-4-oxo-3,4-dihydrophthalazin-1-yl)-2-methyl-1H-indol-1-yl)acetic acid;

2-(2-Methyl-3-(3-(4-methylbenzyl)-4-oxo-3,4-dihydrophthalazin-1-yl)-1H-indol-1-yl)acetic acid;

2-(2-Methyl-3-(3-methyl-4-oxo-3,4-dihydrophthalazin-1-yl)-1H-indol-1-yl)acetic acid;

2-(3-(3-Ethyl-4-oxo-3,4-dihydrophthalazin-1-yl)-2-methyl-1H-indol-1-yl)acetic acid;

2-(3-(3-(Cyclopropylmethyl)-4-oxo-3,4-dihydrophthalazin-1-yl)-2-methyl-1H-indol-1-yl)acetic acid;

2-(2-Methyl-3-(4-oxo-3-(2,2,2-trifluoroethyl)-3,4-dihydrophthalazin-1-yl)-1H-indol-1-yl)-acetic acid;

2-(3-(3-cyclopropyl-4-oxo-3,4-dihydrophthalazin-1-yl)-2-methyl-1H-indol-1-yl)acetic acid;

2-(3-(3-Cyclopentyl-4-oxo-3,4-dihydrophthalazin-1-yl)-2-methyl-1H-indol-1-yl)acetic acid;

2-(3-(1-Benzyl-6-oxo-1,6-dihydropyridazin-3-yl)-2-methyl-1H-indol-1-yl)acetic acid;

2-(3-(1-Isopropyl-6-oxo-1,6-dihydropyridazin-3-yl)-2-methyl-1H-indol-1-yl)acetic acid;

2-(3-(1-Ethyl-6-oxo-1,6-dihydropyridazin-3-yl)-2-methyl-1H-indol-1-yl)acetic acid;

2-(2-Methyl-3-(1-methyl-6-oxo-1,6-dihydropyridazin-3-yl)-1H-indol-1-yl)acetic acid;

2-(3-(1-(Cyclopropylmethyl)-6-oxo-1,6-dihydropyridazin-3-yl)-2-methyl-1H-indol-1-yl)-acetic acid;

2-(2-Methyl-3-(6-oxo-1-(2,2,2-trifluoroethyl)-1,6-dihydropyridazin-3-yl)-1H-indol-1-yl)-acetic acid;

Methyl 2-(3-(1-(2,4-difluorobenzyl)-6-oxo-1,6-dihydropyridazin-3-yl)-2-methyl-1H-indol-1-yl)acetate;

2-(2-Methyl-3-(6-oxo-1-(2,4,5-trifluorobenzyl)-1,6-dihydropyridazin-3-yl)-1H-indol-1-yl)-acetic acid;

2-(3-(1-(2,3-Difluorobenzyl)-6-oxo-1,6-dihydropyridazin-3-yl)-2-methyl-1H-indol-1-yl)-acetic acid;

2-(3-(1-(4-Fluorobenzyl)-6-oxo-1,6-dihydropyridazin-3-yl)-2-methyl-1H-indol-1-yl)acetic acid;

2-(3-(1-(2-Fluorobenzyl)-6-oxo-1,6-dihydropyridazin-3-yl)-2-methyl-1H-indol-1-yl)acetic acid;

2-(3-(1-Benzyl-6-oxo-1,6-dihydropyridazin-3-yl)-2,5-dimethyl-1H-indol-1-yl)acetic acid;

2-(3-(3-Benzyl-4-oxo-3,4-dihydrophthalazin-1-yl)-2,5-dimethyl-1H-indol-1-yl)acetic acid;

2-(5-Fluoro-2-methyl-3-((6-oxo-1-(2,4,5-trifluorobenzyl)-1,6-dihydropyridazin-3-yl)-methyl)-1H-indol-1-yl)acetic acid;

2-(5-Fluoro-2-methyl-3-((6-oxo-1-(pyridin-4-ylmethyl)-1,6-dihydropyridazin-3-yl)methyl)-1H-indol-1-yl)acetic acid;

2-(5-Fluoro-2-methyl-3-((6-oxo-1-(pyridin-3-ylmethyl)-1,6-dihydropyridazin-3-yl)methyl)-1H-indol-1-yl)acetic acid;

2-(5-Fluoro-2-methyl-3-((6-oxo-1-(pyridin-2-ylmethyl)-1,6-dihydropyridazin-3-yl)methyl)-1H-indol-1-yl)acetic acid;

(5-Fluoro-3-{[1-(2-hydroxy-2-methylpropyl)-6-oxo-1,6-dihydropyridazin-3-yl]methyl}-2-methyl-1H-indol-1-yl)acetic acid;

2-(5-Chloro-2-methyl-3-(4-oxo-3-(4-(trifluoromethyl)benzyl)-3,4-dihydrophthalazin-1-yl)-1H-indol-1-yl)acetic acid;

2-(5-Chloro-2-methyl-3-(4-oxo-3-(2-(trifluoromethyl)benzyl)-3,4-dihydrophthalazin-1-yl)-1H-indol-1-yl)acetic acid;

2-(5-Chloro-2-methyl-3-(4-oxo-3-(3-(trifluoromethyl)benzyl)-3,4-dihydrophthalazin-1-yl)-1H-indol-1-yl)acetic acid;

[5-fluoro-2-methyl-3-({6-oxo-1-[3,3,3-trifluoro-2-hydroxy-2-(trifluoromethyl)propyl]-1,6-dihydro-pyridazin-3-yl}methyl)-1H-indol-1-yl]acetic acid;

2-(5-Fluoro-2-methyl-3-(4-oxo-3-(2-(trifluoromethyl)benzyl)-3,4-dihydrophthalazin-1-yl)-1H-indol-1-yl)acetic acid;

2-(5-Fluoro-2-methyl-3-(4-oxo-3-(3-(trifluoromethyl)benzyl)-3,4-dihydrophthalazin-1-yl)-1H-indol-1-yl)acetic acid;

2-(5-Fluoro-2-methyl-3-(4-oxo-3-(4-(trifluoromethyl)benzyl)-3,4-dihydrophthalazin-1-yl)-1H-indol-1-yl)acetic acid;

2-(5-Fluoro-2-methyl-3-(4-oxo-3-phenyl-3,4-dihydrophthalazin-1-yl)-1H-indol-1-yl)acetic acid;

2-(5-Fluoro-2-methyl-3-(4-oxo-3-(4-(trifluoromethyl)phenyl)-3,4-dihydrophthalazin-1-yl)-1H-indol-1-yl)acetic acid;

2-(5-Fluoro-2-methyl-3-(4-oxo-3-(3-(trifluoromethyl)phenyl)-3,4-dihydrophthalazin-1-yl)-1H-indol-1-yl)acetic acid;

2-(3-(3-((5-Chlorobenzo[d]thiazol-2-yl)methyl)-4-oxo-3,4-dihydrophthalazin-1-yl)-5-fluoro-2-methyl-1H-indol-1-yl)acetic acid;

2-(5-Chloro-3-(3-((5-chlorobenzo[d]thiazol-2-yl)methyl)-4-oxo-3,4-dihydrophthalazin-1-yl)-2-methyl-1H-indol-1-yl)acetic acid;

2-(3-(3-(2-(4-Chlorophenoxy)ethyl)-4-oxo-3,4-dihydrophthalazin-1-yl)-5-fluoro-2-methyl-1H-indol-1-yl)acetic acid;

2-(5-Chloro-2-methyl-3-(6-oxo-1-(2-(trifluoromethyl)benzyl)-1,6-dihydropyridazin-3-yl)-1H-indol-1-yl)acetic acid;

2-(5-Chloro-2-methyl-3-(6-oxo-1-(3-(trifluoromethyl)benzyl)-1,6-dihydropyridazin-3-yl)-1H-indol-1-yl)acetic acid;

2-(5-Chloro-2-methyl-3-(6-oxo-1-(4-(trifluoromethyl)benzyl)-1,6-dihydropyridazin-3-yl)-1H-indol-1-yl)acetic acid;

2-(3-(1-Benzyl-6-oxo-1,6-dihydropyridazin-3-yl)-2-methyl-5-(methylsulfonyl)-1H-indol-1-yl)acetic acid;

2-(3-(3-Benzyl-4-oxo-3,4-dihydrophthalazin-1-yl)-2-methyl-5-(methylsulfonyl)-1H-indol-1-yl)acetic acid;

2-(5-Fluoro-2-methyl-3-(6-oxo-1-(2-(trifluoromethyl)benzyl)-1,6-dihydropyridazin-3-yl)-1H-indol-1-yl)acetic acid;

2-(5-Fluoro-2-methyl-3-(6-oxo-1-(3-(trifluoromethyl)benzyl)-1,6-dihydropyridazin-3-yl)-1H-indol-1-yl)acetic acid;

2-(5-Fluoro-2-methyl-3-(6-oxo-1-(3-(trifluoromethyl)benzyl)-1,6-dihydropyridazin-3-yl)-1H-indol-1-yl)acetic acid;

2-(3-(1-Benzyl-6-oxo-1,6-dihydropyridazin-3-yl)-5-bromo-2-methyl-1H-indol-1-yl)acetic acid; 2-(5-Fluoro-2-methyl-3-(3-(2-methyl-2-phenoxypropyl)-4-oxo-3,4-dihydrophthalazin-1-yl)-1H-indol-1-yl)acetic acid;

2-(5-Chloro-2-methyl-3-(4-oxo-3-(2-phenoxyethyl)-3,4-dihydrophthalazin-1-yl)-1H-indol-1-yl)acetic acid;

2-(5-Fluoro-2-methyl-3-(4-oxo-3-(2-phenoxyethyl)-3,4-dihydrophthalazin-1-yl)-1H-indol-1-yl)acetic acid;

2-(2-Methyl-3-(4-oxo-3-(2-phenoxyethyl)-3,4-dihydrophthalazin-1-yl)-1H-indol-1-yl)acetic acid;

2-(3-(3-(4-Fluorophenethyl)-4-oxo-3,4-dihydrophthalazin-1-yl)-2-methyl-1H-indol-1-yl)-acetic acid;

2-(2-Methyl-3-(4-oxo-3-phenethyl-3,4-dihydrophthalazin-1-yl)-1H-indol-1-yl)acetic acid;

2-(5-Fluoro-2-methyl-3-(4-oxo-3-phenethyl-3,4-dihydrophthalazin-1-yl)-1H-indol-1-yl)-acetic acid;

2-(5-Fluoro-3-(3-(4-fluorophenethyl)-4-oxo-3,4-dihydrophthalazin-1-yl)-2-methyl-1H-indol-1-yl)acetic acid;

2-(2-Methyl-3-(6-oxo-1-phenethyl-1,6-dihydropyridazin-3-yl)-1H-indol-1-yl)acetic acid;

2-(3-(1-(4-Fluorophenethyl)-6-oxo-1,6-dihydropyridazin-3-yl)-2-methyl-1H-indol-1-yl)-acetic acid;

2-(5-Fluoro-2-methyl-3-(1-(2-methyl-2-phenoxypropyl)-6-oxo-1,6-dihydropyridazin-3-yl)-1H-indol-1-yl)acetic acid;

2-(3-(1-Benzyl-6-oxo-1,6-dihydropyridazin-3-yl)-7-chloro-5-fluoro-2-methyl-1H-indol-1-yl)acetic acid;

2-(7-Chloro-5-fluoro-3-(1-isopropyl-6-oxo-1,6-dihydropyridazin-3-yl)-2-methyl-1H-indol-1-yl)acetic acid;

2-(3-(1-Benzyl-6-oxo-1,6-dihydropyridazin-3-yl)-5,7-difluoro-2-methyl-1H-indol-1-yl)acetic acid;

2-(3-(1-Benzyl-6-oxo-1,6-dihydropyridazin-3-yl)-5,7-dichloro-2-methyl-1H-indol-1-yl)-acetic acid;

2-(5,7-Dichloro-3-(1-isopropyl-6-oxo-1,6-dihydropyridazin-3-yl)-2-methyl-1H-indol-1-yl)-acetic acid;

2-(3-(1-Benzyl-6-oxo-1,6-dihydropyridazin-3-yl)-5-fluoro-2-methyl-7-(methylsulfonyl)-1H-indol-1-yl)acetic acid;

2-(3-(3-Benzyl-4-oxo-3,4-dihydrophthalazin-1-yl)-5-fluoro-2-methyl-7-(methylsulfonyl)-1H-indol-1-yl)acetic acid;

2-(5-Fluoro-3-((1-(4-(2-hydroxypropan-2-yl)benzyl)-6-oxo-1,6-dihydropyridazin-3-yl)-methyl)-2-methyl-1H-indol-1-yl)acetic acid;

2-(5-Fluoro-3-((1-(4-(1,1,1,3,3,3-hexafluoro-2-hydroxypropan-2-yl)benzyl)-6-oxo-1,6-dihydropyridazin-3-yl)methyl)-2-methyl-1H-indol-1-yl)acetic acid;

2-(5-Fluoro-3-((1-(3-(2-hydroxypropan-2-yl)benzyl)-6-oxo-1,6-dihydropyridazin-3-yl)-methyl)-2-methyl-1H-indol-1-yl)acetic acid;

2-(5-Fluoro-3-((1-((3-fluoropyridin-4-yl)methyl)-6-oxo-1,6-dihydropyridazin-3-yl)methyl)-2-methyl-1H-indol-1-yl)acetic acid;

2-(3-((1-(2,4-Difluorobenzyl)-6-oxo-1,6-dihydropyridazin-3-yl)methyl)-5,7-difluoro-2-methyl-1H-indol-1-yl)acetic acid;

2-(3-(3-Benzyl-4-oxo-3,4-dihydrophthalazin-1-yl)-7-chloro-5-fluoro-2-methyl-1H-indol-1-yl)acetic acid;

2-(3-(3-Benzyl-4-oxo-3,4-dihydrophthalazin-1-yl)-5,7-difluoro-2-methyl-1H-indol-1-yl)-acetic acid;

2-(3-(3-Benzyl-4-oxo-3,4-dihydrophthalazin-1-yl)-5,7-dichloro-2-methyl-1H-indol-1-yl)-acetic acid;

2-(3-(1-Benzyl-6-oxo-1,6-dihydropyridazin-3-yl)-7-bromo-2-methyl-1H-indol-1-yl)acetic acid;

2-(3-(2-Benzyl-1-oxo-1,2-dihydroisoquinolin-4-yl)-2-methyl-1H-indol-1-yl)acetic acid;

2-(3-(2-Benzyl-1-oxo-1,2-dihydroisoquinolin-4-yl)-5-fluoro-2-methyl-1H-indol-1-yl)acetic acid;

2-(5-Fluoro-3-(2-isopropyl-1-oxo-1,2-dihydroisoquinolin-4-yl)-2-methyl-1H-indol-1-yl)-acetic acid;

2-(5-Fluoro-2-methyl-3-(1-oxo-2-(2,2,2-trifluoroethyl)-1,2-dihydroisoquinolin-4-yl)-1H-indol-1-yl)acetic acid;

2-(2-Methyl-3-(1-oxo-2-(2,2,2-trifluoroethyl)-1,2-dihydroisoquinolin-4-yl)-1H-indol-1-yl)-acetic acid;

2-(3-(2-Isopropyl-1-oxo-1,2-dihydroisoquinolin-4-yl)-2-methyl-1H-indol-1-yl)acetic acid;

2-(3-(2-(2,2-Difluoro-2-methoxyethyl)-1-oxo-1,2-dihydroisoquinolin-4-yl)-2-methyl-1H-indol-1-yl)acetic acid;

2-(5-Fluoro-3-(2-(2-hydroxy-2-methylpropyl)-1-oxo-1,2-dihydroisoquinolin-4-yl)-2-methyl-1H-indol-1-yl)acetic acid;

2-(5-Fluoro-2-methyl-3-(1-oxo-2-(3,3,3-trifluoro-2-hydroxy-2-(trifluoromethyl)propyl)-1,2-dihydroisoquinolin-4-yl)-1H-indol-1-yl)acetic acid;

(5-Fluoro-2-methyl-3-(1-oxo-2-(4,4,4-trifluorobutyl)-1,2-dihydroisoquinolin-4-yl)-1H-indol-1-yl)acetic acid;

2-(5-Fluoro-2-methyl-3-(2-neopentyl-1-oxo-1,2-dihydroisoquinolin-4-yl)-1H-indol-1-yl)acetic acid;

2-(3-(3-((4H-1,2,4-Triazol-3-yl)methyl)-4-oxo-3,4-dihydrophthalazin-1-yl)-5-fluoro-2-methyl-1H-indol-1-yl)acetic acid;

2-(3-(3-(2-Amino-2-oxoethyl)-4-oxo-3,4-dihydrophthalazin-1-yl)-5-chloro-2-methyl-1H-indol-1-yl)acetic acid;

2-(5-Chloro-2-methyl-3-(3-((5-methyl-4H-1,2,4-triazol-3-yl)methyl)-4-oxo-3,4-dihydrophthalazin-1-yl)-1H-indol-1-yl)acetic acid;

2-(3-(3-((4H-1,2,4-Triazol-3-yl)methyl)-4-oxo-3,4-dihydrophthalazin-1-yl)-5-chloro-2-methyl-1H-indol-1-yl)acetic acid;

2-(3-(1-((4H-1,2,4-Triazol-3-yl)methyl)-6-oxo-1,6-dihydropyridazin-3-yl)-5-fluoro-2-methyl-1H-indol-1-yl)acetic acid;

2-(5-Fluoro-2-methyl-3-(1-((5-methyl-4H-1,2,4-triazol-3-yl)methyl)-6-oxo-1,6-dihydropyridazin-3-yl)-1H-indol-1-yl)acetic acid;

2-(5-Fluoro-2-methyl-3-(4-oxo-3((1-phenyl-1H-1,2,4-triazol-5-yl)methyl)-3,4-dihydrophthalazin-1-yl)-1H-indol-1-yl)acetic acid;

2-Benzyl-6-(5-fluoro-1-(4-methoxybenzoyl)-2-methyl-1H-indol-3-yl)pyridazin-3(2H)-one;

2-Benzyl-6-(5-fluoro-2-methyl-1-nicotinoyl-1H-indol-3-yl)pyridazin-3(2H)-one;

6-(1-Benzyl-5-fluoro-2-methyl-1H-indol-3-yl)-2-benzylpyridazin-3(2H)-one;

2-(3-(1-Benzyl-6-oxo-1,6-dihydropyridin-3-yl)-2-methyl-1H-indol-1-yl)acetic acid;

2-(3-(1-Benzyl-6-oxo-1,6-dihydropyridin-3-yl)-5-chloro-2-methyl-1H-indol-1-yl)acetic acid;

2-(3-(1-Benzyl-6-oxo-1,6-dihydropyridin-3-yl)-5-fluoro-2-methyl-1H-indol-1-yl)acetic acid;

2-(5-Fluoro-3-(1-(2-fluorobenzyl)-6-oxo-1,6-dihydropyridin-3-yl)-2-methyl-1H-indol-1-yl)-acetic acid;

2-(5-Fluoro-3-(1-(4-fluorobenzyl)-6-oxo-1,6-dihydropyridin-3-yl)-2-methyl-1H-indol-1-yl)-acetic acid;

2-(3-(1-(2,6-Difluorobenzyl)-6-oxo-1,6-dihydropyridin-3-yl)-5-fluoro-2-methyl-1H-indol-1-yl)acetic acid;

2-(3-(142,3-Difluorobenzyl)-6-oxo-1,6-dihydropyridin-3-yl)-5-fluoro-2-methyl-1H-indol-1-yl)acetic acid;

2-(3-(1-(2,4-Difluorobenzyl)-6-oxo-1,6-dihydropyridin-3-yl)-5-fluoro-2-methyl-1H-indol-1-yl)acetic acid;

2-(5-Fluoro-2-methyl-3-(6-oxo-1-(2,4,5-trifluorobenzyl)-1,6-dihydropyridin-3-yl)-1H-indol-1-yl)acetic acid;

2-(5-Fluoro-3-(2-(3-hydroxy-3-methylbutyl)-1-oxo-1,2-dihydroisoquinolin-4-yl)-2-methyl-1H-indol-1-yl)acetic acid;

2-(5-Chloro-3-(1-(2,4-difluorobenzyl)-6-oxo-1,6-dihydropyridin-3-yl)-2-methyl-1H-indol-1-yl)acetic acid;

2-(3-(1-(2,4-Difluorobenzyl)-6-oxo-1,6-dihydropyridin-3-yl)-2-methyl-1H-indol-1-yl)acetic acid;

2-(3-(1-Isopropyl-6-oxo-1,6-dihydropyridin-3-yl)-2-methyl-1H-indol-1-yl)acetic acid;

2-(5-Fluoro-3-(1-isopropyl-6-oxo-1,6-dihydropyridin-3-yl)-2-methyl-1H-indol-1-yl)acetic acid;

2-(5-Chloro-3-(1-isopropyl-6-oxo-1,6-dihydropyridin-3-yl)-2-methyl-1H-indol-1-yl)acetic acid;

2-(2-Methyl-3-(6-oxo-1-(2,2,2-trifluoroethyl)-1,6-dihydropyridin-3-yl)-1H-indol-1-yl)acetic acid;

2-(5-Fluoro-2-methyl-3-(6-oxo-1-(2,2,2-trifluoroethyl)-1,6-dihydropyridin-3-yl)-1H-indol-1-yl)acetic acid;

2-(5-Chloro-2-methyl-3-(6-oxo-1-(2,2,2-trifluoroethyl)-1,6-dihydropyridin-3-yl)-1H-indol-1-yl)acetic acid;

2-(3-(1-(2-Fluorobenzyl)-6-oxo-1,6-dihydropyridin-3-yl)-2-methyl-1H-indol-1-yl)acetic acid;

2-(3-(1-(4-Fluorobenzyl)-6-oxo-1,6-dihydropyridin-3-yl)-2-methyl-1H-indol-1-yl)acetic acid;

2-(3-(1-(2,3-Difluorobenzyl)-6-oxo-1,6-dihydropyridin-3-yl)-2-methyl-1H-indol-1-yl)acetic acid;

2-(3-(1-(2,6-Difluorobenzyl)-6-oxo-1,6-dihydropyridin-3-yl)-2-methyl-1H-indol-1-yl)acetic acid;

2-(2-Methyl-3-(6-oxo-1-(2,4,5-trifluorobenzyl)-1,6-dihydropyridin-3-yl)-1H-indol-1-yl)-acetic acid;

2-(3-(1-Isobutyl-6-oxo-1,6-dihydropyridin-3-yl)-2-methyl-1H-indol-1-yl)acetic acid;

2-(3-(1-Cyclopentyl-6-oxo-1,6-dihydropyridin-3-yl)-2-methyl-1H-indol-1-yl)acetic acid;

2-(5-Chloro-3-(1-(4-fluorobenzyl)-6-oxo-1,6-dihydropyridin-3-yl)-2-methyl-1H-indol-1-yl)-acetic acid;

2-(5-Chloro-3-(1-(2-fluorobenzyl)-6-oxo-1,6-dihydropyridin-3-yl)-2-methyl-1H-indol-1-yl)-acetic acid;

2-(5-Chloro-3-(1-(2,6-difluorobenzyl)-6-oxo-1,6-dihydropyridin-3-yl)-2-methyl-1H-indol-1-yl)acetic acid;

2-(5-Chloro-3-(1-(2,3-difluorobenzyl)-6-oxo-1,6-dihydropyridin-3-yl)-2-methyl-1H-indol-1-yl)acetic acid;

2-(5-Chloro-2-methyl-3-(6-oxo-1-(2,4,5-trifluorobenzyl)-1,6-dihydropyridin-3-yl)-1H-indol-1-yl)acetic acid;

2-(5-Chloro-3-(1-(3,5-difluorobenzyl)-6-oxo-1,6-dihydropyridin-3-yl)-2-methyl-1H-indol-1-yl)acetic acid;

2-(5-Chloro-3-(1-(3-fluorobenzyl)-6-oxo-1,6-dihydropyridin-3-yl)-2-methyl-1H-indol-1-yl)-acetic acid;

2-(2-Methyl-3-(6-oxo-1-(4,4,4-trifluorobutyl)-1,6-dihydropyridin-3-yl)-1H-indol-1-yl)acetic acid;

2-(5-Fluoro-2-methyl-3-(6-oxo-1-(4,4,4-trifluorobutyl)-1,6-dihydropyridin-3-yl)-1H-indol-1-yl)acetic acid;

2-(5-Methoxy-2-methyl-3-(6-oxo-1-(2,2,2-trifluoroethyl)-1,6-dihydropyridin-3-yl)-1H-indol-1-yl)acetic acid;

1-(2,3-Difluorobenzyl)-5-(5-fluoro-2-methyl-1-(2-oxo-2-(pyrrolidin-1-yl)ethyl)-1H-indol-3-yl)pyridin-2(1H)-one;

2-(3-(1-(2,3-Difluorobenzyl)-6-oxo-1,6-dihydropyridin-3-yl)-5-fluoro-2-methyl-1H-indol-1-yl)-N,N-dimethylacetamide;

2-(3-(1-(2,3-Difluorobenzyl)-6-oxo-1,6-dihydropyridin-3-yl)-5-fluoro-2-methyl-1H-indol-1-yl)acetamide;

2-(3-(1-(2,3-Difluorobenzyl)-6-oxo-1,6-dihydropyridin-3-yl)-5-fluoro-2-methyl-1H-indol-1-yl)-N-(methylsulfonyl)acetamide;

3-(3-(1-(2,3-Difluorobenzyl)-6-oxo-1,6-dihydropyridin-3-yl)-5-fluoro-2-methyl-1H-indol-1-yl)propanoic acid;

2-(2,5-Dimethyl-3-(6-oxo-1-(2,2,2-trifluoroethyl)-1,6-dihydropyridin-3-yl)-1H-indol-1-yl)-acetic acid;

1-(2,3-Difluorobenzyl)-5-(5-fluoro-1-(2-hydroxyethyl)-2-methyl-1H-indol-3-yl)pyridin-2(1H)-one;

(S)-2-(3-(1-(2,3-Difluorobenzyl)-6-oxo-1,6-dihydropyridin-3-yl)-5-fluoro-2-methyl-1H-indol-1-yl)propanoic acid;

(R)-2-(3-(1-(2,3-Difluorobenzyl)-6-oxo-1,6-dihydropyridin-3-yl)-5-fluoro-2-methyl-1H-indol-1-yl)propanoic acid;

2-(5-Fluoro-2-methyl-3-(6-oxo-1-(3,3,3-trifluoropropyl)-1,6-dihydropyridin-3-yl)-1H-indol-1-yl)acetic acid;

2-(5-Fluoro-2-methyl-3-(6-oxo-1-(pyridin-4-ylmethyl)-1,6-dihydropyridin-3-yl)-1H-indol-1-yl)acetic acid;

2-(5-Fluoro-2-methyl-3-(6-oxo-1-(pyridin-2-ylmethyl)-1,6-dihydropyridin-3-yl)-1H-indol-1-yl)acetic acid;

2-(3-(1-(2,3-Difluorobenzyl)-6-oxo-1,6-dihydropyridin-3-yl)-5-fluoro-2-methyl-1H-indol-1-yl)acetonitrile;

5-(1-((2H-Tetrazol-5-yl)methyl)-5-fluoro-2-methyl-1H-indol-3-yl)-1-(2,3-difluorobenzyl)-pyridin-2(1H)-one;

2-(3-(1-Benzyl-6-oxo-1,6-dihydropyridin-3-yl)-5-fluoro-2-methyl-1H-indol-1-yl)-N-(phenylsulfonyl)acetamide;

2-(3-(1-Benzyl-6-oxo-1,6-dihydropyridin-3-yl)-5-fluoro-2-methyl-1H-indol-1-yl)-N-(methyl-sulfonyl)acetamide;

2-(3-(1-Benzyl-6-oxo-1,6-dihydropyridin-3-yl)-5-fluoro-2-methyl-1H-indol-1-yl)-N-(o-tolyl-sulfonyl)acetamide;

2-(5-Fluoro-2-methyl-3-(6-oxo-1-(pyridin-3-ylmethyl)-1,6-dihydropyridin-3-yl)-1H-indol-1-yl)acetic acid;

2-(5-Fluoro-2-methyl-3-(6-oxo-1-(4,4,4-trifluoro-3-(trifluoromethyl)butyl)-1,6-dihydro-pyridin-3-yl)-1H-indol-1-yl)acetic acid;

N-(Cyclopropylsulfonyl)-2-(5-fluoro-2-methyl-3-(6-oxo-1-(4,4,4-trifluorobutyl)-1,6-dihydropyridin-3-yl)-1H-indol-1-yl)acetamide;

2-(5-Fluoro-2-methyl-3-(6-oxo-1-(4,4,4-trifluorobutyl)-1,6-dihydropyridin-3-yl)-1H-indol-1-yl)-N-(methylsulfonyl)acetamide;

2-(3-(1-Benzyl-6-oxo-1,6-dihydropyridin-3-yl)-5-fluoro-2-methyl-1H-indol-1-yl)-N-(cyclo-propyl-sulfonyl)acetamide;

2-(3-((1-(2,4-Difluorobenzyl)-6-oxo-1,6-dihydropyridazin-3-yl)methyl)-5-fluoro-2-methyl-1H-indol-1-yl)-N-(methylsulfonyl)acetamide;

2-(3-((1-(3,4-Difluorobenzyl)-6-oxo-1,6-dihydropyridin-3-yl)methyl)-5-fluoro-2-methyl-1H-indol-1-yl)-N-(methylsulfonyl)acetamide;

2-(5-Chloro-2-methyl-3-(4-oxo-3-(2-phenoxyethyl)-3,4-dihydrophthalazin-1-yl)-1H-indol-1-yl)-N-(methylsulfonyl)acetamide;

2-(3-(2-Benzyl-1-oxo-1,2-dihydroisoquinolin-4-yl)-5-fluoro-2-methyl-1H-indol-1-yl)-N-(methylsulfonyl)acetamide;

2-(3-(1-Benzyl-6-oxo-1,6-dihydropyridazin-3-yl)-5-fluoro-2-methyl-1H-indol-1-yl)acetic acid;

2-(3-(1-(2,4-Dichlorobenzyl)-6-oxo-1,6-dihydropyridazin-3-yl)-5-fluoro-2-methyl-1H-indol-1-yl)acetic acid;

[3-(3-Isopropyl-4-oxo-3,4-dihydrophthalazin-1-yl)-2-methyl-1H-indol-1-yl]acetic acid;

{5-Fluoro-2-methyl-3-[3-(2-methylpropyl)-4-oxo-3,4-dihydrophthalazin-1-yl]-1H-indol-1-yl}acetic acid;

[3-(3-Benzyl-4-oxo-3,4-dihydrophthalazin-1-yl)-5-fluoro-2-methyl-1H-indol-1-yl]acetic acid;

{5-Fluoro-3-[3-(3-fluorobenzyl)-4-oxo-3,4-dihydrophthalazin-1-yl]-2-methyl-1H-indol-1-yl}acetic acid;

{5-Fluoro-2-methyl-3-[3-(1-methylethyl)-4-oxo-3,4-dihydrophthalazin-1-yl]-1H-indol-1-yl}-acetic acid;

{5-Chloro-3-[3-(2,4-dichlorobenzyl)-4-oxo-3,4-dihydrophthalazin-1-yl]-2-methyl-1H-indol-1-yl}acetic acid;

(5-Chloro-2-methyl-3-{3-[4-(methylsulfonyl)benzyl]-4-oxo-3,4-dihydrophthalazin-1-yl}-1H-indol-1-yl)acetic acid;

2-(5-Fluoro-3-(1-isobutyl-6-oxo-1,6-dihydropyridazin-3-yl)-2-methyl-1H-indol-1-yl)acetic acid;

[5-Fluoro-3-(1-isopropyl-6-oxo-1,6-dihydropyridazin-3-yl)-2-methyl-1H-indol-1-yl]acetic acid;

(3-{[1-(2,4-Difluorobenzyl)-6-oxo-1,6-dihydropyridazin-3-yl]methyl}-2-methyl-1H-indol-1-yl)acetic acid;

(3-{[1-(2,5-Difluorobenzyl)-6-oxo-1,6-dihydropyridazin-3-yl]methyl}-5-fluoro-2-methyl-1H-indol-1-yl)acetic acid;

{3-[(1-Benzyl-6-oxo-1,6-dihydropyridazin-3-yl)methyl]-5-fluoro-2-methyl-1H-indol-1-yl}-acetic acid;

(3-{[1-(2,6-Difluorobenzyl)-6-oxo-1,6-dihydropyridazin-3-yl]methyl}-5-fluoro-2-methyl-1H-indol-1-yl)acetic acid;

(3-{[1-(2,3-Difluorobenzyl)-6-oxo-1,6-dihydropyridazin-3-yl]methyl}-5-fluoro-2-methyl-1H-indol-1-yl)acetic acid;

(3-{[1-(2-Fluorobenzyl)-6-oxo-1,6-dihydropyridazin-3-yl]methyl}-5-fluoro-2-methyl-1H-indol-1-yl)acetic acid;

(3-{[1-(3-fluorobenzyl)-6-oxo-1,6-dihydropyridazin-3-yl]methyl}-5-fluoro-2-methyl-1H-indol-1-yl)acetic acid;

(3-{[1-(4-Fluorobenzyl)-6-oxo-1,6-dihydropyridazin-3-yl]methyl}-5-fluoro-2-methyl-1H-indol-1-yl)acetic acid;

(3-{[1-(2,2-Dimethylpropyl)-6-oxo-1,6-dihydropyridazin-3-yl]methyl}-5-fluoro-2-methyl-1H-indol-1-yl)acetic acid;

2-(5-Fluoro-2-methyl-3-((6-oxo-1-(4,4,4-trifluorobutyl)-1,6-dihydropyridazin-3-yl)methyl)-1H-indol-1-yl)acetic acid;

(5-Fluoro-2-methyl-3-{[6-oxo-1-(2,2,2-trifluoroethyl)-1,6-dihydropyridazin-3-yl]methyl}-1H-indol-1-yl)acetic acid;

{3-[(1-Benzyl-6-oxo-1,6-dihydropyridin-3-yl)methyl]-5-fluoro-2-methyl-1H-indol-1-yl}-acetic acid;

{3-[(1-Benzyl-6-oxo-1,6-dihydropyridin-3-yl)methyl]-5-chloro-2-methyl-1H-indol-1-yl}-acetic acid;

{3-[(1-Benzyl-6-oxo-1,6-dihydropyridin-3-yl)methyl]-2-methyl-1H-indol-1-yl}acetic acid; and

{3-[3-(2-amino-2-oxoethyl)-4-oxo-3,4-dihydrophthalazin-1-yl]-5-fluoro-2-methyl-1H-indol-1-yl}acetic acid or

a pharmaceutically acceptable salt thereof.

In another embodiments, a pharmaceutical composition comprising a compound of Formula (I) or a pharmaceutically acceptable salt thereof and a pharmaceutically acceptable carrier.

In yet another embodiments, a method of treating a disease or a disorder in a patient, comprising administering to a patient in need thereof a compound of Formula (I) or a pharmaceutically acceptable salt thereof or a pharmaceutical composition.

In some embodiments, the disease or disorder is selected from the group consisting of asthma, chronic obstructive pulmonary disease (COPD), bronchitis, rhinitis, nasal polyposis, sarcoidosis, farmer's lung, fibroid lung, idiopathic intestinal pneumonia, cystic fibrosis, cough, psoriasis, dermatitis, urticaria, cutaneous eosinophilias, chronic sinusitis, eosinophilic esophagitis, eosinophilic gastroenteritis, eosinophilic colitis, eosinophilic fasciitis, lupus, rheumatoid arthritis, inflammatory Bowel disease, Celiac disease, scleroderma, ankylosing spondylitis, autoimmune diseases, allergic diseases and hyper IgE syndrome.

In some embodiments, the treatment of a disease or a disorder further comprises administering an additional therapeutic agent.

In some embodiments, the disease or disorder is characterized by elevated levels of prostaglandin D₂ (PGD₂) or a metabolite thereof.

In some embodiments, the disease or disorder is characterized by elevated levels of a thromboxane metabolite.

In another embodiments, a method of inhibiting the binding of endogenous ligands to the CRTH-2 receptor in a cell, comprising contacting the cell with a therapeutically effective amount of a compound of Formula (I) or a pharmaceutically acceptable salt thereof or a pharmaceutical composition.

In some embodiments, the endogenous ligand is prostaglandin D₂ (PGD₂) or a metabolite thereof.

In some embodiments, the endogenous ligand is a thromboxane metabolite.

The receptor CRTH2 antagonists of the present invention are indole based receptor CRTH2 antagonists compounds, and include all enantiomeric and diasteriomeric forms and salts of compounds having the formula (I).

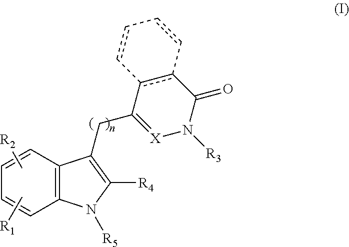

Compounds of the present invention can be prepared in accordance with the procedures outlined herein, from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be readily obtained from the relevant scientific literature or from standard textbooks in the field. It will be appreciated that where typical or preferred process conditions (i.e., reaction temperatures, times, mole ratios of reactants, solvents, pressures, etc.) are given; other process conditions can also be used unless otherwise stated. Optimum reaction conditions can vary with the particular reactants or solvent used. Those skilled in the art will recognize that the nature and order of the synthetic steps presented can be varied for the purpose of optimizing the formation of the compounds described herein.

The processes described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatography such as high-performance liquid chromatograpy (HPLC), gas chromatography (GC), gel-permeation chromatography (GPC), or thin layer chromatography (TLC).

Preparation of the compounds can involve protection and deprotection of various chemical groups. The chemistry of protecting groups can be found, for example, in Greene et al., Protective Groups in Organic Synthesis, 4th. Ed. (John Wiley & Sons, 2007), the entire disclosure of which is incorporated by reference herein for all purposes.

The reactions or the processes described herein can be carried out in suitable solvents, which can be readily selected by one skilled in the art. Suitable solvents typically are substantially nonreactive with the reactants, intermediates, and/or products at the temperatures at which the reactions are carried out, i.e., temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected.

The compounds of these teachings can be prepared by methods known in the art. The reagents used in the preparation of the compounds of these teachings can be either commercially obtained or can be prepared by standard procedures described in the literature. For example, compounds of the present invention can be prepared according to the methods illustrated in the following Synthetic Schemes.

The description of this invention utilizes a variety of abbreviations well known to those skilled in the art, including the following:
“aq” refers to aqueous;
CH₃CN: Acetonitrile
DMF: N,N-Dimethylformamide
DMSO: Dimethylsulfoxide
HCl: Hydrochloric acid
EtOAc: Ethyl acetate
HOAc: Acetic acid
EtOH: Ethanol
HPLC: High performance Liquid Chromatography
K₂CO₃: Potassium carbonate
MeOH: Methanol
MgSO₄: Magnesium sulfate
NaI: Sodium iodide
rt: Room temperature
TEA: Triethylamine
TFA: Trifluoroacetic acid
THF: Tetrahydrofuran
TMS: Trimethylsilyl

### Synthetic Procedures

The reagents used in the preparation of the compounds of this invention can be either commercially obtained or can be prepared by standard procedures described in the literature. In accordance with this invention, compounds in the genus were prepared by the following schemes.

### EXAMPLES

The following non-limiting examples are presented merely to illustrate the present invention. The skilled person will understand that there are numerous equivalents and variations not exemplified but which still form part of the present teachings.

### Example 1

Step 1: Preparation of 1-chloro-4-(5-chloro-2-methyl-1H-indol-3-yl)phthalazine, Intermediate 1. In a 500 mL round-bottomed flask, 5-chloro-2-methylindole (1.00 g, 6.04 mmol) and 1,4-dichlorophthalazine (1.26 g, 6.34 mmol) were taken up in 80 mL dichloroethane. Aluminum chloride (1.13 g, 8.46 mmol) was added, and the mixture refluxed overnight, under a nitrogen-filled balloon. After cooling slightly, the reaction mixture was poured into a mixture of ice and 2 M hydrochloric acid. This was stirred until all the ice had melted, and the layers separated. The aqueous layer was extracted with additional dichloroethane, and the combined organic extracts washed with brine, dried over anhydrous magnesium sulfate, filtered, and evaporated to give material of sufficient purity to be used directly in the next step (1.95 g, 98% yield): ¹H NMR (DMSO-d₆) δ 11.88 (s, 1H), 8.34-8.38 (m, 1H), 8.14-8.19 (m, 1H), 8.04-8.10 (m, 1H), 7.93 (dt, J=8.1, 1.0 Hz, 1H), 7.46 (d, J=8.6 Hz, 1H), 7.20 (d, J=1.8 Hz, 1H), 7.13 (dd, J=8.6, 2.0 Hz, 1H), 2.40 (s, 3H)

Step 2: Preparation of tert-Butyl 2-(5-chloro-3-(4-chlorophthalazin-1-yl)-2-methyl-1H-indol-1-yl)acetate, intermediate 1A. Intermediate 1 (1.95 g, 5.94 mmol), potassium carbonate (1.64 g, 11.9 mmol) and tert-butyl bromoacetate (1.8 mL, 2.3 g, 12 mmol) were taken up in 30 mL DMF in a 250 mL round-bottomed flask and heated at 70° C. overnight. The reaction mixture was then poured into water, extracted into ethyl acetate (3×), washed with brine (3×), dried over anhydrous magnesium sulfate, filtered and evaporated. The crude product was purified by flash chromatography over silica gel (7-60% ethyl acetate in hexanes) to give pure product (1.43 g, 54% yield): ¹H NMR (DMSO-d₆) δ 8.37-8.42 (m, 1H), 8.19 (ddd, J=8.3, 7.1, 1.3 Hz, 1H), 8.09 (ddd, J=8.3, 7.1, 1.3 Hz, 1H), 7.80-7.85 (m, 1H), 7.62 (dd, J=8.1, 1.0 Hz, 1H), 7.19-7.24 (m, 2H), 5.19 (s, 2H), 2.30 (s, 3H), 1.45 (s, 9H).

Step 3: Preparation of tert-butyl 2-(5-chloro-3-(4-hydroxyphthalazin-1-yl)-2-methyl-1H-indol-1-yl)acetate, intermediate 2. In a round-bottomed flask, intermediate 1A was taken up in 100 mL acetic acid, and 20 mL sodium hydroxide was added. The mixture was heated at 70° C. for 1 hour, until LC-MS analysis indicated complete conversion to product. It was then partitioned between 175 mL each ethyl acetate and brine, and the aqueous layer extracted with additional ethyl acetate. The combined organic extracts were washed with water (3×) and brine, dried over anhydrous magnesium sulfate, filtered, evaporated, and azeotroped with toluene to give pure product (2.80 g, 97% yield): ¹H NMR (DMSO-d₆) δ 12.82 (s, 1H), 8.31-8.38 (m, 1H), 7.82-7.90 (m, 2H), 7.57 (d, J=8.6 Hz, 1H), 7.41-7.46 (m, 1H), 7.20 (d, J=2.0 Hz, 1H), 7.17 (dd, J=8.6, 2.0 Hz, 1H), 5.13 (s, 2H), 2.25 (s, 3H), 1.44 (s, 9H).

Step 4: Preparation of 2-(5-Chloro-3-(3-(4-chlorobenzyl)-4-oxo-3,4-dihydrophthalazin-1-yl)-2-methyl-1H-indol-1-yl)acetic acid (1). Intermediate 2 (0.232 g, 0.548 mmol), potassium carbonate (0.265 g, 1.92 mmol) and 4-chlorobenzyl bromide (0.338 g, 1.64 mmol) were taken up in 8 mL DMF and heated at 85° C. for 2.5 hours, until LC-MS analysis showed complete consumption of starting material. The mixture was then cooled to room temperature, poured into 80 mL water, extracted into ethyl acetate, washed with brine, dried over anhydrous magnesium sulfate, filtered, and evaporated. Trifluoroacetic acid was added to the crude ester, and this reaction mixture was stirred for 1 hour. The mixture was then evaporated, and purified by flash chromatography (101 mg, 37% yield): ¹H NMR (DMSO-d₆) δ 13.21 (br. s., 1H), 8.38-8.43 (m, 1H), 7.85-7.94 (m, 2H), 7.56 (d, J=8.6 Hz, 1H), 7.50-7.54 (m, 1H), 7.41 (s, 4H), 7.15 (dd, J=8.7, 2.1 Hz, 1H), 7.11 (d, J=2.0 Hz, 1H), 5.32-5.50 (m, 2H), 5.12 (s, 2H), 2.23 (s, 3H).

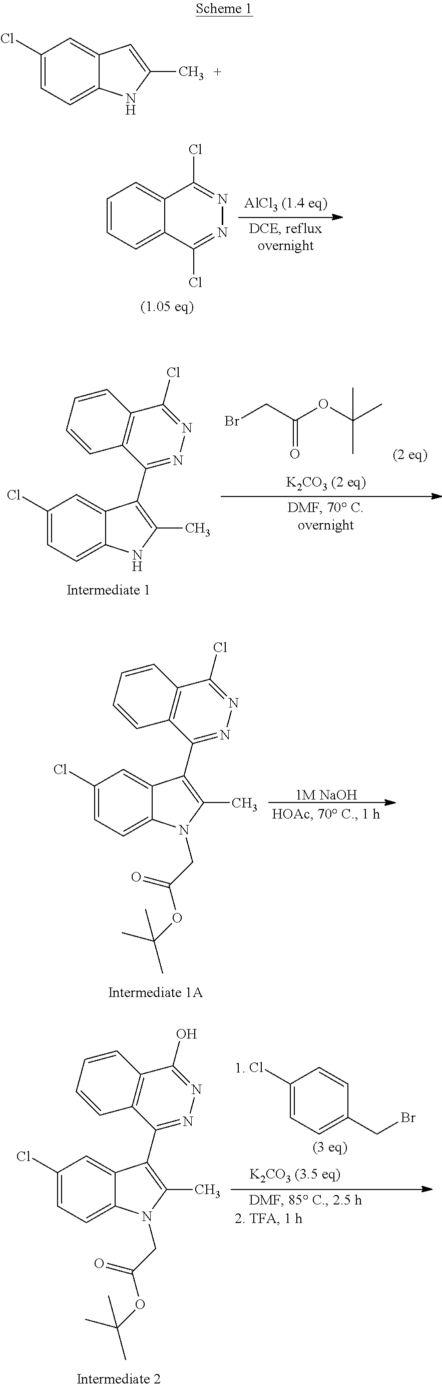

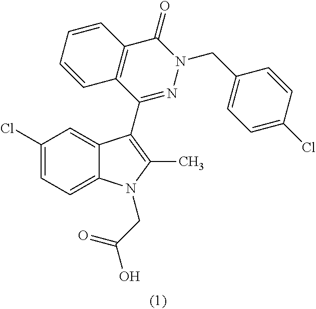

### Example 2

Preparation of 2-(5-Chloro-3-(3-(4-chloro-3-fluorobenzyl)-4-oxo-3,4-dihydrophthalazin-1-yl)-2-methyl-1H-indol-1-yl)acetic acid (2). The title compound was prepared according to the procedure of Example 1; Yield: 29%.

### Example 3

Preparation of 2-(5-Chloro-3-(3-(3-fluoro-4-(trifluoromethyl)benzyl)-4-oxo-3,4-dihydrophthalazin-1-yl)-2-methyl-1H-indol-1-yl)acetic acid (3). The title compound was prepared according to the procedure of Example 1; Yield: 19%.

### Example 4

Step 1: Preparation of 1-Chloro-4-(5-fluoro-2-methyl-1H-indol-3-yl)phthalazine, Intermediate 3. The title compound was prepared according to the procedure of Intermediate 1; Yield: 58%.

Step 2: Preparation of tert-Butyl 2-(3-(4-chlorophthalazin-1-yl)-5-fluoro-2-methyl-1-H-indol-1-yl)acetate, Intermediate 4. The title compound was prepared according to the procedure of intermediate 1A; Yield: 98%.

Step 3: Preparation of tert-Butyl 2-(5-fluoro-3-(4-hydroxyphthalazin-1-yl)-2-methyl-1H-indol-1-yl)acetate, Intermediate 5. The title compound was prepared according to the procedure of intermediate 2; Yield: 80%.

Step 4: Preparation of 2-(3-(3-(4-Chlorobenzyl)-4-oxo-3,4-dihydropthalazin-1-yl)-5-fluoro-2-methyl-1H-indol-1-yl)acetic acid (4). The title compound was prepared according to the procedure of Example 1; Yield: 31%

### Example 5

Preparation of 2-(3-(3-(4-Chloro-3-fluorobenzyl)-4-oxo-3,4-dihydrophthalazin-1-yl)-5-fluoro-2-methyl-1H-indol-1-yl)acetic acid (5). The title compound was prepared according to the procedure of Example 1; Yield: 43%

### Example 6

Preparation of 2-(5-Fluoro-3-(3-(3-fluoro-4-(trifluoromethyl)benzyl)-4-oxo-3,4-dihydrophthalazin-1-yl)-2-methyl-1H-indol-1-yl)acetic acid (6). The title compound was prepared according to the procedure of Example 1; Yield: 37%.

### Example 7

Preparation of 2-(3-(3-(2,4-dichlorobenzyl)-4-oxo-3,4-dihydrophthalazin-1-yl)-5-fluoro-2-methyl-1H-indol-1-yl)acetic acid (7). The title compound was prepared according to the procedure of Example 1; Yield: 39%.

### Example 8

Step 1: Preparation of 5-Chloro-3-(6-chloropyridazin-3-yl)-2-methyl-1H-indole, Intermediate 6. The procedure described above for intermediate 1 was followed, reacting 5-chloro-2-methyl-indole (5.00 g, 30.2 mmol) with 3,6-dichloropyridazine (6.00 g, 40.3 mmol) and aluminum chloride (6.04 g, 45.3 mmol). After the reaction mixture had cooled slightly, it was poured into 300 mL of an ice/2M HCl mixture and stirred until the ice had melted. The precipitate was collected, washed three times with water, and dried under vacuum to give pure product (6.84 g, 81% yield).

Step 2: Preparation of tert-Butyl 2-(5-(chloro-3-(6-chloropyridazin-3-yl)-2-methyl-1H-indol-1-yl)acetate, Intermediate 7. The procedure described above for intermediate 1A was followed, reacting intermediate 6 (6.84 g, 24.6 mmol) with potassium carbonate (11.9 g, 86.1 mmol) and tert-butyl bromoacetate (14.5 mL, 19.2 g, 98.4 mmol) at 80° C. overnight. The reaction mixture was then poured into 2.5 L water, and the precipitate collected, washed with water, and dried under vacuum to give reasonably pure product as an ivory-colored powder (10.27 g, 106% yield).

Step 3: Preparation of tert-Butyl 2-(5-chloro-3-(6-hydroxypyridazin-3-yl)-2-methyl-1H-indol-1-yl)acetate, Intermediate 8. The procedure described above for intermediate 2 was followed, heating intermediate 7 (9.65 g, 24.6 mmol) in 360 mL acetic acid and 70 mL 1 M sodium hydroxide overnight. Some chloride remained, but ester cleavage was starting to occur. The reaction mixture was poured into 1800 mL ice water and stirred briefly; then the precipitate was washed successively with water and hexanes, and dried to give product of sufficient purity to be used in the next step (7.12 g, 77% yield).

Step 4: Preparation of 2-(5-Chloro-3-(1-(4-chloro-3-fluorobenzyl)-6-oxo-1,6-dihydropyridazin-3-yl)-2-methyl-1H-indol-1-yl)acetic acid (8). The procedure described above for 1 was followed, reacting intermediate 8 (0.414 g, 1.11 mmol) with 4-chloro-3-fluorobenzyl bromide (0.744 g, 3.33 mmol) and potassium carbonate (0.537 g, 3.89 mmol), then deprotecting with trifluoroacetic acid and purifying by preparative HPLC (water/acetonitrile with 0.1% formic acid). Pure product was obtained as a pale yellow powder (0.139 g, 27% yield): ¹H NMR (DMSO-d₆) δ 13.21 (br. s., 1H), 7.76 (d, J=9.6 Hz, 1H), 7.59 (t, J=8.0 Hz, 1H), 7.48-7.53 (m, 2H), 7.43 (dd, J=10.2, 1.9 Hz, 1H), 7.22 (dd, J=8.3, 1.3 Hz, 1H), 7.14 (dd, J=8.7, 2.1 Hz, 1H), 7.08 (d, J=9.6 Hz, 1H), 5.36 (s, 2H), 5.08 (s, 2H), 2.42 (s, 3H).

### Example 9

Preparation of 2-(5-Chloro-3-(1-(4-chlorobenzyl)-6-oxo-1,6-dihydropyridazin-3-yl)-2-methyl-1H-indol-1-yl)acetic acid (9). The title compound was prepared according to the procedure of Example 8; Yield: 19%.

### Example 10

Step 1: Preparation of 4-Chloro-2-fluoro-6-propynylaniline, Intermediate 9. In a 350 mL glass pressure vessel with a threaded Teflon cap, 4-chloro-2-fluoro-6-iodoaniline (3.25 g, 12.0 mmol), CuI (30 mg, 0.16 mmol), and Pd(PPh₃)₂Cl₂ (0.101 g, 0.144 mmol) were taken up in 165 mL triethylamine and cooled to −78° C. Propyne (2.7 mL, 1.9 g, 48 mmol) was condensed into a graduated cylinder and added to the reaction vessel. The vessel was then capped, the cooling bath removed, and the reaction mixture allowed to stir while warming to room temperature overnight behind a safety shield. Removal of the triethylamine by evaporation gave a crude material that was purified by flash chromatography over silica gel (1-10% ethyl acetate in hexanes) to give pure product (2.00 g, 91% yield): ¹H NMR (CHLOROFORM-d) δ 7.00-7.04 (m, 1H), 6.95 (dd, J=10.6, 2.3 Hz, 1H), 4.18 (br. s., 2H), 2.13 (s, 3H).

Step 2: Preparation of 5-Chloro-7-fluoro-2-methyl-1H-indole, Intermediate 10. Intermediate 9 (2.00 g, 10.9 mmol) was taken up in 210 mL anhydrous DMF, and CuI (0.228 g, 1.20 mmol) was added. The mixture was refluxed under nitrogen for 1 hour, until t.l.c. analysis (5% ethyl acetate in hexanes) showed complete conversion to product. The reaction mixture was then evaporated, and the crude material purified by flash chromatography over silica gel (1-10% ethyl acetate in hexanes) to give pure product as a pale yellow solid (1.75 g, 88% yield): ¹H NMR (DMSO-d₆) δ 11.58 (br. s., 1H), 7.29 (d, J=1.8 Hz, 1H), 6.94 (dd, J=10.9, 1.8 Hz, 1H), 6.21 (ddd, J=3.4, 1.9, 0.8 Hz, 1H), 2.38 (d, J=0.8 Hz, 3H).

Step 3: Preparation of 5-Chloro-3-(6-chloropyridazin-3-yl)-7-fluoro-2-methyl-1H-indole, Intermediate 11. The title compound was prepared according to the procedure of intermediate 1; Yield: 52%.

Step 4: Preparation of Methyl 2-(5-chloro-3-(6-chloropyridazin-3-yl)-7-fluoro-2-methyl-1H-indol-1-yl)acetate, Intermediate 12. The title compound was prepared according to the procedure of intermediate 1A; Yield: 77%.

Step 5: Preparation of Methyl 2-(5-chloro-7-fluoro-3-(6-hydroxypyridaz in-3-yl)-2-methyl-1H-indol-1-yl)acetate, Intermediate 13. The title compound was prepared according to the procedure of intermediate 2; Yield: 75%.

Step 6: Preparation of 2-(3-(1-Benzyl-6-oxo-1,6-dihydropyridazin-3-yl)-5-chloro-7-fluoro-2-methyl-1H-indol-1-yl)acetic acid (10). The title compound was prepared according to the procedure of example 1 followed by hydrolysis with lithium hydroxide; Yield: 15%.

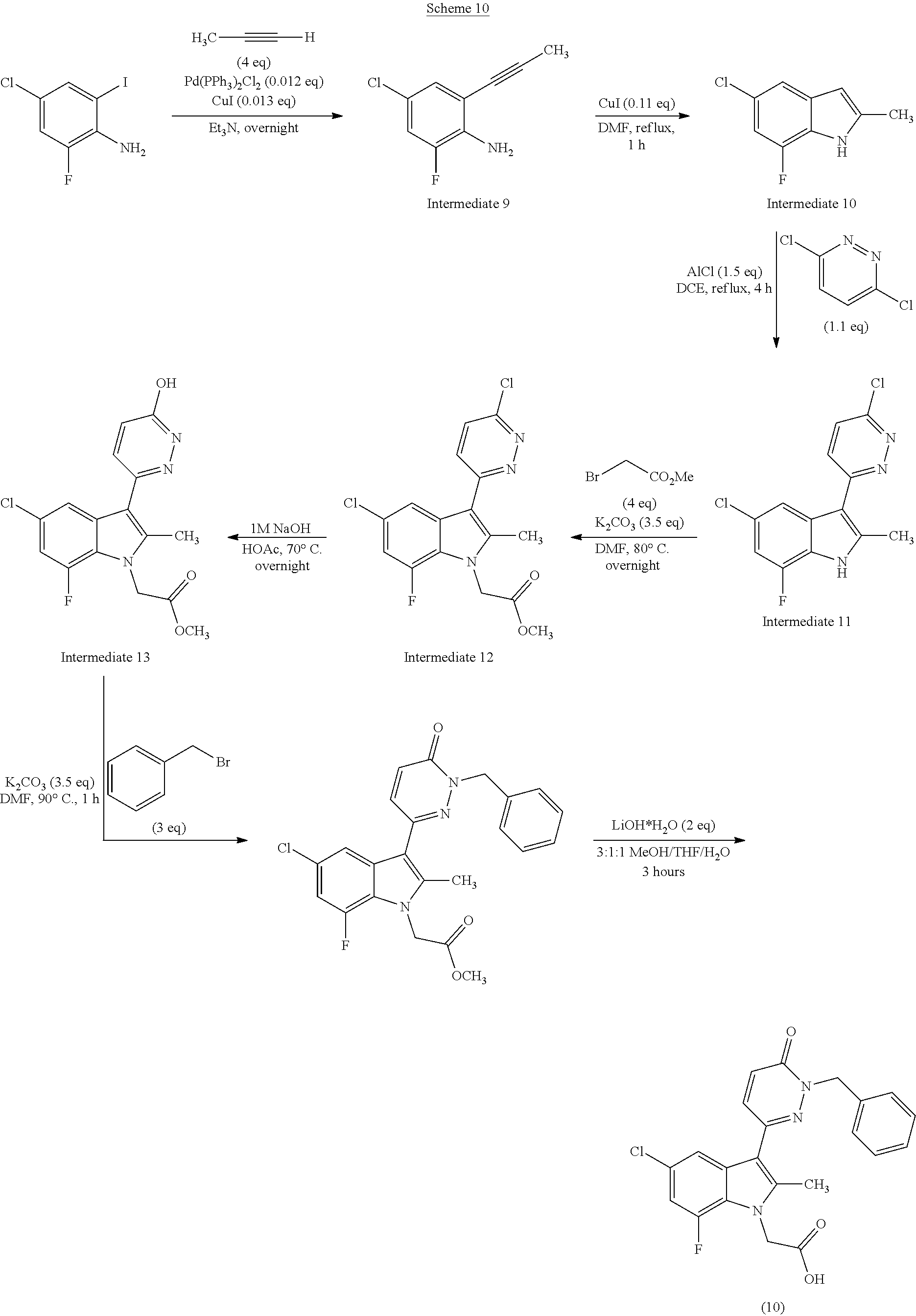

### Example 11

Step 1: Preparation of 1-Chloro-4-(5-chloro-7-fluoro-2-methyl-1H-indol-3-yl)phthalazine, Intermediate 14. The title compound was prepared according to the procedure of intermediate 1; Yield: 74%.

Step 2: Preparation of tert-Butyl 2-(5-chloro-3-(4-chlorophthalazin-1-yl)-7-fluoro-2-methyl-1H-indol-1-yl)acetate, Intermediate 15. The title compound was prepared according to the procedure of intermediate 1A; Yield: 98%.

Step 3: Preparation of tert-Butyl 2-(5-chloro-7-fluoro-3-(4-hydroxyphthalazin-1-yl)-2-methyl-1H-indol-1-yl)acetate, Intermediate 16. The title compound was prepared according to the procedure of intermediate 2; Yield: 98%.

Step 4: Preparation of 2-(3-(3-Benzyl-4-oxo-3,4-dihydrophthalazin-1-yl)-5-chloro-7-fluoro-2-methyl-1H-indol-1-yl)acetic acid (11). The title compound was prepared according to the procedure of Example 1; Yield: 46%.

### Example 12

Step 1: Preparation of 3-(6-Chloropyridazin-3-yl)-5-fluoro-2-methyl-1H-indole, Intermediate 17. The procedure described above for intermediate 1 was followed, reacting 5-fluoro-2-methylindole (4.87 g, 32.6 mmol) with 3,6-dichloropyridazine (6.5 g, 44 mmol) and aluminum chloride (6.52 g, 48.9 mmol). (5.33 g, 62% yield).

Step 2: Preparation of Methyl 2-(3-(6-chloropyridazin-3-yl)-5-fluoro-2-methyl-1H-indol-1-yl)acetate, Intermediate 18. The procedure described above for intermediate 1A was followed, reacting intermediate 17 (5.33 g, 20.4 mmol) with potassium carbonate (9.87 g, 20.4 mmol) and methyl bromoacetate (7.5 mL, 13 g, 82 mmol). (5.58 g, 82% yield).

Step 3: Preparation of Methyl 2-(5-fluoro-3-(6-hydroxypyridaz in-3-yl)-2-methyl-1H-indol-1-yl)acetate, Intermediate 19. The procedure above for intermediate 2 was followed, heating intermediate 18 (5.58 g, 16.7 mmol) in 245 mL acetic acid and 50 mL 1 M sodium hydroxide. (3.78 g, 72% yield).

Step 4: Preparation of 2-(3-(1-(4-chloro-3-fluorobenzyl)-6-oxo-1,6-dihydropyridazin-3-yl)-5-fluoro-2-methyl-1H-indol-1-yl)acetic acid (12). The procedure described above for 1 was followed, reacting intermediate 19 (0.370 g, 1.17 mmol) with 4-chloro-3-fluorobenzyl bromide (0.784 g, 3.51 mmol) and potassium carbonate (0.567 g, 4.10 mmol) at 100° C. for 1 hour. The crude ester was purified by flash chromatography over silica gel (2-20% ethyl acetate in dichloromethane). The purified ester was taken up in 9 mL methanol and 3 mL tetrahydrofuran, and a solution of LiOH*H₂O (98 mg, 2.3 mmol) in 3 mL water was added and the reaction stirred at room temperature. The reaction mixture was evaporated, purified by flash chromatography and preparative (0.165 g, 32% yield): ¹H NMR (DMSO-d₆) δ 7.76 (d, J=9.6 Hz, 1H), 7.58 (t, J=8.1 Hz, 1H), 7.40-7.47 (m, 2H), 7.29 (dd, J=10.1, 2.5 Hz, 1H), 7.21 (dd, J=8.2, 1.4 Hz, 1H), 7.06 (d, J=9.6 Hz, 1H), 6.96 (td, J=9.2, 2.4 Hz, 1H), 5.36 (s, 2H), 4.93 (br. s., 2H), 2.41 (s, 3H).

### Example 13

Preparation of 2-(3-(1-(4-Chlorobenzyl)-6-oxo-1,6-dihydropyridazin-3-yl)-5-fluoro-2-methyl-1H-indol-1-yl)acetic acid (13). The title compound was prepared according to the procedure of Example 12; Yield: 31%.

### Example 14

Step 1: Preparation of 2-Chloro-6-iodo-4-(methylsulfonyl)aniline, Intermediate 20. In a 250 mL round-bottomed flask, 2-chloro-4-(methylsulfonyl)aniline (4.06 g, 19.7 mmol) was taken up in dichloromethane. Bis(pyridine)iodonium (I) tetrafluoroborate (11.0 g, 29.6 mmol) was added, then trifluoromethanesulfonic acid (5.2 mL, 8.9 g, 59 mmol) was added slowly via a syringe. LC-MS analysis 5 minutes after completion of this addition showed complete conversion to product. The reaction mixture was quenched with water, then partitioned between water and dichloromethane, and the aqueous layer extracted with additional dichloromethane. The combined organic extracts were washed with 5% sodium thiosulfate, dried over anhydrous magnesium sulfate, filtered, evaporated, and purified by flash chromatography over silica gel (3.13 g, 48% yield).

Step 2: Preparation of 2-Chloro-4-(methylsulfonyl)-6-propynylaniline, Intermediate 20A. The procedure described above for intermediate 9 was followed, reacting intermediate 20 (1.25 g, 3.77 mmol) with propyne (0.85 mL, 0.60 g, 15 mmol), Pd(PPh₃)Cl₂ (32 mg, 45 μmol) and CuI (9.3 mg, 49 μmol). Flash chromatography over silica gel gave pure product (0.801 g, 87%)

Step 3: Preparation of 7-Chloro-2-methyl-5-(methylsulfonyl)-1H-indole, intermediate 20B. The procedure described above for intermediate 10 was followed, refluxing a DMF solution of intermediate 20A (0.801 g, 3.29 mmol) to which CuI (69 mg, 0.362 mmol) had been added, for 1 hour. The reaction mixture was then poured into 650 mL water, extracted into ethyl acetate, washed with brine, dried over anhydrous magnesium sulfate, filtered, evaporated, and purified by flash chromatography over silica gel (12-100% ethyl acetate in hexanes) to give product of sufficient purity to be used in the next step (0.47 g, 59% yield).

Step 4: Preparation of 1-Chloro-4-(5-chloro-7-fluoro-2-methyl-1H-indol-3-yl)phthalazine, Intermediate 21. The procedure described above for intermediate 1 was followed, reacting intermediate 20B (0.539 g, 2.21 mmol) with 1,4-dichlorophthalazine (0.484 g, 2.43 mmol) and aluminum chloride (0.413 g, 3.09 mmol). Aqueous work-up gave crude material that was purified by flash chromatography over silica gel (12-100% ethyl acetate in hexanes) to give pure product (0.483 g, 54% yield).

Step 5: Preparation of tert-Butyl 2-(7-chloro-3-(4-chlorophthalazin-1-yl)-2-methyl-5-(methylsulfonyl)-1H-indol-1-yl)acetate, Intermediate 22. The procedure described above for intermediate 1A was followed, reacting intermediate 21 (0.483 g, 1.19 mmol) with potassium carbonate (0.329 g, 2.38 mmol) and tert-butyl bromoacetate (0.35 mL, 0.46 g, 2.4 mmol) in DMF at 70° C. for 2 hours, until LC-MS analysis showed complete conversion to product. The reaction mixture was poured into 60 mL ice water and stirred until the ice melted. The precipitate was then collected, washed with water and dried (0.512 g, 83% yield).

Step 6: Preparation of tert-Butyl 2-(7-chloro-3-(4-hydroxyphthalazin-1-yl)-2-methyl-5-(methylsulfonyl)-1H-indol-1-yl)acetate, Intermediate 23. The procedure described above for intermediate 2 was followed, heating intermediate 22 (0.512 g, 0.983 mmol) in 14 mL acetic acid and 2.9 mL 1 M sodium hydroxide for 1 hour. The reaction mixture was then poured into 170 mL ice water, and the off-white precipitate collected and dried under vacuum (0.335 g, 68% yield).

Step 7: 2-(3-(3-Benzyl-4-oxo-3,4-dihydrophthalazin-1-yl)-7-chloro-2-methyl-5-(methylsulfonyl)-1H-indol-1-yl)acetic acid (14). The procedure described above for 1 was followed, reacting intermediate 23 (0.335 g, 0.667 mmol) with benzyl bromide (0.24 mL, 0.34 g, 2.0 mmol) and potassium carbonate (0.323 g, 2.33 mmol). The crude ester was purified by flash chromatography over silica gel (12-100% ethyl acetate in hexanes), then deprotected with trifluoroacetic acid and purified by preparative HPLC (water/acetonitrile with 0.1% formic acid). Lyophilization gave a fluffy white solid (0.125 g, 35% yield): ¹H NMR (DMSO-d₆) δ13.43 (br. s., 1H), 8.39-8.43 (m, 1H), 7.89-7.94 (m, 1H), 7.84-7.89 (m, 1H), 7.78 (d, J=1.5 Hz, 1H), 7.70 (d, J=1.8 Hz, 1H), 7.40-7.46 (m, 3H), 7.33-7.39 (m, 2H), 7.26-7.31 (m, 1H), 5.34-5.50 (m, 4H), 3.17 (s, 3H), 2.29 (s, 3H).

### Example 15

Step 1: Preparation of 2-Fluoro-6-iodo-4-(methylsulfonyl)aniline, Intermediate 24. The procedure described above for intermediate 20 was followed, reacting 2-fluoro-4-(methylsulfonyl)aniline (5.01 g, 26.5 mmol) in 130 mL dichloromethane with bis(pyridine)iodonium (I) tetrafluoroborate (14.8 g, 39.8 mmol) and trifluoromethanesulfonic acid (7.0 mL, 12 g, 80 mmol), which was added dropwise from an addition funnel. As soon as the addition was complete, 130 mL water was added. The layers were separated, and the aqueous layer extracted with additional dichloromethane. The combined organic extracts were washed with 5% sodium thiosulfate, dried over anhydrous magnesium sulfate, filtered, evaporated, and purified by flash chromatography over silica gel (6-50% ethyl acetate in hexanes) to give a pink solid (4.07 g, 49% yield).

Step 2: Preparation of 2-Fluoro-4-(methylsulfonyl)-6-propynylaniline, Intermediate 24A. The procedure described above for intermediate 9 was followed, reacting intermediate 24 (4.07 g, 12.9 mmol) with propyne (2.9 mL, 2.1 g, 52 mmol), Pd(PPh₃)Cl₂ (109 mg, 0.155 mmol) and CuI (32 mg, 0.168 mmol). Flash chromatography over silica gel (6-50% ethyl acetate in hexanes) gave a beige solid (2.70 g, 92% yield).

Step 3: Preparation of 7-Fluoro-2-methyl-5-(methylsulfonyl)-1H-indole, Intermediate 25. The procedure described above for intermediate 10 was followed, cyclizing 24A (2.70 g, 11.9 mmol) in the presence of CuI (249 mg, 1.31 mmol). Flash chromatography over silica gel (12-100% ethyl acetate in hexanes) gave pure product (1.57 g, 58% yield).

Step 4: Preparation of 1-Chloro-4-(7-fluoro-2-methyl-5-(methylsulfonyl)-1H-indol-3-yl)phthalazine, Intermediate 26. The procedure described above for intermediate 1 was followed, reacting intermediate 25 (0.686 g, 3.02 mmol) with 1,4-dichlorophthalazine (0.661 g, 3.32 mmol) and aluminum chloride (0.564 g, 4.23 mmol). The reaction mixture was poured into ice water, rinsing the flask with a small amount of ethyl acetate, stirred vigorously, and filtered to give an ivory precipitate that was washed with water and dried under vacuum (0.579 g, 49% yield).

Step 5: Preparation of tert-Butyl 2-(3-(4-chlorophthalazin-1-yl)-7-fluoro-2-methyl-5-(methyl-sulfonyl)-1H-indol-1-yl)acetate, Intermediate 27. The procedure described above for intermediate 1A was followed, reacting intermediate 26 (0.579 g, 1.49 mmol) with potassium carbonate (0.410 g, 2.97 mmol) and tert-butyl bromoacetate (0.44 mL, 0.58 g, 3.0 mmol) in DMF at 70° C. for 1 hour, until LC-MS analysis showed complete conversion to product. Aqueous work-up gave a yellow foam (0.615 g, 82% yield).

Step 6: Preparation of Tert-Butyl 2-(7-fluoro-3-(4-hydroxyphthalazin-1-yl-2-methyl-5-(methylsulfonyl)-1H-indol-1-yl)acetate, Intermediate 27A. The procedure described above for intermediate 2 was followed, heating intermediate 27 (0.615 g, 1.22 mmol) in 17 mL acetic acid and 4 mL 1 M sodium hydroxide. The reaction mixture was poured into ice water, and the off-white precipitate collected, washed with water and dried under vacuum (0.382 g, 65% yield).

Step 7: 2-(3-(3-Benzyl-4-oxo-3,4-dihydrophthalazin-1-yl)-7-fluoro-2-methyl-5-(methylsulfonyl)-1H-indol-1-yl)acetic acid (15). The procedure described above for 1 was followed, reacting intermediate 27A (0.320 g, 0.659 mmol) with benzyl bromide (0.24 mL, 0.34 g, 2.0 mmol) and potassium carbonate (0.319 g, 2.31 mmol). The crude ester was purified by flash chromatography over silica gel (12-100% ethyl acetate in hexanes), then deprotected with trifluoroacetic acid and purified by preparative HPLC (water/acetonitrile with 0.1% formic acid). Lyophilization gave a fluffy white solid (0.142 g, 42% yield): ¹H NMR (DMSO-d₆) δ 8.40 (dd, J=7.8, 1.3 Hz, 1H), 7.84-7.94 (m, 2H), 7.65 (d, J=1.5 Hz, 1H), 7.46-7.53 (m, 2H), 7.40-7.45 (m, 2H), 7.36 (t, J=7.5 Hz, 2H), 7.25-7.32 (m, 1H), 5.36-5.47 (m, 2H), 5.09 (s, 2H), 3.15 (s, 3H), 2.27 (s, 3H).

### Example 16

Step 1: Preparation of 1-Chloro-4-(5-methoxy-2-methyl-1H-indol-3-yl)phthalazine, Intermediate 28A. The title compound was prepared according to the procedure of intermediate 1; Yield: 9%.

Step 2: Preparation of tert-Butyl 2-(3-(4-chlorophthalazin-1-yl)-5-methoxy-2-methyl-1H-indol-1-yl)acetate, Intermediate 28. The title compound was prepared according to the procedure of intermediate A1; Yield: 88%.

Step 3: Preparation of tert-Butyl 2-(3-(4-hydroxyphthalazin-1-yl)-5-methoxy-2-methyl-1H-indol-1-yl)acetate, Intermediate 29. The title compound was prepared according to the procedure of intermediate 2; Yield: 100%.

Step 4: Preparation of 2-(3-(3-Benzyl-4-oxo-3,4-dihydrophthalazin-1-yl)-5-methoxy-2-methyl-1H-indol-1-yl)acetic acid (16). The title compound was prepared according to the procedure of Example 1; Yield: 38%.

### Example 17

Step 1. Preparation of 3-(6-Chloropyridazin-3-yl)-7-fluoro-2-methyl-5-(methyl-sulfonyl)-1H-indole, Intermediate 30. In a 20 mL Biotage microwave vessel (for 5-10 mL reaction volumes), intermediate 25 (0.835 g, 3.67 mmol) and 3,6-dichloropyridazine (1.64 g, 11.0 mmol) were taken up 10 mL dichloroethane. Aluminum chloride (1.47 g, 11.0 mmol) was added, and the vessel was crimp-sealed and heated in the microwave for 1 hour at 160° C. The contents of the vessel were then poured into 100 mL of an ice/2 M HCl mixture, and the rust-colored precipitate was collected, washed with water and dried under vacuum (0.890 g, 71% yield).

Step 2: Preparation of tert-Butyl 2-(3-(6-chloropyridazin-3-yl)-7-fluoro-2-methyl-5-(methylsulfonyl)-1H-indol-1-yl)acetate, Intermediate 31. The procedure described above for intermediate 1A was followed, reacting intermediate 30 (1.07 g, 3.14 mmol) with potassium carbonate (0.867 g, 6.27 mmol) and tert-butyl bromoacetate (0.93 mL, 1.2 g, 6.3 mmol) in DMF at 70° C. for 100 minutes, until LC-MS analysis showed complete conversion to product. The reaction mixture was poured into 160 mL ice water and the precipitate collected, washed with water and dried under vacuum to give a light brown solid (1.23 g, 86% yield).

Step 3: Preparation of tert-Butyl 2-(7-fluoro-3-(6-hydroxypyridazin-3-yl)-2-methyl-5-(methylsulfonyl)-1H-indol-1-yl)acetate, Intermediate 32. The procedure described above for intermediate 2 was followed, heating intermediate 31 (1.23 g, 2.71 mmol) in 40 mL acetic acid and 8 mL 1 M sodium hydroxide overnight. LC-MS analysis showed that most of the chloride had been consumed. The reaction mixture was poured into ice water, stirred until the ice melted, and the precipitate collected, washed with water and hexanes, and dried under vacuum (0.590 g, 50% yield)

Step 4: Preparation of 2-(3-(1-Benzyl-6-oxo-1,6-dihydropyridazin-3-yl)-7-fluoro-2-methyl-5-(methylsulfonyl)-1H-indol-1-yl)acetic acid (17). The procedure described above for 1 was followed, reacting intermediate 32 (0.590 g, 1.36 mmol) with benzyl bromide (0.48 mL, 0.70 g, 4.0 mmol) and potassium carbonate (0.658 g, 4.76 mmol). The crude ester was purified by flash chromatography over silica gel (5-40% ethyl acetate in dichloromethane), then deprotected with trifluoroacetic acid and purified by preparative HPLC (water/acetonitrile with 0.1% formic acid). Lyophilization gave a fluffy white solid (0.137 g, 21% yield): ¹H NMR (DMSO-d₆) δ 13.65 (br. s., 1H), 8.10 (d, J=1.3 Hz, 1H), 7.78 (d, J=9.9 Hz, 1H), 7.52 (dd, J=11.9, 1.5 Hz, 1H), 7.42-7.47 (m, 2H), 7.34-7.41 (m, 2H), 7.27-7.33 (m, 1H), 7.12 (d, J=9.6 Hz, 1H), 5.33 (s, 2H), 5.12 (s, 2H), 3.23 (s, 3H), 2.45 (s, 3H).

### Example 18

Step 1: Preparation of 3-(6-Chloropyridazin-3-yl)-5-methoxy-2-methyl-1H-indole, Intermediate 33. The title compound was prepared according to the procedure of intermediate 1; Yield: 24%.

Step 2: Preparation of tert-Butyl 2-(3-(6-chloropyridazin-3-yl)-5-methoxy-2-methyl-1H-indol-1-yl)acetate, Intermediate 34. The title compound was prepared according to the procedure of intermediate 1A; Yield: 82%.

Step 3: Preparation of tert-Butyl 2-(3-(6-hydroxypyridazin-3-yl)-5-methoxy-2-methyl-1H-indol-1-yl)acetate, Intermediate 35. The title compound was prepared according to the procedure of intermediate 1; Yield: 56%.

Step 4: Preparation of 2-(3-(1-Benzyl-6-oxo-1,6-dihydropyridazin-3-yl)-5-methoxy-2-methyl-1H-indol-1-yl)acetic acid (18). The title compound was prepared according to the procedure of Example 1; Yield: 24%.

### Example 19

Preparation of 2-(3-(3-Isopropyl-4-oxo-3,4-dihydrophthalazin-1-yl)-5-methoxy-2-methyl-1H-indol-1-yl)acetic acid (19). The title compound was prepared according to the procedure of Example 1; Yield: 30%.

### Example 20

Step 1: Preparation of Methyl 2-(5-fluoro-2-methyl-1H-indol-1-yl)acetate, Intermediate 36. In a 500 mL 2-necked round-bottomed flask, 5-fluoro-2-methylindole (5.00 g) was taken up 100 mL anhydrous DMF, under nitrogen. NaH (1.69 g of a 60 wt % suspension in mineral oil, 1.01 g, 42.2 mmol) was added in small aliquots, and the mixture allowed to stir at room temperature for 30 minutes. Methyl bromoacetate (3.9 mL, 6.5 g, 42 mmol) was added all at once by syringe, and the reaction was allowed to stir overnight. It was then quenched by addition of 20 mL brine, via syringe, and partitioned between 400 mL each ethyl acetate and brine. The aqueous layer was extracted with additional ethyl acetate (2×), and the combined organic extracts washed with brine (3×), dried over anhydrous magnesium sulfate, filtered, evaporated, and purified by flash chromatography over silica gel (2-20% ethyl acetate in hexanes) to give a white solid that gradually turned pink over several days (5.39 g, 69% yield): ¹H NMR (CHLOROFORM-d) δ 7.18 (dd, J=9.6, 2.5 Hz, 1H), 7.08 (dd, J=8.8, 4.3 Hz, 1H), 6.89 (td, J=9.1, 2.5 Hz, 1H), 6.28 (t, J=0.8 Hz, 1H), 4.78 (s, 2H), 3.76 (s, 3H), 2.40 (d, J=1.0 Hz, 3H).

Step 2: Preparation of Methyl 2-(5-fluoro-3-((6-methoxypyridin-3-yl)methyl)-2-methyl-1H-indol-1-yl)acetate, Intermediate 37A. Intermediate 36 (1.00 g, 4.52 mmol) and 6-methoxy-3-pyridinecarboxaldehyde (0.620 g, 4.52 mmol) were taken up in 45 mL anhydrous dichloroethane in a 250 mL 2-necked round-bottomed flask with a condenser, under nitrogen. The solution was cooled to 0° C., and triethylsilane (2.0 mL, 1.5 g, 13 mmol) and trifluoroacetic acid (0.70 mL, 1.0 g, 9.0 mmol) were added via syringe. Stirring was continued for 10 minutes, and the ice bath was then removed and the reaction mixture heated to reflux until LC-MS analysis showed that it was complete. It was then cooled slightly, and partitioned between 45 mL dichloroethane and 25 mL saturated sodium bicarbonate. The organic layer was washed with water and brine, dried over anhydrous magnesium sulfate, filtered, evaporated, and purified by flash chromatography over silica gel (5-40% ethyl acetate in hexanes), to give a white solid (0.861 g, 56% yield): ¹H NMR (DMSO-d₆) δ 8.07 (d, J=2.3 Hz, 1H), 7.46 (dd, J=8.6, 2.5 Hz, 1H), 7.35 (dd, J=8.8, 4.3 Hz, 1H), 7.18 (dd, J=9.9, 2.5 Hz, 1H), 6.87 (td, J=9.2, 2.5 Hz, 1H), 6.69 (d, J=9.1 Hz, 1H), 5.09 (s, 2H), 3.94 (s, 2H), 3.78 (s, 3H), 3.68 (s, 3H), 2.32 (s, 3H).

Step 3: Preparation of Methyl 2-(3-((1-(2,4-difluorobenzyl)-6-oxo-1,6-dihydropyridin-3-yl)methyl)-5-fluoro-2-methyl-1H-indol-1-yl)acetate, Intermediate 37. Intermediate 37 A (0.326 g, 0.954 mmol) and sodium iodide (0.286 g, 1.91 mmol) were taken up in 10 mL anhydrous acetonitrile, and 2,4-difluorobenzyl bromide (0.25 mL, 0.40 g, 1.9 mmol) was added. The mixture was refluxed overnight, then poured into a mixture of 50 mL each brine and 5% sodium thiosulfate and extracted into ethyl acetate (2×). The combined organic extracts were washed with brine, dried over anhydrous magnesium sulfate, filtered, evaporated, and purified by flash chromatography over silica gel (5-40% ethyl acetate in dichloromethane) to give pure product (0.233 g, 56% yield): ¹H NMR (CHLOROFORM-d) δ 7.40 (td, J=8.5, 6.4 Hz, 1H), 7.19 (dd, J=9.3, 2.5 Hz, 1H), 7.09 (dd, J=8.7, 4.2 Hz, 1H), 7.03 (s, 1H), 6.87-6.97 (m, 2H), 6.74-6.85 (m, 2H), 6.50 (d, J=9.3 Hz, 1H), 5.03 (s, 2H), 4.80 (s, 2H), 3.73-3.78 (m, 5H), 2.31 (s, 3H).

Step 4: Preparation of 2-(3-((1-(2,4-Difluorobenzyl)-6-oxo-1,6-dihydropyridin-3-yl)methyl)-5-fluoro-2-methyl-1H-indol-1-yl)acetic acid (20). Intermediate 37 (0.233 g, 0.513 mmol) was taken up in 15 mL methanol and 5 mL tetrahydrofuran. A solution of lithium hydroxide monohydrate (0.430 g, 10.3 mmol) in 5 mL water was added, and the reaction stirred at room temperature for 55 minutes. The reaction mixture was then acidified with concentrated hydrochloric acid, extracted into ethyl acetate (3×), washed with brine, dried over anhydrous magnesium sulfate, filtered, evaporated, and purified by preparative HPLC (water/acetonitrile with 0.1% formic acid). Lyophilization gave a fluffy white solid (0.100 g, 44% yield): ¹H NMR (DMSO-d₆) δ 7.63 (d, J=2.5 Hz, 1H), 7.16-7.29 (m, 4H), 7.13 (dd, J=9.9, 2.5 Hz, 1H), 6.98-7.06 (m, J=8.5, 8.5, 2.6, 0.9 Hz, 1H), 6.82 (td, J=9.2, 2.5 Hz, 1H), 6.31 (d, J=9.3 Hz, 1H), 5.05 (s, 2H), 4.70 (s, 2H), 3.72 (s, 2H), 2.29 (s, 3H).

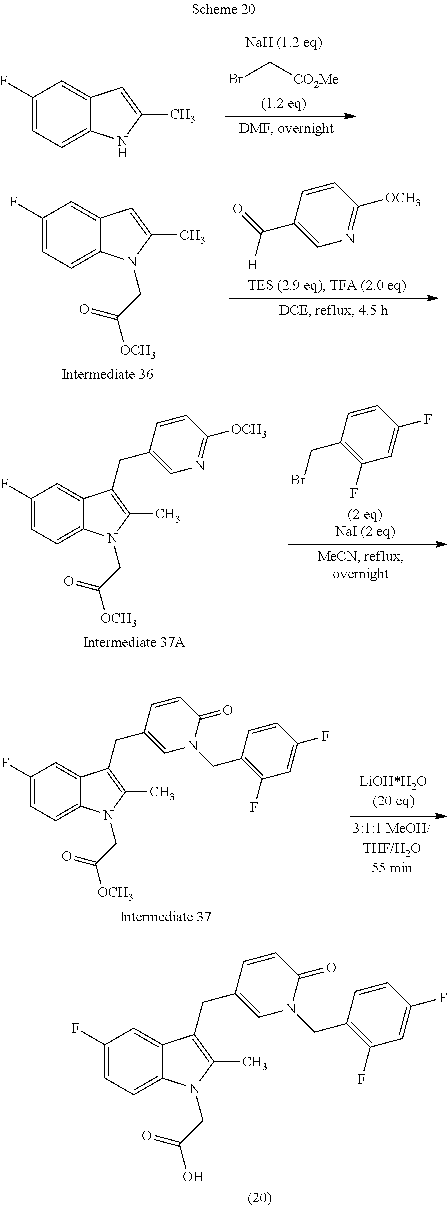

### Example 21

Step 1: Preparation of 6-Oxo-1,6-dihydropyridine-3-carbaldehyde, Intermediate 38. In a flame-dried 250 mL 2-necked round-bottomed flask fitted with a condenser, 6-methoxy-3-pyridinecarboxaldehyde (3.43 g, 24.9 mmol) was taken up in 30 mL anhydrous dichloromethane, under nitrogen. Iodotrimethylsilane (5.0 g, 25 mmol) was added by syringe. The mixture was stirred at room temperature for 2.5 hours, then refluxed for 1.5 hours. After cooling to room temperature, 4.1 mL methanol was added by syringe. The solvent was evaporated, and the residue purified by flash chromatography over silica gel (7-60% acetone in dichloromethane) to give pure product (2.67 g, 87% yield).

Step 2: Preparation of Methyl 2-(2-methyl-1H-indol-1-yl)acetate, Intermediate 38A. The procedure described above for intermediate 36 was followed, reacting 2-methylindole (5.00 g, 38.1 mmol) with NaH (1.83 g of a 60 wt % mineral oil suspension, 1.10 g, 45.7 mmol) and methyl bromoacetate (4.2 mL, 7.0 g, 46 mmol). A viscous yellowish oil that gradually solidified upon standing was obtained (5.06 g, 65% yield).

Step 3: Preparation of Methyl 2-(2-methyl-3-((6-oxo-1,6-dihydropyridin-3-yl)methyl)-1H-indol-1-yl)acetate, Intermediate 38B. The procedure described above for intermediate 37A was followed, reacting intermediate 38A (1.01 g, 4.97 mmol) with intermediate 38 (0.61 g, 4.97 mmol) in the presence of triethylsilane (2.2 mL, 1.6 g, 14 mmol) and trifluoroacetic acid (0.77 mL, 1.1 g, 9.9 mmol). Flash chromatography over silica gel (12-100% acetone in dichloromethane) gave a pink solid (0.943 g, 61% yield).

Step 4: Preparation of Methyl 2-(3-((1-(2,4-difluorobenzyl)-6-oxo-1,6-dihydro-pyridin-3-yl)methyl)-2-methyl-1H-indol-1-yl)acetate, Intermediate 39. Intermediate 38B (0.309 g, 0.994 mmol) was taken up in 15 mL DMF, and potassium carbonate (0.481 g, 3.48 mmol) and 2,4-difluorobenzyl bromide (0.38 mL, 0.62 g, 3.0 mmol) were added. The mixture was heated at 100° C. for 1 hour, until LC-MS analysis showed complete consumption of starting material. It was then poured into 150 mL ice water, extracted into ethyl acetate (2×), washed with brine (3×), dried over anhydrous magnesium sulfate, filtered, evaporated and purified by flash chromatography over silica gel (5-40% ethyl acetate in dichloromethane) to give pure product (0.307 g, 73% yield).

Step 5: Preparation of 2-(3-((1-(2,4-Difluorobenzyl)-6-oxo-1,6-dihydropyridin-3-yl)methyl)-2-methyl-1H-indol-1-yl)acetic acid (21). The procedure described above for 20 was followed, reacting intermediate 39 (0.307 g, 0.702 mmol) with lithium hydroxide monohydrate (0.587 g, 14.0 mmol). Fluffy, peach-colored solid (85 mg, 29% yield): ¹H NMR (DMSO-d₆) δ 12.99 (s, 1H), 7.63 (d, J=1.3 Hz, 1H), 7.39 (d, J=7.6 Hz, 1H), 7.32 (d, J=8.1 Hz, 1H), 7.15-7.30 (m, 3H), 7.00-7.08 (m, 2H), 6.90-6.98 (m, 1H), 6.30 (d, J=9.3 Hz, 1H), 5.05 (s, 2H), 4.93 (s, 2H), 3.76 (s, 2H), 2.31 (s, 3H).

### Example 22

Step 1: Preparation of Methyl 2-(3-((1-isopropyl-6-oxo-1,6-dihydropyridin-3-yl)methyl-2-methyl-1H-indol-1-yl)acetate, Intermediate 40 and Methyl 2-(3-((6-isopropoxypyridin-3-yl)methyl)-2-methyl-1H-indol-1-yl)acetate, intermediate 41. The procedure described above for intermediate 39 was followed, reacting intermediate 38B (0.559 g, 1.80 mmol) with potassium carbonate (0.871 g, 6.30 mmol) and 2-bromopropane (0.51 mL, 0.66 g, 5.4 mmol). Pure intermediate 40 was isolated (82 mg, 13% yield. Intermediate 41 was isolated as side-product (0.295 g, 47% yield).

Step 2: Preparation of 2-(3-((1-Isopropyl-6-oxo-1,6-dihydropyridin-3-yl)methyl-2-methyl-1H-indol-1-yl)acetic acid (22). The procedure described above for 20 was followed, reacting intermediate 40 (82 mg, 0.23 mmol) with lithium hydroxide monohydrate (0.194 g, 4.63 mmol). Fluffy, off-white solid (26 mg, 34% yield).

### Example 23

Step 1: Preparation of Methyl 2-(5-fluoro-2-methyl-346-oxo-1-(2,4,5-trifluorobenzyl)-1,6-dihydropyridin-3-yl)methyl)-1H-indol-1-yl)acetate, Intermediate 42. The procedure described above for intermediate 37 was followed, reacting intermediate 37A (0.326 g, 0.954 mmol) with 2,4,5-trifluorobenzyl bromide (0.430 g, 1.91 mmol) and NaI (0.286 g, 1.91 mmol). 0.300 g, 69% yield.

Step 2: Preparation of 2-(5-Fluoro-2-methyl-3-((6-oxo-1-(2,4,5-trifluorobenzyl)-1,6-dihydro-pyridin-3-yl)methyl)-1H-indol-1-yl)acetic acid (23). The procedure described above for 20 was followed, reacting intermediate 42 (0.300 g, 0.634 mmol) with lithium hydroxide monohydrate (0.530 g, 12.7 mmol). Fluffy, off-white solid (0.141 g, 49% yield): ¹H NMR (DMSO-d₆) δ 7.65 (s, 1H), 7.54 (td, J=10.2, 6.8 Hz, 1H), 7.32 (dd, J=8.8, 4.5 Hz, 1H), 7.18-7.28 (m, 2H), 7.15 (dd, J=10.0, 2.4 Hz, 1H), 6.85 (td, J=9.2, 2.5 Hz, 1H), 6.32 (d, J=9.3 Hz, 1H), 5.04 (s, 2H), 4.89 (s, 2H), 3.74 (s, 2H), 2.31 (s, 3H).

### Example 24

Step 1: Preparation of Methyl 2-(2-methyl-3-((6-oxo-1-(2,4,5-trifluorobenzyl)-1,6-dihydropyridin-3-yl)methyl-1H-indol-1-yl)acetate, Intermediate 43. The procedure described above for intermediate 39 was followed, reacting intermediate 38B (0.309 g, 0.994 mmol) with potassium carbonate (0.481 g, 3.48 mmol) and 2,4,5-trifluorobenzyl bromide (0.671 g, 2.98 mmol). Pure product was isolated (0.319 g, 73% yield).

Step 2: Preparation of 2-(2-Methyl-3-((6-oxo-1-(2,4,5-trifluorobenzyl)-1,6-dihydropyridin-3-yl)methyl-1H-indol-1-yl)acetic acid (24). The procedure described above for 20 was followed, reacting intermediate 43 (0.319 g, 0.703 mmol) with lithium hydroxide monohydrate (0.590 g, 14.1 mmol). Two successive preparative HPLC purifications (water/acetonitrile with 0.1% formic acid), followed by lyophilization, gave a fluffy, pale yellow solid (63 mg, 20% yield).

### Example 25

Step 1: Preparation of Methyl 2-(3-((1-(2,5-difluorobenzyl)-6-oxo-1,6-dihydropyridin-3-yl)-methyl)2-methyl-1H-indol-1-yl)acetate, Intermediate 44. The procedure described above for intermediate 39 was followed, reacting intermediate 38B (0.309 g, 0.994 mmol) with potassium carbonate (0.481 g, 3.48 mmol) and 2,5-difluorobenzyl bromide (0.38 mL, 0.62 g, 3.0 mmol). Pure product was isolated (0.289 g, 69% yield).

Step 2: Preparation of 2-(3-((1-(2,5-Difluorobenzyl)-6-oxo-1,6-dihydropyridin-3-yl)methyl)-2-methyl-1H-indol-1-yl)acetic acid. (25). The procedure described above for 20 was followed, reacting intermediate 44 (0.289 g, 0.662 mmol) with lithium hydroxide monohydrate (0.555 g, 13.2 mmol). 0.131 g, 47% yield: ¹H NMR (DMSO-d₆) δ 13.11 (s, 1H), 7.68 (s, 1H), 7.40 (d, J=7.6 Hz, 1H), 7.25-7.34 (m, 2H), 7.16-7.24 (m, 2H), 7.03 (td, J=7.6, 1.0 Hz, 1H), 6.84-6.96 (m, 2H), 6.32 (d, J=9.3 Hz, 1H), 5.07 (s, 2H), 4.90 (s, 2H), 3.77 (s, 2H), 2.32 (s, 3H).

### Example 26

Step 1: Preparation of Methyl 2-(3-((1-(2,5-difluorobenzyl)-6-oxo-1,6-dihydro-pyridin-3-yl)methyl)-5-fluoro-2-methyl-1H-indol-1-yl)acetate, Intermediate 45. The procedure described above for intermediate 37 was followed, reacting intermediate 37A (0.324 g, 0.946 mmol) with 2,5-difluorobenzyl bromide (0.24 mL, 0.39 g, 1.9 mmol) and sodium iodide (0.284 g, 1.89 mmol). 0.294 g, 68% yield.

Step 2: Preparation of 2-(3-((1-(2,5-Difluorobenzyl)-6-oxo-1,6-dihydropyridin-3-yl)methyl)-5-fluoro-2-methyl-1H-indol-1-yl)acetic acid. (26). The procedure described above for 20 was followed, reacting intermediate 45 (0.294 g, 0.647 mmol) with lithium hydroxide monohydrate (0.543 g, 12.9 mmol). Ivory-colored powder (0.105 g, 37% yield): ¹H NMR (DMSO-d₆) δ 13.27 (s, 1H), 7.69 (d, J=1.8 Hz, 1H), 7.14-7.36 (m, 5H), 6.80-6.91 (m, 2H), 6.33 (d, J=9.3 Hz, 1H), 5.07 (s, 2H), 4.90 (s, 2H), 3.74 (s, 2H), 2.31 (s, 3H).

### Example 27

Step 1: Preparation of Methyl 2-(5-fluoro-2-methyl-3-((6-oxo-1,6-dihydropyridin-3-yl)methyl)-1H-indol-1-yl)acetate, Intermediate 46A. The procedure described above for intermediate 37A was followed, reacting intermediate 37A (2.52 g, 11.4 mmol) with intermediate 38 (1.40 g, 11.4 mmol) in the presence of triethylsilane (5.1 mL, 3.7 g, 32 mmol) and trifluoroacetic acid (1.8 mL, 2.6 g, 23 mmol). Due to the relative insolubility of the product, the work-up was modified as follows: the cooled reaction mixture was partitioned between saturated sodium bicarbonate and dichloromethane, and the organic layer washed with water and evaporated (product was already starting to precipitate out, so drying over magnesium sulfate and filtering was concluded to be a bad idea). The crude product was purified by recrystallization from acetonitrile, collecting two crops of pure product (2.82 g, 76% yield).

Step 2: Preparation of Methyl 2-(5-fluoro-3-((6-isopropoxypyridin-3-yl)methyl)-2-methyl-1H-indol-1-yl)acetate, Intermediate 46 and Methyl 2-(5-fluoro-3-((1-isopropyl-6-oxo-1,6-dihydro-pyridin-3-yl)methyl-2-methyl-1H-indol-1-yl)acetate, Intermediate 47. The procedure described above for intermediate 39 was followed, reacting intermediate 46A (2.14 g, 6.53 mmol) with potassium carbonate (3.16 g, 22.9 mmol) and 2-bromopropane (1.8 mL, 2.4 g, 20 mmol). Pure intermediate 46 was isolated (1.37 g, 57% yield): ¹H NMR (DMSO-d₆) δ 8.04 (d, J=2.5 Hz, 1H), 7.43 (dd, J=8.6, 2.5 Hz, 1H), 7.35 (dd, J=8.8, 4.5 Hz, 1H), 7.19 (dd, J=9.9, 2.5 Hz, 1H), 6.87 (td, J=9.2, 2.5 Hz, 1H), 6.60 (d, J=8.6 Hz, 1H), 5.10-5.22 (m, 1H), 5.09 (s, 2H), 3.92 (s, 2H), 3.68 (s, 3H), 2.33 (s, 3H), 1.23 (d, J=6.3 Hz, 6H). Pure intermediate 47 was isolated methyl 2-(5-fluoro-3-((1-isopropyl-6-oxo-1,6-dihydropyridin-3-yl)methyl-2-methyl-1H-indol-1-yl)acetate 0.175 g (7.2%).

Step 3: Preparation of 2-(5-fluoro-3-((1-isopropyl-6-oxo-1,6-dihydropyridin-3-yl)methyl)-2-methyl-1H-indol-1-yl)acetic acid (27) The procedure described above for 20 was followed, reacting intermediate 47 (0.175 g, 0.473 mmol) with lithium hydroxide monohydrate (0.397 g, 9.46 mmol). Fluffy, white solid (52 mg, 31% yield): ¹H NMR (DMSO-d₆) δ 7.66 (d, J=2.3 Hz, 1H), 7.31 (dd, J=8.8, 4.5 Hz, 1H), 7.24 (dd, J=10.1, 2.5 Hz, 1H), 7.10 (dd, J=9.3, 2.5 Hz, 1H), 6.84 (td, J=9.2, 2.5 Hz, 1H), 6.24 (d, J=9.3 Hz, 1H), 4.96-5.11 (m, J=13.7, 6.9, 6.9, 6.9, 6.9 Hz, 1H), 4.84 (s, 2H), 3.76 (s, 2H), 2.32 (s, 3H), 1.25 (d, J=6.8 Hz, 6H).

### Example 28

Step 1: Preparation of Methyl 2-(3-((1-(2,3-difluorobenzyl)-6-oxo-1,6-dihydro-pyridin-3-yl)methyl)-5-fluoro-2-methyl-1H-indol-1-yl)acetate, Intermediate 48. The procedure described above for intermediate 39 was followed, reacting intermediate 46A (0.373 g, 1.14 mmol) with potassium carbonate (0.551 g, 3.99 mmol) and 2,3-difluorobenzyl bromide (0.43 mL, 0.71 g, 3.4 mmol). Pure product was isolated (0.292 g, 56% yield).

Step 2: Preparation of 2-(3-((1-(2,3-Difluorobenzyl)-6-oxo-1,6-dihydropyridin-3-yl)methyl)-5-fluoro-2-methyl-1H-indol-1-yl)acetic acid (28). The procedure described above for 20 was followed, reacting intermediate 48 (0.292 g, 0.643 mmol) with lithium hydroxide monohydrate (0.539 g, 12.9 mmol). The crude product was purified by recrystallization from acetonitrile/ethanol to give fine pale pink needles (0.174 g, 61% yield): ¹H NMR (DMSO-d₆) δ 12.99 (br. s., 1H), 7.69 (d, J=2.0 Hz, 1H), 7.30-7.41 (m, 2H), 7.09-7.25 (m, 3H), 6.82-6.93 (m, 2H), 6.33 (d, J=9.3 Hz, 1H), 5.13 (s, 2H), 4.95 (s, 2H), 3.74 (s, 2H), 2.31 (s, 3H).

### Example 29

Step 1: Preparation of Methyl 2-(3-((1-(2,3-difluorobenzyl)-6-oxo-1,6-dihydro-pyridin-3-yl)methyl)-2-methyl-1H-indol-1-yl)acetate, Intermediate 49. The procedure described above for intermediate 39 was followed, reacting intermediate 38B (0.367 g, 1.18 mmol) with potassium carbonate (0.571 g, 4.13 mmol) and 2,3-difluorobenzyl bromide (0.45 mL, 0.73 g, 3.5 mmol). Pure product was isolated (0.277 g, 54% yield).

Step 2: Preparation of 2-(3-(1-(2,3-Difluorobenzyl)-6-oxo-1,6-dihydropyridin-3-yl)methyl)-2-methyl-1H-indol-1-yl)acetic acid (29). The procedure described above for 20 was followed, reacting intermediate 49 (0.277 g, 0.634 mmol) with lithium hydroxide monohydrate (0.532 g, 12.7 mmol). Two successive preparative HPLC purifications, followed by lyophilization, gave a fluffy yellow solid (70 mg, 26% yield): ¹H NMR (DMSO-d₆) δ 7.68 (d, J=1.8 Hz, 1H), 7.33-7.42 (m, 2H), 7.31 (d, J=8.1 Hz, 1H), 7.22 (dd, J=9.3, 2.5 Hz, 1H), 7.11-7.18 (m, J=8.1, 8.1, 5.1, 1.5 Hz, 1H), 7.03 (t, J=7.7 Hz, 1H), 6.86-6.95 (m, 2H), 6.31 (d, J=9.3 Hz, 1H), 5.13 (s, 2H), 4.88 (s, 2H), 3.77 (s, 2H), 2.31 (s, 3H).

### Example 30

Step 1: Preparation of Methyl 2-(3-((1-(3,4-difluorobenzyl)-6-oxo-1,6-dihydro-pyridin-3-yl)methyl)-2-methyl-1H-indol-1-yl)acetate, Intermediate 50. The procedure described above for intermediate 39 was followed, reacting intermediate 38B (0.367 g, 1.18 mmol) with potassium carbonate (0.571 g, 4.13 mmol) and 3,4-difluorobenzyl bromide (0.45 mL, 0.73 g, 3.5 mmol). Pure product was isolated (0.371 g, 72% yield).

Step 2: Preparation of 2-(3-((1-(3,4-Difluorobenzyl)-6-oxo-1,6-dihydropyridin-3-yl)methyl)-2-methyl-1H-indol-1-yl)acetic acid (30). The procedure described above for 20 was followed, reacting intermediate 50 (0.371 g, 0.849 mmol) with lithium hydroxide monohydrate (0.713 g, 17.0 mmol). The crude product was purified by recrystallization from acetonitrile to give fine, fluffy pink-tinged white crystals (0.237 g, 66%yield). ¹H NMR (DMSO-d₆) δ 12.96 (s, 1H), 7.76 (d, J=2.0 Hz, 1H), 7.28-7.45 (m, 4H), 7.18 (dd, J=9.3, 2.5 Hz, 1H), 7.10-7.16 (m, 1H), 7.03 (ddd, J=8.1, 7.1, 1.0 Hz, 1H), 6.90-6.96 (m, 1H), 6.31 (d, J=9.3 Hz, 1H), 5.02 (s, 2H), 4.93 (s, 2H), 3.75 (s, 2H), 2.32 (s, 3H).

### Example 31

Step 1: Preparation of Methyl 2-(3-((1-(2-fluorobenzyl)-6-oxo-1,6-dihydro-pyridin-3-yl)methyl)-2-methyl-1H-indol-1-yl)acetate, Intermediate 51. The procedure described above for intermediate 39 was followed, reacting intermediate 38B (0.365 g, 1.18 mmol) with potassium carbonate (0.571 g, 4.13 mmol) and 2-fluorobenzyl bromide (0.43 mL, 0.67 g, 3.5 mmol). Pure product was isolated (0.336 g, 68% yield).

Step 2: Preparation of 2-(3-((1-(2-Fluorobenzyl)-6-oxo-1,6-dihydropyridin-3-yl)methyl)-2-methyl-1H-indol-1-yl)acetic acid (31). The procedure described above for 20 was followed, reacting intermediate 51 (0.336 g, 0.802 mmol) with lithium hydroxide monohydrate (0.673 g, 16.0 mmol). The crude product was purified by recrystallization from acetonitrile/ethanol, to give an off-white powder (0.213 g, 66% yield): ¹H NMR (DMSO-d₆) δ 12.96 (br. s., 1H), 7.65 (d, J=2.3 Hz, 1H), 7.29-7.43 (m, 3H), 7.18-7.25 (m, 2H), 7.11-7.17 (m, 1H), 7.00-7.10 (m, 2H), 6.90-6.97 (m, 1H), 6.31 (d, J=9.3 Hz, 1H), 5.09 (s, 2H), 4.93 (s, 2H), 3.77 (s, 2H), 2.31 (s, 3H).

### Example 32

Step 1: Preparation of Methyl 2-(5-fluoro-3-((1-(2-fluorobenzyl)-6-oxo-1,6-dihydropyridin-3-yl)methyl)-2-methyl-1H-indol-1-yl)acetate, Intermediate 52. The procedure described above for intermediate 39 was followed, reacting intermediate 46A (0.370 g, 1.13 mmol) with potassium carbonate (0.547 g, 3.96 mmol) and 2-fluorobenzyl bromide (0.41 mL, 0.64 g, 3.4 mmol). Pure product was isolated (0.326 g, 66% yield).

Step 2: Preparation of 2-(5-Fluoro-3-((1-(2-fluorobenzyl)-6-oxo-1,6-dihydropyridin-3-yl)methyl)-2-methyl-1H-indol-1-yl)acetic acid (32). The procedure described above for 20 was followed, reacting intermediate 52 (0.326 g, 0.746 mmol) with lithium hydroxide monohydrate (0.626 g, 14.9 mmol). The crude product was purified by recrystallization from acetonitrile/ethanol to give an off-white powder (0.185 g, 39% yield): ¹H NMR (DMSO-d₆) δ 12.99 (br. s., 1H), 7.67 (d, J=2.0 Hz, 1H), 7.30-7.38 (m, 2H), 7.16-7.24 (m, 3H), 7.05-7.16 (m, 2H), 6.86 (td, J=9.1, 2.5 Hz, 1H), 6.32 (d, J=9.3 Hz, 1H), 5.09 (s, 2H), 4.94 (s, 2H), 3.74 (s, 2H), 2.30 (s, 3H).

### Example 33

Step 1: Preparation of Methyl 2-(3-((1-(3,4-difluorobenzyl)-6-oxo-1,6-dihydro-pyridin-3-yl)methyl)-5-fluoro-2-methyl-1H-indol-1-yl)acetate, Intermediate 53. The procedure described above for intermediate 39 was followed, reacting intermediate 46A (0.408 g, 1.24 mmol) with potassium carbonate (0.600 g, 4.34 mmol) and 3,4-difluorobenzyl bromide (0.48 mL, 0.77 g, 3.7 mmol). Pure product was isolated (0.363 g, 64% yield).

Step 2: Preparation of 2-(3-((1-(3,4-difluorobenzyl)-6-oxo-1,6-dihydropyridin-3-yl)methyl)-5-fluoro-2-methyl-1H-indol-1-yl)acetic acid (33). The procedure described above for 20 was followed, reacting intermediate 53 (0.444 g, 0.977 mmol) with lithium hydroxide monohydrate (0.820 g, 19.5 mmol). The crude product was purified by recrystallization from acetonitrile to give fluffy white crystals (0.302 g, 70% yield): ¹H NMR (DMSO-d₆) δ 13.00 (br. s., 1H), 7.77 (d, J=2.5 Hz, 1H), 7.31-7.42 (m, 3H), 7.11-7.21 (m, 3H), 6.86 (td, J=9.1, 2.5 Hz, 1H), 6.32 (d, J=9.3 Hz, 1H), 5.03 (s, 2H), 4.94 (s, 2H), 3.72 (s, 2H), 2.31 (s, 3H).

### Example 34

Step 1: Preparation of Methyl 2-(3-((1-(3-fluorobenzyl)-6-oxo-1,6-dihydro-pyridin-3-yl)methyl)-2-methyl-1H-indol-1-yl)acetate, Intermediate 54. The procedure described above for intermediate 39 was followed, reacting intermediate 38B (0.365 g, 1.18 mmol) with potassium carbonate (0.571 g, 4.13 mmol) and 3-fluorobenzyl bromide (0.43 mL, 0.67 g, 3.5 mmol). Pure product was isolated (0.356 g, 72% yield).

Step 2: Preparation of 2-(3-((1-(3-Fluorobenzyl)-6-oxo-1,6-dihydropyridin-3-yl)methyl)-2-methyl-1H-indol-1-yl)acetic acid (34). The procedure described above for 20 was followed, reacting intermediate 54 (0.356 g, 0.851 mmol) with lithium hydroxide monohydrate (0.714 g, 17.0 mmol). The crude product was purified by recrystallization from acetonitrile/ethanol to give an off-white powder (0.133 g, 39% yield): ¹H NMR (DMSO-d₆) δ 12.93 (br. s., 1H), 7.74 (d, J=2.0 Hz, 1H), 7.34-7.42 (m, 2H), 7.32 (d, J=8.1 Hz, 1H), 7.19 (dd, J=9.3, 2.5 Hz, 1H), 7.06-7.15 (m, 3H), 7.03 (ddd, J=8.1, 7.1, 1.0 Hz, 1H), 6.92 (ddd, J=7.8, 7.0, 0.9 Hz, 1H), 6.32 (d, J=9.3 Hz, 1H), 5.06 (s, 2H), 4.92 (s, 2H), 3.76 (s, 2H), 2.32 (s, 3H).

### Example 35

Step 1: Preparation of Methyl 2-(3-((1-(3,5-difluorobenzyl)-6-oxo-1,6-dihydro-pyridin-3-yl)methyl)-2-methyl-1H-indol-1-yl)acetate, Intermediate 55. The procedure described above for intermediate 39 was followed, reacting intermediate 38B (0.270 g, 0.870 mmol) with potassium carbonate (0.421 g, 3.05 mmol) and 3,5-difluorobenzyl bromide (0.34 mL, 0.54 g, 2.6 mmol). Pure product was isolated (0.232 g, 61% yield).

Step 2: Preparation of 2-(3-((1-(3,5-Difluorobenzyl)-6-oxo-1,6-dihydropyridin-3-yl)methyl)-2-methyl-1H-indol-1-yl)acetic acid (35). The procedure described above for 20 was followed, reacting intermediate 55 (0.232 g, 0.532 mmol) with lithium hydroxide monohydrate (0.446 g, 10.6 mmol). The crude product was purified by recrystallization from acetonitrile/ethanol to give a white powder (0.128 g, 57% yield): ¹H NMR (DMSO-d₆) δ 12.96 (br. s., 1H), 7.78 (s, 1H), 7.41 (d, J=7.8 Hz, 1H), 7.32 (d, J=8.3 Hz, 1H), 7.13-7.22 (m, 2H), 7.03 (t, J=7.7 Hz, 1H), 6.98 (d, J=6.8 Hz, 2H), 6.88-6.94 (m, 1H), 6.33 (d, J=9.3 Hz, 1H), 5.06 (s, 2H), 4.93 (s, 2H), 3.76 (s, 2H), 2.33 (s, 3H).

### Example 36

Step 1: Preparation of Methyl 2-(3-((1-(3,5-difluorobenzyl)-6-oxo-1,6-dihydro-pyridin-3-yl)methyl)-5-fluoro-2-methyl-1H-indol-1-yl)acetate, Intermediate 56. The procedure described above for intermediate 37 was followed, reacting intermediate 37B (0.288 g, 0.841 mmol) with 3,5-difluorobenzyl bromide (0.22 mL, 0.35 g, 1.7 mmol) and sodium iodide (0.252 g, 1.68 mmol). 0.288 g, 75% yield.

Step 2: Preparation of 2-(3-((1-(3,5-Difluorobenzyl)-6-oxo-1,6-dihydropyridin-3-yl)methyl)-5-fluoro-2-methyl-1H-indol-1-yl)acetic acid (36). The procedure described above for 20 was followed, reacting intermediate 56 (0.288 g, 0.634 mmol) with lithium hydroxide monohydrate (0.532 g, 12.7 mmol). The crude product was purified by recrystallization from acetonitrile/ethanol to give a white solid (0.203 g, 73% yield): ¹H NMR (DMSO-d₆) δ 13.00 (br. s., 1H), 7.79 (d, J=2.3 Hz, 1H), 7.34 (dd, J=9.0, 4.4 Hz, 1H), 7.12-7.23 (m, 3H), 6.93-7.00 (m, 2H), 6.86 (td, J=9.2, 2.5 Hz, 1H), 6.34 (d, J=9.3 Hz, 1H), 5.06 (s, 2H), 4.94 (s, 2H), 3.73 (s, 2H), 2.32 (s, 3H).

### Example 37

Step 1: Preparation of Methyl 2-(((6-methoxypyridin-3-yl)methyl)-2-methyl-1H-indol-1-yl)-acetate, Intermediate 57A. The procedure described above for intermediate 37B was followed, reacting intermediate 38A (6.64 g, 32.7 mmol) with 6-methoxy-3-pyridinecarboxaldehyde (4.48 g, 32.7 mmol) in the presence of triethylsilane (14.6 mL, 10.6 g, 91.6 mmol) and trifluoroacetic acid (5.0 mL, 7.5 g, 65.4 mmol). Flash chromatography over silica gel (5-40% ethyl acetate in hexanes) gave a white solid (8.77 g, 83% yield).

Step 2: Preparation of Methyl 2-(3-((1-(2,6-difluorobenzyl)-6-oxo-1,6-dihydro-pyridin-3-yl)-methyl)-2-methyl-1H-indol-1-yl)acetate, Intermediate 57. The procedure described above for intermediate 37 was followed, reacting intermediate 57A (0.284 g, 0.876 mmol) with 2,6-difluorobenzyl bromide (0.363 g, 1.75 mmol) and NaI (0.262 g, 1.75 mmol). 0.255 g, 67% yield.

Step 3: Preparation of 2-(3-((1-(2,6-Difluorobenzyl)-6-oxo-1,6-dihydropyridin-3-yl)methyl)-2-methyl-1H-indol-1-yl)acetic acid (37). The procedure described above for 20 was followed, reacting intermediate 57 (0.255 g, 0.584 mmol) with lithium hydroxide monohydrate (0.490 g, 11.7 mmol). The crude product was purified by recrystallization from acetonitrile/ethanol to give a pink-tinged white powder (0.138 g, 56% yield): ¹H NMR (DMSO-d₆) δ 12.96 (br. s., 1H), 7.57 (br. s., 1H), 7.35-7.46 (m, 2H), 7.32 (d, J=8.6 Hz, 1H), 7.17 (d, J=9.3 Hz, 1H), 7.05 (dt, J=14.8, 7.6 Hz, 3H), 6.91-6.98 (m, 1H), 6.23 (d, J=9.1 Hz, 1H), 5.07 (s, 2H), 4.93 (s, 2H), 3.76 (s, 2H), 2.30 (s, 3H).

### Example 38

Step 1: Preparation of Methyl 2-(3-((1-(2,6-difluorobenzyl)-6-oxo-1,6-dihydro-pyridin-3-yl)methyl)-5-fluoro-2-methyl-1H-indol-1-yl)acetate, Intermediate 58. The procedure described above for intermediate 37 was followed, reacting intermediate 37B (0.288 g, 0.84 mmol) with 2,6-difluorobenzyl bromide (0.348 g, 1.68 mmol) and NaI (0.252 g, 1.68 mmol). 0.276 g, 72% yield.

Step 2: Preparation of 2-(3-((1-(2,6-Difluorobenzyl)-6-oxo-1,6-dihydropyridin-3-yl)methyl)-5-fluoro-2-methyl-1H-indol-1-yl)acetic acid (38). The procedure described above for 20 was followed, reacting intermediate 58 (0.276 g, 0.607 mmol) with lithium hydroxide monohydrate (0.510 g, 12.1 mmol). The crude product was purified by recrystallization from acetonitrile/ethanol to give a white powder (0.159 g, 60% yield): ¹H NMR (DMSO-d₆) δ 12.98 (br. s., 1H), 7.56 (s, 1H), 7.40 (tt, J=8.3, 6.6 Hz, 1H), 7.34 (dd, J=8.8, 4.3 Hz, 1H), 7.16 (td, J=9.3, 2.5 Hz, 2H), 7.01-7.09 (m, 2H), 6.86 (td, J=9.2, 2.7 Hz, 1H), 6.25 (d, J=9.3 Hz, 1H), 5.07 (s, 2H), 4.94 (s, 2H), 3.74 (s, 2H), 2.29 (s, 3H).

### Example 39

Step 1: Preparation of Methyl 2-(5-fluoro-3-((1-(3-fluorobenzyl)-6-oxo-1,6-dihydropyridin-3-yl)methyl)-2-methyl-1H-indol-1-yl)acetate, Intermediate 59. The procedure described above for intermediate 37 was followed, reacting intermediate 37B (0.300 g, 0.876 mmol) with 3-fluorobenzyl bromide (0.21 mL, 0.33 g, 1.8 mmol) and sodium iodide (0.262 g, 1.75 mmol). 0.239 g, 63% yield.

Step 2: Preparation of 2-(5-Fluoro-3-((1-(3-fluorobenzyl)-6-oxo-1,6-dihydropyridin-3-yl)methyl)-2-methyl-1H-indol-1-yl)acetic acid. (39). The procedure described above for 20 was followed, reacting intermediate 59 (0.239 g, 0.548 mmol) with lithium hydroxide monohydrate (0.460 g, 11.0 mmol). The crude product was purified by recrystallization from acetonitrile/ethanol to give a white solid (0.158 g, 68% yield): ¹H NMR (DMSO-d₆) δ 7.76 (d, J=2.0 Hz, 1H), 7.31-7.40 (m, 2H), 7.19 (dd, J=9.5, 2.7 Hz, 2H), 7.05-7.14 (m, 3H), 6.86 (td, J=9.2, 2.5 Hz, 1H), 6.33 (d, J=9.1 Hz, 1H), 5.06 (s, 2H), 4.94 (s, 2H), 3.73 (s, 2H), 2.31 (s, 3H).

### Example 40

Step 1: Preparation of Methyl 2-(3-((1-(4-difluorobenzyl)-6-oxo-1,6-dihydro-pyridin-3-yl)methyl)-2-methyl-1H-indol-1-yl)acetate, Intermediate 60. The procedure described above for intermediate 37 was followed, reacting intermediate 57A (0.297 g, 0.915 mmol) with 4-fluorobenzyl bromide (0.22 mL, 0.35 g, 1.8 mmol) and sodium iodide (0.274 g, 1.83 mmol). 0.179 g, 47% yield.

Step 2: Preparation of 2-(3-((1-(4-Fluorobenzyl)-6-oxo-1,6-dihydropyridin-3-yl)methyl)-2-methyl-1H-indol-1-yl)acetic acid (40). The procedure described above for 20 was followed, reacting intermediate 60 (0.179 g, 0.428 mmol) with lithium hydroxide monohydrate (0.360 g, 8.57 mmol). The crude product was purified by recrystallization from acetonitrile to give a mauve powder (72 mg, 42% yield): ¹H NMR (DMSO-d₆) δ 12.94 (br. s., 1H), 7.71 (d, J=2.0 Hz, 1H), 7.38 (d, J=7.6 Hz, 1H), 7.29-7.36 (m, 3H), 7.11-7.21 (m, 3H), 7.03 (ddd, J=8.1, 7.0, 1.3 Hz, 1H), 6.93 (ddd, J=7.9, 7.0, 1.0 Hz, 1H), 6.30 (d, J=9.3 Hz, 1H), 5.02 (s, 2H), 4.92 (s, 2H), 3.75 (s, 2H), 2.30 (s, 3H).

### Example 41

Step 1: Preparation of Methyl 2-(5-fluoro-3-((1-(4-fluorobenzyl)-6-oxo-1,6-dihydropyridin-3-yl)methyl)-2-methyl-1H-indol-1-yl)acetate, Intermediate 61. The procedure described above for intermediate 37 was followed, reacting intermediate 37B (0.300 g, 0.876 mmol) with 4-fluorobenzyl bromide (0.22 mL, 0.33 g, 1.8 mmol) and sodium iodide (0.262 g, 1.75 mmol). 0.207 g, 54% yield.

Step 2: Preparation of 2-(5-Fluoro-3-((1-(4-fluorobenzyl)-6-oxo-1,6-dihydropyridin-3-yl)methyl)-2-methyl-1H-indol-1-yl)acetic acid (41). The procedure described above for 20 was followed, reacting intermediate 61 (0.207 g, 0.474 mmol) with lithium hydroxide monohydrate (0.398 g, 9.48 mmol). The crude product was purified by recrystallization from acetonitrile/ethanol to give a white solid (0.111 g, 55% yield): ¹H NMR (DMSO-d₆) δ 12.97 (br. s., 1H), 7.73 (d, J=2.0 Hz, 1H), 7.30-7.37 (m, 3H), 7.09-7.20 (m, 4H), 6.86 (td, J=9.1, 2.5 Hz, 1H), 6.31 (d, J=9.3 Hz, 1H), 5.03 (s, 2H), 4.93 (s, 2H), 3.72 (s, 2H), 2.30 (s, 3H).

### Example 42

Step 1: Preparation of Methyl 2-(5-chloro-2-methyl-1H-indol-1-yl)acetate, Intermediate 62A. The procedure described above for intermediate 37A was followed, reacting 5-chloro-2-methylindole (5.00 g, 30.2 mmol) with NaH (1.45 g of a 60 wt % mineral oil suspension, 0.87 g, 36.2 mmol) and methyl bromoacetate (3.3 mL, 5.5 g, 36 mmol). A white solid was obtained (3.60 g, 50% yield).

Step 2: Preparation of Methyl 2-(5-chloro-3-((6-methoxypyridin-3-yl)methyl)-1H-indol-1-yl)-acetate, Intermediate 62B. The procedure described above for intermediate 37B was followed, reacting intermediate 62A (3.54 g, 14.9 mmol) with 6-methoxy-3-pyridinecarboxaldehyde (2.04 g, 14.9 mmol) in the presence of triethylsilane (6.7 mL, 4.9 g, 42 mmol) and trifluoroacetic acid (2.3 mL, 3.4 g, 30 mmol). Flash chromatography over silica gel (5-40% ethyl acetate in hexanes) gave a white solid (2.07 g, 39% yield).

Step 3: Preparation of Methyl 2-(5-chloro-3-((1-(2,4-difluorobenzyl)-6-oxo-1,6-dihydropyridin-3-yl)methyl)-2-methyl-1H-indol-1-yl)acetate, Intermediate 62. The procedure described above for intermediate 37 was followed, reacting intermediate 62B (0.291 g, 0.810 mmol) with 2,4-difluorobenzyl bromide (0.21 mL, 0.34 g, 1.6 mmol) and sodium iodide (0.243 g, 1.62 mmol). 0.268 g, 70% yield.

Step 4: Preparation of 2-(5-Chloro-3-((1-(2,4-difluorobenzyl)-6-oxo-1,6-dihydropyridin-3-yl)-methyl)-2-methyl-1H-indol-1-yl)acetic acid. (42). The procedure described above for 20 was followed, reacting intermediate 62 (0.268 g, 0.568 mmol) with lithium hydroxide monohydrate (0.477 g, 11.4 mmol). The crude product was purified by recrystallization from acetonitrile/ethanol to give tiny white crystals (0.170 g, 66% yield): ¹H NMR (DMSO-d₆) δ 13.00 (br. s., 1H), 7.63 (d, J=2.3 Hz, 1H), 7.42 (d, J=2.0 Hz, 1H), 7.38 (d, J=8.6 Hz, 1H), 7.17-7.27 (m, 3H), 6.99-7.06 (m, 2H), 6.32 (d, J=9.3 Hz, 1H), 5.05 (s, 2H), 4.95 (s, 2H), 3.76 (s, 2H), 2.31 (s, 3H).

### Example 43

Step 1: Preparation of Methyl 2-(5-chloro-3-((1-(2,3-difluorobenzyl)-6-oxo-1,6-dihydropyridin-3-yl)methyl)-2-methyl-1H-indol-1-yl)acetate, Intermediate 63. The procedure described above for intermediate 37 was followed, reacting intermediate 62B (0.291 g, 0.810 mmol) with 2,3-difluorobenzyl bromide (0.21 mL, 0.34 g, 1.6 mmol) and NaI (0.243 g, 1.62 mmol). 0.302 g, 79% yield.

Step 2: Preparation of 2-(5-Chloro-3-((1-(2,3-difluorobenzyl)-6-oxo-1,6-dihydropyridin-3-yl)-methyl)-2-methyl-1H-indol-1-yl)acetic acid (43). The procedure described above for 20 was followed, reacting intermediate 63 (0.302 g, 0.642 mmol) with lithium hydroxide monohydrate (0.539 g, 12.8 mmol). The crude product was purified by recrystallization from acetonitrile/ethanol to give fluffy white crystals (0.181 g, 62% yield): ¹H NMR (DMSO-d₆) δ 13.01 (br. s., 1H), 7.69 (d, J=2.5 Hz, 1H), 7.44 (d, J=2.0 Hz, 1H), 7.32-7.41 (m, 2H), 7.20 (dd, J=9.3, 2.8 Hz, 1H), 7.11-7.18 (m, J=8.1, 8.1, 5.1, 1.8 Hz, 1H), 7.03 (dd, J=8.7, 2.1 Hz, 1H), 6.87-6.94 (m, 1H), 6.33 (d, J=9.3 Hz, 1H), 5.13 (s, 2H), 4.95 (s, 2H), 3.77 (s, 2H), 2.32 (s, 3H).

### Example 44

Step 1: Preparation of Methyl 2-(5-chloro-2-methyl-3-((6-oxo-1-(2,4,5-trifluoro-benzyl)-1,6-dihydropyridin-3-yl)methyl)-1H-indol-1-yl)acetate, Intermediate 64. The procedure described above for intermediate 37 was followed, reacting intermediate 62B (0.280 g, 0.779 mmol) with 2,4,5-trifluorobenzyl bromide (0.351 g, 1.56 mmol) and NaI (0.234 g, 1.56 mmol). 0.274 g, 72% yield.

Step 2: Preparation of 2-(5-Chloro-2-methyl-3-((6-oxo-1-(2,4,5-trifluorobenzyl)-1,6-dihydro-pyridin-3-yl)methyl)-1H-indol-1-yl)acetic acid. (44). The procedure described above for 20 was followed, reacting intermediate 64 (0.274 g, 0.560 mmol) with lithium hydroxide monohydrate (0.470 g, 11.2 mmol). The crude product was purified by recrystallization from acetonitrile/ethanol to give fine white crystals (0.166 g, 62% yield): ¹H NMR (DMSO-d₆) δ 13.00 (s, 1H), 7.64 (d, J=2.3 Hz, 1H), 7.54 (ddd, J=10.7, 9.8, 6.8 Hz, 1H), 7.41 (d, J=1.8 Hz, 1H), 7.37 (d, J=8.6 Hz, 1H), 7.22-7.28 (m, 1H), 7.19 (dd, J=9.3, 2.5 Hz, 1H), 7.03 (dd, J=8.6, 2.0 Hz, 1H), 6.32 (d, J=9.3 Hz, 1H), 5.04 (s, 2H), 4.95 (s, 2H), 3.76 (s, 2H), 2.32 (s, 3H).

### Example 45

Step 1: Preparation of Methyl 2-(5-chloro-3-((1-(3-fluorobenzyl)-6-oxo-1,6-dihydropyridin-3-yl)methyl)-2-methyl-1H-indol-1-yl)acetate, Intermediate 65. The procedure described above for intermediate 37 was followed, reacting intermediate 62B (0.221 g, 0.615 mmol) with 3-fluorobenzyl bromide (0.15 mL, 0.23 g, 1.23 mmol) and NaI (0.184 g, 1.23 mmol). 0.174 g, 62% yield.

Step 2: Preparation of 2-(5-Chloro-3-((1-(3-fluorobenzyl)-6-oxo-1,6-dihydropyridin-3-yl)methyl)-2-methyl-1H-indol-1-yl)acetic acid (45). The procedure described above for 20 was followed, reacting intermediate 65 (0.174 g, 0.384 mmol) with lithium hydroxide monohydrate (0.322 g, 7.67 mmol). The crude product was purified by recrystallization from CH₃CN:EtOH to give fluffy white crystals (0.101 g, 60% yield): ¹H NMR (DMSO-d₆) δ 13.03 (br. s., 1H), 7.78 (d, J=2.0 Hz, 1H), 7.45 (d, J=2.0 Hz, 1H), 7.33-7.41 (m, 2H), 7.17 (dd, J=9.3, 2.5 Hz, 1H), 7.06-7.14 (m, 3H), 7.03 (dd,

J=8.7, 2.1 Hz, 1H), 6.33 (d, J=9.3 Hz, 1H), 5.06 (s, 2H), 4.95 (s, 2H), 3.75 (s, 2H), 2.31 (s, 3H).

### Example 46

Step 1: Preparation of Methyl 2-(5-fluoro-2-methyl-3-((6-oxo-1-(2,2,2-trifluoro-ethyl)-1,6-ihydropyridin-3-yl)methyl-1H-indol-1-yl)acetate, Intermediate 66. In a 100 mL round-bottomed flask under nitrogen, intermediate 46A (0.575 g, 1.75 mmol) and cesium carbonate (2.85 g, 8.76 mmol) were taken up in 25 mL anhydrous DMF, and 1,1,1-trifluoro-2-iodoethane (0.85 mL, 1.8 g, 8.8 mmol) was added. The mixture was heated to 55° C. for 80 minutes. LC-MS analysis indicated that starting material still remained, so additional iodide (0.85 mL) was added, and heating continued overnight until almost complete conversion to product had occurred. The cooled reaction mixture was poured into 250 mL water and extracted into ethyl acetate (2×); the combined organic extracts were washed with brine (3×), dried over anhydrous magnesium sulfate, filtered, evaporated, and purified by flash chromatography over silica gel (5-40% ethyl acetate in dichloromethane) to give pure product (0.314 g, 44% yield).

Step 2: Preparation of 2-(5-Fluoro-2-methyl-3-((6-oxo-1-(2,2,2-trifluoroethyl)-1,6-dihydropyridin-3-yl)methyl-1H-indol-1-yl)acetic acid (46). The procedure described above for 20 was followed, reacting intermediate 66 (0.314 g, 0.764 mmol) with lithium hydroxide monohydrate (0.642 g, 15.3 mmol). The crude product was purified by recrystallization from acetonitrile to give a fluffy white solid (98 mg, 32% yield): ¹H NMR (DMSO-d₆) δ 13.00 (br. s., 1H), 7.58 (s, 1H), 7.35 (dd, J=8.8, 4.3 Hz, 1H), 7.18-7.28 (m, 2H), 6.87 (td, J=9.2, 2.5 Hz, 1H), 6.38 (d, J=9.9 Hz, 1H), 4.95 (s, 2H), 4.80 (q, J=9.3 Hz, 2H), 3.75 (s, 2H), 2.32 (s, 3H).

### Example 47

Step 1: Preparation of tert-Butyl 2-(3-(3-benzyl-4-oxo-3,4-dihydrophthalazin-1-yl)-2-methyl-1H-indol-1-yl)acetate, Intermediate 66A. The procedure described above for intermediate 39 was followed, reacting tert-butyl 2-(3-(4-hydroxyphthalazin-1-yl)-2-methyl-1H-indol-1-yl)acetate (5.00 g, 12.8 mmol) with potassium carbonate (6.19 g, 44.8 mmol) and benzyl bromide (4.6 mL, 6.6 g, 38 mmol). The crude product was purified by flash chromatography over silica gel (6-50% ethyl acetate in hexanes (3.46 g, 56% yield).

Step 2: Preparation of 2-(3-(3-Benzyl-4-oxo-3,4-dihydrophthalazin-1-yl)-2-methyl-1H-indol-1-yl)-acetic acid, Intermediate 66B. Intermediate 66A (2.88 g, 6.01 mmol) was taken up in 160 mL trifluoroacetic acid and stirred at room temperature for 2 hours, until LC-MS analysis showed complete consumption of the ester. The reaction mixture was then evaporated, and the residue partitioned between ethyl acetate and brine. The aqueous layer was extracted with additional ethyl acetate, and the combined organic extracts washed with brine, dried over anhydrous magnesium sulfate, filtered, and evaporated to give pure product (2.44 g, 96% yield).

Step 3: Preparation of 2-(3-(3-Benzyl-4-oxo-3,4-dihydrophthalazin-1-yl)-2-methyl-1H-indol-1-yl)-acetamide (47). In a 25 mL round-bottomed flask, intermediate 66B (0.300 g, 0.708 mmol), ammonium chloride (0.152 g, 2.83 mmol), and BOP (0.344 g, 0.779 mmol) were taken up in 6 mL DMF. 4-methylmorpholine (0.40 mL, 0.36 g, 3.60 mmol) was added, and the mixture was allowed to stir at room temperature over the weekend. It was then poured into 60 mL water, and the off-white precipitate collected, washed three times with water, dried under vacuum, and purified by flash chromatography over silica gel (6-50% ethyl acetate in hexanes). Lyophilization gave a fluffy white solid (0.151 g, 51% yield): ¹H NMR (DMSO-d₆) δ 8.37-8.42 (m, 1H), 7.82-7.92 (m, 2H), 7.70 (br. s., 1H), 7.60 (dt, J=7.8, 0.8 Hz, 1H), 7.43-7.47 (m, 1H), 7.26-7.41 (m, 6H), 7.10-7.18 (m, 2H), 6.99 (ddd, J=8.0, 6.9, 1.0 Hz, 1H), 5.34-5.49 (m, 2H), 4.87 (s, 2H), 2.24 (s, 3H).

### Example 48

Preparation of 2-(3-(3-Benzyl-4-oxo-3,4-dihydrophthalazin-1-yl)-2-methyl-1H-indol-1-yl)-N,N-dimethylacetamide (48). In a 25 mL round-bottomed flask, intermediate 66B (0.300 g, 0.708 mmol), dimethylamine hydrochloride (64 mg, 0.78 mmol) and BOP (0.345 g, 0.779 mmol) were taken up in 6 mL DMF. 4-methylmorpholine (0.17 mL, 0.16 g, 1.6 mmol) was added, and the reaction stirred at room temperature for 1 hour, until LC-MS analysis showed complete conversion to product. The reaction mixture was poured into 60 mL water and the white precipitate washed with water three times, dried under vacuum, purified by flash chromatography over silica gel (6-50% ethyl acetate in hexanes), and lyophilized. 0.245 g, 77% yield: ¹H NMR (DMSO-d₆) δ 8.37-8.43 (m, 1H), 7.83-7.92 (m, 2H), 7.57 (dt, J=7.5, 0.9 Hz, 1H), 7.44-7.49 (m, 1H), 7.32-7.41 (m, 4H), 7.25-7.31 (m, 1H), 7.08-7.16 (m, 2H), 6.97 (td, J=7.5, 0.8 Hz, 1H), 5.34-5.50 (m, 2H), 5.15-5.29 (m, 2H), 3.19 (s, 3H), 2.89 (s, 3H), 2.17 (s, 3H).

### Example 49

Preparation of 2-Benzyl-4-(2-methyl-1-(2-oxo-2-(pyrrolidin-1-yl)ethyl)-1H-indol-3-yl)phthalazin-1(2H)-one (49). The procedure described above for 48 was followed, reacting intermediate 66B (0.300 g, 0.708 mmol) with pyrrolidine (65 μL, 55 mg, 0.78 mmol), BOP (0.345 g, 0.779 mmol) and 4-methylmorpholine (86 μL, 79 mg, 1.6 mmol). 0.266 g, 79% yield: ¹H NMR (DMSO-d₆) δ 3.38-8.42 (m, 1H), 7.82-7.92 (m, J=7.3, 7.3, 7.3, 7.3, 1.5 Hz, 2H), 7.55-7.60 (m, 1H), 7.46-7.51 (m, 1H), 7.32-7.42 (m, 4H), 7.25-7.31 (m, 1H), 7.09-7.15 (m, 2H), 6.94-7.01 (m, 1H), 5.35-5.50 (m, 2H), 5.07-5.20 (m, 2H), 3.68 (t, J=6.8 Hz, 2H), 3.32-3.38 (m, 2H), 2.19 (s, 3H), 1.98 (quin, J=6.8 Hz, 2H), 1.82 (quin, J=6.8 Hz, 2H).

### Example 50

Preparation of 2-(3-(3-Benzyl-4-oxo-3,4-dihydrophthalazin-1-yl)-2-methyl-1H-indol-1-yl)-N-(methylsulfonyl)acetamide (50). The procedure described above for 48 was followed, reacting intermediate 66B (0.200 g, 0.472 mmol) with methanesulfonamide (49 mg, 0.52 mmol), BOP (0.230 g, 0.520 mmol) and diisopropylethylamine (181 μL, 134 mg, 1.04 mmol). To work up the reaction, it was poured into 60 mL water, and partitioned between 100 mL each brine and ethyl acetate. The aqueous layer was extracted with additional ethyl acetate, and the combined organic extracts washed with brine (3×), dried over anhydrous magnesium sulfate, filtered, and evaporated. Flash chromatography over silica gel (2-20% methanol in dichloromethane), followed by preparative HPLC (water/acetonitrile with 0.1% formic acid) and lyophilization gave a fluffy white solid (17.5 mg, 7.4% yield): ¹14 NMR (DMSO-d₆) δ 12.33 (br. s., 1H), 8.35-8.46 (m, 1H), 7.82-7.95 (m, J=7.3, 7.3, 7.3, 7.3, 1.5 Hz, 2H), 7.57 (dt, J=7.6, 0.9 Hz, 1H), 7.46 (d, J=8.1 Hz, 1H), 7.32-7.41 (m, 4H), 7.26-7.31 (m, 1H), 7.12-7.19 (m, 2H), 6.98-7.03 (m, 1H), 5.35-5.49 (m, 2H), 5.05 (s, 2H), 3.21 (s, 3H), 2.23 (s, 3H)

### Example 51

Step 1: Preparation of 1-Chloro-4-(2-methyl-1H-indol-3-yl)phthalazine, Intermediate 67. The procedure described above for intermediate 1 was followed, reacting 2-methylindole (5.00 g, 38.1 mmol) with 1,4-dichlorophthalazine (8.35 g, 41.9 mmol) and aluminum chloride (7.12 g, 53.4 mmol). The cooled reaction mixture was poured into 1500 mL ice water, and the dark red precipitate collected, washed with water, and dried under vacuum. 8.24 g, 74% yield. Step 2: Preparation of 2-(3-(4-Chlorophthalazin-1-yl)-2-methyl-1H-indol-1-yl)-acetonitrile, Intermediate 68. In a 2-necked 100 mL round-bottomed flask, under nitrogen, intermediate 67 (1.00 g, 3.40 mmol) was taken up in 20 mL anhydrous DMF. Sodium hydride (163 mg, 98.0 mg, 4.09 mmol) was added in small portions, and the mixture allowed to stir for 30 minutes. Bromoacetonitrile (0.27 mL, 0.49 g, 4.1 mmol) was added, and the reaction allowed to stir overnight. LC-MS analysis indicated that the reaction was only 40% complete, but it was worked up anyway. It was quenched with 10 mL saturated ammonium chloride, and then partitioned between 200 mL each EtOAc and brine. The aqueous layer was extracted with additional ethyl acetate (60 mL), and the combined organic extracts washed with brine (3×70 mL), dried over anhydrous magnesium sulfate, filtered, evaporated and purified by flash chromatography over silica gel (10-80% EtOAc in hexanes). 0.399 g, 35% yield.

Step 3: Preparation of 2-(3-(4-Hydroxyphthalazin-1-yl)-2-methyl-1H-indol-1-yl)-acetonitrile, Intermediate 69. The procedure described above for intermediate 2 was followed, heating intermediate 68 (0.399 g, 1.20 mmol) in 19 mL acetic acid and 3 mL 1 M sodium hydroxide until LC-MS analysis showed complete conversion to product. The reaction mixture was then poured into 220 mL ice water, and the off-white precipitate collected, washed with water, and dried under vacuum to give product of sufficient purity to be used in the next Step (0.251 g, 67% yield).

Step 4: Preparation of 2-(3-(3-Benzyl-4-oxo-3,4-dihydrophthalazin-1-yl)-2-methyl-1H-indol-1-yl)-acetonitrile, Intermediate 70. The procedure described above for intermediate 39 was followed, reacting intermediate 69 (0.250 g, 0.795 mmol) with potassium carbonate (0.385 g, 2.78 mmol) and benzyl bromide (0.28 mL, 0.41 g, 2.4 mmol). The crude product was purified by flash chromatography over silica gel (7-60% EtOAc in hexanes) to give pure product (0.236 g, 73% yield).

Step 5: Preparation of 4-(1-((2H-Tetrazol-5-yl)methyl)-2-methyl-1H-indol-3-yl)-2-benzyl-phthalazin-1(2H)-one (51). In a 10 mL round-bottomed flask, intermediate 70 (0.236 g, 0.583 mmol), zinc bromide (0.131 g, 0.583 mmol) and sodium azide (42 mg, 0.642 mmol) were taken up in 3 mL isopropanol and 1.2 mL water. The mixture was refluxed overnight, until LC-MS analysis showed complete conversion to product, and cooled to room temperature. It was partitioned between ethyl acetate and 2 M hydrochloric acid, and the aqueous layer extracted with additional ethyl acetate. The combined organic extracts were evaporated, and the residue taken up in 40 mL 0.25 M NaOH and stirred for 2 hours. Although the reference had suggested that a precipitate of zinc hydroxide would form, only a faint cloudiness that could not be removed by filtration was observed. Thus, the filtered (and still cloudy) solution was acidified with concentrated hydrochloric acid, and the off-white precipitate collected, washed three times with 2 M hydrochloric acid, and dried under vacuum. It was then purified by preparative HPLC (water/acetonitrile with 0.1% formic acid) and lyophilized to give pure product (0.120 g, 46% yield): ¹H NMR (DMSO-d₆) δ 8.37-8.42 (m, 1H), 7.81-7.92 (m, J=18.4, 7.4, 7.4, 1.4 Hz, 2H), 7.55-7.62 (m, 2H), 7.32-7.41 (m, 4H), 7.25-7.31 (m, 1H), 7.10-7.19 (m, 2H), 6.95-7.04 (m, 1H), 5.80 (s, 2H), 5.34-5.49 (m, 2H), 2.37 (s, 3H).

### Example 52

Preparation of 2-Benzyl-4-(1-(2-hydroxyethyl)-2-methyl-1H-indol-3-yl)phthalazin-1(2H)-one (52). In a flame-dried 2-necked 15 mL round-bottomed flask, under nitrogen, intermediate 66B (0.200 g, 0.472 mmol) and triethylamine (66 μL, 48 mg, 0.47 mmol) were taken up in 1.4 mL anhydrous tetrahydrofuran and cooled to 0° C. with an ice water bath. A solution of ethyl chloroformate (45 μL, 51 mg, 0.47 mmol) in 0.3 mL anhydrous tetrahydrofuran was added dropwise via syringe. The mixture was stirred for 3 hours, at which point sodium borohydride (36 mg, 0.95 mmol) was added, and the ice bath was removed. The reaction was stirred for 25 minutes. LC-MS analysis showed the presence of the desired product, but no mixed anhydride intermediate or acid starting material. The reaction mixture was partitioned between 5 mL each ethyl acetate and brine, and the aqueous layer extracted with additional ethyl acetate. The combined organic extracts were washed with 5 mL brine, dried over anhydrous magnesium sulfate, filtered, evaporated, and purified by flash chromatography over silica gel (12-100% ethyl acetate in hexanes). Additional purification by preparative HPLC (water/acetonitrile with 0.1% formic acid), followed by lyophilization, gave pure product (29 mg, 15% yield): ¹H NMR (DMSO-d₆) δ 8.36-8.42 (m, 1H), 7.80-7.92 (m, J=18.4, 7.4, 7.4, 1.4 Hz, 2H), 7.59 (dt, J=7.8, 0.8 Hz, 1H), 7.52 (d, J=8.1 Hz, 1H), 7.32-7.40 (m, 4H), 7.26-7.31 (m, 1H), 7.09-7.17 (m, 2H), 6.97 (ddd, J=8.0, 7.1, 0.9 Hz, 1H), 5.34-5.49 (m, 2H), 4.97 (t, J=5.3 Hz, 1H), 4.30 (t, J=5.7 Hz, 2H), 3.75 (q, J=5.2 Hz, 2H), 2.33 (s, 3H).

### Example 53

Step 1: Preparation of Methyl 2-(5-chloro-2-methyl-346-oxo-1,6-dihydropyridin-3-yl)methyl)-1H-indol-1-yl)acetate, Intermediate 71A. The procedure described above for intermediate 46A was followed, reacting intermediate 62A (4.55 g, 19.1 mmol) with intermediate 38 (2.36 g, 19.1 mmol) in the presence of triethylsilane (8.5 mL, 6.2 g, 54 mmol) and TFA (2.9 mL, 4.4 g, 38 mmol). The crude product was triturated with 200 mL boiling acetonitrile. After allowing the filtrate to stand, additional product precipitated out. Both lots of product were pure (2.84 g, 43% yield).

Step 2: Preparation of Methyl 2-(5-chloro-2-methyl-346-oxo-1-(2,2,2-trifluoro-ethyl)-1,6-dihydro-pyridin-3-yl)methyl)-1H-indol-1-yl)acetate, Intermediate 71. The procedure described above for intermediate 66 was followed, reacting intermediate 71A (0.596 g, 1.73 mmol) with cesium carbonate (2.82 g, 8.65 mmol) and 1,1,1-trifluoro-2-iodoethane (1.7 mL, 3.6 g, l7 mmol). 0.262 g, 35% yield.

Step 3: Preparation of 2-(5-Chloro-2-methyl-346-oxo-1-(2,2,2-trifluoroethyl)-1,6-dihydropyridin-3-yl)methyl)-1H-indol-1-yl)acetic acid (53). The procedure described above for 20 was followed, reacting intermediate 71 (0.262 g, 0.614 mmol) with lithium hydroxide monohydrate (0.726 g, 17.3 mmol). The crude product was purified by recrystallization from acetonitrile/ethanol to give a fluffy white solid (89 mg, 35% yield): ¹H NMR (DMSO-d₆) 6: 13.01 (br. s., 1H), 7.58 (s, 1H), 7.47 (d, J=2.0 Hz, 1H), 7.38 (d, J=8.8 Hz, 1H), 7.24 (dd, J=9.3, 2.5 Hz, 1H), 7.04 (dd, J=8.7, 2.1 Hz, 1H), 6.39 (d, J=9.6 Hz, 1H), 4.96 (s, 2H), 4.79 (q, J=9.1 Hz, 2H), 3.77 (s, 2H), 2.32 (s, 3H).

### Example 54

Step 1: Preparation of Methyl 2-(5-fluoro-2-methyl-3-((6-oxo-1-(4,4,4-trifluoro-butyl)-1,6-dihydro-pyridin-3-yl)methyl)-1H-indol-1-yl)acetic acid, Intermediate 72. The procedure described above for intermediate 39 was followed, reacting intermediate 46A (0.403 g, 1.23 mmol) with cesium carbonate (2.00 g, 6.14 mmol) and 4-bromo-1,1,1-trifluorobutane (0.75 mL, 1.2 g, 6.1 mmol) in DMF at 85° C. The crude product was purified by flash chromatography over silica gel (6-50% ethyl acetate in dichloromethane) 0.207 g, 38% yield.

Step 2: Preparation of 2-(5-fluoro-2-methyl-346-oxo-1-(4,4,4-trifluorobutyl)-1,6-dihydropyridin-3-yl)methyl)-1H-indol-1-yl)acetic acid (54). The procedure described above for 20 was followed, reacting intermediate 72 (0.207 g, 0.473 mmol) with lithium hydroxide monohydrate (0.515 g, 12.3 mmol). The crude product was purified by recrystallization from acetonitrile to give a pale pink solid (79 mg, 40% yield): ¹H NMR (DMSO-d₆) δ 13.00 (br. s., 1H), 7.62 (d, J=2.0 Hz, 1H), 7.35 (dd, J=9.0, 4.4 Hz, 1H), 7.22 (dd, J=10.1, 2.5 Hz, 1H), 7.17 (dd, J=9.3, 2.5 Hz, 1H), 6.86 (td, J=9.1, 2.5 Hz, 1H), 6.29 (d, J=9.3 Hz, 1H), 4.95 (s, 2H), 3.90 (t, J=6.9 Hz, 2H), 3.73 (s, 2H), 2.32 (s, 3H), 2.16-2.31 (m, 2H), 1.84 (quin, J=7.6 Hz, 2H).

### Example 55

Step 1: Preparation of Methyl 6-oxo-1,6-dihydropyridazine-3-carboxylate, Intermediate 73. The procedure described in WO2006/34440 was followed. 6-oxo-1,6-dihydropyridazine-3-carboxylic acid monohydrate (8.91 g, 56.4 mmol) was taken up in 90 mL methanol. Thionyl chloride (0.66 mL, 1.1 g, 9.0 mmol) was added, and the mixture was refluxed overnight, until LC-MS analysis indicated that most or all of the acid had been esterified. The reaction mixture was cooled to room temperature, then chilled in the fridge, and the white crystalline precipitate was collected and dried under vacuum. 7.07 g, 81% yield: ¹H NMR (DMSO-d₆) δ 13.60 (br. s., 1H), 7.82 (d, J=10.1 Hz, 1H), 6.96 (d, J=9.9 Hz, 1H), 3.84 (s, 3H).

Step 2: Preparation of Methyl 1-(2,4-difluorobenzyl)-6-oxo-1,6-dihydropyridazine-3-carboxylate, Intermediate 73A. The procedure described above for intermediate 39 was followed, reacting intermediate 73 (1.00 g, 6.49 mmol) with potassium carbonate (3.14 g, 22.7 mmol) and 2,4-difluorobenzyl bromide (2.5 mL, 4.0 g, 19 mmol). The crude product was purified by flash chromatography over silica gel (12-100% ethyl acetate in hexanes). 1.21 g, 67% yield: ¹H NMR (DMSO-d₆) δ 7.72-8.01 (m, 1H), 7.18-7.55 (m, 2H), 6.95-7.19 (m, 2H), 5.34 (s, 2H), 3.85 (s, 3H).

Step 3: Preparation of 2-(2,4-Difluorobenzyl)-6-(hydroxymethyl)pyridazin-3(2H)-one, Intermediate 74. In a 100 mL round-bottomed flask with a condenser, intermediate 73A (1.13 g, 4.03 mmol) and sodium borohydride (0.153 g, 4.03 mmol) were taken up in 30 mL anhydrous tetrahydrofuran. The mixture was heated to reflux, and 5.2 mL anhydrous methanol was added dropwise over 1 hour, using a syringe pump. Reflux was then continued for 1 additional hour. After cooling to room temperature, the reaction was quenched with 0.7 mL water, and the solvent was removed. The residue was taken up in 35 mL 0.5 M hydrochloric acid and extracted into dichloromethane (3×). The combined organic extracts were washed with water and brine, dried over anhydrous magnesium sulfate, filtered, evaporated, and purified by flash chromatography over silica gel (1% methanol in ethyl acetate) to give product of sufficient purity to be used in the next step (0.458 g, 45% yield): ¹H NMR (DMSO-d₆) δ 7.50 (d, J=9.6 Hz, 1H), 7.18-7.33 (m, 2H), 7.02-7.09 (m, 1H), 7.00 (d, J=9.6 Hz, 1H), 5.50 (t, J=6.1 Hz, 1H), 5.22 (s, 2H), 4.31 (d, J=6.1 Hz, 2H).

Step 4: Preparation of 1-(2,4-Difluorobenzyl)-6-oxo-1,6-dihydropyridazine-3-carbaldehyde, Intermediate 75. In a 250 mL round-bottomed flask with a condenser, intermediate 74 (0.458 g, 1.82 mmol) was taken up in 55 mL anhydrous toluene, and manganese dioxide (2.37 g, 27.2 mmol) was added. The mixture was refluxed overnight, under nitrogen, cooled to room temperature, and filtered through Celite, washing with toluene. The toluene was then evaporated to give product of sufficient purity to be used directly in the next step (0.311 g, 69% yield): ¹H NMR (DMSO-d₆) δ 9.64 (d, J=0.8 Hz, 1H), 7.80 (d, J=9.6 Hz, 1H), 7.43 (td, J=8.7, 6.4 Hz, 1H), 7.29 (ddd, J=10.5, 9.3, 2.7 Hz, 1H), 7.05-7.13 (m, 2H), 5.39 (s, 2H).

Step 5: Preparation of 2-Methyl-4-nitro-1H-indole, Intermediate 76. In a 125 mL Erlenmeyer flask, 3-nitroaniline (1.00 g, 7.24 mmol) and acetone (0.74 mL, 0.59 g, 10 mmol) were taken up in 20 mL dimethylsulfoxide. Potassium tert-butoxide (1.95 g, 17.4 mmol) was added, and the reaction mixture stirred at room temperature for 2 hours, until LC-MS analysis indicated that most of the starting material had been consumed. Saturated ammonium chloride (85 mL) was added, and the product extracted into ethyl acetate (3×75 mL). The combined organic extracts were washed with water (60 mL), dried over anhydrous magnesium sulfate, filtered, evaporated, and purified by flash chromatography over silica gel (6-50% EtOAc in hexanes). 0.56 g, 44% yield: ¹H NMR (DMSO-d₆) δ 11.85 (br. s., 1H), 7.99 (dd, J=8.1, 1.0 Hz, 1H), 7.74 (dt, J=8.0, 0.8 Hz, 1H), 7.19 (t, J=8.0 Hz, 1H), 6.80 (t, J=0.9 Hz, 1H), 2.49 (d, J=0.8 Hz, 3H).

Step 6: Preparation of Methyl 2-(2-methyl-4-nitro-1H-indol-1-yl)acetate, Intermediate 76A. The procedure described above for intermediate 36 was followed, reacting intermediate 76 (0.56 g, 3.2 mmol) with sodium hydride (0.153 g of a 60 wt % mineral oil suspension, 92.0 mg, 3.81 mmol) and methyl bromoacetate (0.36 mL, 0.58 g, 3.8 mmol) for 2.5 hours, until LC-MS analysis showed complete conversion to product. The crude product was purified by flash chromatography over silica gel (6-50% ethyl acetate in hexanes to give a bright yellow solid (0.643 g, 81% yield): ¹H NMR (DMSO-d₆) δ 8.04 (dd, J=8.1, 0.8 Hz, 1H), 7.93 (dt, J=8.1, 0.8 Hz, 1H), 7.26 (t, J=8.1 Hz, 1H), 6.93 (t, J=0.9 Hz, 1H), 5.27 (s, 2H), 3.70 (s, 3H), 2.44 (d, J=1.0 Hz, 3H).

Step 7: Preparation of Ethyl 2-(4-amino-2-methyl-1H-indol-1-yl)acetate, Intermediate 76B. In a 20 mL microwave vial (for 5-10 mL reaction volumes), intermediate 76A (0.585 g, 2.36 mmol) was taken up in 5 mL ethanol, and tin(II) chloride dihydrate (2.66 g, 11.8 mmol) was added. The vial was crimp-sealed and heated in a Biotage microwave until complete reduction of the nitro group had occurred (heating was accomplished in 5 minute blocks, starting at 110° C. and increasing in 10 degree increments to 150° C.). Transesterification to the ethyl ester had also occurred. The contents of the vial were poured into 25 mL ice water, rinsing with additional water, and saturated sodium bicarbonate was added to neutralize the suspension. The product was extracted into chloroform (5×40 mL), dried over anhydrous magnesium sulfate, filtered, evaporated and purified by flash chromatography over silica gel (6-50% EtOAc in hexanes) to give a light golden-brown solid (0.320 g, 59% yield): ¹H NMR (DMSO-d₆) δ 6.70-6.76 (m, 1H), 6.49 (d, J=8.1 Hz, 1H), 6.27-6.29 (m, 1H), 6.15 (dd, J=7.6, 0.8 Hz, 1H), 5.06 (s, 2H), 4.89 (s, 2H), 4.13 (q, J=7.2 Hz, 2H), 2.27 (d, J=1.0 Hz, 3H), 1.20 (t, J=7.1 Hz, 3H).

Step 8: Preparation of Ethyl 2-(4-acetamido-2-methyl-1H-indol-1-yl)acetate, Intermediate 76C. Intermediate 76B (0.266 g, 1.15 mmol) was taken up in 1 mL acetic acid, and acetic anhydride (0.5 mL, 0.5 g, 5 mmol) was added. The reaction was stirred for 10 minutes at room temperature, until LC-MS analysis indicated complete conversion to product. Water (10 mL) was added, and the white precipitate was collected, washed with water, and dried under vacuum. 0.241 g, 77% yield: ¹H NMR (DMSO-d₆) δ 9.51 (s, 1H), 7.55 (d, J=7.6 Hz, 1H), 7.07 (d, J=8.1 Hz, 1H), 6.92-7.01 (m, 1H), 6.50 (s, 1H), 5.03 (s, 2H), 4.14 (q, J=7.1 Hz, 2H), 2.33 (d, J=0.8 Hz, 3H), 2.12 (s, 3H), 1.20 (t, J=7.1 Hz, 3H).

Step 9: Preparation of 2-(4-Acetamido-3-((1-(2,4-difluorobenzyl)-6-oxo-1,6-dihydropyridazin-3-yl)-methyl)-2-methyl-1H-indol-1-yl)acetic acid (55). The procedure described above for intermediate 46A was followed, reacting intermediate 76C (0.173 g, 0.631 mmol) with intermediate 75 (0.158 g, 0.631 mmol) in the presence of triethylsilane (0.28 mL, 0.21 g, 1.8 mmol) and trifluoroacetic acid (97 μL, 0.14 g, 1.3 mmol). The crude ester was purified by flash chromatography over silica gel (2% methanol in ethyl acetate). It was then taken up in 5 mL tetrahydrofuran and 15 mL methanol, and a solution of lithium hydroxide monohydrate (0.265 g, 6.31 mmol) in 5 mL water was added. The reaction mixture was stirred for 1 hour, until LC-MS analysis indicate complete hydrolysis of the ester. It was then acidified with concentrated HCl and partitioned between EtOAc and brine, and the aqueous layer extracted with additional EtOAc (2×). The combined organic extracts were washed with brine, dried over anhydrous MgSO₄, filtered, and evaporated. The crude acid was recrystallized from CH₃CN/EtOH to give a grayish-blue powder (44 mg, 15% yield): ¹H NMR (DMSO-d₆) δ 12.99 (s., 1H), 9.40 (s, 1H), 7.20-7.31 (m, 3H), 6.93-7.06 (m, 3H), 6.79-6.86 (m, 2H), 5.23 (s, 2H), 4.95 (s, 2H), 4.04 (s, 2H), 2.26 (s, 3H), 1.93 (s, 3H).

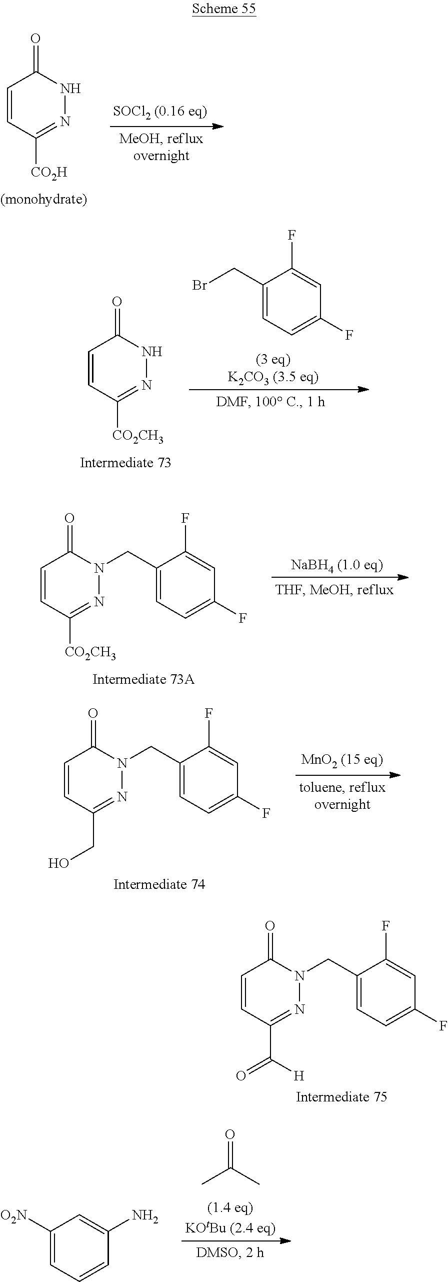

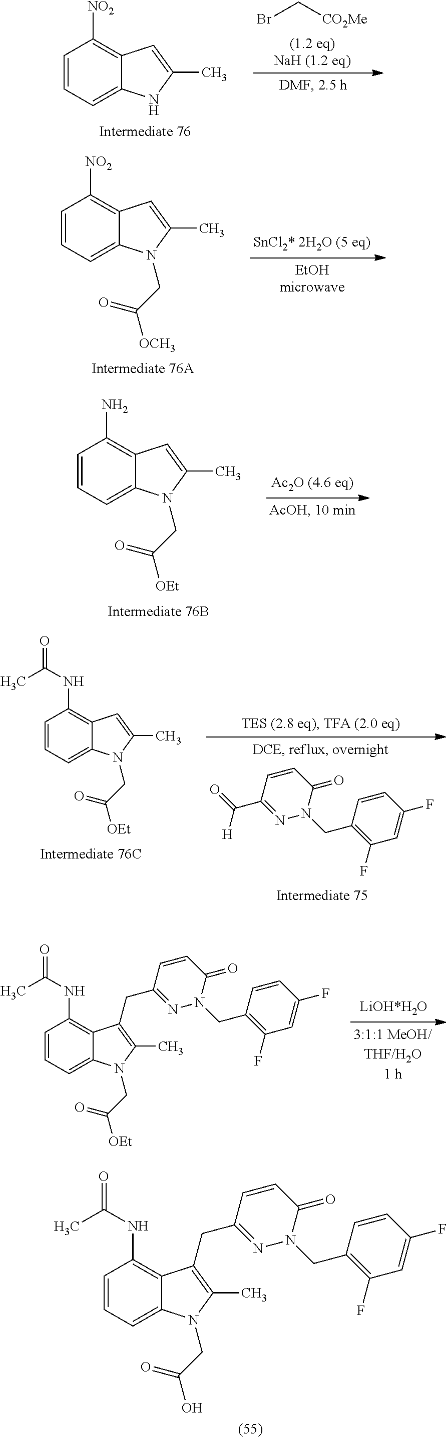

### Example 56

Step 1: Preparation of (2,4-difluorobenzyl)hydrazine, Intermediate 77. A 2-necked 250 mL round-bottomed flask was charged with hydrazine (40 mL, 41 g, 1.3 mol) and cooled to 0° C. with an ice water bath. A solution of 2,4-difluorobenzyl bromide (15.5 mL, 25.0 g, 121 mmol) in 20 mL anhydrous methanol was added from an addition funnel over 1 hour. The ice water bath was then removed, and the solution allowed to stir at room temperature overnight. The methanol was removed by evaporation, and the product extracted out of the hydrazine with ether (3×). The ethereal solution was evaporated to give a clear yellowish oil of sufficient purity to be used directly in the next step (18.2 g, 96% yield): ¹H NMR (DMSO-d₆) δ 7.41-7.53 (m, 1H), 7.15 (ddd, J=10.4, 9.5, 2.7 Hz, 1H), 7.00-7.07 (m, 1H), 3.74 (s, 2H), 3.45 (br. s., 3H).

Step 2: Preparation of Methyl 1-(2,4-difluorobenzyl)-6-oxo-1,4,5,6-tetrahydro-pyridazine-3-carboxylate, Intermediate 78. In a 3-necked 1 L round-bottomed flask with a condenser, (2,4-difluorobenzyl)hydrazine (17.9 g, 0.113 mmol) and dimethyl 2-oxoglutarate (14.9 mL, 17.9 g, 103 mmol) were taken up in 230 mL ethanol, and 15 drops of concentrated hydrochloric acid were added. The solution was heated to reflux overnight. LC-MS analysis showed some product, but also a considerable amount of uncyclized intermediate, so additional concentrated hydrochloric acid was added (0.1 mL), and the solution refluxed for an additional day, until most or all of the intermediate was gone. The mixture was then cooled to room temperature and the solvent evaporated. The residue was crystallized from methanol/acetonitrile, and the evaporated filtrate recrystallized again from methanol. The evaporated filtrate from this second recrystallization was purified by flash chromatography over silica gel. Methyl ester and ethyl ester (transesterification product) were both isolated. Methyl ester: 23.9 g (82% yield): ¹H NMR (DMSO-d₆) δ 7.31 (td, J=8.7, 6.6 Hz, 1H), 7.24 (ddd, J=10.6, 9.3, 2.5 Hz, 1H), 7.02-7.08 (m, J=8.6, 8.6, 2.6, 1.0 Hz, 1H), 4.93 (s, 2H), 3.75 (s, 3H), 2.81-2.88 (m, 2H), 2.54-2.61 (m, 2H). Intermediate 79. Ethyl 1-(2,4-difluorobenzyl)-6-oxo-1,4,5,6-tetrahydropyridazine-3-carboxylate 1.86 g (6.1% yield): ¹H NMR (DMSO-d₆) δ 7.32 (td, J=8.7, 6.7 Hz, 1H), 7.24 (ddd, J=10.6, 9.3, 2.5 Hz, 1H), 7.01-7.08 (m, J=8.5, 8.5, 2.7, 1.3 Hz, 1H), 4.93 (s, 2H), 4.21 (q, J=7.1 Hz, 2H), 2.80-2.88 (m, 2H), 2.54-2.60 (m, 2H), 1.24 (t, J=7.1 Hz, 3H).

Step 3: Preparation of 2-(2,4-Difluorobenzyl)-6-(hydroxymethyl)-4,5-dihydro-pyridazin-3(2H)-one, Intermediate 80. The procedure described above for intermediate 74 was followed, reacting intermediate 79 (8.06 g, 28.6 mmol) with sodium borohydride (1.08 g, 28.6 mmol). 3.46 g, 48% yield: ¹H NMR (DMSO-d₆) δ 7.29 (td, J=8.7, 6.7 Hz, 1H), 7.21 (ddd, J=10.5, 9.4, 2.5 Hz, 1H), 7.00-7.07 (m, J=8.6, 8.6, 2.6, 1.0 Hz, 1H), 5.22 (t, J=5.9 Hz, 1H), 4.82 (s, 2H), 4.00 (d, J=5.8 Hz, 2H), 2.53-2.60 (m, 2H), 2.41-2.47 (m, 2H).

Step 4: Preparation of 1-(2,4-Difluorobenzyl)-6-oxo-1,6-dihydropyridazine-3-carbaldehyde, Intermediate 81. The procedure described above for intermediate 75 was followed, reacting intermediate 80 (3.46 g, 13.6 mmol) with manganese dioxide (17 g, 200 mmol). ¹H NMR analysis indicated that the crystalline white solid contained about 10 mol % of the unsaturated aldehyde intermediate (intermediate 82). No attempt was made to separate the two, due to stability concerns. 1.65 g, 49% yield: ¹H NMR (DMSO-d₆) δ 9.64 (d, J=1.0 Hz, 1H), 7.80 (d, J=9.6 Hz, 1H), 7.43 (td, J=8.7, 6.6 Hz, 1H), 7.29 (ddd, J=10.6, 9.3, 2.5 Hz, 1H), 7.05-7.12 (m, 2H), 5.39 (s, 2H). Intermediate 82. 1-(2,4-Difluorobenzyl)-6-oxo-1,4,5,6-tetrahydropyridazine-3-carbaldehyde. Aromatic ¹H NMR peaks overlap with those for intermediate 81. ¹H NMR (DMSO-d₆) δ 9.40 (s, 1H), 5.00 (s, 2H), 2.69-2.74 (m, 2H), 2.56-2.61 (m, 2H).

Step 5: Preparation of Methyl 2-(3-((1-(2,4-difluorobenzyl)-6-oxo-1,4,5,6-tetrahydropyridazin-3-yl)methyl)-5-fluoro-2-methyl-1H-indol-1-yl)acetate, Intermediate 83. In a 250 mL round-bottomed flask, under nitrogen, intermediate 37A (1.57 g, 7.09 mmol) and the intermediate 81/82 mixture (1.95 g, 7.79 mmol) were taken up in anhydrous dichloromethane and cooled to 0° C. Triethylsilane (4.0 mL, 2.9 g, 25 mmol) was added by syringe, and trifluoroacetic acid (1.6 mL, 2.4 g, 21 mmol) was then added dropwise by syringe. The ice bath was removed, and the reaction mixture allowed to stir for 3 days, until LC-MS analysis showed complete conversion to product. Special attention was taken to ensure that the intermediate formed by addition of two indoles to one aldehyde had been consumed. The solution was poured into 140 mL saturated sodium bicarbonate, rinsing the flask with 45 mL dichloromethane. The layers were separated, and the aqueous layer extracted with additional dichloromethane (2×45 mL). The combined organic extracts were washed with water and brine, dried over anhydrous magnesium sulfate, filtered and evaporated. The crude ester was purified by flash chromatography over silica gel (12-100% ethyl acetate in hexanes) to separate the title compound from intermediate 84, which was the main component of the mixture. Fractions containing primarily intermediate 83 were evaporated and combined with similar fractions isolated from another run on a similar scale, and the mixture was purified a second time (12-100% ethyl acetate in hexanes). The ester still was not pure. It was added to similar material isolated from a third run, and purified a third time (5-40% ethyl acetate in dichloromethane). Finally, pure ester was obtained. 0.163 g: ¹H NMR (DMSO-d₆) δ 7.35 (dd, J=8.8, 4.3 Hz, 1H), 7.29 (td, J=8.6, 6.6 Hz, 1H), 7.21 (ddd, J=10.5, 9.5, 2.5 Hz, 1H), 6.97-7.07 (m, 2H), 6.87 (td, J=9.2, 2.5 Hz, 1H), 5.08 (s, 2H), 4.86 (s, 2H), 3.66 (s, 3H), 3.63 (s, 2H), 2.29 (s, 4H), 2.22 (s, 3H).

Step 6: Preparation of 2-(3-((1-(2,4-difluorobenzyl)-6-oxo-1,4,5,6-tetrahydropyridaz in-3-yl)-methyl)-5-fluoro-2-methyl-1H-indol-1-yl)acetic acid (56). Intermediate 83 (0.163 g, 0.357 mmol) was taken up in 3 mL tetrahydrofuran and 1.5 mL methanol, and a solution of lithium hydroxide monohydrate (75 mg, 1.8 mmol) in 1.5 mL water was added. The reaction was stirred for 2 hours, then poured into 30 mL 1 M HCl, extracted into ethyl acetate (3×10 mL), washed with water and brine, dried over anhydrous magnesium sulfate, filtered and evaporated. Preparative HPLC (water/methanol with 0.1% formic acid), followed by lyophilization, gave a white solid (98.5 mg, 62% yield): ¹H NMR (DMSO-d₆) δ 7.25-7.35 (m, 2H), 7.17-7.24 (m, 1H), 6.97-7.05 (m, 2H), 6.85 (td, J=9.2, 2.4 Hz, 1H), 4.86 (s, 4H), 3.63 (s, 2H), 2.29 (s, 4H), 2.23 (s, 3H).

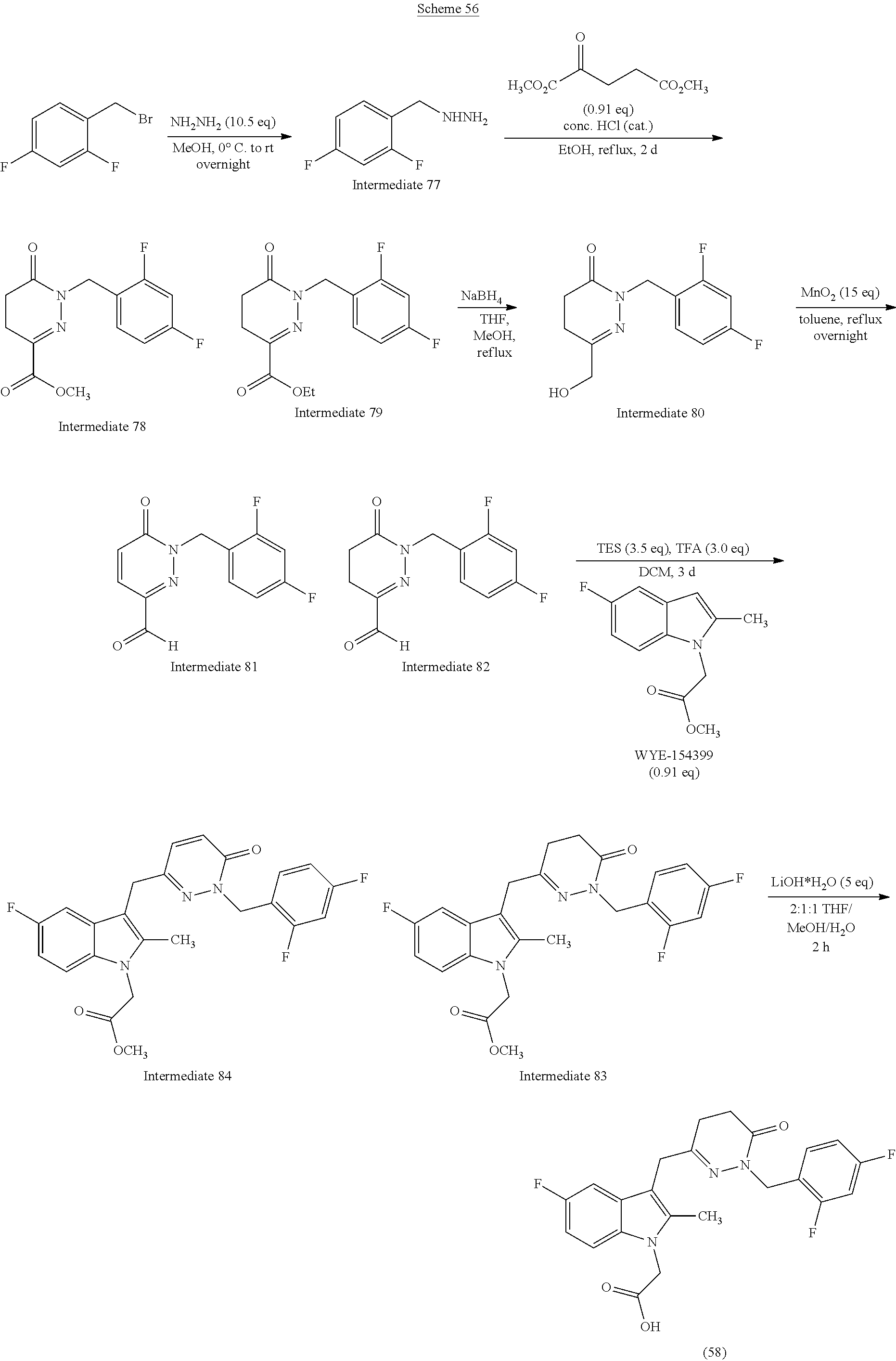

### Example 57

Step 1: Preparation of Ethyl 6-oxo-1-(2,2,2-trifluoroethyl)-1,4,5,6-tetrahydro-pyridazine-3-carboxylate, Intermediate 85. The procedure described above for intermediate 78 was followed, reacting dimethyl 2-oxoglutarate (10.0 g, 57.4 mmol) with (2,2,2-trifluoroethyl)hydrazine (8.0 mL, 10 g of 70wt % aq. solution, 7.2 g, 63 mmol). The crude product was purified by flash chromatography over silica gel (10-80% EtOAc in hexanes) to give a yellow solid (9.24 g, 64% yield).

Step 2: Preparation of 6-(Hydroxymethyl)-2-(2,2,2-trifluoroethyl)-4,5-dihydro-pyridazin-3 (2H)-one, Intermediate 86. The procedure described above for intermediate 74 was followed, reacting intermediate 85 (1.00 g, 3.97 mmol) with sodium borohydride (0.225 g, 5.95 mmol), except that reflux was continued for only 30 min. after the MeOH addition was complete. 0.202 g, 24% yield.

Step 3: Preparation of 6-Oxo-1-(2,2,2-trifluoroethyl)-1,4,5,6-tetrahydropyridazine-3-carbaldehyde, Intermediate 87. In a 2-necked 100 mL round-bottomed flask fitted with an addition funnel, under nitrogen, oxalyl chloride (0.56 mL, 0.81 g, 6.4 mmol) was taken up in 11 mL anhydrous dichloromethane, and the solution cooled to −78° C. (dry ice/acetone bath). A solution of dimethyl sulfoxide (0.95 mL, 1.0 g, 13 mmol) in 3 mL anhydrous dichloromethane was added in rapid drops from the addition funnel. The reaction mixture was stirred for 20 minutes, then a solution of intermediate 86 (0.841 g, 4.00 mmol) in 3 mL anhydrous dichloromethane was added over 10 minutes. The reaction mixture was now stirred for 1 hour at −78° C. Triethylamine (3.9 mL, 2.8 g, 28 mmol) was added dropwise, and stirring continuted for an additional 20 minutes. Stirring became quite difficult due to the formation of a thick precipitate. The cooling bath was removed, and the reaction allowed to warm to room temperature. Water (20 mL) was added, and the layers were separated. The aqueous layer was extracted with additional dichloromethane (2×10 mL), and the combined organic extracts washed with brine (2×10 mL). The dichloromethane solution was dried over anhydrous magnesium sulfate, filtered, and evaporated. The residue was taken up in 75 mL dichloromethane, and washed successively with 20 mL each of 0.5 M hydrochloric acid, water, 5% sodium carbonate, water, and brined. The solution was then dried over anhydrous magnesium sulfate, filtered, and evaporated to give a golden-brown oil (0.544 g, 65% yield).

Step 4: Preparation of 2-(5-Fluoro-2-methyl-346-oxo-1-(2,2,2-trifluoroethyl)-1,4,5,6-tetrahydro-pyridazin-3-yl)methyl)-1H-indol-1-yl)acetate, Intermediate 88. The procedure described above for intermediate 83 was followed, reacting intermediate 37A (0.526 g, 2.38 mmol) with intermediate 87 (0.544 g, 2.61 mmol) in the presence of triethylsilane (1.3 mL, 0.97 g, 8.3 mmol) and trifluoroacetic acid (0.55 mL, 0.81 g, 7.1 mmol). The crude product was purified by flash chromatography over silica gel (12-100% ethyl acetate in hexanes) to give pure material (0.742 g, 76% yield).

Step 5: Preparation of 2-(5-Fluoro-2-methyl-346-oxo-1-(2,2,2-trifluoroethyl)-1,4,5,6-tetrahydro-pyridazin-3-yl)methyl)-1H-indol-1-yl)acetic acid (57). The procedure described above for 56 was followed, reacting intermediate 88 (0.742 g, 1.80 mmol) with lithium hydroxide monohydrate (0.377 g, 8.98 mmol). Recrystallization from ethanol/water gave a white solid (0.120 g, 17%yield): ¹H NMR (DMSO-d₆) δ 12.98 (br. s., 1H), 7.37 (dd, J=8.8, 4.5 Hz, 1H), 7.22 (dd, J=9.9, 2.5 Hz, 1H), 6.89 (td, J=9.2, 2.5 Hz, 1H), 4.96 (s, 2H), 4.48 (q, J=9.3 Hz, 2H), 3.69 (s, 2H), 2.25-2.37 (m, 7H).

### Example 58

Step 1: Preparation of Methyl 6-oxo-1,4,5,6-tetrahydropyridazine-3-carboxylate, Intermediate 89. The procedure described above for intermediate 78 was followed, reacting dimethyl 2-oxoglutarate (38.5 g, 221 mmol) with hydrazine (7.6 mL, 7.8 g, 240 mmol). Upon partial evaporation of the cooled reaction mixture, a significant amount of mustard-yellow solid precipitated out, so this was collected and dried under vacuum. The evaporated filtrate was purified by flash chromatography over silica gel (12-100% ethyl acetate in hexanes). Both lots were pure enough to be used in the next step, although the recrystallized material did contain ethyl ester from transesterification as well as the desired methyl ester (26.7 g, 77% yield).

Step 2: Preparation of Methyl 6-oxo-1-(4,4,4-trifluorobutyl)-1,4,5,6-tetrahydro-pyridazine-3-carboxylate, Intermediate 90. The procedure described above for intermediate 39 was followed, reacting intermediate 89 (10.0 g, 64.0 mmol) with potassium carbonate (35 g, 260 mmol) and 4-bromo-1,1,1-trifluorobutane (15.7 mL, 24.5 g, 128 mmol) in DMF at 50° C. The crude product was purified by flash chromatography over silica gel (7-60% ethyl acetate in hexanes). 12.01 g, 70% yield.

Step 3: Preparation of 6-(Hydroxymethyl)-2-(4,4,4-trifluorobutyl)-4,5-dihydropyridazin-3(2H)-one, Intermediate 90A. The procedure described above for intermediate 74 was followed, reacting intermediate 90 (0.912 g, 3.43 mmol) with sodium borohydride (0.194 g, 5.14 mmol), except that reflux was continued for only 15 minutes after the methanol addition was complete. A viscous yellow oil was isolated (0.38 g, 47% yield).

Step 4: Preparation of 6-Oxo-1-(4,4,4-trifluorobutyl)-1,4,5,6-tetrahydropyridazine-3-carbaldehyde, Intermediate 91. The procedure described above for intermediate 87 was followed, reacting intermediate 90A (3.91 g, 16.4 mmol) with oxalyl chloride (1.7 mL, 2.4 g, 19 mmol) and dimethylsulfoxide (2.8 mL, 3.1 g, 39 mmol) for 30 minutes, then with triethylamine (11.4 mL, 8.30 g, 82.0 mmol) for 10 minutes. 2.53 g, 65% yield.

Step 5: Preparation of Methyl 2-(5-fluoro-2-methyl-346-oxo-1-(4,4,4-trifluoro-butyl)-1,4,5,6-tetrahydropyridazin-3-yl)methyl)-1H-indol-1-yl)acetate, Intermediate 92. The procedure described above for intermediate 83 was followed, reacting intermediate 37A (1.05 g, 4.73 mmol) with intermediate 91 (1.23 g, 5.21 mmol) in the presence of triethylsilane (2.7 mL, 1.9 g, 17 mmol) and trifluoroacetic acid (1.1 mL, 1.6 g, 14 mmol). The crude product was purified by flash chromatography over silica gel (12-100% EtOAc in hexanes) to give pure ester (1.80 g, 86% yield).

Step 6: Preparation of 2-(5-Fluoro-2-methyl-3-((6-oxo-1-(4,4,4-trifluorobutyl)-1,4,5,6-tetrahydro-pyridazin-3-yl)methyl)-1H-indol-1-yl)acetic acid (58). The procedure described for 56 was followed, reacting intermediate 92 (1.80 g, 4.08 mmol) with lithium hydroxide monohydrate (0.856 g, 20.4 mmol). Recrystallization from EtOH gave white solid (1.11 g, 64% yield). ¹H NMR (DMSO-d₆) 613.00 (s., 1H), 7.36 (dd, J=9, 4.4 Hz, 1H), 7.21 (dd, J=9.9, 2.5 Hz, 1H), 6.88 (td, J=9.2, 2.5 Hz, 1H), 4.96 (s, 2H), 3.71 (t, J=6.8 Hz, 2H), 3.67 (s, 2H), 2.30 (s, 3H), 2.17-2.29 (m, 6H), 1.74-1.83 (m, 2H).

### Example 59

Step 1: Preparation of tert-butyl 2-(5-chloro-3-(3-(2,5-difluorobenzyl)-4-oxo-3,4-dihydrophthalazin-1-yl)-2-methyl-1H-indol-1-yl)acetate, Intermediate 93. To a 100 mL round bottom flask under a nitrogen atmosphere was added intermediate 2, tert-butyl 2-(5-chloro-2-methyl-3-(4-oxo-3,4-dihydrophthalazin-1-yl)-1H-indol-1-yl)acetate (0.400 g, 0.95 mmol, 1.0 equiv), 2,5-difluorobenzyl bromide (0.390 g, 1.89 mmol, 2.0equiv), potassium carbonate (0.326 g, 2.38 mmol, 2.5 equiv) and 40 mL DMF. The resulting suspension was heated to 85° C. for 16 hours. The mixture was then allowed to cool to room temperature and then poured into 200 mL water. This was extracted with three 50 mL portions of EtOAc. The combined organic layers were washed with water and brine and dried over MgSO₄. Filtration and concentration in vacuo gave a tan solid. This crude material was purified by silica gel chromatography to give the desired product as a tan solid (0.338 g, 65%).

Step 2: Preparation of 2-(5-chloro-3-(3-(2,5-difluorobenzyl)-4-oxo-3,4-dihydrophthalaz in-1-yl)-2-methyl-1H-indol-1-yl)acetic acid (59). To a 100 mL round bottom flask under an atmosphere of nitrogen was added intermediate 93, tert-butyl 2-(5-chloro-3-(3-(2,5-difluorobenzyl)-4-oxo-3,4-dihydrophthalazin-1-yl)-2-methyl-1H-indol-1-yl)acetate (0.338 g, 0.62 mmol, 1.0 equiv) and 25 mL of TFA. The resulting solution was allowed to stir at room temperature for 3 hours. All volatiles were then removed in vacuo and the residue was purified by reverse phase HPLC. The isolated material was then suspended in acetonitrile/water, frozen and lyophilized to give the desired product as a white lyophilized powder (0.204 g, 67%). ¹14 NMR (400 MHz, DMSO-d₆) δ 1.96-2.12 (m, 3H) 4.93 (d, J=1.3 Hz, 2H) 5.27 (dd, J=14.6 Hz, 2H) 6.86-6.98 (m, 2H) 6.98-7.17 (m, 2H) 7.27-7.43 (m, 2H) 7.60-7.82 (m, 2H) 8.09-8.31 (m, 1H) 13.01 (s, 1H).

### Example 60

Preparation of 2-(5-chloro-2-methyl-3-(4-oxo-3-(2,4,5-trifluorobenzyl)-3,4-dihydro-phthalazin-1-yl)-1H-indol-1-yl)acetic acid (60). The title compound was prepared according to the procedure of Example 1. Yield 64%

### Example 61

Preparation of 2-(5-chloro-3-(3-(2,4-difluorobenzyl)-4-oxo-3,4-dihydrophthalazin-1-yl)-2-methyl-1H-indol-1-yl)acetic acid (61). The title compound was prepared according to the procedure of Example 1. Yield 53%.

### Example 62

Preparation of 2-(3-(3-(2,5-difluorobenzyl)-4-oxo-3,4-dihydrophthalazin-1-yl)-5-fluoro-2-methyl-1H-indol-1-yl)acetic acid (62). The title compound was prepared according to the procedure of Example 1. Yield 64%

### Example 63

Preparation of 2-(5-fluoro-2-methyl-3-(4-oxo-3-(2,4,5-trifluorobenzyl)-3,4-dihydro-phthalazin-1-yl)-1H-indol-1-yl)acetic acid (63). The title compound was prepared according to the procedure of Example 1. Yield 65%.

### Example 64

Preparation of 2-(3-(3-(2,4-difluorobenzyl)-4-oxo-3,4-dihydrophthalazin-1-yl)-5-fluoro-2-methyl-1H-indol-1-yl)acetic acid (64). The title compound was prepared according to the procedure of Example 1. Yield 50%.

### Example 65

Preparation of 2-(5-fluoro-2-methyl-3-(3-(4-(methylsulfonyl)benzyl)-4-oxo-3,4-dihydro-phthalazin-1-yl)-1H-indol-1-yl)acetic acid (65). The title compound was prepared according to the procedure of Example 1. Yield 53%

### Example 66

Preparation of 2-(5-chloro-2-methyl-3-(1-(4-(methylsulfonyl)benzyl)-6-oxo-1,6-dihydropyridazin-3-yl)-1H-indol-1-yl)acetic acid (66). The title compound was prepared according to the procedure of Example 1. Yield 31%.

### Example 67

Preparation of 2-(5-chloro-3-(1-(2,5-difluorobenzyl)-6-oxo-1,6-dihydropyridazin-3-yl)-2-methyl-1H-indol-1-yl)acetic acid (67). The title compound was prepared according to the procedure of Example 1; Yield 21%

### Example 68

Preparation of 2-(5-chloro-3-(1-(2,4-difluorobenzyl)-6-oxo-1,6-dihydropyridazin-3-yl)-2-methyl-1H-indol-1-yl)acetic acid (68). The title compound was prepared according to the procedure of Example 1; Yield 20%.

### Example 69

Preparation of 2-(5-chloro-2-methyl-3-(6-oxo-1-(2,4,5-trifluorobenzyl)-1,6-dihydropyridazin-3-yl)-1H-indol-1-yl)acetic acid (69). The title compound was prepared according to the procedure of Example 1; Yield 20%.

### Example 70

Preparation of 2-(5-fluoro-2-methyl-3-(6-oxo-1-(2,4,5-trifluorobenzyl)-1,6-dihydro-pyridazin-3-yl)-1H-indol-1-yl)acetic acid (70). The title compound was prepared according to the procedure of Example 12. Yield 55%.

### Example 71

Preparation of 2-(3-(1-(2,4-difluorobenzyl)-6-oxo-1,6-dihydropyridazin-3-yl)-5-fluoro-2-methyl-1H-indol-1-yl)acetic acid (71). The title compound was prepared according to the procedure of Example 12. Yield 63%.

### Example 72

Preparation of 2-(3-(142,5-difluorobenzyl)-6-oxo-1,6-dihydropyridazin-3-yl)-5-fluoro-2-methyl-1H-indol-1-yl)acetic acid (72). The title compound was prepared according to the procedure of Example 12. Yield 65%.

### Example 73

Preparation of 2-(5-fluoro-2-methyl-3-(1-(4-(methylsulfonyl)benzyl)-6-oxo-1,6-dihydro-pyridazin-3-yl)-1H-indol-1-yl)acetic acid (73). The title compound was prepared according to the procedure of Example 1. Yield 52%.

### Example 74

Step 1: Preparation of methyl 2-(2-methyl-1H-indol-1-yl)acetate, Intermediate 94. To a 500 mL round bottom flask under an atmosphere of nitrogen was added 2-methylindole (10.0 g, 76.23 mmol, 1.0 equiv) and 200 mL DMF. To this was added sodium hydride (3.66 g, 91.48 mmol, 1.2 equiv) and the resulting suspension was allowed to stir at room temperature for 30 minutes. Methyl bromoacetate (8.4 mL, 91.48 mmol, 1.2 equiv) was then added and the mixture was allowed to stir for 16 hours. The reaction was quenched with water and then poured into 700 mL of water. This was extracted with three 200 mL portions of ethyl acetate. The combined organic layers were washed with water and brine, then dried over MgSO₄. Filtration and concentration in vacuo gave the crude product which was purified by silica gel chromatography to give the desired product as a white solid (3.0 g, 20%).

Step 2: Preparation of methyl 2-(3-((l-benzyl-6-oxo-1,6-dihydropyridazin-3-yl)-methyl)-2-methyl-1H-indol-1-yl)acetate, Intermediate 96. To a 250 mL round bottom flask under an atmosphere of nitrogen was added intermediate 104 methyl 2-(2-methyl-1H-indol-1-yl)acetate (3.0 g, 14.77 mmol, 1.0 equiv), intermediate 95, 1-benzyl-6-oxo-1,6-dihydropyridazine-3-carbaldehyde (3.46 g, 16.24 mmol, 1.1 equiv) and 100 mL anhydrous methylene chloride. The resulting solution was cooled to 0° C. in an ice/water bath and triethylsilane (8.26 mL, 51.69 mmol, 3.5 equiv) and trifluoroacetic acid (3.3 mL, 44.30 mmol, 3.0 equiv) were added dropwise. The mixture was allowed to warm to room temperature and then stirred for 24 hours. The mixture was then poured into sat NaHCO_{3(aq)} and the aqueous layer extracted with two 50 mL portions of methylene chloride. The combined organic layers were then washed with water and brine, and dried over magnesium sulfate. Filtration and removal of solvent in vacuo gave the crude material which was then purified by silica gel chromatography to give a white solid (2.52 g, 42%).

Step 3: Preparation of 2-(2-methyl-3-((6-oxo-1,6-dihydropyridazin-3-yl)methyl)-1H-indol-1-yl)-acetic acid, Intermediate 96A. To a 5 mL microwave reaction vessel was added intermediate 96, methyl 2-(3-((l-benzyl-6-oxo-1,6-dihydropyridazin-3-yl)methyl)-2-methyl-1H-indol-1-yl)acetate (0.190 g, 0.47 mmol, 1.0 equiv), aluminum trichloride (0.375 g, 2.84 mmol, 6.0 equiv) and 5 mL toluene. The vessel was sealed and the mixture heated to 140° C. in a microwave reactor for one hour. The mixture was then poured into 50 mL water and extracted with three 50 mL portions of ethyl acetate. The combined organic layers were washed with water and brine, then dried over MgSO₄. Filtration and concentration in vacuo gave the crude product. This was purified by reverse phase HPLC to give the product as a tan solid (0.030 g, 22%).

Step 4: Preparation of 2-fluorobenzyl 2-(3-((1-(2-fluorobenzyl)-6-oxo-1,6-dihydropyridazin-3-yl)methyl)-2-methyl-1H-indol-1-yl)acetate, Intermediate 97. To a 100 mL round bottom flask under a nitrogen atmosphere was added intermediate 96A, 2-(2-methyl-3-((6-oxo-1,6-dihydropyridazin-3-yl)methyl)-1H-indol-1-yl)acetic acid (0.066 g, 0.22 mmol, 1.0 equiv), 2-fluorobenzyl bromide (0.127 g, 0.67 mmol, 3.0 equiv), potassium carbonate (0.123 g, 0.89 mmol, 4.0 equiv) and 40 mL DMF. The resulting suspension was heated to 85° C. for 16 hours. The mixture was then allowed to cool to room temperature and then poured into 200 mL water. This was extracted with three 50 mL portions of ethyl acetate. The combined organic layers were washed with water and brine and dried over MgSO₄. Filtration and concentration in vacuo gave a tan solid. The crude material was purified by silica gel chromatography to give a white solid (0.031 g, 27%).

Step 5: Preparation of 2-(3-((1-(2-fluorobenzyl)-6-oxo-1,6-dihydropyridazin-3-yl)methyl)-2-methyl-1H-indol-1-yl)acetic acid (74). To a 50 mL round bottom flask under an atmosphere of nitrogen was added intermediate 97, 2-fluorobenzyl 2-(3-((1-(2-fluorobenzyl)-6-oxo-1,6-dihydropyridazin-3-yl)methyl)-2-methyl-1H-indol-1-yl)acetate (0.031 g, 0.06 mmol, 1.0 equiv) and 20 mL methanol. To this was added 0.12 mL of 5.0 N NaOH. The resulting mixture was allowed to stir for 16 hours at room temperature. Poured mixture into 100 mL 1.2 N HCl and extracted with three 50 mL portions of ethyl acetate. The combined organic layers were washed with water and brine and dried over MgSO₄. Filtration and concentration in vacuo gave a tan solid. The crude material was purified by reverse phase HPLC. The isolated material was then suspended in acetonitrile/water, frozen and lyophilized to give the desired product as a white lyophilized powder (0.028 g, 100%). ¹H NMR (400 MHz, DMSO-d₆) δ 2.30 (s, 3H) 3.94 (s, 2H) 4.91 (s, 2H) 5.30 (s, 2H) 6.84 (d, J=9.6 Hz, 1H) 6.90 (dd, J=7.6, 1.1 Hz, 1H) 7.03 (td, J=7.6, 1.1 Hz, 1H) 7.14 (d, J=8.0 Hz, 1H) 7.17 (dd, J=7.3, 1.3 Hz, 1H) 7.19-7.27 (m, 2H) 7.31 (d, J=9.1 Hz, 2H) 7.34-7.43 (m, 1H).

### Example 75

Step 1: Preparation of 3-fluorobenzyl 2-(3-((1-(3-fluorobenzyl)-6-oxo-1,6-dihydropyridazin-3-yl)methyl)-2-methyl-1H-indol-1-yl)acetate, Intermediate 98. To a 100 mL round bottom flask under a nitrogen atmosphere was added intermediate 96A, 2-(2-methyl-34(6-oxo-1,6-dihydropyridazin-3-yl)methyl)-1H-indol-1-yl)acetic acid (0.030 g, 0.10 mmol, 1.0 equiv), 3-fluorobenzyl bromide (0.058 g, 0.30 mmol, 3.0 equiv), potassium carbonate (0.055 g, 0.40 mmol, 4.0 equiv) and 40 mL DMF. The resulting suspension was heated to 85° C. for 16 hours. The mixture was then allowed to cool to room temperature and then poured into 200 mL water. This was extracted with three 50 mL portions of ethyl acetate. The combined organic layers were washed with water and brine and dried over MgSO₄. Filtration and concentration in vacuo gave a tan solid. The crude material was purified by silica gel chromatography to give a white solid (0.033 g, 64%).

Step 2: Preparation of 2-(3-((1-(3-fluorobenzyl)-6-oxo-1,6-dihydropyridazin-3-yl)methyl)-2-methyl-1H-indol-1-yl)acetic acid (75). To a 50 mL round bottom flask under an atmosphere of nitrogen was added intermediate 98, 3-fluorobenzyl 2-(3-((1-(3-fluorobenzyl)-6-oxo-1,6-dihydropyridazin-3-yl)methyl)-2-methyl-1H-indol-1-yl)acetate (0.033 g, 0.06 mmol, 1.0 equiv) and 20 mL methanol. To this was added 0.13 mL of 5.0 N NaOH. The resulting mixture was allowed to stir for 16 hours at room temperature. Poured mixture into 100 mL 1.2 N HCl and extracted with three 50 mL portions of ethyl acetate. The combined organic layers were washed with water and brine and dried over MgSO₄. Filtration and concentration in vacuo gave a tan solid. The crude material was purified by reverse phase HPLC. The isolated material was then suspended in acetonitrile/water, frozen and lyophilized to give the desired product as a white lyophilized powder (0.023 g, 95%). ¹H NMR (400 MHz, DMSO-d₆) δ 2.33 (s, 3H) 3.98 (s, 2H) 4.93 (s, 2H) 5.25 (s, 2H) 6.84 (d, J=9.6 Hz, 1H) 6.87-6.94 (m, 1H) 7.03 (td, J=7.6, 1.3 Hz, 1H) 7.08-7.22 (m, 3H) 7.26-7.47 (m, 4H).

### Example 76

Step 4: Preparation of 4-fluorobenzyl 2-(3-((1-(4-fluorobenzyl)-6-oxo-1,6-dihydropyridazin-3-yl)-methyl)-2-methyl-1H-indol-1-yl)acetate, Intermediate 99. To a 100 mL round bottom flask under a nitrogen atmosphere was added intermediate 96A, 2-(2-methyl-3-((6-oxo-1,6-dihydropyridazin-3-yl)methyl)-1H-indol-1-yl)acetic acid (0.022 g, 0.07 mmol, 1.0 equiv), 4-fluorobenzyl bromide (0.042 g, 0.22 mmol, 3.0 equiv), K₂CO₃ (0.041 g, 0.29 mmol, 4.0 equiv) and 40 mL DMF. The resulting suspension was heated to 85° C. for 16 hours. The mixture was then allowed to cool to room temperature and then poured into 200 mL water. This was extracted with three 50 mL portions of ethyl acetate. The combined organic layers were washed with water and brine and dried over MgSO₄. Filtration and concentration in vacuo gave a tan solid. The crude material was used.

Step 2: Preparation of 2-(3-((1-(4-fluorobenzyl)-6-oxo-1,6-dihydropyridazin-3-yl)methyl)-2-methyl-1H-indol-1-yl)acetic acid (76). To a 50 mL round bottom flask under an atmosphere of nitrogen was added intermediate 99, 4-fluorobenzyl 2-(3-((1-(4-fluorobenzyl)-6-oxo-1,6-dihydropyridazin-3-yl)methyl)-2-methyl-1H-indol-1-yl)acetate (0.036 g, 0.06 mmol, 1.0 equiv) and 20 mL methanol. To this was added 0.13 mL of 5.0N NaOH. The resulting mixture was allowed to stir for 16 hours at room temperature. Poured mixture into 100 mL 1.2 N HCl and extracted with three 50 mL portions of ethyl acetate. The combined organic layers were washed with water and brine and dried over MgSO₄. Filtration and concentration in vacuo gave a tan solid. The crude material was purified by reverse phase HPLC. The isolated material was then suspended in acetonitrile/water, frozen and lyophilized to give the desired product as a white lyophilized powder (0.022 g, 95%). ¹H NMR (400 MHz, DMSO-d₆) δ 2.32 (s, 3H) 3.97 (s, 2H) 4.91 (s, 2H) 5.21 (s, 2H) 6.83 (d, J=9.6 Hz, 1H) 6.87-6.95 (m, 1H) 6.98-7.08 (m, 1H) 7.08-7.23 (m, 3H) 7.26-7.45 (m, 4H).

### Example 77

Step 1: Preparation of methyl 1-benzyl-6-oxo-1,4,5,6-tetrahydropyridazine-3-carboxylate, Intermediate 100. To a 500 mL round bottom flask equipped with condenser and under a nitrogen atmosphere was added dimethyl 2-oxoglutarate (5.0 g, 28.71 mmol, 1.0 equiv) and benzyl hydrazine dihydrochloride (6.16 g, 31.58 mmol, 1.1 equiv). 250 mL of anhydrous ethanol were added, followed by 15 drops 12 N HCl. The mixture was heated to reflux and allowed to stir at this temperature for 15 hours. At this time, the solvent was removed in vacuo and the crude material purified by silica gel chromatography to give the product as a tan solid (5.61 g, 79%).

Step 2: Preparation of 2-benzyl-6-(hydroxymethyl)-4,5-dihydropyridazin-3(2H)-one, Intermediate 101. To a 500 mL round bottom flask equipped with condenser and under a nitrogen atmosphere was added intermediate 110, 2-benzyl-6-(hydroxymethyl)-4,5-dihydropyridazin-3(2H)-one (5.61 g, 22.80 mmol, 1.0 equiv, sodium borohydride (0.867 g, 22.80 mmol, 1.0 equiv) and 200 mL anhydrous THF. The mixture was heated to reflux and 35 mL of anhydrous methanol was added dropwise over 1 hour. The mixture was refluxed for an additional hour, then allowed to cool to room temperature. 5 mL water was added, and the mixture concentrated in vacuo. 250 mL 0.6 N HCl was added, and the resulting suspension was extracted with three 100 mL portions of methylene chloride. The organic layer was then washed with water and brine, and then dried over magnesium sulfate. Filtration and removal of solvent in vacuo gave a crude material that was purified by silica gel chromatography (1% methanol/ethyl acetate)to give the desired product as a tan solid (2.81 g, 57%).

Step 3: Preparation of 1-benzyl-6-oxo-1,6-dihydropyridazine-3-carbaldehyde, Intermediate 102. To a 500 mL round bottom flask equipped with condenser and under a nitrogen atmosphere was added intermediate 111, 2-benzyl-6-(hydroxymethyl)-4,5-dihydropyridazin-3(2H)-one (2.81 g, 12.95 mmol, 1.0 equiv), manganese dioxide (16.82 g, 0.194 mol, 15.0 equiv) and 300 mL anhydrous toluene. The resulting suspension was heated to reflux and allowed to stir for 20 hours. At this time, the mixture was allowed to cool to room temperature, filtered through celite and the solvent removed in vacuo to give the desired product as a light yellow oil which solidified upon standing (0.548 g, 20%).

Step 4: Preparation of Methyl 2-(3-((l-benzyl-6-oxo-1,6-dihydropyridazin-3-yl)-methyl)-2-methyl-1H-indol-1-yl)acetate, Intermediate 103. To a 250 mL round bottom flask under an atmosphere of nitrogen was added intermediate 94, methyl 2-(2-methyl-1H-indol-1-yl)acetate (0.300 g, 1.48 mmol, 1.0 equiv), intermediate 102, 1-benzyl-6-oxo-1,6-dihydropyridazine-3-carbaldehyde (0.350 g, 1.63 mmol, 1.1 equiv) and 100 mL anhydrous methylene chloride. The resulting solution was cooled to 0° C. in an ice/water bath and triethylsilane (0.83 mL, 5.17 mmol, 3.5 equiv) and trifluoroacetic acid (0.33 mL, 4.43 mmol, 3.0 equiv) were added dropwise. The mixture was allowed to warm to room temperature and then stirred for 24 hours. The mixture was then poured into sat NaHCO₃₀₀ and the aqueous layer extracted with two 50 mL portions of methylene chloride. The combined organic layers were then washed with water and brine, and dried over magnesium sulfate. Filtration and removal of solvent in vacuo gave the crude material which was then purified by silica gel chromatography to give a white solid (0.524 g, 88%).

Step 5: Preparation of 2-(3-((l-benzyl-6-oxo-1,6-dihydropyridazin-3-yl)methyl)-2-methyl-1H-indol-1-yl)acetic acid (77). To a 100 mL round bottom flask was added intermediate 103 (0.524 g, 1.31 mmol, 1.0 equiv) and 20 mL THF. To this was added a solution of Lithium hydroxide (0.157 g, 6.53 mmol, 5.0 equiv) in 10 mL water. To the resulting biphasic mixture was added methanol dropwise until a single layer formed. The resulting solution was stirred for 2 hours at room temperature. It was then poured into 1.2 N HCl_{(aq)} and the aqueous layer was extracted with three 50 mL portions of ethyl acetate. The combined organic layers were washed with water and brine and dried over magnesium sulfate. Filtration and removal of solvent in vacuo gave the crude material. This was purified by reverse phase HPLC and the isolated product lyophilized to give a white powder (0.254 g, 50%). ¹H NMR (400 MHz, DMSO-d₆) δ 2.32 (s, 3H) 3.97 (s, 2H) 4.93 (s, 2H) 5.24 (s, 2H) 6.83 (d, J=9.6 Hz, 1H) 6.87-6.95 (m, 1H) 6.99-7.07 (m, 1H) 7.12 (d, J=9.3 Hz, 1H) 7.24-7.42 (m, 7H).

### Example 78

Step 1: Preparation of 1-Benzyl-6-oxo-1,4,5,6-tetrahydropyridazine-3-carbaldehyde, Intermediate 102. The procedure described above for intermediate 87 was followed, reacting intermediate 101 (3.88 g, 17.8 mmol) with oxalyl chloride (10.2 mL of a 2.0 M dichloromethane solution, 20.4 mmol) and dimethylsulfoxide (3.0 mL, 3.3 g, 43 mmol), then with triethylamine (12.4 mL, 8.99 g, 89.0 mmol). 3.06 g, 80% yield.

Step 2: Preparation of Methyl 2-(3-((l-benzyl-6-oxo-1,4,5,6-tetrahydropyridazin-3-yl)methyl)-5-fluoro-2-methyl-1H-indol-1-yl)acetate, Intermediate 103. The procedure described above for intermediate 37A was followed, reacting intermediate 36 (1.85 g, 8.37 mmol) with intermediate 102 (1.81 g, 8.37 mmol) in the presence of triethylsilane (3.7 mL, 2.7 g, 23 mmol) and trifluoroacetic acid (1.3 mL, 1.9 g, 17 mmol). 2.22 g, 63% yield.

Step 3: Preparation of 2-(3-((l-Benzyl-6-oxo-1,4,5,6-tetrahydropyridazin-3-yl)methyl)-5-fluoro-2-methyl-1H-indol-1-yl)acetic acid (78). The procedure described above for 20 was followed, reacting intermediate 103 (2.22 g, 5.27 mmol) with lithium hydroxide monohydrate (3.51 g, 84 mmol). The crude product was recrystallized from acetonitrile/ethanol. A small impurity was still present. The recrystallized product and evaporated filtrate were each purified separately by preparative HPLC (water/acetonitrile with 0.1% formic acid). Each set of purified fractions was partially evaporated to remove acetonitrile, then acidified to pH 1 with 0.5 M hydrochloric acid, extracted into ethyl acetate (3×), washed with water, dried over anhydrous magnesium sulfate, filtered and evaporated. Each batch was then recrystallized from acetonitrile/ethanol. Finally, the pure recrystallized material from the two batches was re-combined (585 mg, 27% yield): ¹H NMR (DMSO-d₆) δ 12.99 (s, 1H), 7.36 (dd, J=8.8, 4.5 Hz, 1H), 7.28-7.34 (m, 2H), 7.22-7.28 (m, 3H), 7.14 (dd, J=9.9, 2.5 Hz, 1H), 6.87 (td, J=9.2, 2.5 Hz, 1H), 4.94 (s, 2H), 4.84 (s, 2H), 3.65 (s, 2H), 2.26-2.32 (m, 4H), 2.24 (s, 3H).

### Example 79

Step 1: Preparation of 2,3-difluorobenzyl 2-(3-((1-(2,3-difluorobenzyl)-6-oxo-1,6-dihydro-pyridazin-3-yl)methyl)-2-methyl-1H-indol-1-yl)acetate, Intermediate 104. To a 100 mL round bottom flask under a nitrogen atmosphere was added intermediate 96A, 2-(2-methyl-3-((6-oxo-1,6-dihydropyridazin-3-yl)methyl)-1H-indol-1-yl)acetic acid (0.074 g, 0.25 mmol, 1.0 equiv), 2,3-difluorobenzyl bromide (0.095 mL, 0.747 mmol, 3.0 equiv), potassium carbonate (0.138 g, 0.996 mmol, 4.0 equiv) and 40 mL DMF. The resulting suspension was heated to 85° C. for 16 hours. The mixture was then allowed to cool to room temperature and then poured into 200 mL water. This was extracted with three 50 mL portions of ethyl acetate. The combined organic layers were washed with water and brine and dried over MgSO₄. Filtration and concentration in vacuo gave a tan solid. This material was purified by silica gel chromatography to give a tan solid (0.153 g, 89%).

Step 2: Preparation of 2-(3-((1-(2,3-difluorobenzyl)-6-oxo-1,6-dihydropyridazin-3-yl)methyl)-2-methyl-1H-indol-1-yl)acetic acid (79). To a 100 mL round bottom flask was added intermediate 104 (0.153 g, 0.287 mmol, 1.0 equiv) and 20 mL THF. To this was added a solution of Lithium hydroxide (0.033 g, 1.39 mmol, 5.0 equiv) in 10 mL water. To the resulting biphasic mixture was added methanol dropwise until a single layer formed. The resulting solution was stirred for 2 hours at room temperature. It was then poured into 1.2 N HCl_{(aq)} and the aqueous layer was extracted with three 50 mL portions of ethyl acetate. The combined organic layers were washed with water and brine and dried over magnesium sulfate. Filtration and removal of solvent in vacuo gave the crude material. This was purified by reverse phase HPLC and the isolated product lyophilized to give a white powder (0.072 g, 61%). ¹H NMR (400 MHz, DMSO-d₆) δ 2.31 (s, 3H) 3.94 (s, 2H) 4.92 (s, 2H) 5.33 (s, 2H) 6.75-6.94 (m, 2H) 6.96-7.10 (m, 2H) 7.12-7.23 (m, 2H) 7.31 (t, J=8.1 Hz, 2H) 7.36-7.51 (m, 1H).

### Example 80

Step 1: Preparation of methyl 2-(3-((1-benzyl-6-oxo-1,6-dihydropyridazin-3-yl)-methyl)-5-chloro-2-methyl-1H-indol-1-yl)acetate, Intermediate 105. To a 250 mL round bottom flask under an atmosphere of nitrogen was added methyl 2-(5-chloro-2-methyl-1H-indol-1-yl)acetate (0.403 g, 1.70 mmol, 1.0 equiv), intermediate 95, 1-benzyl-6-oxo-1,6-dihydropyridazine-3-carbaldehyde (0.400 g, 1.87 mmol, 1.1 equiv) and 100 mL anhydrous methylene chloride. The resulting solution was cooled to 0° C. in an ice/water bath and triethylsilane (0.95 mL, 5.94 mmol, 3.5 equiv) and trifluoroacetic acid (0.38 mL, 5.10 mmol, 3.0 equiv) were added dropwise. The mixture was allowed to warm to room temperature and then stirred for 24 hours. The mixture was then poured into sat NaHCO_{3(aq)} and the aqueous layer extracted with two 50 mL portions of methylene chloride. The combined organic layers were then washed with water and brine, and dried over magnesium sulfate. Filtration and removal of solvent in vacuo gave the crude material which was then purified by silica gel chromatography to give a white solid (0.471 g, 63%).

Step 2: Preparation of 2-(3-((l-benzyl-6-oxo-1,6-dihydropyridazin-3-yl)methyl)-5-chloro-2-methyl-1H-indol-1-yl)acetic acid (80). To a 100 mL round bottom flask was added intermediate 105, methyl 2-(3-((1-benzyl-6-oxo-1,6-dihydropyridazin-3-yl)methyl)-5-chloro-2-methyl-1H-indol-1-yl)acetate (0.561 g, 1.28 mmol, 1.0 equiv) and 20 mL THF. To this was added a solution of Lithium hydroxide (0.154 g, 6.42 mmol, 5.0 equiv) in 10 mL water. To the resulting biphasic mixture was added methanol dropwise until a single layer formed. The resulting solution was stirred for 2 hours at room temperature. It was then poured into 1.2 N HCl_{(aq)} and the aqueous layer was extracted with three 50 mL portions of ethyl acetate. The combined organic layers were washed with water and brine and dried over magnesium sulfate. Filtration and removal of solvent in vacuo gave the crude material. This was purified by reverse phase HPLC and the isolated product lyophilized to give a white powder (0.128 g, 54%). ¹H NMR (400 MHz, DMSO-d₆) δ 2.31 (s, 3H) 3.98 (s, 2H) 4.95 (s, 2H) 5.21 (s, 2H) 6.85 (d, J=9.6 Hz, 1H) 7.05 (dd, J=8.7, 2.1 Hz, 1H) 7.16 (d, J=9.6 Hz, 1H) 7.24-7.36 (m, 5H) 7.39 (d, J=8.8 Hz, 1H) 7.49 (d, J=2.3 Hz, 1H).

### Example 81

Step 1: Preparation of (4-(trifluoromethyl)benzyl)hydrazine, Intermediate 106. To a 50 mL round bottom flask under a nitrogen atmosphere added hydrazine (6.8 mL, 0.216 mol, 10.5 equiv), and then cooled the flask to 0° C. in an ice/water bath. Added 4-trifluoromethyl-benzyl bromide (4.92 g, 20.58 mmol, 1.0 equiv) in 15 mL methanol dropwise over 15 minutes. The reaction mixture was then allowed to warm to room temperature and stir for 2 hours. The methanol was removed in vacuo and the hydrazine layer was extracted with three 30 mL portions of diethyl ether. The combined organic layers were concentrated in vacuo to give the desired product as a colorless oil (3.62 g, 92%).

Step 2: Preparation of Methyl 6-oxo-1-(4-(trifluoromethyl)benzyl)-1,4,5,6-tetra-hydropyridazine-3-carboxylate, Intermediate 107. To a 250 mL round bottom flask equipped with condenser and under a nitrogen atmosphere was added dimethyl 2-oxoglutarate (3.01 g, 17.31 mmol, 1.0 equiv) and intermediate 106, 4-(trifluoromethyl)benzyl)hydrazine (3.62 g, 19.04 mmol, 1.1 equiv). 250 mL of any ethanol were added, followed by 15 drops 12N HCl. The mixture was heated to reflux and allowed to stir at this temperature for 15 hours. The solvent was removed in vacuo and the crude material purified by silica gel chromatography to give the product as a tan solid (4.31 g, 79%).

Step 3: Preparation of 6-(hydroxymethyl)-2-(4-(trifluoromethyl)benzyl)-4,5-dihydropyridazin-3(2H)-one, Intermediate 108. To a 500 mL round bottom flask equipped with condenser and under a nitrogen atmosphere was added intermediate 107, methyl 6-oxo-1-(4-(trifluoromethyl)benzyl)-1,4,5,6-tetrahydropyridazine-3-carboxylate (4.31 g, 13.73 mmol, 1.0 equiv, sodium borohydride (0.522 g, 13.73 mmol, 1.0 equiv) and 200 mL anhydrous THF. The mixture was heated to reflux and 35 mL of anhydrous methanol was added dropwise over 1 hour. The mixture was refluxed for an additional hour, then allowed to cool to room temperature. 5 mL water was added, and the mixture concentrated in vacuo. 250 mL 0.6 N HCl was added, and the resulting suspension was extracted with three 100 mL portions of methylene chloride. The organic layer was then washed with water and brine, and then dried over magnesium sulfate. Filtration and removal of solvent in vacuo gave a crude material that was purified by silica gel chromatography (1% methanol/ethyl acetate)to give the desired product as a tan solid (1.52 g, 39%).

Step 4: Preparation of 6-oxo-1-(4-(trifluoromethyl)benzyl)-1,6-dihydro-pyridazine-3-carbaldehyde, Intermediate 109. To a 500 mL round bottom flask equipped with condenser and under a nitrogen atmosphere was added intermediate 108, 6-(hydroxymethyl)-2-(4-(trifluoromethyl)benzyl)-4,5-dihydro-pyridazin-3(2H)-one (1.52 g, 5.33 mmol, 1.0 equiv), manganese dioxide (6.96 g, 80.0 mmol, 15.0 equiv) and 300 mL anhydrous toluene. The resulting suspension was heated to reflux and allowed to stir for 20 hours. At this time, the mixture was allowed to cool to room temperature, filtered through celite and the solvent removed in vacuo to give the desired product as a light yellow oil which solidified upon standing (0.570 g, 38%).

Step 5: Preparation of methyl 2-(5-chloro-2-methyl-346-oxo-1-(4-(trifluoro-methyl)benzyl)-1,6- dihydropyridazin-3-yl)methyl)-1H-indol-1-yl)acetate, Intermediate 110. To a 250 mL round bottom flask under an atmosphere of nitrogen was added methyl 2-(5-chloro-2-methyl-1H-indol-1-yl)acetate (0.231 g, 0.97 mmol, 1.0 equiv), intermediate 109, 6-oxo-1-(4-(trifluoromethyl)benzyl)-1,6-dihydropyridazine-3-carbaldehyde (0.300 g, 1.07 mmol, 1.1 equiv) and 100 mL anhydrous methylene chloride. The resulting solution was cooled to 0° C. in an ice/water bath and triethylsilane (0.55 mL, 3.40 mmol, 3.5 equiv) and trifluoroacetic acid (0.22 mL, 2.91 mmol, 3.0 equiv) were added dropwise. The mixture was allowed to warm to room temperature and then stirred for 24 hours. The mixture was then poured into sat NaHCO_{3(aq)} and the aqueous layer extracted with two 50 mL portions of methylene chloride. The combined organic layers were then washed with water and brine, and dried over magnesium sulfate. Filtration and removal of solvent in vacuo gave the crude material which was then purified by silica gel chromatography to give a white solid (0.402 g, 82%).

Step 6: Preparation of 2-(5-chloro-2-methyl-346-oxo-1-(4-(trifluoromethyl)benzyl)-1,6-dihydro-pyridazin-3-yl)methyl)-1H-indol-1-yl)acetic acid (81). To a 100 mL round bottom flask was added intermediate 110, methyl 2-(5-chloro-2-methyl-3-((6-oxo-1-(4-(trifluoromethyl)benzyl)-1,6-dihydropyridazin-3-yl)methyl)-1H-indol-1-yl)acetate (0.402 g, 0.80 mmol, 1.0 equiv) and 20 mL THF. To this was added a solution of Lithium hydroxide (0.096 g, 4.0 mmol, 5.0 equiv) in 10 mL water. To the resulting biphasic mixture was added methanol dropwise until a single layer formed. The resulting solution was stirred for 2 hours at room temperature. It was then poured into 1.2 N HCl_{(aq)} and the aqueous layer was extracted with three 50 mL portions of ethyl acetate. The combined organic layers were washed with water and brine and dried over magnesium sulfate. Filtration and removal of solvent in vacuo gave the crude material. This was purified by reverse phase HPLC and the isolated product lyophilized to give a white powder (0.180 g, 46%). ¹H NMR (400 MHz, DMSO-d₆) δ 2.31 (s, 3H) 3.98 (s, 2H) 4.96 (s, 2H) 5.32 (s, 2H) 6.89 (d, J=9.6 Hz, 1H) 7.04 (dd, J=8.7, 2.1 Hz, 1H) 7.19 (d, J=9.6 Hz, 1H) 7.33-7.44 (m, 2H) 7.49 (d, J=7.8 Hz, 2H) 7.69 (d, J=8.1 Hz, 2H) 13.07 (br. s., 1H).

### Example 82

Step 1: Preparation of methyl 2-(5-fluoro-2-methyl-3-((6-oxo-1-(4-(trifluoro-methyl)benzyl)-1,6-dihydropyridazin-3-yl)methyl)-1H-indol-1-yl)acetate, Intermediate 111. To a 250 mL round bottom flask under an atmosphere of nitrogen was added methyl 2-(5-fluoro-2-methyl-1H-indol-1-yl)acetate (0.165 g, 0.75 mmol, 1.0 equiv), intermediate 109, 6-oxo-1-(4-(trifluoromethyl)benzyl)-1,6-dihydropyridazine-3-carbaldehyde (0.231 g, 0.82 mmol, 1.1 equiv) and 100 mL anhydrous methylene chloride. The resulting solution was cooled to 0° C. in an ice/water bath and triethylsilane (0.42 mL, 2.62 mmol, 3.5 equiv) and trifluoroacetic acid (0.17 mL, 2.24 mmol, 3.0 equiv) were added dropwise. The mixture was allowed to warm to room temperature and then stirred for 24 hours. The mixture was then poured into sat NaHCO_{3(aq)} and the aqueous layer extracted with two 50 mL portions of methylene chloride. The combined organic layers were then washed with water and brine, and dried over magnesium sulfate. Filtration and removal of solvent in vacuo gave the crude material which was then purified by silica gel chromatography to give a white solid (0.345 g, 86%).

Step 2: Preparation of 2-(5-fluoro-2-methyl-3-((6-oxo-1-(4-(trifluoromethyl)benzyl)-1,6-dihydro-pyridazin-3-yl)methyl)-1H-indol-1-yl)acetic acid (82). To a 100 mL round bottom flask was added intermediate 111, methyl 2-(5-fluoro-2-methyl-3-((6-oxo-1-(4-(trifluoromethyl)benzyl)-1,6-dihydropyridazin-3-yl)methyl)-1H-indol-1-yl)acetate (0.345 g, 0.71 mmol, 1.0 equiv) and 20 mL THF. To this was added a solution of Lithium hydroxide (0.085 g, 3.54 mmol, 5.0 equiv) in 10 mL water. To the resulting biphasic mixture was added methanol dropwise until a single layer formed. The resulting solution was stirred for 2 hours at room temperature. It was then poured into 1.2 N HCl_{(aq)} and the aqueous layer was extracted with three 50 mL portions of ethyl acetate. The combined organic layers were washed with water and brine and dried over magnesium sulfate. Filtration and removal of solvent in vacuo gave the crude material. This was purified by reverse phase HPLC and the isolated product lyophilized to give a white powder (0.214 g, 34%).

### Example 83

Step 1: Preparation of methyl 2-(5-fluoro-2-methyl-346-oxo-1,6-dihydropyridazin-3-yl)methyl)-1H-indol-1-yl)acetate, Intermediate 112. To a 500 mL round bottom flask under an atmosphere of nitrogen was added methyl 2-(5-fluoro-2-methyl-1H-indol-1-yl)acetate (3.57 g, 16.12 mmol, 1.0 equiv), 6-oxo-1,6-dihydropyridazine-3-carbaldehyde (2.0 g, 16.12 mmol, 1.0 equiv) and 200 mL anhydrous methylene chloride. The resulting solution was cooled to 0° C. in an ice/water bath and triethylsilane (9.01 mL, 56.4 mmol, 3.5 equiv) and trifluoroacetic acid (3.72 mL, 48.3 mmol, 3.0 equiv) were added dropwise. The mixture was allowed to warm to room temperature and then stirred for 24 hours. The mixture was then poured into sat NaHCO_{3(aq)} and the aqueous layer extracted with two 50 mL portions of methylene chloride. The combined organic layers were then washed with water and brine, and dried over magnesium sulfate. Filtration and removal of solvent in vacuo gave the crude material which was then purified by silica gel chromatography to give a white solid (2.03 g, 38%). ¹H NMR (400 MHz, DMSO-d₆) δ 2.32 (s, 3H) 3.68 (s, 3H) 3.93 (s, 2H) 5.09 (s, 2H) 6.76 (dd, J=9.6, 2.0 Hz, 1H) 6.89 (td, J=9.2, 2.7 Hz, 1H) 7.17 (d, J=9.9 Hz, 1H) 7.21 (dd, J=9.9, 2.5 Hz, 1H) 7.37 (dd, J=8.8, 4.3 Hz, 1H) 12.75 (s, 1H).

Step 2: Preparation of methyl 2-(3-((1-(2,4-difluorobenzyl)-6-oxo-1,6-dihydropyridazin-3-yl)methyl)-5-fluoro-2-methyl-1H-indol-1-yl)acetate, Intermediate 113. To a 100 mL round bottom flask under a nitrogen atmosphere was added intermediate 112, methyl 2-(5-fluoro-2-methyl-3-((6-oxo-1,6-dihydropyridazin-3-yl)methyl)-1H-indol-1-yl)acetate (2.03 g, 6.16 mmol, 1.0 equiv), 2,4-difluorobenzyl bromide (1.58 mL, 12.33 mmol, 2.0 equiv), potassium carbonate (2.56 g, 18.49 mmol, 3.0 equiv) and 50 mL DMF. The resulting suspension was heated to 85° C. for 16 hours. The mixture was then allowed to cool to room temperature and then poured into 200 mL water. This was extracted with three 50 mL portions of ethyl acetate. The combined organic layers were washed with water and brine and dried over MgSO₄. Filtration and concentration in vacuo gave a tan solid. The crude material was purified by silica gel chromatography to give a white solid (2.35 g, 80%). ¹H NMR (400 MHz, CHLOROFORM-d) δ 2.32 (s, 3H) 3.75 (s, 3H) 3.93 (s, 2H) 4.77 (s, 2H) 5.35 (s, 2H) 6.77 (d, J=9.6 Hz, 1H) 6.79-6.85 (m, 2H) 6.89 (td, J=9.1, 2.5 Hz, 1H) 6.96 (d, J=9.3 Hz, 1H) 7.01-7.11 (m, 2H) 7.29-7.38 (m, 1H).

Step 3: Preparation of 2-(3-((1-(2,4-difluorobenzyl)-6-oxo-1,6-dihydropyridazin-3-yl)methyl)-5-fluoro-2-methyl-1H-indol-1-yl)acetic acid (83). To a 100 mL round bottom flask was added intermediate 113, methyl 2-(3-((1-(2,4-difluorobenzyl)-6-oxo-1,6-dihydropyridazin-3-yl)methyl)-5-fluoro-2-methyl-1H-indol-1-yl)acetate (2.35 g, 5.16 mmol, 1.0 equiv) and 20 mL THF. To this was added a solution of Lithium hydroxide (0.618 g, 25.8 mmol, 5.0 equiv) in 10 mL water. To the resulting biphasic mixture was added methanol dropwise until a single layer formed. The resulting solution was stirred for 2 hours at room temperature. It was then poured into 1.2 N HCl_{(aq)} and the aqueous layer was extracted with three 50 mL portions of ethyl acetate. The combined organic layers were washed with water and brine and dried over magnesium sulfate. Filtration and removal of solvent in vacuo gave the crude material. This was purified by reverse phase HPLC and the isolated product lyophilized to give a white powder (1.97 g, 86%). ¹H NMR (400 MHz, DMSO-d₆) δ 2.28 (s, 3H) 3.91 (s, 2H) 4.94 (s, 2H) 5.25 (s, 2H) 6.80-6.91 (m, 2H) 7.03 (m, J=8.6, 8.6, 2.6, 1.0 Hz, 1H) 7.09 (dd, J=9.7, 2.4 Hz, 1H) 7.17 (d, J=9.6 Hz, 1H) 7.21-7.28 (m, 1H) 7.28-7.38 (m, 2H) 13.00 (s, 1H).

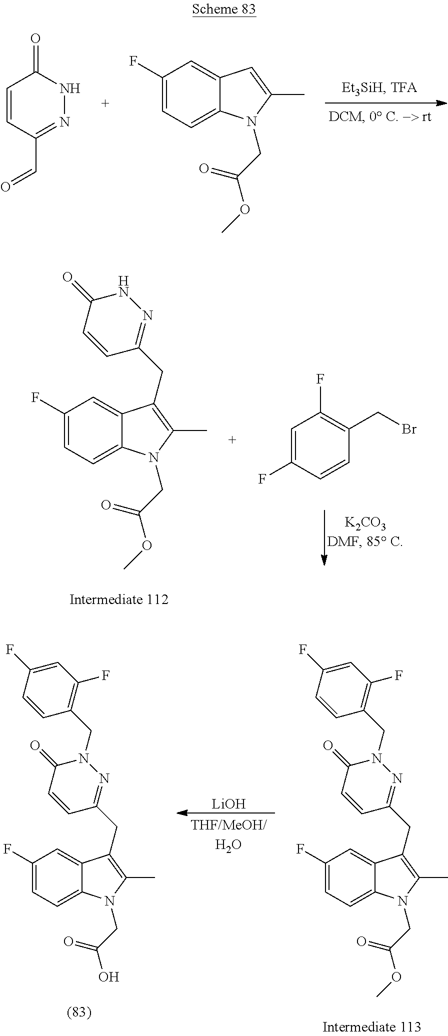

### Example 84

Step 1: Preparation of methyl 2-(5-chloro-2-methyl-1H-indol-3-yl)acetate, Intermediate 114. To a 250 mL round bottom flask under an atmosphere of nitrogen was added 5-chloro-2-methylindole (5.0 g, 30.2 mmol, 1.0 equiv) and 100 mL THF. The resulting solution was cooled to −78° C. in a dry ice/acetone bath, and n-butyllithium (21 mL of 1.51M solution, 31.72 mmol, 1.05 equiv) was added dropwise over 30 minutes. This was allowed to stir at −78° C. for 30 minutes, at which point zinc chloride (4.12 g, 30.2 mmol, 1.0 equiv) was added as a solution in 5 mL THF dropwise. The resulting mixture was allowed to warm to room temperature. Methyl bromoacetate (2.78 mL, 30.2 mmol, 1.0 equiv) was then added and the reaction allowed to stir fro 24 hours. The reaction mixture was then poured into 500 mL saturated aqueous ammonium chloride and extracted with three 100 mL portions of ethyl acetate. The combined organic layers were washed with water and brine and dried over MgSO₄. Filtration and concentration in vacuo gave the crude material which was purified by silica gel chromatography to give the desired product as a yellow oil (4.53 g, 63%). ¹H NMR (400 MHz, CHLOROFORM-d) δ 2.35 (s, 3H) 3.64 (s, 2H) 3.68 (s, 3H) 6.99-7.07 (m, 1H) 7.09-7.14 (m, 1H) 7.46 (d, J=2.0 Hz, 1H) 7.95 (br. s., 1H).

Step 2: Preparation of 2-(5-chloro-2-methyl-1H-indol-3-yl)acetic acid, Intermediate 115. To a 250 mL round bottom flask was added intermediate 114, methyl 2-(5-chloro-2-methyl-1H-indol-3-yl)acetate (4.53 g, 19.07 mmol, 1.0 equiv) and 50 mL THF. To this was added a solution of Lithium hydroxide (2.28 g, 95.37 mmol, 5.0 equiv) in 25 mL water. To the resulting biphasic mixture was added methanol dropwise until a single layer formed. The resulting solution was stirred for 2 hours at room temperature. It was then poured into 1.2 N HCl_{(aq)} and the aqueous layer was extracted with three 100 mL portions of ethyl acetate. The combined organic layers were washed with water and brine and dried over magnesium sulfate. Filtration and removal of solvent in vacuo gave the desired product (3.59 g, 84%). ¹H NMR (400 MHz, DMSO-d₆) δ 2.32 (s, 3H) 3.56 (s, 2H) 6.98 (dd, J=8.5, 2.1 Hz, 1H) 7.25 (d, J=8.6 Hz, 1H) 7.40 (d, J=2.0 Hz, 1H) 11.05 (s, 1H) 12.12 (s, 1H).

Step 3: Preparation of 2-(2-(5-chloro-2-methyl-1H-indol-3-yl)acetyl)benzoic acid, Intermediate 116. To a 100 mL round bottom flask under an atmosphere of nitrogen added intermediate 115, 2-(5-chloro-2-methyl-1H-indol-3-yl)acetic acid (3.59 g, 16.13 mmol, 1.0 equiv), phthalic anhydride (2.39 g, 16.13 mmol, 1.0 equiv) and sodium acetate (7.94 g, 96.81 mmol, 6.0 equiv). 40 mL of toluene was added, and the suspension was sonicated for 5 minutes. The toluene was then removed in vacuo and the resulting powder heated neat to 200° C. for 16 hours. After cooling to room temperature, the resulting brown solid was washed with 1.2N HCl and water and dried. The resulting material was carried on crude (4.08 g, 77%).

Step 4: Preparation of 4-((5-chloro-2-methyl-1H-indol-3-yl)methyl)phthalazin-1(2H)-one, Intermediate 117. To a 500 mL round bottom flask under an atmosphere of nitrogen was added intermediate 126, 2-(2-(5-chloro-2-methyl-1H-indol-3-yl)acetyl)benzoic acid (4.08 g, 12.48 mmol, 1.0 equiv), anhydrous hydrazine (0.78 mL, 24.95 mL, 2.0 equiv) and 250 mL isopropanol. The resulting mixture was heated to reflux and allowed to stir 16 hours. The solvent was then removed in vacuo and the residue purified by silica gel chromatography to give the desired product as a brown solid (0.755 g, 19%). ¹H NMR (400 MHz, DMSO-d₆) δ 2.40 (s, 3H) 4.32 (s, 2H) 6.94 (dd, J=8.5, 2.1 Hz, 1H) 7.22 (d, J=8.6 Hz, 1H) 7.38-7.46 (m, 1H) 7.75-7.82 (m, 1H) 7.86 (td, J=7.6, 1.5 Hz, 1H) 7.92-7.98 (m, 1H) 8.24 (dd, J=7.8, 1.5 Hz, 1H) 11.06 (s, 1H) 12.52 (s, 1H).

Step 5: Preparation of 2-benzyl-4-((5-chloro-2-methyl-1H-indol-3-yl)methyl)-phthalazin-1(2H)-one, Intermediate 118. To a 100 mL round bottom flask under a nitrogen atmosphere was added intermediate 117, 4-((5-chloro-2-methyl-1H-indol-3-yl)methyl)phthalazin-1(2H)-one (0.755 g, 2.34 mmol, 1.0 equiv), benzyl bromide (0.56 mL, 4.67 mmol, 2.0 equiv), potassium carbonate (0.806 g, 5.84 mmol, 2.5 equiv) and 50 mL DMF. The resulting suspension was heated to 85° C. for 16 hours. The mixture was then allowed to cool to room temperature and then poured into 200 mL water. This was extracted with three 50 mL portions of ethyl acetate. The combined organic layers were washed with water and brine and dried over MgSO₄. Filtration and concentration in vacuo gave a tan solid. The crude material was purified by silica gel chromatography to give a white solid (0.200 g, 21%).

Step 6: Preparation of methyl 2-(3-((3-benzyl-4-oxo-3,4-dihydrophthalazin-1-yl)-methyl)-5-chloro-2-methyl-1H-indol-1-yl)acetate, Intermediate 119. To a 100 mL round bottom flask under a nitrogen atmosphere was added intermediate 118, 2-benzyl-4-((5-chloro-2-methyl-1H-indol-3-yl)methyl)-phthalazin-1(2H)-one (0.128 g, 0.31 mmol, 1.0 equiv), methyl bromoacetate (0.11 mL, 1.24 mmol, 4.0 equiv), potassium carbonate (0.256 g, 1.86 mmol, 6.0 equiv) and 50 mL DMF. The resulting suspension was heated to 85° C. for 16 hours. The mixture was then allowed to cool to room temperature and then poured into 200 mL water. This was extracted with three 50 mL portions of ethyl acetate. The combined organic layers were washed with water and brine and dried over MgSO₄. Filtration and concentration in vacuo gave a tan solid. The crude material was purified by silica gel chromatography to give a white solid (0.081 g, 54%). ¹H NMR (400 MHz, CHLOROFORM-d) δ 0.88 (s, 1H) 2.25 (s, 3H) 3.70 (s, 3H) 4.31 (s, 2H) 4.74 (s, 2H) 5.37 (s, 2H) 6.97-7.13 (m, 2H) 7.21-7.34 (m, 4H) 7.44 (dd, J=8.1, 1.5 Hz, 2H) 7.55 (d, J=1.5 Hz, 1H) 7.62-7.70 (m, 2H) 7.73-7.81 (m, 1H) 8.34-8.48 (m, 1H).

Step 7: Preparation of 2-(3-((3-benzyl-4-oxo-3,4-dihydrophthalaz in-1-yl)methyl)-5-chloro-2-methyl-1H-indol-1-yl)acetic acid (84). To a 100 mL round bottom flask was added intermediate 119, methyl 2-(3-((3-benzyl-4-oxo-3,4-dihydrophthalazin-1-yl)methyl)-5-chloro-2-methyl-1H-indol-1-yl)acetate (0.155 g, 0.32 mmol, 1.0 equiv) and 20 mL THF. To this was added a solution of Lithium hydroxide (0.038 g, 1.60 mmol, 5.0 equiv) in 10 mL water. To the resulting biphasic mixture was added methanol dropwise until a single layer formed. The resulting solution was stirred for 2 hours at room temperature. It was then poured into 1.2 N HCl_{(aq)} and the aqueous layer was extracted with three 50 mL portions of ethyl acetate. The combined organic layers were washed with water and brine and dried over magnesium sulfate. Filtration and removal of solvent in vacuo gave the crude material. This was purified by reverse phase HPLC and the isolated product lyophilized to give a white powder (0.083 g, 55%). ¹H NMR (400 MHz, DMSO-d₆) δ 2.32 (s, 3H) 4.39 (s, 2H) 4.93 (s, 2H) 5.30 (s, 2H) 7.01 (dd, J=8.7, 2.1 Hz, 1H) 7.20-7.42 (m, 6H) 7.52 (d, J=1.8 Hz, 1H) 7.83 (m, J=7.5, 7 .5, 7.5, 7.5, 1.6 Hz, 2H) 7.96 (dd, J=6.9, 1.1 Hz, 1H) 8.28 (dd,

J=7.8, 1.5 Hz, 1H) 13.09 (br. s., 1H).

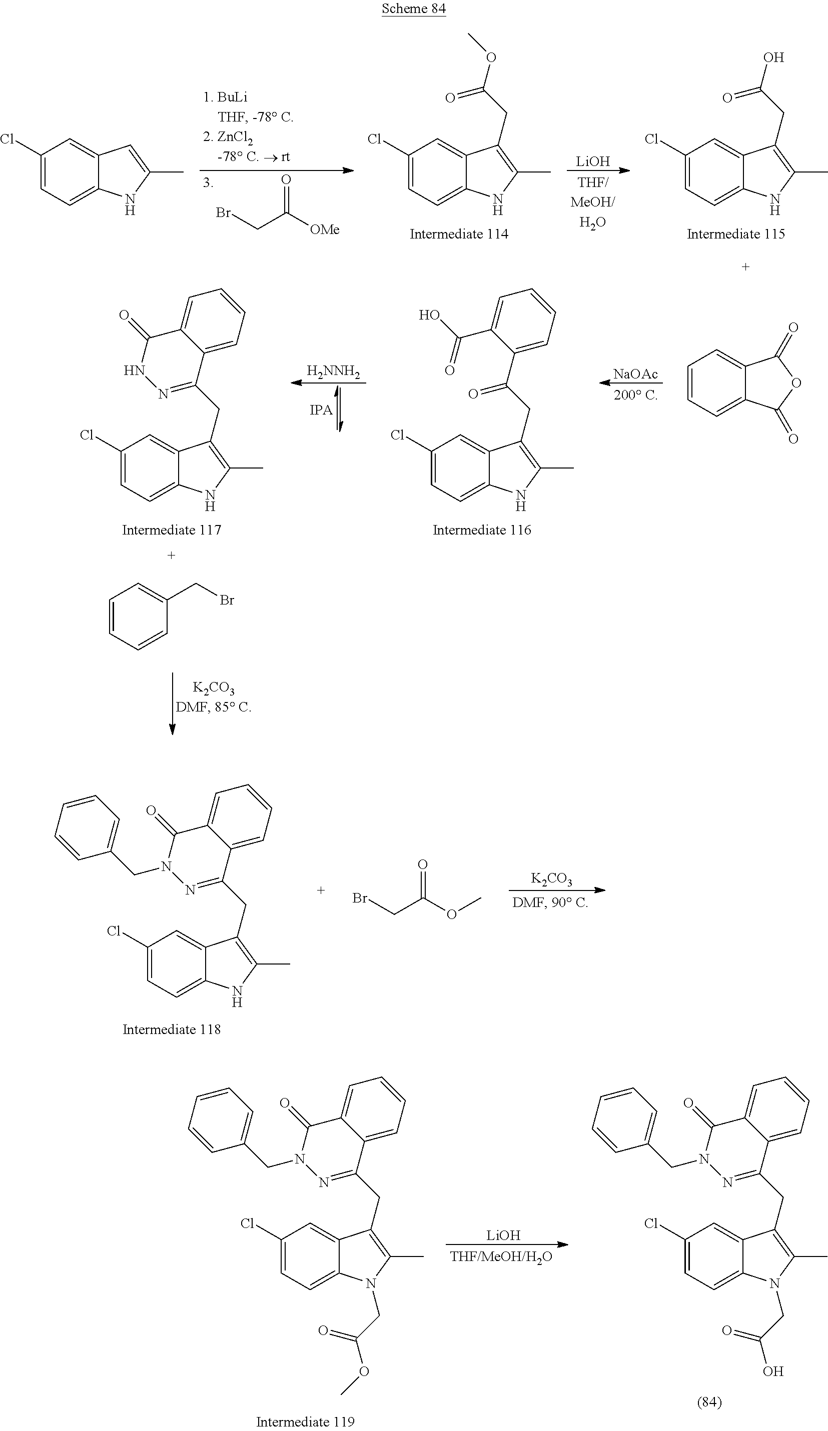

### Example 85

Step 1: Preparation of 2-benzylisoquinolin-1(2H)-one, Intermediate 120. To a 100 mL round bottom flask under an atmosphere of nitrogen was added isocarbostyril (1.0 g, 6.88 mmol, 1.0 equiv) and 50 mL DMF. The resulting solution was cooled to 0° C. in an ice/water bath and 60% sodium hydride in mineral oil (0.303 g, 7.58 mmol, 1.1 equiv) was added in portions. The resulting suspension was allowed to stir at 0° C. for one hour. Benzyl bromide (3.3 mL, 27.56 mmol, 4.0 equiv) was added and the reaction allowed to warm to room temperature. This suspension was allowed to stir an additional 16 hours. It was then poured into 500 mL water and extracted with three 100 mL portions of ethyl acetate. The combined organic layers were washed with water and brine and dried over MgSO₄. Filtration and concentration in vacuo gave the crude material, which was purified by silica gel chromatography. The desired product was isolated as a white solid (1.47 g, 91%). ¹H NMR (400 MHz, CHLOROFORM-d) δ 5.22 (s, 2H) 6.48 (dd, J=7.3, 0.5 Hz, 1H) 7.08 (d, J=7.3 Hz, 1H) 7.23-7.37 (m, 5H) 7.45-7.53 (m, 2H) 7.58-7.68 (m, 1H) 8.47 (dt, J=8.5, 0.8 Hz, 1H).

Step 2: Preparation of 2-benzyl-1-oxo-1,2-dihydroisoquinoline-4-carbaldehyde, Intermediate 121. To a 250 mL round bottom flask equipped with a condenser and under an atmosphere of nitrogen was added 1.44 mL of DMF. This was cooled to 0° C. in an ice/water bath and phosphorus oxychloride (0.43 mL, 4.68 mmol, 1.1 equiv) was added dropwise. A solution of intermediate 130, 2-benzylisoquinolin-1(2H)-one (1.0 g, 4.26 mmol, 1.0 equiv) in a solution of 50 mL DMF was then added dropwise over 30 minutes. At this time the mixture was heated to 100° C. and allowed to stir for 16 hours. The mixture was then poured into 500 mL ice water and extracted with three 100 mL portions of ethyl acetate. The combined organic layers were washed with water and brine and dried over MgSO₄. Filtration and concentration in vacuo gave the crude material which was purified by silica gel chromatography. The desired product was isolated as a white solid (0.257 g, 23%). ¹H NMR (400 MHz, CHLOROFORM-d) δ 5.84-5.93 (m, 0H) 7.28-7.42 (m, 5H) 7.58 (ddd, J=8.1, 7.1, 1.1 Hz, 1H) 7.70 (s, 1H) 7.76 (ddd, J=8.3, 7.1, 1.5 Hz, 1H) 8.45 (dd, J=8.1, 1.5 Hz, 1H) 8.94-9.03 (m, 1H) 9.71 (s, 1H).

Step 3: Preparation of methyl 2-(3-((2-benzyl-1-oxo-1,2-dihydroisoquinolin-4-yl)methyl)-5-fluoro-2-methyl-1H-indol-1-yl)acetate, Intermediate 122. To a 100 mL round bottom flask under an atmosphere of nitrogen was added intermediate 121, 2-benzyl-1-oxo-1,2-dihydroisoquinoline-4-carbaldehyde (0.250 g, 0.95 mmol, 1.1 equiv), methyl 2-(5-fluoro-2-methyl-1H-indol-1-yl)acetate (0.191 g, 0.86 mmol, 1.0 equiv) and 50 mL anhydrous methylene chloride. The resulting solution was cooled to 0° C. in an ice/water bath and triethylsilane (0.48 mL, 3.02 mmol, 3.5 equiv) and trifluoroacetic acid (0.20 mL, 2.59 mmol, 3.0 equiv) were added dropwise. The mixture was allowed to warm to room temperature and then stirred for 24 hours. The mixture was then poured into sat NaHCO_{3(aq)} and the aqueous layer extracted with two 50 mL portions of methylene chloride. The combined organic layers were then washed with water and brine, and dried over magnesium sulfate. Filtration and removal of solvent in vacuo gave the crude material which was then purified by silica gel chromatography to give a white solid (0.369 g, 92%). ¹H NMR (400 MHz, DMSO-d₆) δ 2.27 (s, 3H) 3.66 (s, 3H) 4.08 (s, 2H) 5.10 (s, 2H) 5.10 (s, 2H) 6.86 (td, J=9.2, 2.5 Hz, 1H) 7.11 (dd, J=9.9, 2.5 Hz, 1H) 7.18-7.32 (m, 6H) 7.36 (dd, J=8.8, 4.5 Hz, 1H) 7.48-7.55 (m, 1H) 7.71 (td, J=7.6, 1.4 Hz, 1H) 7.76-7.81 (m, 1H) 8.27 (dd, J=8.0, 1.4 Hz, 1H).

Step 4: Preparation of 2-(3-((2-benzyl-1-oxo-1,2-dihydroisoquinolin-4-yl)methyl)-5-fluoro-2-methyl-1H-indol-1-yl)acetic acid (85). To a 100 mL round bottom flask was added intermediate 122, methyl 2-(3-((2-benzyl-1-oxo-1,2-dihydroisoquinolin-4-yl)methyl)-5-fluoro-2-methyl-1H-indol-1-yl)acetate (0.360 g, 0.77 mmol, 1.0 equiv) and 20 mL THF. To this was added a solution of Lithium hydroxide (0.092 g, 3.85 mmol, 5.0 equiv) in 10 mL water. To the resulting biphasic mixture was added methanol dropwise until a single layer formed. The resulting solution was stirred for 2 hours at room temperature. It was then poured into 1.2 N HCl_{(aq)} and the aqueous layer was extracted with three 50 mL portions of ethyl acetate. The combined organic layers were washed with water and brine and dried over magnesium sulfate. Filtration and removal of solvent in vacuo gave the crude material. This was purified by reverse phase HPLC and the isolated product lyophilized to give a white powder (0.219 g, 63%). ¹H NMR (400 MHz, DMSO-d₆) δ 2.26 (s, 3H) 4.08 (s, 2H) 4.97 (s, 2H) 5.08 (s, 2H) 6.85 (td, J=9.1, 2.5 Hz, 1H) 7.10 (dd, J=10.1, 2.5 Hz, 1H) 7.17 (s, 1H) 7.20-7.31 (m, 5H) 7.36 (dd, J=8.8, 4.5 Hz, 1H) 7.47-7.55 (m, 1H) 7.71 (td, J=7.6, 1.5 Hz, 1H) 7.77-7.83 (m, 1H) 8.27 (dd, J=8.1, 1.3 Hz, 1H) 13.03 (br. s., 1H).

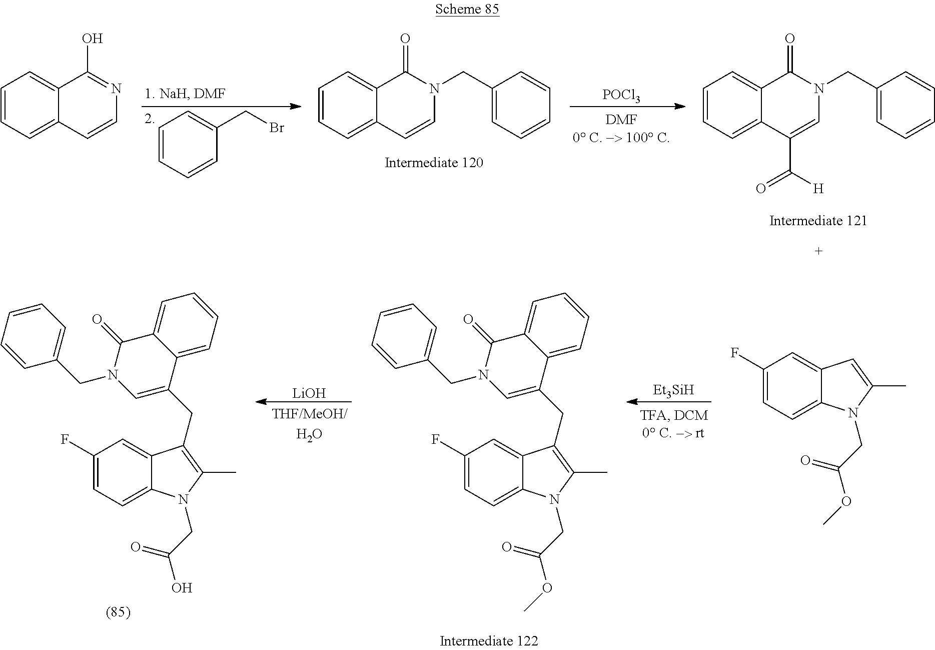

### Example 86

Step 1: Preparation of methyl 2-(3-((2-benzyl-1-oxo-1,2-dihydroisoquinolin-4-yl)methyl)-2-methyl-1H-indol-1-yl)acetate, Intermediate 123. To a 100 mL round bottom flask under an atmosphere of nitrogen was added intermediate 121, 2-benzyl-1-oxo-1,2-dihydroisoquinoline-4-carbaldehyde (0.300 g, 1.14 mmol, 1.1 equiv), methyl 2-(2-methyl-1H-indol-1-yl)acetate (0.211 g, 1.04 mmol, 1.0 equiv) and 50 mL anhydrous methylene chloride. The resulting solution was cooled to 0° C. in an ice/water bath and triethylsilane (0.58 mL, 3.63 mmol, 3.5 equiv) and trifluoroacetic acid (0.23 mL, 3.12 mmol, 3.0 equiv) were added dropwise. The mixture was allowed to warm to room temperature and then stirred for 24 hours. The mixture was then poured into sat NaHCO_{3(aq)} and the aqueous layer extracted with two 50 mL portions of methylene chloride. The combined organic layers were then washed with water and brine, and dried over magnesium sulfate. Filtration and removal of solvent in vacuo gave the crude material which was then purified by silica gel chromatography to give a white solid (0.276 g, 59%).

Step 2: Preparation of 2-(3-((2-benzyl-1-oxo-1,2-dihydroisoquinolin-4-yl)methyl)-2-methyl-1H-indol-1-yl)acetic acid (86). To a 100 mL round bottom flask was added intermediate 123, methyl 2-(3-((2-benzyl-1-oxo-1,2-dihydroisoquinolin-4-yl)methyl)-2-methyl-1H-indol-1-yl)acetate (0.276 g, 0.61 mmol, 1.0 equiv) and 20 mL THF. To this was added a solution of Lithium hydroxide (0.073 g, 3.85 mmol, 5.0 equiv) in 10 mL water. To the resulting biphasic mixture was added methanol dropwise until a single layer formed. The resulting solution was stirred for 2 hours at room temperature. It was then poured into 1.2 N HCl_{(aq)} and the aqueous layer was extracted with three 50 mL portions of ethyl acetate. The combined organic layers were washed with water and brine and dried over magnesium sulfate. Filtration and removal of solvent in vacuo gave the crude material. This was purified by reverse phase HPLC and the isolated product lyophilized to give a white powder (0.194 g, 75%). ¹H NMR (400 MHz, DMSO-d₆) δ 2.28 (s, 3H) 4.10 (s, 2H) 4.94 (s, 2H) 5.09 (s, 2H) 6.79-6.89 (m, 1H) 7.01 (ddd, J=8.1, 7.0, 1.3 Hz, 1H) 7.17-7.36 (m, 8H) 7.50 (td, J=7.6, 1.0 Hz, 1H) 7.69 (ddd, J=8.2, 6.9, 1.5 Hz, 1H) 7.81 (d, J=7.8 Hz, 1H) 8.27 (dd, J=8.2, 1.4 Hz, 1H) 13.02 (br. s., 1H).

### Example 87

Step 1: Preparation of methyl 2-(3-((2-benzyl-1-oxo-1,2-dihydroisoquinolin-4-yl)methyl)-5-chloro-2-methyl-1H-indol-1-yl)acetate, Intermediate 124. To a 100 mL round bottom flask under an atmosphere of nitrogen was added intermediate 131, 2-benzyl-1-oxo-1,2-dihydroisoquinoline-4-carbaldehyde (0.327 g, 1.24 mmol, 1.1 equiv), methyl 2-(5-chloro-2-methyl-1H-indol-1-yl)acetate (0.268 g, 1.13 mmol, 1.0 equiv) and 50 mL anhydrous methylene chloride. The resulting solution was cooled to 0° C. in an ice/water bath and triethylsilane (0.63 mL, 3.96 mmol, 3.5 equiv) and trifluoroacetic acid (0.25 mL, 3.39 mmol, 3.0 equiv) were added dropwise. The mixture was allowed to warm to room temperature and then stirred for 24 hours. The mixture was then poured into sat NaHCO₃₀₀ and the aqueous layer extracted with two 50 mL portions of methylene chloride. The combined organic layers were then washed with water and brine, and dried over magnesium sulfate. Filtration and removal of solvent in vacuo gave the crude material which was then purified by silica gel chromatography to give a white solid (0.333 g, 61%).

Step 2: Preparation of 2-(3-((2-benzyl-1-oxo-1,2-dihydroisoquinolin-4-yl)methyl)-5-chloro-2-methyl-1H-indol-1-yl)acetic acid (87). To a 100 mL round bottom flask was added intermediate 124, methyl 2-(3-((2-benzyl-1-oxo-1,2-dihydroisoquinolin-4-yl)methyl)-5-chloro-2-methyl-1H-indol-1-yl)acetate (0.333 g, 0.69 mmol, 1.0 equiv) and 20 mL THF. To this was added a solution of Lithium hydroxide (0.083 g, 3.47 mmol, 5.0 equiv) in 10 mL water. To the resulting biphasic mixture was added methanol dropwise until a single layer formed. The resulting solution was stirred for 2 hours at room temperature. It was then poured into 1.2 N HCl_{(aq)} and the aqueous layer was extracted with three 50 mL portions of ethyl acetate. The combined organic layers were washed with water and brine and dried over magnesium sulfate. Filtration and removal of solvent in vacuo gave the crude material. This was purified by reverse phase HPLC and the isolated product lyophilized to give a white powder (0.130 g, 40%). ¹H NMR (400 MHz, DMSO-d₆) δ 2.26 (s, 3H) 4.10 (s, 2H) 4.99 (s, 2H) 5.06 (s, 2H) 7.03 (dd, J=8.6, 2.0 Hz, 1H) 7.13 (s, 1H) 7.17-7.33 (m, 5H) 7.36-7.44 (m, 2H) 7.49-7.56 (m, 1H) 7.71 (td, J=7.6, 1.4 Hz, 1H) 7.78-7.84 (m, 1H) 8.27 (dd, J=8.1, 1.0 Hz, 1H) 13.09 (br. s., 1H).

### Example 88

Step 1: Preparation of 5-fluoro-3-iodo-2-methyl-1H-indole, Intermediate 125. To a 100 mL round-bottomed flask under an atmosphere of nitrogen was added 5-fluoro-2-methyl-1H-indole (5.0 g, 33.5 mmol) and potassium hydroxide (1.881 g, 33.5 mmol) in 25 mL DMF to give a orange solution. Iodine (8.51 g, 33.5 mmol) was added in portions. The resulting mixture was allowed to stir for 16 hours at room temperature. The mixture was then poured into 500 mL water and extracted with 3 100 mL portions of ethyl acetate. The combined organic layers were washed with water and brine, dried over MgSO₄ and the solvent was removed in vacuo to give the crude material as a dark brown oil. Purified by silica gel chromatography (6-50% Ethyl acetate/Hexane, 340 g SNAP column) to give a dark brown solid (8.32 g, 90%). ¹H NMR (400 MHz, CHLOROFORM-d) δ 2.45 (s, 3H) 6.89 (td, J=9.0, 2.5 Hz, 1H) 7.02 (dd, J=9.3, 2.5 Hz, 1H) 7.15 (dd, J=8.6, 4.0 Hz, 1H) 8.14 (br. s., 1H).

Step 2: Preparation of methyl 2-(5-fluoro-3-iodo-2-methyl-1H-indol-1-yl)acetate, Intermediate 126. To a 500 mL round-bottomed flask under an atmosphere of nitrogen was added 5-fluoro-3-iodo-2-methyl-1H-indole (8.3 g, 30.2 mmol), methyl 2-bromoacetate (11.10 mL, 121 mmol), and potassium carbonate (20.85 g, 151 mmol) in 150 mL DMF to give a brown suspension. This was heated to 90 C and allowed to stir for 16 hours. The mixture was cooled to room temperature and poured into 800 mL water. This was extracted with three 250 mL portions of ethyl acetate. The combined organic layers were washed with water and brine, the dried over MgSO₄. Filtration and concentration in vacuo gave the crude material which was purified by silica gel chromatography (6-50% EtOAc/Hex; 340 g SNAP column) to give a tan solid (8.08 g, 77%). ¹H NMR (400 MHz, CHLOROFORM-d) δ 2.44 (s, 3H) 3.74 (s, 3H) 4.82 (s, 2H) 6.89-6.97 (m, 1H) 7.02-7.09 (m, 2H).

Step 3: Preparation of 5,6,7,8-tetrahydroisoquinolin-1(2H)-one, Intermediate 127. To a 100 mL round-bottomed flask under an atmosphere of nitrogen was added isoquinolin-1(2H)-one (0.5 g, 3.44 mmol) in 20 mL of acetic acid to give a colorless solution. Platinum(IV) oxide (0.235 g, 1.033 mmol) was added. Hydrogen gas was sparged through the solution via a needle attached to a balloon for 10 minutes. The reaction was then allowed to stir for 16 hours under one atmosphere of nitrogen. The catalyst was filtered off and the acetic acid removed by azeotroping with hexane. The residue was purified by silica gel chromatography (1-10% MeOH/CH₂Cl₂; 50 g SNAP column) to give the desired product as a white solid (0.100 g, 20%). ¹H NMR (400 MHz, chloroform-d) δ 1.65-1.83 (m, 4H) 2.47-2.65 (m, 4H) 6.02 (d, J=6.6 Hz, 1H) 7.17 (d, J=6.6 Hz, 1H) 12.92 (br. s., 1H).

Step 4: Preparation of 2-benzyl-5,6,7,8-tetrahydroisoquinolin-1(2H)-one, Intermediate 128. To a 250 mL round-bottomed flask under an atmosphere of nitrogen was added intermediate 137, 5,6,7,8-tetrahydroisoquinolin-1(2H)-one (0.592 g, 3.97 mmol) and cesium carbonate (1.293 g, 3.97 mmol) in 40 mL DMF to give a colorless suspension. Benzyl bromide (0.471 mL, 3.97 mmol) was added and the mixture heated to 50 C. The reaction was allowed to stir 16 hours. The mixture was poured into water and extracted with 3 100 mL portions of ethyl acetate. The combined organic layers were washed with water and brine, then dried over MgSO₄ and filtered. The filtrate was concentrated in vacuo and the residue was purified by silica gel chromatography (1:1Hex/EtOAc; 40+M column) to give the desired product as a white solid (0.691 g, 73%). ¹H NMR (400 MHz, CHLOROFORM-d) δ 1.62-1.84 (m, 4H) 2.48-2.54 (m, 2H) 2.56 (t, J=5.8 Hz, 2H) 5.91 (d, J=7.1 Hz, 1H) 7.04 (d, J=7.1 Hz, 1H) 7.22-7.37 (m, 5H).

Step 5: Preparation of 2-benzyl-4-iodo-5,6,7,8-tetrahydroisoquinolin-1(2H)-one, Intermediate 129. To a 50 mL round-bottomed flask under an atmosphere of nitrogen and cooled to 0 C was added intermediate 57, 2-benzyl-5,6,7,8-tetrahydroisoquinolin-1(2H)-one (0.457 g, 1.910 mmol), silver trifluoromethanesulfonate (0.491 g, 1.910 mmol), and potassium hydroxide (0.107 g, 1.910 mmol) in 10 mL diethyl ether to give a white suspension. Iodine (0.485 g, 1.910 mmol) was added. The mixture was allowed to stir at 0 C for 2 hours. At that point, the mixture was diluted with 10 mL ether, and filtered. The organic layer was washed with sodium metabisulfate, water and brine and dried over MgSO4. This was then filtered, concentrated and purified by silica gel chromatography (12-100% EtOAc/Hex) to give the desired product as a yellow oil (0.368g, 53%). ¹H NMR (400 MHz, CHLOROFORM-d) δ 1.58-1.80 (m, 4H) 2.43 (t, J=5.1 Hz, 2H) 2.57 (t, J=5.3 Hz, 2H) 5.08 (s, 2H) 7.23-7.38 (m, 5H) 7.51 (s, 1H).

Step 6: Preparation of 2-benzyl-4-(4,4,5,5-tetramethyl-1,3,2-dioxaborolan-2-yl)-5,6,7,8-tetrahydroisoquinolin-1(2H)-one, Intermediate 130. To a 50 mL round bottom flask under an atmosphere of nitrogen was added 4,4,4′,4′5,5,5′,5′-octamethyl-2,2′-bi(1,3,2-dioxaborolane) (0.280 g, 1.102 mmol), intermediate 129, 2-benzyl-4-iodo-4a,5,6,7,8,8a-hexahydroisoquinolin-1(2H)-one (0.368 g, 1.002 mmol), and potassium acetate (0.295 g, 3.01 mmol) in 10 mL DMSO to give a orange solution. PdCl2(dppf)-CH2Cl2 adduct (0.049 g, 0.060 mmol) was added. The reaction was heated to 80° C. and allowed to stir for 16 hours. The mixture was poured into 100 mL water, and extracted with 3 50 mL portions of ethyl acetate. The organic layer was washed with water and brine, dried over magnesium sulfate, filtered and concentrated. The residue was purified by silica gel chromatography (12-100% ethyl acetate/hexane) to give the desired product as a yellow oil (0.261g, 71%). ¹H NMR (400 MHz, CHLOROFORM-d) δ 1.28 (s, 12H) 1.62-1.80 (m, 4H) 2.46-2.60 (m, 2H) 2.73-2.85 (m, 2H) 5.12 (s, 2H) 7.21-7.36 (m, 5H) 7.67 (s, 1H).

Step 7: Preparation of 2-(3-(2-benzyl-1-oxo-1,2,5,6,7,8-hexahydroisoquinolin-4-yl)-5-fluoro-2-methyl-1H-indol-1-yl)acetic acid (88). To a 5 mL microwave vessel under an atmosphere of nitrogen was added intermediate 126, methyl 2-(5-fluoro-3-iodo-2-methyl-1H-indol-1-yl)acetate (0.496 g, 1.429 mmol), intermediate 130, 2-benzyl-4-(4,4,5,5-tetramethyl-1,3,2-dioxaborolan-2-yl)-5,6,7,8-tetrahydroisoquinolin-1(2H)-one (0.261 g, 0.715 mmol), and potassium phosphate tribasic monohydrate (0.303 g, 1.429 mmol) in 4 mL Butan-1-ol and 1.6 mL water to give a tan suspension. The vial was purged with nitrogen and palladium(II) acetate (8.02 mg, 0.036 mmol) was added. The vessel was sealed and heated to 100 C in an oil bath and allowed to stir 16 hours. It was then diluted with 150 mL water and extracted with 3100 mL portions of ethyl acetate. The combined organic layers were washed with water and brine, dried over magnesium sulfate, filtered and concentrated to give a yellow oil. The residue was purified by silica gel chromatography (12-100% Ethyl acetate/hexane; 340 g SNAP column) to give the desired product as a white powder (0.018 g, 3%). ¹H NMR (400 MHz, DMSO-d₆) δ 1.47-1.66 (m, 2H) 1.68-1.80 (m, 2H) 2.05-2.31 (m, 5H), 2.42-2.68 (m, 2H) 5.07 (s, 2H) 5.20 (d, J=4.8 Hz, 2H) 6.90 (dd, J=9.6, 2.3 Hz, 1H) 6.98 (td, J=9.1, 2.5 Hz, 1H) 7.29-7.37 (m, 1H) 7.37-7.44 (m, 4H) 7.49 (dd, J=8.7, 4.4 Hz, 1H) 7.54 (s, 1H).

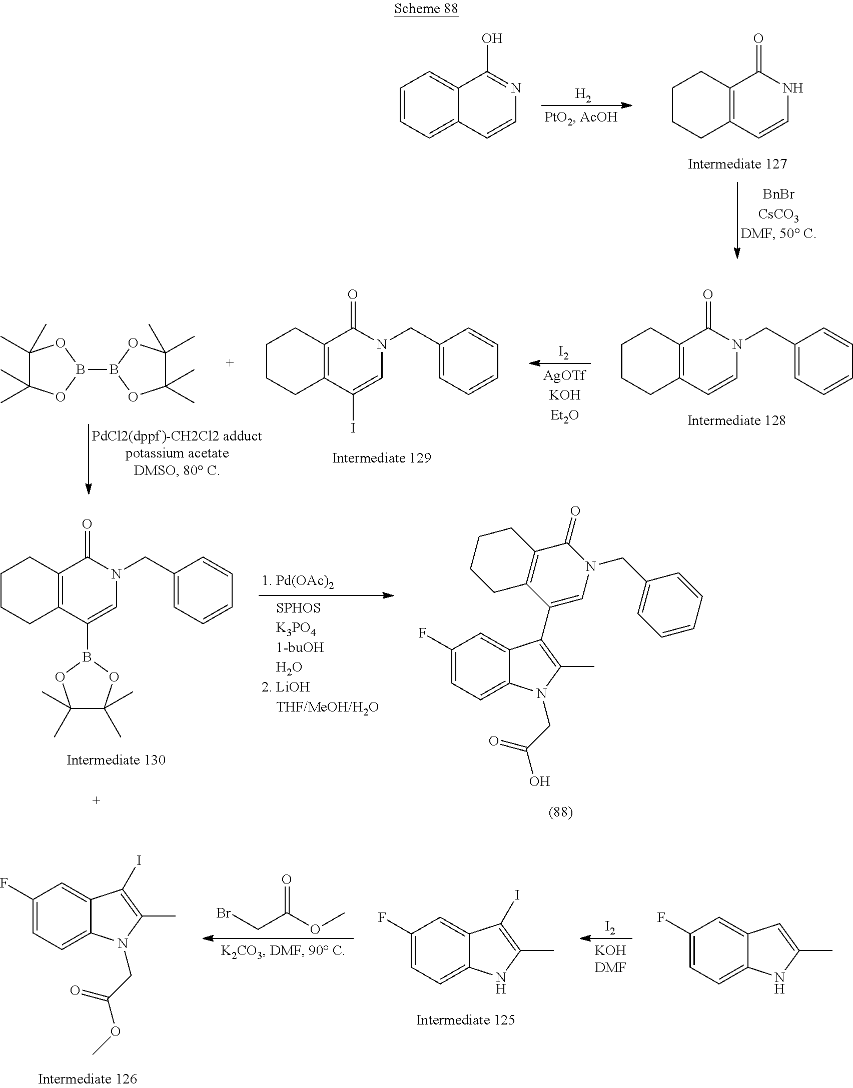

### Example 89

Preparation of 2-(5-fluoro-2-methyl-3-(1-oxo-2-(4,4,4-trifluorobutyl)-1,2,5,6,7,8-hexahydro-isoquinolin-4-yl)-1H-indol-1-yl)acetic acid (89). The title compound was prepared according to the procedure of Example 88; Yield 33%. ¹H NMR (400 MHz, DMSO-d₆) δ 1.42-1.60 (m, 2H) 1.61-1.74 (m, 2H) 1.90 (quin, J=7.6 Hz, 2H) 2.04-2.23 (m, 5H) 2.23-2.36 (m, 2H) 2.40-2.45 (m, 2H) 3.89-4.03 (m, 2H) 5.02 (d, J=1.3 Hz, 2H) 6.84-6.97 (m, 2H) 7.38 (s, 1H) 7.43 (dd, J=8.7, 4.4 Hz, 1H) 13.08 (br. s., 1H).

### Example 90

Preparation of 2-(5-fluoro-2-methyl-3-(1-oxo-2-(2,2,2-trifluoroethyl)-1,2,5,6,7,8-hexahydro-isoquinolin-4-yl)-1H-indol-1-yl)acetic acid (90). The title compound was prepared according to the procedure of Example 88; Yield 25%. ¹H NMR (400 MHz, DMSO-d₆) δ 1.45-1.63 (m, 2H) 1.64-1.75 (m, 2H) 2.04-2.28 (m, 5H) 2.39-2.53 (m, 2H) 4.78-4.98 (m, 2H) 5.03 (d, J=1.5 Hz, 2H) 6.85-6.97 (m, 2H) 7.37 (s, 1H) 7.45 (dd, J=8.8, 4.3 Hz, 1H) 13.09 (br. s., 1H).

### Example 91

Preparation of 2-(5-fluoro-3-(2-isopropyl-1-oxo-1,2,5,6,7,8-hexahydroisoquinolin-4-yl)-2-methyl-1H-indol-1-yl)acetic acid (91). The title compound was prepared according to the procedure of Example 88; Yield 23%. ¹H NMR (400 MHz, DMSO-d₆) δ 1.30 (d, 6H) 1.41-1.61 (m, 2H) 1.68 (quin, J=5.9 Hz, 2H) 2.01-2.25 (m, 5H) 2.44 (d, J=5.6 Hz, 2H) 5.02 (d, J=1.0 Hz, 2H) 5.14 (quin, J=6.8 Hz, 1H) 6.84-6.97 (m, 2H) 7.29 (s, 1H) 7.43 (dd, J=9.0, 4.4 Hz, 1H) 13.07 (s, 1H).

### Example 92

Preparation of 2-(5-fluoro-3-(2-(2-hydroxy-2-methylpropyl)-1-oxo-1,2,5,6,7,8-hexahydro-iso-quinolin-4-yl)-2-methyl-1H-indol-1-yl)acetic acid (92). The title compound was prepared according to the procedure of Example 88; Yield 21%. ¹H NMR (400 MHz, DMSO-d₆) δ 1.10 (d, 6H) 1.46-1.62 (m, 2H) 1.69 (quin, J=5.9 Hz, 2H) 2.09-2.28 (m, 5H) 2.40-2.49 (m, 2H) 3.96 (q, J=13.2 Hz, 2H) 4.91 (s, 1H) 5.02 (d, J=2.5 Hz, 2H) 6.86-6.97 (m, 2H) 7.35 (s, 1H) 7.44 (dd, J=9.0, 4.2 Hz, 1H) 13.09 (br. s., 1H).

### Example 93

Preparation of 2-(5-fluoro-2-methyl-3-(1-oxo-2-phenethyl-1,2,5,6,7,8-hexahydro-iso-quinolin-4-yl)-1H-indol-1-yl)acetic acid (93). The title compound was prepared according to the procedure of Example 88; Yield 62%. ¹H NMR (400 MHz, DMSO-d₆) δ 1.47-1.59 (m, 2H) 1.62-1.76 (m, 2H) 2.00-2.14 (m, 5H) 2.43-2.49 (m, 2H) 2.99 (t, J=7.2 Hz, 2H) 4.13 (t, J=7.3 Hz, 2H) 4.97 (d, J=1.0 Hz, 2H) 6.71 (dd, J=9.7, 2.4 Hz, 1H) 6.90 (td, J=9.2, 2.5 Hz, 1H) 7.12 (s, 1H) 7.16-7.24 (m, 3H) 7.24-7.31 (m, 2H) 7.40 (dd, J=8.8, 4.3 Hz, 1H).

### Example 94

Preparation of 2-(3-(2-(2,4-difluorobenzyl)-1-oxo-1,2,5,6,7,8-hexahydroisoquinolin-4-yl)-5-fluoro-2-methyl-1H-indol-1-yl)acetic acid (94). The title compound was prepared according to the procedure of Example 88; Yield 29%. ¹H NMR (400 MHz, DMSO-d₆) δ 1.44-1.60 (m, 2H) 1.62-1.72 (m, 2H) 2.04-2.15 (m, 1H) 2.18 (s, 3H) 2.22-2.33 (m, 1H) 2.37-2.47 (m, 2H) 5.03 (s, 2H) 5.13 (d, J=5.6 Hz, 2H) 6.86-6.97 (m, 2H) 7.09 (m, J=8.5, 8.5, 2.6, 0.9 Hz, 1H) 7.23-7.37 (m, 2H) 7.41-7.48 (m, 2H).

### Example 95

Preparation of 2-(5-fluoro-2-methyl-3-(1-oxo-2-(pyridin-2-ylmethyl)-1,2-dihydro-isoquinolin-4-yl)-1H-indol-1-yl)acetic acid (95). The title compound was prepared according to the procedure of Example 88; Yield 39%. ¹H NMR (400 MHz, DMSO-d₆) δ ppm 2.23 (s, 3H) 5.07 (s, 2H) 5.38 (s, 2H) 6.87 (dd, J=9.9, 2.5 Hz, 1H) 6.96 (td, J=9.2, 2.7 Hz, 1H) 7.24-7.36 (m, 3H) 7.47-7.58 (m, 3H) 7.63-7.69 (m, 1H) 7.79 (td, J=7.7 , 1.8 Hz, 1H) 8.32 (dt, J=8.1, 0.8 Hz, 1H) 8.52 (ddd, J=4.8, 1.8, 1.0 Hz, 1H).

### Example 96

Preparation of 2-(5-fluoro-2-methyl-3-(1-oxo-2-(4,4,4-trifluoro-3-(trifluoromethyl)butyl)-1,2-dihydroisoquinolin-4-yl)-1H-indol-1-yl)acetic acid (96). The title compound was prepared according to the procedure of Example 88; Yield 38%. ¹H NMR (400 MHz, DMSO-d₆) δ 2.21 (s, 3H) 2.23-2.36 (m, 2H) 4.11-4.19 (m, 1H) 4.22 (t, J=6.4 Hz, 2H) 5.08 (s, 2H) 6.87 (dd, J=9.9, 2.5 Hz, 1H) 6.96 (td, J=9.2, 2.7 Hz, 1H) 7.22 (d, J=7.6 Hz, 1H) 7.47-7.58 (m, 3H) 7.61-7.68 (m, 1H) 8.35 (dd, J=8.0, 0.9 Hz, 1H).

### Example 97

Step 1: Preparation of tent-butyl 2-(5 -chloro-3-(3-(2,3-difluorobenzyl)-4-oxo-3,4-dihydrophthalazin-1-yl)-2-methyl-1H-indol-1-yl)acetate, Intermediate 131. To a 100 mL round bottom flask was added intermediate 2 (0.400 g, 0.95 mmol, 1.0 eq.), potassium carbonate (0.328 g, 2.375 mmol, 2.5 eq.) and DMF (10 mL, 0.1 M). The flask was purged with nitrogen and 1-(bromomethyl)-2,3-difluorobenzene (0.242 mL, 1.90 mmol, 2.0 eq.) was added and the reaction stirred at 90° C. overnight. The reaction was cooled to room temperature, extracted with ethyl acetate, washed with brine, dried over MgSO₄, and concentrated in vacuo. The resulting material was carried on crude.

Step 2: Preparation of 2-(5-chloro-3-(3-(2,3-difluorobenzyl)-4-oxo-3,4-dihydrophthalazin-1-yl)-2-methyl-1H-indol-1-yl)acetic acid (97). To a 100 mL round bottom flask which contained intermediate 131 was added trifluoroacetic acid (3 mL). The reaction stirred at room temperature for 3 hrs. Water was added, the reaction was extracted with ethyl acetate, washed with brine, dried over MgSO₄, and concentrated in vacuo. The resulting material was purified via reverse phase HPLC (Gilson acidic) yielding 97 as a white solid (109.0 mg, 23.2% over two steps). ¹H NMR (400 MHz, chloroform-d) δ 8.53 (d, J=7.8 Hz, 1H) 7.74-7.82 (m, 1H) 7.67-7.74 (m, 1H) 7.60 (d, J=8.1 Hz, 1H) 7.14-7.21 (m, 3H) 6.99-7.11 (m, 3H) 5.48-5.66 (m, 2H) 4.82-4.97 (m, 2H) 2.29 (s, 3H).

### Example 98

Preparation of 2-(5 -chloro-3 -(3 -(2-fluorobenzyl)-4-oxo-3,4-dihydrophthalazin-1-yl)-2-methyl-1H-indol-1-yl)acetic acid (98). The title compound was prepared according to the procedure of Example 97; Yield: 20.4%.

### Example 99

Preparation of 2-(5-chloro-3-(3-((5-fluorobenzo[d]thiazol-2-yl)methyl)-4-oxo-3,4-dihydrophthalazin-1-yl)-2-methyl-1H-indol-1-yl)acetic acid (99). The title compound was prepared according to the procedure of Example 97; Yield: 10.1%

### Example 100

Preparation of 2-(5-chloro-2-methyl-3-(4-oxo-3-((5-(trifluoromethyl)benzo[d]thiazol-2-yl)-methyl)-3,4-dihydrophthalazin-1-yl)-1H-indol-1-yl)acetic acid (100). The title compound was prepared according to the procedure of Example 97; Yield: 25.3%

### Example 101

Preparation of 2-(5-chloro-3-(3-(2,6-difluorobenzyl)-4-oxo-3,4-dihydrophthalazin-1-yl)-2-methyl-1H-indol-1-yl)acetic acid (101). The title compound was prepared according to the procedure of Example 97; Yield: 12.6%

### Example 102

Preparation of 2-(5-chloro-2-methyl-3-(4-oxo-3-(4-(trifluoromethoxy)benzyl)-3,4-dihydrophthalazin-1-yl)-1H-indol-1-yl)acetic acid (102). The title compound was prepared according to the procedure of Example 97; Yield: 29.8%

### Example 103

Preparation of 2-(5-chloro-2-methyl-3-(4-oxo-3-(quinolin-2-ylmethyl)-3,4-dihydro-phthalazin-1-yl)-1H-indol-1-yl)acetic acid (103). The title compound was prepared according to the procedure of Example 97; Yield: 41.8%

### Example 104

Preparation of 2-(5-chloro-2-methyl-3-(3-((2-methylquinolin-4-yl)methyl)-4-oxo-3,4-dihydro-phthalazin-1-yl)-1H-indol-1-yl)acetic acid (104). The title compound was prepared according to the procedure of Example 97 by reacting intermediate 2 with 4-(chloromethyl)-2-methylquinoline; Yield: 38.9%

### Example 105

Preparation of 2-(5-chloro-2-methyl-3-(3-methyl-4-oxo-3,4-dihydrophthalazin-1-yl)-1H-indol-1-yl)-acetic acid (105). The title compound was prepared according to the procedure of Example 97;Yield: 38.4%

### Example 106

Preparation of 2-(5-chloro-3-(3-ethyl-4-oxo-3,4-dihydrophthalazin-1-yl)-2-methyl-1H-indol-1-acetic acid (106). The title compound was prepared according to the procedure of Example 97; Yield: 45.1%

### Example 107

Preparation of 2-(5-chloro-3-(3-isopropyl-4-oxo-3,4-dihydrophthalazin-1-yl)-2-methyl-1H-indol-1-yl)acetic acid (107). The title compound was prepared according to the procedure of Example 97; Yield: 44.8%

### Example 108

Preparation of 2-(5-chloro-3-(3-(cyclopropylmethyl)-4-oxo-3,4-dihydrophthalazin-1-yl)-2-methyl-1H-indol-1-yl)acetic acid (108). The title compound was prepared according to the procedure of Example 97; Yield: 29.2%

### Example 109

Preparation of 2-(5-chloro-2-methyl-3-(4-oxo-3-(2,2,2-trifluoroethyl)-3,4-dihydro-phthalazin-1-yl)-1H-indol-1-yl)acetic acid (109). The title compound was prepared according to the procedure of Example 97; Yield: 54.5%

### Example 110

Preparation of 2-(3-(3-(benzo[d]thiazol-2-ylmethyl)-4-oxo-3,4-dihydrophthalazin-1-yl)-5-fluoro-2- methyl-1H-indol-1-yl)acetic acid (110). The title compound was prepared according to the procedure of Example 97; Yield: 41.4%

### Example 111

Preparation of 2-(5-fluoro-3-(3-(4-fluorobenzyl)-4-oxo-3,4-dihydrophthalazin-1-yl)-2-methyl-1H-indol-1-yl)acetic acid (111). The title compound was prepared according to the procedure of Example 97; Yield: 37.5%

### Example 112

Preparation of 2-(3-(3-(2,3-difluorobenzyl)-4-oxo-3,4-dihydrophthalazin-1-yl)-5-fluoro-2-methyl-1H-indol-1-yl)acetic acid (112). The title compound was prepared according to the procedure of Example 97; Yield: 44.9%

### Example 113

Preparation of 2-(5-fluoro-3-(3-(2-fluorobenzyl)-4-oxo-3,4-dihydrophthalazin-1-yl)-2-methyl-1H-indol-1-yl)acetic acid (113). The title compound was prepared according to the procedure of Example 97; Yield: 33.4%

### Example 114

Preparation of 2-(5-fluoro-3-(3-((5-fluorobenzo[d]thiazol-2-yl)methyl)-4-oxo-3,4-dihydro-phthalazin-1-yl)-2-methyl-1H-indol-1-yl)acetic acid (114). The title compound was prepared according to the procedure of Example 97; Yield: 29%

### Example 115

Preparation of 2-(5-fluoro-2-methyl-3-(4-oxo-3-((5-(trifluoromethyl)benzo[d]thiazol-2-yl)-methyl)-3,4-dihydrophthalazin-1-yl)-1H-indol-1-yl)acetic acid (115). The title compound was prepared according to the procedure of Example 97; Yield: 18.9%

### Example 116

Preparation of 2-(3-(3-(2,6-difluorobenzyl)-4-oxo-3,4-dihydrophthalazin-1-yl)-5-fluoro-2-methyl-1H-indol-1-yl)acetic acid (116). The title compound was prepared according to the procedure of Example 97; Yield: 27.6%

### Example 117

Preparation of 2-(5-fluoro-2-methyl-3-(4-oxo-3-(4-(trifluoromethoxy)benzyl)-3,4-dihydro-phthalazin-1-yl)-1H-indol-1-yl)acetic acid (117). The title compound was prepared according to the procedure of Example 97; Yield: 27.9%

### Example 118

Preparation of 2-(5-fluoro-2-methyl-3-(4-oxo-3-(quinolin-2-ylmethyl)-3,4-dihydro-phthalazin-1-yl)-1H-indol-1-yl)acetic acid (118). The title compound was prepared according to the procedure of Example 97; Yield: 36.5%

### Example 119

Preparation of 2-(5-fluoro-2-methyl-3-(3-((2-methylquinolin-4-yl)methyl)-4-oxo-3,4-dihydro-phthalazin-1-yl)-1H-indol-1-yl)acetic acid (119). The title compound was prepared according to the procedure of Example 97; Yield: 44.6%

### Example 120

Preparation of 2-(5-fluoro-2-methyl-3-(3-methyl-4-oxo-3,4-dihydrophthalazin-1-yl)-1H-indol-1-yl)acetic acid (120). The title compound was prepared according to the procedure of Example 97; Yield: 40.1%

### Example 121

Preparation of 2-(3-(3-ethyl-4-oxo-3,4-dihydrophthalazin-1-yl)-5-fluoro-2-methyl-1H-indol-1-yl)acetic acid (121). The title compound was prepared according to the procedure of Example 97; Yield: 23.0%

### Example 122

Preparation of 2-(3-(3-(cyclopropylmethyl)-4-oxo-3,4-dihydrophthalazin-1-yl)-5-fluoro-2-methyl-1H-indol-1-yl)acetic acid (122). The title compound was prepared according to the procedure of

Example 97; Yield: 12.5%

### Example 123

Step 1: Preparation of tricyclopropylbismuth, Intermediate 132. Bismuth chloride (2.50 g, 7.93 mmol, 1.0 eq.) was dissolved in anhydrous THF (100 mL, 0.08M) and cooled to −10° C. Cyclopropylmagnesium bromide (52.4 mL, 26.2 mmol, 0.5M in THF) was slowly added dropwise under nitrogen via syringe. The reaction mixture was stirred at room temperature for one hour and heated at 70° C. for 30 minutes. After cooling, to room temperature, the solution was cannulated under nitrogen over a biphasic solution of brine (200 mL) and ether (200 mL). The heterogeneous solution was stirred five minutes, transferred to a separation funnel and diluted with ethyl acetate (100 mL). The organic phase was collected, dried over MgSO₄ and concentrated in vacuo yielding a yellow oil. This material was triturated with ether and hexanes yielding an off-white solid (1.54 g, 58.5%).

Step 2: Preparation of tert-butyl 2-(3-(3-cyclopropyl-4-oxo-3,4-dihydrophthalazin-1-yl)-5-fluoro-2-methyl-1H-indol-1-yl)acetate, Intermediate 133. In a sealed tube, intermediate 5, tert-butyl 2-(5-fluoro-3-(4-hydroxyphthalazin-1-yl)-2-methyl-1H-indol-1-yl)acetate (0.350 g, 0.859 mmol, 1.0 eq.) was diluted in dichloromethane (9 mL 0.1 M). Copper acetate (0.234 g, 1.29 mmol, 1.5 eq.) was added, followed by pyridine (0.208 mL, 2.58 mmol, 3.0 eq.) and intermediate 122, tricyclopropylbismuth (0.713 g, 2.15 mmol, 2.5 eq.). The tube was purged with nitrogen, sealed, and stirred at 50° C. overnight. The reaction mixture was cooled to room temperature, extracted with ethyl acetate, washed with brine, dried over MgSO₄, and concentrated in vacuo. The resulting material was carried on crude.

Step 3: Preparation of 2-(3-(3-cyclopropyl-4-oxo-3,4-dihydrophthalazin-1-yl)-5-fluoro-2-methyl-1H-indol-1-yl)acetic acid (123). Intermediate 133 was dissolved in trifluoroacetic acid (5 mL) and stirred for 3 hours at room temperature. Water was added, and the reaction was extracted with ethyl acetate, washed with brine, dried over MgSO₄, and concentrated in vacuo. The resulting material was purified via reverse phase HPLC (Gilson acidic) yielding 123 as a solid (26.2 mg, 7.8% over two steps). ¹H NMR (400 MHz, MeOD) δ 8.36 (d, J=7.6 Hz, 1H) 7.73-7.81 (m, 1H) 7.66-7.72 (m, 1H) 7.56 (d, J=8.1 Hz, 1H) 7.27 (dd, J=8.8, 4.0 Hz, 1H) 6.84 (td, J=9.1, 2.3 Hz, 1H) 6.74 (dd, J=9.5, 2.4 Hz, 1H) 4.91-4.99 (m, 2H) 4.06 (dt, J=7.5, 3.7 Hz, 1H) 2.23 (s, 3H) 1.04-1.15 (m, 2H) 0.88-1.01 (m, 2H).

### Example 124

Preparation of 2-(5-fluoro-2-methyl-3-(4-oxo-3-(2,2,2-trifluoroethyl)-3,4-dihydro-phthalazin-1-yl)-1H-indol-1-yl)acetic acid (124). The title compound was prepared according to the procedure of Example 97; Yield: 24.2%

### Example 125

Preparation of 2-(5-fluoro-2-methyl-3-(4-oxo-3-(4,4,4-trifluorobutyl)-3,4-dihydro-phthalazin-1-yl)-1H-indol-1-yl)acetic acid (125). The title compound was prepared according to the procedure of Example 97; Yield: 35.9%

### Example 126

Preparation of 2-(5-fluoro-2-methyl-3-(3-neopentyl-4-oxo-3,4-dihydrophthalazin-1-yl)-1H-indol-1-yl)acetic acid (126). The title compound was prepared according to the procedure of Example 97; Yield: 18.9%

### Example 127

Preparation of 2-(5-fluoro-2-methyl-3-(4-oxo-3-(3,3,3-trifluoropropyl)-3,4-dihydro-phthalazin-1-yl)-1H-indol-1-yl)acetic acid (127). The title compound was prepared according to the procedure of Example 97; Yield: 14.9%

### Example 128

Step 1: Preparation of tert-butyl 2-(5-fluoro-2-methyl-3-(4-oxo-3-(2-oxobutyl)-3,4-dihydro-phthalazin-1-yl)-1H-indol-1-yl)acetate, Intermediate 134. To a 100 mL round bottom flask was added intermediate 5, tert-butyl 2-(5-fluoro-3-(4-hydroxyphthalazin-1-yl)-2-methyl-1H-indol-1-yl)acetate (1.00 g, 2.45 mmol, 1.0 eq.), potassium carbonate (0.339 g, 6.14 mmol, 2.5 eq.) and DMF (25 mL, 0.1 M). The flask was purged with nitrogen and 1-bromobutan-2-one (0.752 mL, 7.36 mmol, 3.0 eq.) was added and the reaction stirred at 90° C. overnight. The reaction was cooled to room temperature, extracted with ethyl acetate, washed with brine, dried over MgSO₄, and concentrated in vacuo. The resulting material was purified via silica gel chromatography (Biotage 12-100% ethyl acetate in hexanes) to yield intermediate 134, as pale yellow solid (0.924 g, 79.0%).

Step 2: Preparation of tert-butyl 2-(3-(3-(2-ethyl-2-hydroxybutyl)-4-oxo-3,4-dihydrophthalazin-1-yl)-5-fluoro-2-methyl-1H-indol-1-yl)acetate, Intermediate 135. To a dry 250 mL rbf which contained intermediate 134, tert-butyl 2-(5-fluoro-2-methyl-3-(4-oxo-3-(2-oxobutyl)-3,4-dihydro-phthalazin-1-yl)-1H-indol-1-yl)acetate (1.410 g, 2.95 mmol, 1.0 eq.) was added anhydrous THF (29.5 mL, 0.1 M) via a syringe. The reaction was cooled to −78° C. and 1.0 M ethylmagnesium bromide (5.91 mL, 5.91 mmol 2.0 eq) was added dropwise. The reaction was allowed to warm to room temperature and stirred for 24 hours. The reaction was quenched with saturated ammonium chloride, extracted with ethyl acetate, washed with brine, dried over MgSO₄, and concentrated. The resulting material was purified via silica gel chromatography (Biotage 12-100% ethyl acetate in hexanes) yielding intermediate 199 (0.315 g, 21.0%).

Step 3: Preparation of 2-(3-(3-(2-ethyl-2-hydroxybutyl)-4-oxo-3,4-dihydrophthalazin-1-yl)-5-fluoro-2-methyl-1H-indol-1-yl)acetic acid (128). Intermediate 135, (0.315 g, 0.620 mmol, 1.0 eq.) was dissolved in trifluoroacetic acid (5 mL) and stirred for 3 hours at room temperature. Water was added, and the reaction was extracted with ethyl acetate, washed with brine, dried over MgSO₄, and concentrated in vacuo. The resulting material was purified via reverse phase HPLC (Gilson acidic) yielding 128 as a white solid (136.0 mg, 48.6%). ¹H NMR (400 MHz, MeOD) δ 8.45-8.53 (m, 1H) 7.79-7.93 (m, 2H) 7.64-7.71 (m, 1H) 7.39 (dd, J=8.7, 4.2 Hz, 1H) 6.87-7.01 (m, 2H) 5.07 (d, J=5.1 Hz, 2H) 4.31-4.53 (m, 2H) 2.36 (s, 3H) 1.54-1.73 (m, 4H) 0.97 (t, J=7.5 Hz, 6H).

### Example 129

Preparation of 2-(5-fluoro-3-(3-(2-hydroxy-2-methylpropyl)-4-oxo-3,4-dihydrophthalazin-1-yl)-2-methyl-1H-indol-1-yl)acetic acid (129). To a 50 mL conical flask was added 5, tert-butyl 2-(5-fluoro-3-(4-hydroxyphthalazin-1-yl)-2-methyl-1H-indol-1-yl)acetate (0.140 g, 0.344 mmol, 1.0 eq.), 1 M NaOH (0.6 mL, 0.600 mmol), 1,4-Dioxane (1.185 mL, 0.3 M), and finally 2,2-dimethyloxirane (0.033 g, 0.464 mmol, 1.35 eq.). The flask was equipped with a condenser under nitrogen and allowed to reflux overnight. The reaction was cooled to room temperature, extracted with ethyl acetate, washed with brine, dried over MgSO₄, and concentrated. The resulting yellow oil was purified via silica gel chromatography (Biotage 12-100% ethyl acetate in hexanes with 1% acetic acid modifier) yielding 129 as a white solid (30.7mg, 21.1%). ¹H NMR (400 MHz, DMSO-d₆) δ 12.46 (br. s., 1H) 8.38 (dd, J=7.6, 1.3 Hz, 1H) 7.85 (m, J=7.4, 7.4, 7.4, 7.4, 1.4 Hz, 2H) 7.50-7.57 (m, 1H) 7.37 (dd, J=8.7, 4.4 Hz, 1H) 6.85-6.98 (m, 2H) 4.73 (s, 1H) 4.54 (s, 2H) 4.16-4.32 (m, 2H) 2.26 (s, 3H) 1.21 (d, J=3.8Hz, 6H).

### Example 130

Step 1: Preparation of 4-bromo-2-methylbutan-2-ol, Intermediate 136. In a 250 mL round bottom flask, methyl 3-bromopropanoate (5.0 mL, 45.8 mmol, 1.0 eq.) was taken up in dry ether (55.2 mL, 0.83 M) under nitrogen and cooled to −20° C. To this, 3.0 M methylmagnesium bromide (45.8 ml, 137 mmol, 3.0 eq) was added in dropwise fashion and the resulting mixture was stirred for an hour. The reaction was quenched with aqueous ammonium chloride. The resulting white suspension was extracted with ether multiple times. The combined organics were washed with brine, dried over MgSO₄, and concentrated to yield 4-bromo-2-methylbutan-2-ol as an oil (5.49 g, 71.7%)

Step 2: Preparation of tert-butyl 2-(5-fluoro-3-(3-(3-hydroxy-3-methylbutyl)-4-oxo-3,4-dihydro-phthalazin-1-yl)-2-methyl-1H-indol-1-yl)acetate, Intermediate 137. To a 100 mL round bottom flask was added intermediate 5, tert-butyl 2-(5-fluoro-3-(4-hydroxyphthalazin-1-yl)-2-methyl-1H-indol-1-yl)acetate (0.500 g, 1.23 mmol, 1.0 eq.), cesium carbonate (1.00 g, 3.07 mmol, 2.5 eq.) and DMF (12 mL, 0.1 M). The flask was purged with nitrogen and intermediate 136 (0.615 g, 3.68 mmol, 3.0 eq.) was added and the reaction stirred at 90° C. overnight. The reaction was cooled to room temperature, extracted with ethyl acetate, washed with brine, dried over MgSO₄, and concentrated in vacuo. The resulting material was carried on crude.

Step 3: Preparation of 2-(5-fluoro-2-methyl-3-(3-(3-methylbut-2-enyl)-4-oxo-3,4-dihydro-phthalazin-1-yl)-1H-indol-1-yl)acetic acid (130). Intermediate 137 was dissolved in trifluoroacetic acid (5 mL) and stirred overnight at room temperature. Water was added, and the reaction was extracted with ethyl acetate, washed with brine, dried over MgSO₄, and concentrated in vacuo. The resulting material was purified via reverse phase HPLC (Gilson acidic) yielding 130 as a white solid (85.6 mg, 16.6% over two steps). ¹H NMR (400 MHz, DMSO-d₆) δ 13.36 (br. s., 1H) 8.53 (d, J=7.1 Hz, 1H) 8.20 (td, J=7.6, 1.1 Hz, 1H) 7.94-8.01 (m, 1H) 7.72 (dd, J=9.1, 4.3 Hz, 1H) 7.33 (dd, J=9.6, 2.5 Hz, 1H) 7.24 (d, J=7.8 Hz, 1H) 7.11 (td, J=9.2, 2.5 Hz, 1H) 5.13-5.33 (m, 2H) 4.52 (t, J=7.1 Hz, 2H) 2.28 (s, 3H) 1.58 (s, 3H) 1.47 (s, 3H).

### Example 131

Preparation of 2-(5-fluoro-2-methyl-3-(4-oxo-3-(3,3,3-trifluoro-2-hydroxy-2-(trifluoro-methyl)propyl)-3,4-dihydrophthalazin-1-yl)-1H-indol-1-yl)acetic acid (131). The title compound was prepared according to the procedure of Example 129.

### Example 132

Preparation of 2-(5-fluoro-3-(3-(3-hydroxy-3-methylbutyl)-4-oxo-3,4-dihydrophthalazin-1-yl)-2-methyl-1H-indol-1-yl)acetic acid (132): The title compound was prepared by reacting intermediate 137 with TFA. Yield: 53.5%

### Example 133

Preparation of 2-(5-fluoro-2-methyl-3-(4-oxo-3-(2-oxobutyl)-3,4-dihydrophthalazin-1-yl)-1H-indol-1-yl)acetic acid (133). The title compound was prepared by reacting intermediate 137 with TFA. Yield: 42.3%

### Example 134

Preparation of 2-(5-fluoro-2-methyl-3-(4-oxo-3-(pyridin-4-ylmethyl)-3,4-dihydrophthalazin-1-yl)-1H-indol-1-yl)acetic acid (134). The title compound was prepared according to the procedure of Example 97; Yield: 30.8%.

### Example 135

Preparation of 2-(5-fluoro-2-methyl-3-(4-oxo-3-(pyridin-3-ylmethyl)-3,4-dihydrophthalazin-1-yl)-1H-indol-1-yl)acetic acid (135). The title compound was prepared according to the procedure of Example 97; Yield: 30.6%.

### Example 136

Preparation of 2-(5-fluoro-2-methyl-3-(4-oxo-3-(pyridin-2-ylmethyl)-3,4-dihydrophthalazin-1-yl)-1H-indol-1-yl)acetic acid (136). The title compound was prepared according to the procedure of Example 97; Yield: 38.7%.

### Example 137

Preparation of 2-(5-fluoro-2-methyl-3-(4-oxo-3-(4,4,4-trifluoro-3-(trifluoromethyl)butyl)-3,4-dihydrophthalazin-1-yl)-1H-indol-1-yl)acetic acid (137). The title compound was prepared according to the procedure of Example 97; Yield: 16.3%.

### Example 138

Step 1: Preparation of (3-fluoropyridin-4-yl)methanol, Intermediate 138, To a 50 mL conical vial equipped with a condenser was added 3-fluoroisonicotinaldehyde (0.5 ml, 5.02 mmol, 1.0 eq), tetrahydrofuran (20.06 mL, 0.25 M), sodium borohydride (0.190 g, 5.02 mmol, 1.0 eq.), and MeOH (4.01 mL, 1.25 M). The reaction was heated to reflux and stirred overnight. The reaction was cooled to room temperature, extracted with ethyl acetate, washed with brine, dried over MgSO₄, and concentrated in vacuo. The resulting white solid was intermediate 50, (3-fluoropyridin-4-yl)methanol (0.580 g, 91.0%)

Step 2: Preparation of tert-butyl 2-(5-fluoro-3-(3-((3-fluoropyridin-4-yl)methyl)-4-oxo-3,4-dihydro-phthalazin-1-yl)-2-methyl-1H-indol-1-yl)acetate, Intermediate 139. To a 100 mL round bottom flask was added intermediate 5, tert-butyl 2-(5-fluoro-2-methyl-3-(4-oxo-3,4-dihydrophthalazin-1-yl)-1H-indol-1-yl)acetate (0.400 g, 0.982 mmol, 1.0 eq.), intermediate 138, (3-fluoropyridin-4-yl)methanol (0.250 g, 1.963 mmol, 2.0 eq.), triphenyl-phosphine (0.541 g, 2.062 mmol, 2.1 eq.). The flask was purged with nitrogen and the reaction was cooled to 0° C. DIAD (0.401 mL, 2.062 mmol, 2.1 eq.) was added via syringe. The ice bath warmed to room temperature and the reaction stirred overnight. The reaction was extracted with ethyl acetate, washed with brine, dried over MgSO₄, and concentrated in vacuo. The resulting material was run through silica gel chromatography (Biotage 12-100% ethyl acetate in hexanes) yielding impure intermediate 139 (0.230 g, 45.4%).

Step 3: Preparation of 2-(5-fluoro-3-(3-((3-fluoropyridin-4-yl)methyl)-4-oxo-3,4-dihydrophthalazin-1-yl)-2-methyl-1H-indol-1-yl)acetic acid (138). The title compound was prepared according to the procedure of Example 97; Yield: 29.7%. ¹H NMR (400MHz, DMSO-d₆) δ 13.19 (s., 1H) 8.58 (d, J=1.5 Hz, 1H) 8.36-8.43 (m, 2H) 7.86-7.96 (m, 2H) 7.51-7.58 (m, 2H) 7.38 (dd, J=6.2, 5.2 Hz, 1H) 6.99 (td, J=9.2, 2.4 Hz, 1H) 6.90 (dd, J=9.9, 2.5 Hz, 1H) 5.53 (d, J=5.6 Hz, 2H) 5.10 (s, 2H) 2.22 (s, 3H).

### Example 139

Step 1: Preparation of methyl 2-(5-chloro-3-(6-chloropyridazin-3-yl)-2-methyl-1H-indol-1-yl)-acetate, Intermediate 140. Intermediate 140 was prepared according to method for intermediate 18.

Step 2: Preparation of methyl 2-(5-chloro-3-(6-hydroxypyridazin-3-yl)-2-methyl-1H-indol-1yl)-acetate, Intermediate 141. Intermediate 141 was prepared according to the method for intermediate 19. Yield: 94.1%

Step 3: Preparation of methyl 2-(5-chloro-3-(1-(4-fluorobenzyl)-6-oxo-1,6-dihydropyridazin-3-yl)-2-methyl-1H-indol-1-yl)acetate, Intermediate 142. Intermediate 142 was prepared according to the method for example 12.

Step 4: Preparation of 2-(5-chloro-3-(1-(4-fluorobenzyl)-6-oxo-1,6-dihydropyridazin-3-yl)-2- methyl-1H-indol-1-yl)acetic acid (139). It was prepared according to the method for Example 12. ¹H NMR (400 MHz, MeOD) δ 7.98 (s, 1H) 7.77 (d, J=9.6 Hz, 1H) 7.59 (d, J=2.0 Hz, 1H) 7.47-7.55 (m, 2H) 7.32 (d, J=8.6 Hz, 1H) 7.06-7.16 (m, 3H) 5.41 (s, 2H) 4.89 (s, 2H) 2.48 (s, 3H).

### Example 140

Preparation of 2-(5-chloro-3-(1-(2-(4-chlorophenoxy)ethyl)-6-oxo-1,6-dihydropyridazin-3-yl)-2-methyl-1H-indol-1-yl)acetic acid (140). The title compound was prepared according to the procedure of Example 139; Yield: 19.1%.

### Example 141

Preparation of 2-(3-(1-(benzo[d]thiazol-2-ylmethyl)-6-oxo-1,6-dihydropyridazin-3-yl)-5-chloro-2-methyl-1H-indol-1-yl)acetic acid (141). The title compound was prepared according to the procedure of Example 139; Yield: 6.7%.

### Example 142

Preparation of 2-(5-chloro-3-(1-(4-fluoro-2-(trifluoromethyl)benzyl)-6-oxo-1,6-dihydro-pyridazin-3-yl)-2-methyl-1H-indol-1-yl)acetic acid (142). The title compound was prepared according to the procedure of Example 139; Yield: 15.7%.

### Example 143

Preparation of 2-(5-chloro-3-(1-(3-fluoro-2-(trifluoromethyl)benzyl)-6-oxo-1,6-dihydro-pyridazin-3-yl)-2-methyl-1H-indol-1-yl)acetic acid (143). The title compound was prepared according to the procedure of Example 139; Yield: 21.4%.

### Example 144

Preparation of 2-(5-chloro-2-methyl-3-(6-oxo-1-(quinolin-2-ylmethyl)-1,6-dihydro-pyridazin-3-yl)-1H-indol-1-yl)acetic acid (144). The title compound was prepared according to the procedure of Example 139; Yield: 24.3%.

### Example 145

Step 1: Preparation of methyl 2-(5-chloro-2-methyl-3-(142-methylquinolin-4-yl)methyl)-6-oxo-1,6-dihydropyridazin-3-yl)-1H-indol-1-yl)acetate, Intermediate 143. To a 100 mL round bottom flask was added intermediate 141, methyl 2-(5-chloro-3-(6-hydroxypyridazin-3-yl)-2-methyl-1H-indol-1-yl)acetate (0.375 g, 1.13 mmol, 1.0 eq.), potassium carbonate (0.937 g, 6.78 mmol, 6.0 eq.), and 4-(chloromethyl)-2-methylquinoline (0.432 g, 2.26 mmol, 2.0 eq.). The flask was purged with nitrogen and DMF (12 mL, 0.1 M) was added. The reaction was stirred at 90° C. overnight. It was cooled to room temperature, extracted with ethyl acetate, washed with brine, dried over MgSO₄, and concentrated in vacuo. The resulting material was carried on crude.

Step 2: Preparation of 2-(5-chloro-2-methyl-3-(1-((2-methylquinolin-4-yl)methyl)-6-oxo-1,6-dihydropyridazin-3-yl)-1H-indol-1-yl)acetic acid (145). To a 100 mL round bottom flask which contained crude intermediate 143, was added THF (4 mL), lithium hydroxide in water, and finally methanol to make the mixture homogeneous. The reaction stirred at room temperature for 3 hours. It was acidified with concentrated HCl, extracted with ethyl acetate, washed with brine, dried over MgSO₄, and concentrated in vacuo. The resulting material was purified using reverse phase HPLC (Gilson acidic) yielding 145 as a yellow solid (85.0 mg, 15.9% over two steps). ¹H NMR (400 MHz, DMSO-d₆) δ 13.21 (br. s., 1H) 8.21 (d, J=8.3 Hz, 1H) 7.98 (d, J=7.6 Hz, 1H) 7.81 (d, J=9.9 Hz, 1H) 7.74 (td, J=7.6, 1.3 Hz, 1H) 7.56-7.62 (m, 1H) 7.47-7.52 (m, 2H) 7.25 (s, 1H) 7.10-7.19 (m, 2H) 5.84 (s, 2H) 5.05 (s, 2H) 2.64 (s, 3H) 2.30 (s, 3H).

### Example 146

Step 1: Preparation of methyl 2-(5-chloro-2-methyl-3-(1-(4-methylbenzyl)-6-oxo-1,6-dihydro-pyridazin-3-yl)-1H-indol-1-yl)acetate, Intermediate 144. To a 100 mL round bottom flask was added intermediate 141, methyl 2-(5-chloro-3-(6-hydroxypyridazin-3-yl)-2-methyl-1H-indol-1-yl)acetate (0.350 g, 1.05 mmol, 1.0 eq.), potassium carbonate (0.365 g, 2.64 mmol, 2.5 eq.), 1-(bromomethyl)-4-methylbenzene (0.390 g, 2.11 mmol, 2.0 eq.). The flask was purged with nitrogen and DMF (10 mL, 0.1 M) was added. The reaction was stirred at 90° C. overnight. It was cooled to room temperature, extracted with ethyl acetate, washed with brine, dried over MgSO₄, and concentrated in vacuo. The resulting material was carried on crude.

Step 2: Preparation of 2-(5-chloro-2-methyl-3-(1-(4-methylbenzyl)-6-oxo-1,6-dihydropyridazin-3-yl)-1H-indol-1-yl)acetic acid (146). To a 100 mL round bottom flask which contained crude intermediate 144, was added THF (4 mL), lithium hydroxide in water, and finally methanol to make the mixture homogeneous. The reaction stirred at room temperature for 3 hours. It was acidified with concentrated HCl, extracted with ethyl acetate, washed with brine, dried over MgSO₄, and concentrated in vacuo. The resulting material was purified using reverse phase HPLC (Gilson acidic) yielding 146 as a yellow solid (130.5 mg, 29.5% over two steps). ¹H NMR (400 MHz, DMSO-d₆) δ 7.73 (d, J=9.6 Hz, 1H) 7.58 (d, J=2.0 Hz, 1H) 7.50 (d, J=8.8 Hz, 1H) 7.29 (d, J=8.1 Hz, 2H) 7.13-7.21 (m, 3H) 7.05 (d, J=9.6 Hz, 1H) 5.29 (s, 2H) 5.06 (s, 2H) 2.42 (s, 3H) 2.28 (s, 3H).

### Example 147

Preparation of 2-(5-chloro-3-(1-(4-isopropylbenzyl)-6-oxo-1,6-dihydropyridazin-3-yl)-2-methyl-1H-indol-1-yl)acetic acid (147). The title compound was prepared according to the procedure of Example 139; Yield: 29.5%.

### Example 148

Preparation of 2-(5-chloro-3-(1-(4-methoxybenzyl)-6-oxo-1,6-dihydropyridazin-3-yl)-2-methyl-1H-indol-1-yl)acetic acid (148). The title compound was prepared according to the procedure of Example 139; Yield: 19.76%.

### Example 149

Preparation of 2-(5-chloro-2-methyl-3-(1-methyl-6-oxo-1,6-dihydropyridazin-3-yl)-1H-indol-1-yl)acetic acid (149). The title compound was prepared according to the procedure of Example 120 and hydrolyzed according to Example 139; Yield: 26.3%.

### Example 150

Preparation of 2-(5-chloro-3-(1-ethyl-6-oxo-1,6-dihydropyridazin-3-yl)-2-methyl-1H-indol-1-yl)acetic acid (150). The title compound was prepared according to the procedure of Example 121 and hydrolyzed according to Example 139; Yield: 39.8%.

### Example 151

Preparation of 2-(5-fluoro-3-(1-(4-fluorobenzyl)-6-oxo-1,6-dihydropyridazin-3-yl)-2-methyl-1H-indol-1-yl)acetic acid (151). The title compound was prepared according to the procedure of Example 139; Yield: 18.4%.

### Example 152

Preparation of 2-(3-(1-(2-(4-chlorophenoxy)ethyl)-6-oxo-1,6-dihydropyridazin-3-yl)-5-fluoro-2-methyl-1H-indol-1-yl)acetic acid (152). The title compound was prepared according to the procedure of Example 139; Yield: 30.2%.

### Example 153

Preparation of 2-(3-(1-(benzo[d]thiazol-2-ylmethyl)-6-oxo-1,6-dihydropyridazin-3-yl)-5-fluoro-2-methyl-1H-indol-1-yl)acetic acid (153). The title compound was prepared according to the procedure of Example 139; Yield: 7.8%.

### Example 154

Preparation of 2-(5-fluoro-3-(1-(3-fluorobenzyl)-6-oxo-1,6-dihydropyridazin-3-yl)-2-methyl-1-H-indol-1-yl)acetic acid (154). The title compound was prepared according to the procedure of Example 139; Yield: 13.4%.

### Example 155

Preparation of 2-(5-fluoro-3-(1-(4-fluoro-2-(trifluoromethyl)benzyl)-6-oxo-1,6-dihydro-pyridazin-3-yl)-2-methyl-1H-indol-1-yl)acetic acid (155). The title compound was prepared according to the procedure of Example 139; Yield: 17.4%.

### Example 156

Preparation of 2-(5-fluoro-3-(1-(3-fluoro-2-(trifluoromethyl)benzyl)-6-oxo-1,6-dihydro-pyridazin-3-yl)-2-methyl-1H-indol-1-yl)acetic acid (156). The title compound was prepared according to the procedure of Example 139; Yield: 23.8%.

### Example 157

Preparation of 2-(5-fluoro-2-methyl-3-(6-oxo-1-(quinolin-2-ylmethyl)-1,6-dihydro-pyridazin-3-yl)-1H-indol-1-yl)acetic acid (157). The title compound was prepared according to the procedure of Example 139; Yield: 54%.

### Example 158

Preparation of 2-(5-fluoro-2-methyl-3-(1-((2-methylquinolin-4-yl)methyl)-6-oxo-1,6-dihydro-pyridazin-3-yl)-1H-indol-1-yl)acetic acid (158). The title compound was prepared according to the procedure of Example 139; Yield: 46.8%.

### Example 159

Preparation of 2-(5-fluoro-2-methyl-3-(1-methyl-6-oxo-1,6-dihydropyridazin-3-yl)-1H-indol-1-yl)acetic acid (159). The title compound was prepared according to the procedure of Example 120 and hydrolyzed according to the procedure of Example 139; Yield: 47.5%

### Example 160

Preparation of 2-(3-(1-ethyl-6-oxo-1,6-dihydropyridazin-3-yl)-5-fluoro-2-methyl-1H-indol-1-yl)acetic acid (160). The title compound was prepared according to the procedure of Example 121 and hydrolyzed according to the procedure of Example 139; Yield: 26.3%

### Example 161

Preparation of 2-(3-(1-(cyclopropylmethyl)-6-oxo-1,6-dihydropyridazin-3-yl)-5-fluoro-2-methyl-1H-indol-1-yl)acetic acid (161). The title compound was prepared according to the procedure of Example 139; Yield: 29.2%.

### Example 162

Preparation of 2-(5-fluoro-2-methyl-3-(6-oxo-1-(2,2,2-trifluoro ethyl)-1,6-dihydropyridazin-3-yl)-1H-indol-1-yl)acetic acid (162). The title compound was prepared according to the procedure of Example 139; Yield: 29.2%.

### Example 163

Preparation of 2-(5-fluoro-2-methyl-3-(6-oxo-1-(4,4,4-trifluorobutyl)-1,6-dihydropyridazin-3-yl)-1H-indol-1-yl)acetic acid (163). The title compound was prepared according to the procedure of Example 139; Yield: 24.8%.

### Example 164

Preparation of 2-(5-fluoro-2-methyl-3-(6-oxo-1-(3,3,3-trifluoropropyl)-1,6-dihydro-pyridazin-3-yl)-1H-indol-1-yl)acetic acid (164). The title compound was prepared according to the procedure of Example 139; Yield: 14.7%.

### Example 165

Preparation of 2-(5-fluoro-2-methyl-3-(1-neopentyl-6-oxo-1,6-dihydropyridazin-3-yl)-1H-indol-1-yl)acetic acid (165). The title compound was prepared according to the procedure of Example 139; Yield: 3.9%.

### Example 166

Step 1: Preparation of tert-butyl 2-(3-(4-chlorophthalazin-1-yl)-2-methyl-1H-indol-1-yl)acetate, Intermediate 145. The title compound was prepared according to the procedure of intermediate 1A.

Step 2: Preparation of tert-butyl 2-(3-(4-hydroxyphthalazin-1-yl)-2-methyl-1H-indol-1-yl)acetate, Intermediate 146. The title compound was prepared according to the procedure of intermediate 2; Yield 67.2%.

Step 3: Preparation of tert-butyl 2-(3-(3-(4-fluorobenzyl)-4-oxo-3,4-dihydrophthalazin-1-yl)-2-methyl-1H-indol-1-yl)acetate, Intermediate 147. The title compound was prepared according to the procedure of Example 97.

Step 4: Preparation of 2-(3-(3-(4-fluorobenzyl)-4-oxo-3,4-dihydrophthalazin-1-yl)-2-methyl-1H-indol-1-yl)acetic acid (166). The title compound was prepared according to the procedure of Example 97; Yield 40.3%.

### Example 167

Preparation of 2-(3-(3-(benzo[d]thiazol-2-ylmethyl)-4-oxo-3,4-dihydrophthalazin-1-yl)-2-methyl-1H-indol-1-yl)acetic acid (167). The title compound was prepared according to the procedure of Example 166; Yield 58.4%.

### Example 168

Preparation of 2-(2-methyl-3-(3-(4-(methylsulfonyl)benzyl)-4-oxo-3,4-dihydrophthalazin-1-yl)-1H-indol-1-yl)acetic acid (168). The title compound was prepared according to the procedure of Example 166; Yield 45.1%.

### Example 169

Preparation of 2-(2-methyl-3-(4-oxo-3-(quinolin-2-ylmethyl)-3,4-dihydrophthalazin-1-yl)-1H-indol-1-yl)acetic acid (169). The title compound was prepared according to the procedure of Example 166; Yield 37.6%

### Example 170

Preparation of 2-(2-methyl-3-(4-oxo-3-(4-(trifluoromethoxy)benzyl)-3,4-dihydrophthalazin-1-yl)-1H-indol-1-yl)acetic acid (170). The title compound was prepared according to the procedure of Example 166; Yield 48.4%.

### Example 171

Preparation of 2-(2-methyl-3-(4-oxo-3-(4-(trifluoromethyl)benzyl)-3,4-dihydrophthalazin-1-yl)-1H-indol-1-yl)acetic acid (171). The title compound was prepared according to the procedure of Example 166; Yield 50.8%.

### Example 172

Preparation of 2-(3-(3-(2,6-difluorobenzyl)-4-oxo-3,4-dihydrophthalazin-1-yl)-2-methyl-1H-indol-1-yl)acetic acid (172). The title compound was prepared according to the procedure of Example 166; Yield 34.4%.

### Example 173

Preparation of 2-(2-methyl-3-(3-(4-methylbenzyl)-4-oxo-3,4-dihydrophthalazin-1-yl)-1H-indol-1-yl)acetic acid (173). The title compound was prepared according to the procedure of Example 166; Yield 22.5%.

### Example 174

Preparation of 2-(2-methyl-3-(3-methyl-4-oxo-3,4-dihydrophthalazin-1-yl)-1H-indol-1-yl)-acetic acid (174). The title compound was prepared according to the procedure of Example 166; Yield 40.2%.

### Example 175

Preparation of 2-(3-(3-ethyl-4-oxo-3,4-dihydrophthalazin-1-yl)-2-methyl-1H-indol-1-yl)-acetic acid (175). The title compound was prepared according to the procedure of Example 166; Yield 15.7%.

### Example 176

Preparation of 2-(3-(3-(cyclopropylmethyl)-4-oxo-3,4-dihydrophthalazin-1-yl)-2-methyl-1H-indol-1-yl)acetic acid (176). The title compound was prepared according to the procedure of Example 166; Yield 7.2%.

### Example 177

Preparation of 2-(2-methyl-3-(4-oxo-3-(2,2,2-trifluoro ethyl)-3,4-dihydrophthalazin-1-yl)-1H-indol-1-yl)acetic acid (177). The title compound was prepared according to the procedure of Example 166; Yield 8.3%

### Example 178

Preparation of 2-(3-(3-cyclopropyl-4-oxo-3,4-dihydrophthalazin-1-yl)-2-methyl-1H-indol-1-yl)-acetic acid (178). The title compound was prepared according to the procedure of Example 123;Yield 2.8%

### Example 179

Preparation of 2-(3-(3-cyclopentyl-4-oxo-3,4-dihydrophthalazin-1-yl)-2-methyl-1H-indol-1-yl)-acetic acid (179). A microwave vial containing intermediate 146, (0.500 g, 1.28 mmol, 1.0 eq.) and potassium carbonate (0.442 g, 3.20 mmol, 2.5 eq.) was sealed and purged with nitrogen. NMP (12 mL, 0.1 M) and bromocyclopentane (1.37 mL, 12.8 mmol, 10.0 eq.) were added via syringe. The vial was purged again with nitrogen and subject to the microwave at 150° C. for 10 minutes. The reaction mixture was extracted with ethyl acetate, washed with brine, dried over MgSO₄, and concentrated in vacuo. The resulting material was purified via reverse phase HPLC (Gilson acidic) yielding 179 as a solid (26.2 mg, 5.1%). ¹H NMR (400 MHz, MeOD) δ 8.45 (d, J=7.1 Hz, 1H) 7.83-7.90 (m, 1H) 7.75-7.82 (m, 1H) 7.64 (d, J=7.6 Hz, 1H) 7.32-7.43 (m, 1H) 7.13-7.24 (m, 2H) 6.97-7.08 (m, 1H) 5.18-5.29 (m, 1H) 5.05 (d, J=6.1 Hz, 2H) 2.33 (s, 3H) 1.78-1.91 (m, 2H) 1.52-1.74 (m, 6H).

### Example 180

Step 1: Preparation of 3-(6-chloropyridazin-3-yl)-2-methyl-1H-indole (Intermediate 148). The title compound was prepared according to the procedure of intermediate 1.

Step 2: Preparation of methyl 2-(3-(6-chloropyridazin-3-yl)-2-methyl-1H-indol-1-yl)acetate, Intermediate 149. The title compound was prepared according to the procedure of intermediate 136.

Step 3: Preparation of methyl 2-(3-(6-hydroxypyridazin-3-yl)-2-methyl-1H-indol-1-yl)acetate (Intermediate 150). The title compound was prepared according to the procedure of intermediate 2. Yield 64.2%

Step 4: Preparation of methyl 2-(3-(1-benzyl-6-oxo-1,6-dihydropyridazin-3-yl)-2-methyl-1H-indol-1-yl)acetate, Intermediate 151. The title compound was prepared according to the procedure of Example 1.

Step 5: Preparation of 2-(3-(1-benzyl-6-oxo-1,6-dihydropyridazin-3-yl)-2-methyl-1H-indol-1-yl)-acetic acid (180). The title compound was prepared according to the procedure of Example 139.

### Example 181

Preparation of 2-(3-(1-isopropyl-6-oxo-1,6-dihydropyridazin-3-yl)-2-methyl-1H-indol-1-yl)-acetic acid (181). The title compound was prepared according to the procedure of Example 180; Yield 21.6%.

### Example 182

Preparation of 2-(3-(1-ethyl-6-oxo-1,6-dihydropyridazin-3-yl)-2-methyl-1H-indol-1-yl)-acetic acid (182). The title compound was prepared according to the procedure of Example 121 and hydrolyzed according to the procedure of Example 139; Yield 29.9%

### Example 183

Preparation of 2-(2-methyl-3-(1-methyl-6-oxo-1,6-dihydropyridazin-3-yl)-1H-indol-1-yl)-acetic acid (183). The title compound was prepared according to the procedure of Example 120 and hydrolyzed according to the procedure of Example 139; Yield 40%

### Example 184

Preparation of 2-(3-(1-(cyclopropylmethyl)-6-oxo-1,6-dihydropyridazin-3-yl)-2-methyl-1H-indol-1-yl)acetic acid (184). The title compound was prepared according to the procedure of Example 180; Yield 29.8%.

### Example 185

Preparation of 2-(2-methyl-3-(6-oxo-1-(2,2,2-trifluoroethyl)-1,6-dihydropyridazin-3-yl)-1H-indol-1-yl)acetic acid (185). The title compound was prepared according to the procedure of Example 180; Yield 25.2%.

### Example 186

Preparation of methyl 2-(3-(1-(2,4-difluorobenzyl)-6-oxo-1,6-dihydropyridazin-3-yl)-2-methyl-1H-indol-1-yl)acetate (186). The title compound was prepared according to the procedure of Example 180; Yield 34.8%.

### Example 187

Preparation of 2-(2-methyl-3-(6-oxo-1-(2,4,5-trifluorobenzyl)-1,6-dihydropyridazin-3-yl)-1H-indol-1-yl)acetic acid (187). The title compound was prepared according to the procedure of Example 180; Yield 32.2%.

### Example 188

Preparation of 2-(3-(1-(2,3-difluorobenzyl)-6-oxo-1,6-dihydropyridazin-3-yl)-2-methyl-1H-indol-1-yl)acetic acid (188). The title compound was prepared according to the procedure of Example 180; Yield 39.3%.

### Example 189

Preparation of 2-(3-(1-(4-fluorobenzyl)-6-oxo-1,6-dihydropyridazin-3-yl)-2-methyl-1H-indol-1-yl)acetic acid (189). The title compound was prepared according to the procedure of Example 180; Yield 21.8%.

### Example 190

Preparation of 2-(3-(1-(2-fluorobenzyl)-6-oxo-1,6-dihydropyridazin-3-yl)-2-methyl-1H-indol-1-yl)acetic acid (190). The title compound was prepared according to the procedure of Example 180; Yield 35.6%.

### Example 191

Step 1: Preparation of 3-(6-chloropyridazin-3-yl)-2,5-dimethyl-1H-indole, Intermediate 152. The title compound was prepared according to the procedure of intermediate 1.

Step 2: Preparation of methyl 2-(3-(6-chloropyridazin-3-yl)-2,5-dimethyl-1H-indol-1-yl)acetate, Intermediate 153. The title compound was prepared according to the procedure of intermediate 1A.

Step 3: Preparation of methyl 2-(3-(6-hydroxypyridazin-3-yl)-2,5-dimethyl-1H-indol-1-yl)acetate, Intermediate 154. The title compound was prepared according to the procedure of intermediate 2 Yield 63.4%.

Step 4: Preparation of methyl 2-(3-(1-benzyl-6-oxo-1,6-dihydropyridazin-3-yl)-2,5-dimethyl-1H-indol-1-yl)acetate, Intermediate 155. The title compound was prepared according to the procedure of Example 180.

Step 5: Preparation of 2-(3-(1-benzyl-6-oxo-1,6-dihydropyridazin-3-yl)-2,5-dimethyl-1H-indol-1-yl)acetic acid (191). The title compound was prepared according to the procedure of Example 180; Yield: 23.5%

### Example 192

Step 1: Preparation of 1-chloro-4-(2,5-dimethyl-1H-indol-3-yl)phthalazine (Intermediate 156). The title compound was prepared according to the procedure of intermediate 1.

Step 2: Preparation of tert-butyl 2-(3-(4-chlorophthalazin-1-yl)-2,5-dimethyl-1H-indol-1-yl)acetate, Intermediate 157. The title compound was prepared according to the procedure of intermediate 1A.

Step 3: Preparation of tert-butyl 2-(3-(4-hydroxyphthalazin-1-yl)-2,5-dimethyl-1H-indol-1-yl)-acetate, Intermediate 158. The title compound was prepared according to the procedure of intermediate 2; Yield 76.6%.

Step 4: Preparation of tert-butyl 2-(3-(3-benzyl-4-oxo-3,4-dihydrophthalazin-1-yl)-2,5-dimethyl-1H-indol-1-yl)acetate, Intermediate 159. The title compound was prepared according to the procedure of Example 97.

Step 5: Preparation of 2-(3-(3-benzyl-4-oxo-3 ,4-dihydrophthalazin-1-yl)-2,5-dimethyl-1H-indol-1-yl)acetic acid (192). The title compound was prepared according to the procedure of Example 97; Yield 18.7%.

### Example 193

Step 1: Preparation of methyl 6-oxo-1-(2,4,5-trifluorobenzyl)-1,4,5,6-tetrahydropyridazine-3-carboxylate, Intermediate 160. The procedure described above for intermediate 39 was followed, reacting intermediate 89 with potassium carbonate and 1-(bromomethyl)-2,4,5-trifluorobenzene to yield intermediate 275 as a white solid (38.1%).

Step 2: Preparation of 6-(hydroxymethyl)-2-(2,4,5-trifluorobenzyl)-4,5-dihydropyridazin-3(2H)-one, Intermediate 161. The procedure described above for intermediate 74 was followed, reacting intermediate 275 with sodium borohydride resulting in intermediate 276 (30.1%)

Step 3: Preparation of 6-oxo-1-(2,4,5-trifluorobenzyl)-1,6-dihydropyridazine-3-carbaldehyde, Intermediate 162. The procedure described above for intermediate 75 was followed, reacting intermediate 161 and manganese dioxide resulting in 162 (58.8%).

Step 4: Preparation of methyl 2-(5-fluoro-2-methyl-3-((6-oxo-1-(2,4,5-trifluorobenzyl)-1,6-dihydro-pyridazin-3-yl)methyl-1H-indol-1-yl)acetate, Intermediate 163. The procedure described above for intermediate 83 was followed, reacting intermediate 162, intermediate 36, triethylsilane and TFA. The resulting material was carried on crude.

Step 5: Preparation of 2-(5-fluoro-2-methyl-3-((6-oxo-1-(2,4,5-trifluorobenzyl)-1,6-dihydro-pyridazin-3-yl)methyl)-1H-indol-1-yl)acetic acid (193). The procedure described above for Example 139 was followed reacting intermediate 163 with lithium hydroxide yielding 193 as a solid (19.8% over two steps). ¹H NMR (400MHz, DMSO-d₆) δ 7.52-7.60 (m, 1H) 7.30-7.38 (m, 2H) 7.18 (d, J=9.6 Hz, 1H) 7.03 (dd, J=10.0, 2.4 Hz, 1H) 6.82-6.89 (m, 2H) 5.24 (s, 2H) 4.89 (s, 2H) 3.91 (s, 2H) 2.29 (s, 3H).

### Example 194

Step 1: Preparation of methyl 2-(5-fluoro-2-methyl-3-((6-oxo-1-(pyridin-4-ylmethyl)-1,6-dihydro-pyridazin-3-yl)methyl)-1H-indol-1-yl)acetate, Intermediate 164. To a 100 mL round bottom flask was added methyl 2-(5-fluoro-2-methyl-3-((6-oxo-1,6-dihydropyridazin-3-yl)methyl)-1H-indol-1-yl)acetate (0.175 g, 0.531 mmol, 1.0 eq.), cesium carbonate (0.866 g, 2.66 mmol, 5.0 eq.), and 4-(bromomethyl)pyridine hydrobromide (0.336 g, 1.33 mmol, 2.5 eq.). The flask was purged with nitrogen and DMF (8 mL, 0.07 M) was added. The reaction was stirred at 90° C. overnight. It was cooled to room temperature, extracted with ethyl acetate, washed with brine, dried over MgSO₄, and concentrated in vacuo. The resulting material was carried on crude.

Step 2: Preparation of 2-(5-fluoro-2-methyl-3-((6-oxo-1-(pyridin-4-ylmethyl)-1,6-dihydro-pyridazin-3-yl)methyl)-1H-indol-1-yl)acetic acid (194). The procedure described above for example 139 was followed reacting intermediate 164, with lithium hydroxide yielding 194 as an off-white solid (17.9% over two steps). ¹H NMR (400 MHz, MeOD) δ 8.44-8.55 (m, 2H) 7.34 (d, J=5.6 Hz, 2H) 7.28 (d, J=9.6 Hz, 1H) 7.22 (dd, J=8.7, 4.2 Hz, 1H) 7.04 (dd, J=9.5, 2.4 Hz, 1H) 6.82-6.90 (m, 2H) 5.39 (s, 2H) 4.90 (s, 2H) 4.04 (s, 2H) 2.37 (s, 3H).

### Example 195

Step 1: Preparation of methyl 2-(5-fluoro-2-methyl-3-((6-oxo-1-(pyridin-3-ylmethyl)-1,6-dihydro-pyridazin-3-yl)methyl)-1H-indol-1-yl)acetate, Intermediate 165. The procedure described above for intermediate 164 was followed reacting methyl 2-(5-fluoro-2-methyl-3-((6-oxo-1,6-dihydropyridazin-3-yl)methyl)-1H-indol-1-yl)acetate, cesium carbonate and 3-(bromomethyl)-pyridine hydrobromide. The resulting material was carried on crude.

Step 2: Preparation of 2-(5-fluoro-2-methyl-3-((6-oxo-1-(pyridin-3-ylmethyl)-1,6-dihydropyridazin-3-yl)methyl)-1H-indol-1-yl)acetic acid (195). The procedure described above for example 139 was followed reacting intermediate 165, with lithium hydroxide yielding 195 as a yellow solid (11.4% over two steps). ¹H NMR (400 MHz, DMSO-d₆) δ 8.57 (d, J=2.3 Hz, 1H) 8.50 (dd, J=4.9, 1.6 Hz, 1H) 7.69 (dt, J=7.8, 2.1 Hz, 1H) 7.31-7.41 (m, 2H) 7.12-7.20 (m, 2H) 6.81-6.91 (m, 2H) 5.26 (s, 2H) 4.89 (s, 2H) 3.95 (s, 2H) 2.29 (s, 3H).

### Example 196

Step 1: Preparation of methyl 2-(5-fluoro-2-methyl-3-((6-oxo-1-(pyridin-2-ylmethyl)-1,6-dihydro-pyridazin-3-yl)methyl)-1H-indol-1-yl)acetate, Intermediate 166. The procedure described above for intermediate 164 was followed reacting methyl 2-(5-fluoro-2-methyl-3-((6-oxo-1,6-dihydropyridazin-3-yl)methyl)-1H-indol-1-yl)acetate, cesium carbonate and 2-(bromomethyl)-pyridine hydrobromide. The resulting material was carried on crude.

Step 2: Preparation of 2-(5-fluoro-2-methyl-3-((6-oxo-1-(pyridin-2-ylmethyl)-1,6-dihydropyridazin-3-yl)methyl)-1H-indol-1-yl)acetic acid (196). The procedure described above for example 139 was followed reacting intermediate 166, with lithium hydroxide yielding 196 as a white solid (17.2% over two steps). ¹H NMR (400 MHz, DMSO-d₆) δ 13.12 (br. s., 1H) 8.50 (td, J=2.9, 1.8 Hz, 1H) 7.75 (td, J=7.6, 1.9 Hz, 1H) 7.34 (dd, J=8.8, 4.3 Hz, 1H) 7.30 (ddd, J=7.5, 4.9, 1.0 Hz, 1H) 7.13-7.20 (m, 3H) 6.84-6.90 (m, 2H) 5.35 (s, 2H) 4.91 (s, 2H), 3.94 (s, 2H), 2.29 (s, 3H).

### Example 197

Preparation of (5-fluoro-3-{[1-(2-hydroxy-2-methylpropyl)-6-oxo-1,6-dihydropyridazin-3-yl]-methyl}-2-methyl-1H-indol-1-yl)acetic acid (197). The procedure described above for 129 was followed, reacting methyl 2-(5-fluoro-2-methyl-3-((6-oxo-1,6-dihydropyridazin-3-yl)methyl)-1H-indol-1-yl)acetate with 2,2-dimethyloxirane and purifying (33.3% yield). ¹H NMR (400 MHz, DMSO-d₆) δ 13.02 (br. s., 1H), 7.36 (dd, J=4.42, 8.97 Hz, 1H), 7.25 (dd, J=2.53, 9.85 Hz, 1H), 7.14 (d, J=9.60 Hz, 1H), 6.88 (td, J=2.53, 9.22 Hz, 1H), 6.83 (d, J=9.60 Hz, 1H), 4.95 (s, 2H), 4.72 (s, 1H), 4.06 (s, 2H), 3.95 (s, 2H), 2.34 (s, 3H), 1.11 (s, 6H)

### Example 198

Preparation of 2-(5-Chloro-2-methyl-3-(4-oxo-3-(4-(trifluoromethyl)benzyl)-3,4-dihydrophthalazin-1-yl)-1H-indol-1-yl)acetic acid (198). The title compound was prepared according to the procedure of Example 97; Yield 45%.

### Example 199

Preparation of 2-(5-chloro-2-methyl-3-(4-oxo-3-(2-(trifluoromethyl)benzyl)-3,4-dihydrophthalazin-1-yl)-1H-indol-1-yl)acetic acid (199). The title compound was prepared according to the procedure of Example 97; Yield 48%.

### Example 200

Preparation of 2-(5-Chloro-2-methyl-3-(4-oxo-3-(3-(trifluoromethyl)benzyl)-3,4-dihydro-phthalazin-1-yl)-1H-indol-1-yl)acetic acid (200). The title compound was prepared according to the procedure of Example 97; Yield 45%.

### Example 201

Preparation of [5-fluoro-2-methyl-3-({6-oxo-1-[3,3,3-trifluoro-2-hydroxy-2-(trifluoromethyl)-propyl]-1,6-dihydro-pyridazin-3-yl}methyl)-1H-indol-1-yl]acetic acid (201). The procedure described above for Example 131 was followed, reacting methyl 2-(5-fluoro-2-methyl-3-((6-oxo-1,6-dihydro-pyridazin-3-yl)methyl)-1H-indol-1-yl)acetate with 2,2-bis(trifluoro-methyl)oxirane and purifying (32.9% yield). ¹H NMR (400 MHz, DMSO-d₆) δ 13.02 (br. s., 1H), 8.50 (s, 1H), 7.36 (dd, J=4.55, 8.84 Hz, 1H), 7.25 (dd, J=2.27, 9.85 Hz, 1H), 7.21 (d, J=9.60 Hz, 1H), 6.96 (d, J=9.35 Hz, 1H), 6.88 (td, J=2.53, 9.22 Hz, 1H), 4.95 (s, 2H), 4.76 (s, 2H), 3.98 (s, 2H), 2.33 (s, 3H)

### Example 202

Preparation of 2-(5-fluoro-2-methyl-3-(4-oxo-3-(2-(trifluoromethyl)benzyl)-3,4-dihydrophthalazin-1-yl)-1H-indol-1-yl)acetic acid (202). The title compound was prepared according to the procedure of Example 97; Yield 43%.

### Example 203

Preparation of 2-(5-fluoro-2-methyl-3-(4-oxo-3-(3-(trifluoromethyl)benzyl)-3,4-dihydro-phthalazin-1-yl)-1H-indol-1-yl)acetic acid (203). The title compound was prepared according to the procedure of Example 97; Yield 46%.

### Example 204

Preparation of 2-(5-fluoro-2-methyl-3-(4-oxo-3-(4-(trifluoromethyl)benzyl)-3,4-dihydrophthalazin-1-yl)-1H-indol-1-yl)acetic acid (204). The title compound was prepared according to the procedure of Example 97; Yield 46%.

### Example 205

Preparation of 2-(5-fluoro-2-methyl-3-(4-oxo-3-phenyl-3,4-dihydrophthalazin-1-yl)-1H-indol-1-yl)-acetic acid (205). A mixture of intermediate 5, (0.54 g, 1.3 mmol), bromobenzene (0.15 mL, 1.43 mmol), quinolin-8-ol (28 mg, 0.195 mmol), copper iodide (0.37 g, 1.95 mmol), potassium carbonate (0.27 g, 1.95 mmol), and 4 mL DMSO was heated in microwave at 150° C. for 30 min. Water (30 mL) and ethyl acetate (50 mL) were added. The organic layer was washed with brine and dried over magnesium sulfate, filtered, and the solvent removed in vacuo to give a yellow oil. The crude oil was stirred with 3 mL TFA at 25° C. for 4 h. The reaction mixture was concentrated to yield a yellow oil. This was purified by HPLC to yield the desired product as a white powder (77 mg, 24%). 1H NMR (400 MHz, chloroform-d) δ ppm 2.43 (s, 3H), 4.84 (d, J=17.94 Hz, 1H), 4.91 (d, J=17.94 Hz, 1H), 6.92-7.03 (m, 2H), 7.24 (dd, J=8.84, 4.04 Hz, 1H), 7.36-7.42 (m, 1H), 7.47-7.53 (m, 2H), 7.67-7.76 (m, 3H), 7.76-7.81 (m, 1H), 7.82-7.88 (m, 1H), 8.58 (dd, J=8.08, 1.26 Hz, 1H).

### Example 206

Preparation of 2-(5-fluoro-2-methyl-3-(4-oxo-3-(4-(trifluoromethyl)phenyl)-3,4-dihydrophthalazin-1-yl)-1H-indol-1-yl)acetic acid (206). Synthesized by method used for 205, using as starting materials intermediate 5 (0.54 g, 1.3 mmol), 1-bromo-4-(trifluoromethyl)benzene (0.320 g, 1.43 mmol), quinolin-8-ol (28 mg, 0.195 mmol), copper iodide (0.37 g, 1.95 mmol), potessium carbonate (0.27 g, 1.95 mmol), potassium carbonate (0.63 g, 3.62 mmol). The desired product was isolated as a white powder (0.373 g, 58%). 1H NMR (400 MHz, MeOD) δ ppm 2.64 (s, 3H), 5.27 (d, J=7.33 Hz, 2H), 7.17 (d, J=9.60 Hz, 2H), 7.58-7.66 (m, 1H), 8.00 (d, J=7.58 Hz, 1H), 8.06 (d, J=8.59 Hz, 2H), 8.09-8.18 (m, 2H), 8.22 (d, J=8.84 Hz, 2H), 8.78 (d, J=7.58 Hz, 1H).

### Example 207

Preparation of 2-(5-fluoro-2-methyl-3-(4-oxo-3-(3-(trifluoromethyl)phenyl)-3,4-dihydrophthalazin-1-yl)-1H-indol-1-yl)acetic acid (207). Synthesized by method used for 205, using as starting materials intermediate 5, (0.54 g, 1.3 mmol), 1-bromo-3-(trifluoromethyl)benzene (0.320 g, 1.43 mmol), quinolin-8-ol (28 mg, 0.195 mmol), copper iodide (0.37 g, 1.95 mmol), potessium carbonate (0.27 g, 1.95 mmol), potassium carbonate (0.60 g, 3.62 mmol). The desired product was isolated as a white powder (0.373 g, 55%).1H NMR (400 MHz, MeOD) δ ppm 2.64 (s, 3H), 5.31 (d, J=6.57 Hz, 2H), 7.16-7.23 (m, 2H), 7.63 (dd, J=9.85, 4.29 Hz, 1H), 7.93-8.02 (m, 3H), 8.08-8.20 (m, 2H), 8.23-8.30 (m, 1H), 8.33-8.37 (m, 1H), 8.78 (dd, J=7.58, 1.77 Hz, 1H).

### Example 208

Preparation of 2-(3-(3-((5-chlorobenzo[d]thiazol-2-yl)methyl)-4-oxo-3,4-dihydrophthalazin-1-yl)-5-fluoro-2-methyl-1H-indol-1-yl)acetic acid (208). The title compound was prepared according to the procedure of Example 97; Yield 52%.

### Example 209

Preparation of 2-(5-chloro-3-(3-((5-chlorobenzo[d]thiazol-2-yl)methyl)-4-oxo-3,4-dihydro-phthalazin-1-yl)-2-methyl-1H-indol-1-yl)acetic acid (209). The title compound was prepared according to the procedure of Example 97; Yield 58%.

### Example 210

Preparation of 2-(3-(3-(2-(4-chlorophenoxy)ethyl)-4-oxo-3,4-dihydrophthalazin-1-yl)-5-fluoro-2-methyl-1H-indol-1-yl)acetic acid (210). The title compound was prepared according to the procedure of Example 97; Yield 59%.

### Example 211

Preparation of 2-(5-chloro-2-methyl-3-(6-oxo-1-(2-(trifluoromethyl)benzyl)-1,6-dihydro-pyridazin-3-yl)-1H-indol-1-yl)acetic acid (211). The title compound was prepared according to the procedure of Example 139; Yield 52%.

### Example 212

Preparation of 2-(5-chloro-2-methyl-3-(6-oxo-1-(3-(trifluoromethyl)benzyl)-1,6-dihydropyridazin-3-yl)-1H-indol-1-yl)acetic acid (212). The title compound was prepared according to the procedure of Example 139; Yield 52%

### Example 213

Preparation of 2-(5-chloro-2-methyl-3-(6-oxo-1-(4-(trifluoromethyl)benzyl)-1,6-dihydropyridazin-3-yl)-1H-indol-1-yl)acetic acid (213). The title compound was prepared according to the procedure of Example 139; Yield 58%

### Example 214

Preparation of 2-(3-(1-benzyl-6-oxo-1,6-dihydropyridazin-3-yl)-2-methyl-5-(methylsulfonyl)-1H-indol-1-yl)acetic acid (214). The title compound was prepared according to the procedure of Example 139; Yield 28%

### Example 215

Preparation of 2-(3-(3-benzyl-4-oxo-3,4-dihydrophthalazin-1-yl)-2-methyl-5-(methylsulfonyl)-1H-indol-1-yl)acetic acid (215). The title compound was prepared according to the procedure of Example 97; Yield 29%

### Example 216

Preparation of 2-(5-fluoro-2-methyl-3-(6-oxo-1-(2-(trifluoromethyl)benzyl)-1,6-dihydropyridazin-3-yl)-1H-indol-1-yl)acetic acid (216). The title compound was prepared according to the procedure of Example 97; Yield 55%

### Example 217

Preparation of 2-(5-fluoro-2-methyl-3-(6-oxo-1-(3-(trifluoromethyl)benzyl)-1,6-dihydropyridazin-3-yl)-1H-indol-1-yl)acetic acid (217). The title compound was prepared according to the procedure of Example 139; Yield 62%

### Example 218

Preparation of 2-(5-fluoro-2-methyl-3-(6-oxo-1-(3-(trifluoromethyl)benzyl)-1,6-dihydropyridazin-3-yl)-1H-indol-1-yl)acetic acid (218). The title compound was prepared according to the procedure of Example 139; Yield 60%

### Example 219

Preparation of 2-(3-(1-benzyl-6-oxo-1,6-dihydropyridazin-3-yl)-5-bromo-2-methyl-1H-indol-1-yl)-acetic acid (219). The title compound was prepared according to the procedure of Example 139; Yield 48%

### Example 220

Preparation of 2-(5-fluoro-2-methyl-3-(3-(2-methyl-2-phenoxypropyl)-4-oxo-3,4-dihydrophthalazin-1-yl)-1H-indol-1-yl)acetic acid (220). To a mixture of intermediate 5, (0.60 g, 1.47 mmol), 2-methyl-2-phenoxypropan-1-ol, triphenylphosphine (1.16 g, 4.42 mmol), and 8 mL dry DMF was added diisopropyl diazene-1,2-dicarboxylate (0.89 g, 4.42 mmol) at 25° C. The reaction mixture was stirred at 80° C. for 16 h. Water (30 mL) and ethyl acetate (50 mL) were added. The organic layer was washed with brine and dried over magnesium sulfate, filtered, and the solvent removed in vacuo to give yellow oil. The resulting yellow oil was stirred with 5 mL TFA at 25° C. for 4 h. Concentration of the reaction mixture yielded a yellow oil, which was purified by HPLC to offer the desired product (88 mg, 12%) as a white solid. 1H NMR (400 MHz, CHLOROFORM-d) δ ppm 1.40 (s, 6H), 2.32 (s, 3H), 4.57 (d, J=13.64 Hz, 1H), 4.85 (d, J=13.64 Hz, 1H), 4.87 (d, J=18.44 Hz, 1H), 4.94 (d, J=18.44 Hz, 1H), 6.81-6.86 (m, 1H), 6.89-7.09 (m, 5H), 7.17-7.25 (m, 2H), 7.71 (d, J=8.84 Hz, 1H), 7.78-7.83 (m, 1H), 7.85-7.90 (m, 1H), 8.58 (d, J=7.58 Hz, 1H).

### Example 221

Preparation of 2-(5-chloro-2-methyl-3-(4-oxo-3-(2-phenoxyethyl)-3,4-dihydrophthalazin-1-yl)-1H-indol-1-yl)acetic acid (221). The title compound was prepared according to the procedure of Example 97; Yield 67%

### Example 222

Preparation of 2-(5-fluoro-2-methyl-3-(4-oxo-3-(2-phenoxyethyl)-3,4-dihydrophthalazin-1-yl)-1H-indol-1-yl)acetic acid (222). The title compound was prepared according to the procedure of Example 97; Yield 61%

### Example 223

Preparation of 2-(2-methyl-3-(4-oxo-3-(2-phenoxyethyl)-3,4-dihydrophthalazin-1-yl)-1H-indol-1-yl)acetic acid (223). The title compound was prepared according to the procedure of Example 97; Yield 42%

### Example 224

Preparation of 2-(3-(3-(4-fluorophenethyl)-4-oxo-3,4-dihydrophthalazin-1-yl)-2-methyl-1H-indol-1-yl)acetic acid (224). The title compound was prepared according to the procedure of Example 97; Yield 37%

### Example 225

Preparation of 2-(2-methyl-3-(4-oxo-3-phenethyl-3,4-dihydrophthalazin-1-yl)-1H-indol-1-yl)acetic acid (225). The title compound was prepared according to the procedure of Example 97; Yield 34%

### Example 226

Preparation of 2-(5-fluoro-2-methyl-3-(4-oxo-3-phenethyl-3,4-dihydrophthalazin-1-yl)-1H-indol-1-yl)acetic acid (226). The title compound was prepared according to the procedure of Example 97; Yield 48%

### Example 227

Preparation of 2-(5-fluoro-3-(3-(4-fluorophenethyl)-4-oxo-3,4-dihydrophthalazin-1-yl)-2-methyl-1H-indol-1-yl)acetic acid (227). The title compound was prepared according to the procedure of Example 97; Yield 55%

### Example 228

Preparation of 2-(2-methyl-3-(6-oxo-1-phenethyl-1,6-dihydropyridazin-3-yl)-1H-indol-1-yl)acetic acid (228). The title compound was prepared according to procedure of Example 139; Yield 44%.

### Example 229

Preparation of 2-(3-(1-(4-fluorophenethyl)-6-oxo-1,6-dihydropyridazin-3-yl)-2-methyl-1H-indol-1-yl)acetic acid (229). The title compound was prepared according to the procedure of Example 139; Yield 46%

### Example 230

Preparation of 2-(5-fluoro-2-methyl-3-(1-(2-methyl-2-phenoxypropyl)-6-oxo-1,6-dihydropyridazin-3-yl)-1H-indol-1-yl)acetic acid (230). The title compound was prepared according to the procedure of Example 220; Yield 15%.

### Example 231

Preparation of 2-(3-(1-benzyl-6-oxo-1,6-dihydropyridazin-3-yl)-7-chloro-5-fluoro-2-methyl-1H-indol-1-yl)acetic acid (231) Synthesized by method used for 139. 7-Chloro-5-fluoro-2-methyl-1H-indole was prepared from 2-chloro-4-fluoroaniline according to the procedure described in Org. Lett. 2008, 113 for iodonization, and J. Org. Chem. 1996, 61, 3804 for indole formation. Yield 31%.

### Example 232

Preparation of 2-(7-chloro-5-fluoro-3-(1-isopropyl-6-oxo-1,6-dihydropyridazin-3-yl)-2-methyl-1H-indol-1-yl)acetic acid (232). The title compound was prepared according to the procedure of Example 231; Yield 29%

### Example 233

Preparation of 2-(3-(1-benzyl-6-oxo-1,6-dihydropyridazin-3-yl)-5,7-difluoro-2-methyl-1H-indol-1-yl)acetic acid (233) Synthesized by method used for 139. 5,7-Difluoro-2-methyl-1H-indole was prepared from 2,4-difluoroaniline according to the procedure described in Org. Lett. 2008, 113 for iodonization, and J. Org. Chem. 1996, 61, 3804 for indole formation Yield 24%.

### Example 234

Preparation of 2-(3-(1-benzyl-6-oxo-1,6-dihydropyridazin-3-yl)-5,7-dichloro-2-methyl-1H-indol-1-yl)acetic acid (234). Synthesized by method used for 139. 5,7-Dichloro-2-methyl-1H-indole was prepared from 2,4-dichloroaniline according to the procedure described in Org. Lett. 2008, 113 for iodonization, and J. Org. Chem. 1996, 61, 3804 for indole formation. Yield 35%.

### Example 235

Preparation of 2-(5,7-dichloro-3-(1-isopropyl-6-oxo-1,6-dihydropyridazin-3-yl)-2-methyl-1H-indol-1-yl)acetic acid (235). The title compound was prepared according to the procedure of Example 234; Yield 30%

### Example 236

Preparation of 2-(3-(1-benzyl-6-oxo-1,6-dihydropyridazin-3-yl)-5-fluoro-2-methyl-7-(methyl-sulfonyl)-1H-indol-1-yl)acetic acid (236). 5-Fluoro-2-methyl-7-(methylsulfonyl)-1H-indole was prepared from 2-bromo-4-fluoroaniline according to the procedure described in J. Org. Chem. 2006, 70, 2696 for methylsulfone formation, Org. Lett. 2008, 113 for iodination, and J. Org. Chem. 1996, 61, 3804 for indole formation. A mixture of 3,6-dichloropyridazine (0.94 g, 6.38 mmol), 5-fluoro-2-methyl-7-(methylsulfonyl)-1H-indole (0.726 g, 3.19 mmol), aluminum chloride (0.93 g, 7.02 mmol), and 10 mL 1,2-dichloroethane was stirred at 160° C. under microwave for 75 min. Crushed ice (20 mL) was added to the mixture and stirring was continued for 15 minutes. Ethyl acetate (30 mL) was then added. The organic layer was washed with brine and dried over magnesium sulfate, filtered, and the solvent removed in vacuo to give 3-(6-chloropyridazin-3-yl)-5-fluoro-2-methyl-7-(methylsulfonyl)-1H-indole (0.81 g, 75%) as a light yellow solid, which was used for the next step without further purification. The crude 3-(6-chloropyridazin-3-yl)-5-fluoro-2-methyl-7-(methylsulfonyl)-1H-indole (0.70 g, 3.08 mmol), NaH (0.16 g, 60% wt, 4.0 mmol), and 5 mL dry DMF was stirred under nitrogen at 25° C. for 1 h. Methyl 2-bromoacetate (0.42 mL, 4.5 mmol) was added and the reaction mixture was stirred at 70° C. for 2 h. Water (10 mL) and ethyl acetate (30 mL) were added. The organic layer was washed with brine and dried over magnesium sulfate, filtered, and the solvent removed in vacuo to give 3-(6-chloropyridazin-3-yl)-5-fluoro-2-methyl-7-(methylsulfonyl)-1H-indole (0.62 g, 60%) as a light yellow solid, which was used for the next step without further purification. 3-(6-chloropyridazin-3-yl)-5-fluoro-2-methyl-7-(methylsulfonyl)-1H-indole was converted to methyl 2-(5-fluoro-3-(6-hydroxypyridazin-3-yl)-2-methyl-7-(methylsulfonyl)-1H-indol-1-yl)acetate using procedure for intermediate 2. Yield 25%.

### Example 237

Preparation of 2-(3-(3-benzyl-4-oxo-3,4-dihydrophthalazin-1-yl)-5-fluoro-2-methyl-7-(methyl-sulfonyl)-1H-indol-1-yl)acetic acid (237). The title compound was prepared according to the procedure of Example 236 followed by hydrolysis according to Example 97; Yield 30%

### Example 238

Preparation of 2-(5-fluoro-3-((1-(4-(2-hydroxypropan-2-yl)benzyl)-6-oxo-1,6-dihydropyridazin-3-yl)methyl)-2-methyl-1H-indol-1-yl)acetic acid (238). A mixture of methyl 2-(5-fluoro-2-methyl-3-((6-oxo-1,6-dihydropyridazin-3-yl)methyl)-1H-indol-1-yl)acetate, 2-(4-(bromomethyl)phenyl)-propan-2-ol (Bioorg. Med. Chem. Lett. 2004, 14, 3195) and potassium carbonate were reacted following the procedure for intermediate 123 to give methyl 2-(5-fluoro-3-((1-(4-(2-hydroxypropan-2-yl)benzyl)-6-oxo-1,6-dihydropyridazin-3-yl)-methyl)-2-methyl-1H-indol-1-yl)acetate (100%) as a light purple solid, which was used for the next step without further purification, which was hydrolyzed with lithium hydroxide following procedure for example 83 to give the desired product 238 (30%) as a white solid. 1H NMR (400 MHz, MeOD) δ ppm 1.40 (s, 6H), 2.24 (s, 3H), 3.92 (s, 2H), 4.79 (s, 2H), 5.20 (s, 2H), 6.71 (d, J=9.35 Hz, 1H), 6.71-6.77 (m, 1H), 6.99 (dd, J=9.60, 2.27 Hz, 1H), 7.10 (d, J=9.35 Hz, 1H), 7.09-7.13 (m, 1H), 7.22 (d, J=8.59 Hz, 2H), 7.32-7.36 (m, 2H).

### Example 239

Preparation of 2-(5-fluoro-3-((1-(4-(1,1,1,3,3,3-hexafluoro-2-hydroxypropan-2-yl)benzyl)-6-oxo-1,6-dihydropyridazin-3-yl)methyl)-2-methyl-1H-indol-1-yl)acetic acid (239). Synthesized by method used for 238. Methyl 2-(5-fluoro-2-methyl-3-((6-oxo-1,6-dihydropyridazin-3-yl)methyl)-1H-indol-1-yl)acetate (0.220 g, 0.50 mmol) was converted to the desired product (100 mg, 35%) as a white solid. 1H NMR (400 MHz, MeOD) δ ppm 2.35 (s, 3H), 4.06 (s, 2H), 4.84 (s, 2H), 5.39 (s, 2H), 6.83-6.89 (m, 1H), 6.86 (d, J=9.60 Hz, 1H), 7.14 (dd, J=9.85, 2.53 Hz, 1H), 7.23 (dd, J=8.84, 4.29 Hz, 1H), 7.27 (d, J=9.60 Hz, 1H), 7.48 (d, J=8.59 Hz, 2H), 7.71 (d, J=8.34 Hz, 2H).

### Example 240

Preparation of 2-(5-fluoro-3-((1-(3-(2-hydroxypropan-2-yl)benzyl)-6-oxo-1,6-dihydropyridazin-3-yl)methyl)-2-methyl-1H-indol-1-yl)acetic acid (240). Synthesized by method used for 238. Methyl 2-(5-fluoro-2-methyl-3-((6-oxo-1,6-dihydropyridazin-3-yl)methyl)-1H-indol-1-yl)acetate (0.220 g, 0.50 mmol) was converted to the desired product (93 mg, 28%) as a white solid. 1H NMR (400 MHz, MeOD) δ ppm 1.51 (s 6H), 2.36 (s, 3H), 4.05 (s, 2H), 4.91 (s, 2H), 5.35 (s, 2H), 6.83-6.89 (m, 2H), 7.09 (dd, J=9.35, 2.27 Hz, 1H), 7.21-7.25 (m, 3H), 7.27-7.32 (m, 1H), 7.42-7.45 (m, 1H), 7.54-7.56 (m, 1H).

### Example 241

Preparation of 2-(5-fluoro-3-((1-((3-fluoropyridin-4-yl)methyl)-6-oxo-1,6-dihydropyridazin-3-yl)-methyl)-2-methyl-1H-indol-1-yl)acetic acid (241). Synthesized by method used for 230. (3-Fluoropyridin-4-yl)methanol was prepared from 3-fluoroisonicotinaldehyde (Synthesis, 2008, 2, 245). Methyl 2-(5-fluoro-2-methyl-346-oxo-1,6-dihydropyridazin-3-yl)methyl)-1H-indol-1-yl)-acetate (0.220 g, 0.50 mmol) was converted to the desired product (38 mg, 18%) as a white solid. 1H NMR (400 MHz, MeOD) δ ppm 2.38 (s, 3H), 4.04 (s, 2H), 4.84 (s, 2H), 5.50 (s, 2H), 6.82-6.91 (m, 2H), 7.00-7.05 (m, 1H), 7.20-7.26 (m, 2H), 7.29-7.34 (m, 1H), 8.30-8.34 (m, 1H), 8.46-8.50 (m, 1H).

### Example 242

Preparation of 2-(3-((1-(2,4-difluorobenzyl)-6-oxo-1,6-dihydropyridazin-3-yl)methyl)-5,7-difluoro-2-methyl-1H-indol-1-yl)acetic acid (242). Intermediate 167, methyl 2-(5,7-difluoro-2-methyl-3-((6-oxo-1,6-dihydropyridazin-3-yl)methyl)-1H-indol-1-yl)acetate was prepared from 5,7-difluoro-2-methyl-1H-indole (see formation of 233) by the method used for 238. Intermediate 167 (173 mg, 0.5 mmol) was converted to the desired product (57 mg, 25%) as a white solid. 1H NMR (400 MHz, MeOD) δ ppm 2.22 (s, 3H), 3.86 (s, 2H), 4.84 (s, 2H), 5.24 (s, 2H), 6.49-6.56 (m, 1H), 6.70-6.86 (m, 3H), 6.74 (d, J=9.60 Hz, 1H), 7.11 (d, J=9.60 Hz, 1H), 7.18-7.26 (m, 1H).

### Example 243

Preparation of 2-(3-(3-benzyl-4-oxo-3,4-dihydrophthalazin-1-yl)-7-chloro-5-fluoro-2-methyl-1H-indol-1-yl)acetic acid (243). 7-Chloro-5-fluoro-2-methyl-1H-indole (0.70 g, 3.82 mmol) was converted to intermediate 168, tert-butyl 2-(7-chloro-5-fluoro-3-(4-hydroxyphthalazin-1-yl)-2-methyl-1H-indol-1-yl)acetate (0.78 g, 60%) by the method used for intermediate 2, using 1,4-dichlorophthalazine instead of 3,6-dichloropyridazine. Intermediate 168 (0.54 g, 1.22 mmol) was transformed to 243 (174 mg, 30%) by the method used for 2, as a white solid. 1H NMR (400 MHz, DMSO-d₆) δ ppm 2.23 (s, 3H), 5.29 (d, J=18.95 Hz, 1H), 5.36 (d, J=18.95 Hz, 1H), 5.41 (d, J=14.90 Hz, 1H), 5.42 (d, J=14.90 Hz, 1H), 6.89 (dd, J=9.09, 2.53 Hz, 1H), 7.16 (dd, J=9.09, 2.53 Hz, 1H), 7.28-7.32 (m, 1H), 7.32-7.40 (m, 4H), 7.44-7.48 (m, 1H), 7.85-7.94 (m, 2H), 8.39-8.41 (m, 1H).

### Example 244

Preparation of 2-(3-(3-benzyl-4-oxo-3,4-dihydrophthalazin-1-yl)-5,7-difluoro-2-methyl-1H-indol-1-yl)acetic acid (244). 5,7-Difluoro-2-methyl-1H-indole (0.70 g, 4.20 mmol) was converted to intermediate 169, tert-butyl 2-(5,7-difluoro-3-(4-hydroxyphthalazin-1-yl)-2-methyl-1H-indol-1-yl)acetate (1.29 g, 60%) by the method used for intermediate 2, using 1,4-dichlorophthalazine instead of 3,6-dichloropyridazine. Intermediate 169 (0.45 g, 1.05 mmol) was transformed to 244 (145 mg, 30%) by the method used for 2, Scheme 2, as a white solid. 1H NMR (400 MHz, MeOD) δ ppm 2.47 (s, 3H), 5.30 (d, J=18.95 Hz, 1H), 5.37 (d, J=18.95 Hz, 1H), 5.67 )d, J=14.15 Hz, 1H), 5.75 (d, J=14.15 Hz, 1H), 6.83-6.87 (m, 1H), 6.95-7.03 (m, 1H), 7.48-7.59 (m, 3H), 7.65-7.69 (m, 2H), 7.86-7.90 (m, 1H), 8.03-8.14 (m, 2H), 8.68-8.72 (m, 1H).

### Example 245

Preparation of 2-(3-(3-benzyl-4-oxo-3,4-dihydrophthalazin-1-yl)-5,7-dichloro-2-methyl-1H-indol-1-yl)acetic acid (245). 5,7-Dichloro-2-methyl-1H-indole (0.81 g, 4.07 mmol) was converted to intermediate 170, tert-butyl 2-(5,7-dichloro-3-(4-hydroxyphthalazin-1-yl)-2-methyl-1H-indol-1-yl)acetate (1.11 g, 60%) by the method used for intermediate 2, using 1,4-dichlorophthalazine instead of 3,6-dichloropyridazine. Intermediate 170 (0.52 g, 1.13 mmol) was transformed to 245 (166 mg, 30%) by the method used for 2, Scheme 2, as a white solid. 1H NMR (400 MHz, DMSO-d₆) δ ppm 2.23 (s, 3H), 5.29 (s, 1H), 5.33 (s, 1H), 5.37 (d, J=14.90 Hz, 1H), 5.47 (d, J=14.90 Hz, 1H), 7.15 (d, J=2.02 Hz, 1H), 7.26 (d, J=2.02 Hz, 1H), 7.28-7.32 (m, 1H), 7.33-7.42 (m, 4H), 7.43-7.46 (m, 1H), 7.84-7.94 (m, 2H), 8.39-8.43 (m, 1H).

### Example 246

Preparation of 2-(3-(1-benzyl-6-oxo-1,6-dihydropyridazin-3-yl)-7-bromo-2-methyl-1H-indol-1-yl)-acetic acid (246). Synthesized by the method used for 139. Intermediate 171, methyl 2-(7-bromo-3-(6-hydroxypyridazin-3-yl)-2-methyl-1H-indol-1-yl)acetate (0.23 g, 0.64 mmol) was converted to the desired product (75 mg, 26%) as a white powder. ¹H NMR (400 MHz, CHLOROFORM-d) δ ppm 2.29 (s, 3H), 5.33 (bs, 2H), 5.35 (s, 2H), 6.85-6.90 (m, 1H), 7.11 (d, J=9.60 Hz, 1H), 7.21-7.29 (m, 4H), 7.37-7.41 (m, 2H), 7.43 (d, J=9.60 Hz, 2H).

### Example 247

Step 1: Preparation of tent-butyl 3-bromo-2-methyl-1H-indole-1-carboxylate, Intermediate 172. To a 1000 mL round bottom flask containing 2-methylindole (5 g, 38 mmol) and DMF (127 mL) was added bromine (2.0 mL, 38 mmol). After 15 min, the reaction was diluted with EtOAc (800 mL) and washed with water (500 mL), brine (500 mL) and dried (MgSO₄). The suspension was filtered and concentrated. The residue was dissolved in THF (381 mL) and treated with BOC₂O (8.3 g, 38 mmol) and DMAP (232 mg, 1.9 mmol). After 3 h, the reaction was concentrated to remove the THF. The residue was diluted with EtOAc (500 mL) and washed with water (250 mL) and brine (250 mL). The organic layer was dried (MgSO₄), filtered and concentrated. The crude material was purified by Biotage.

Step 2: Preparation of tert-butyl 3-(isoquinolin-4-yl)-2-methyl-1H-indole-1-carboxylate, Intermediate 173. To a microwave vial containing tert-butyl 3-bromo-2-methyl-1H-indole-1-carboxylate (946 mg, 3.1 mmol), isoquinolin-4-ylboronic acid (500 mg, 3.1 mmol), Na₂CO₃ (640 mg, 6.1 mmol), and THF−H₂O (20 mL, 1:1) was added Pd(PPh₃)₄ (200 mg, 0.15 mmol). The vessel was sealed and heated at 150 degrees for 15 min. The reaction was filtered through filter paper and diluted with EtOAc (200 ml) and water (100 mL). The organic layer was dried (MgSO₄), filtered and concentrated. The crude material was purified by silica gel chromatography.

Step 3: Preparation of tert-butyl 3-(2-benzyl-1-oxo-1,2-dihydroisoquinolin-4-yl)-2-methyl-1H-indole-1-carboxylate, Intermediate 174. To a flask containing tert-butyl 3-(isoquinolin-4-yl)-2-methyl-1H-indole-1-carboxylate (150 mg, 0.42 mmol) in CH₃CN (4.5 mL) was added benzyliodide (100 mg, 0.46 mmol). The reaction was heated at reflux for 3 h. The solution was cooled and diluted with EtOAc (100 mL). The organic layer was washed with water (50 mL) and brine (50 mL). The organic layer was dried (MgSO₄), filtered and concentrated to give an amber oil. To the amber oil was added hexane (˜5-10 mL) and the suspension was agitated and stirred until a powdery light brown solid appeared. The hexane was decanted and the solid was dried in the vacuum oven. To the material was added water (2 mL) and THF (2 mL). An aqueous solution of KOH (1.8 M, 1.68 mmol, 0.93 mL) was added immediately followed by addition of a solution of K₃Fe(CN)₆ (415 mg, 1.26 mmol) in water (0.5 M, 2.5 mL) and DMF. The reaction was diluted with EtOAc (50 mL) and washed with water (50 mL). and taken into the next step without further purification.

Step 4: Preparation of 2-(3-(2-benzyl-1-oxo-1,2-dihydroisoquinolin-4-yl)-2-methyl-1H-indol-1-yl)-acetic acid (247). To a flask containing tert-butyl 3-(2-benzyl-1-oxo-1,2-dihydroisoquinolin-4-yl)-2-methyl-1H-indole-1-carboxylate (195 mg, 0.42 mmol) was added TFA (2 mL). The reaction was stirred for 20 min and then concentrated. The residue was dissolved in EtOAc (50 mL) and washed with H₂O (25 mL). The organic layer was dried (MgSO₄), filtered and concentrated. The crude material was dissolved in DMF (4 mL) and treated with methylbromoacetate (116 μL, 1.3 mmol) and potassium carbonate (232 mg, 1.7 mmol). The reaction was heated to 90 degrees for 4 h and cooled to room temperature. The solution was diluted with EtOAc (75 mL) and washed with water (3×50 mL). The organic layer was concentrated to remove the EtOAc. The residue was dissolved in THF—MeOH—H₂O (15 mL, 1:1:1) and treated with 1M NaOH (2 mL). The reaction was stirred for 2-3 h and then concentrated to remove the volatile solvent. The aqueous layer was made acidic by addition of 1M HCl. The product was extracted with EtOAc (3×15 mL) and purified by reverse phase HPLC to give a white solid (37%, 5 steps). ¹H NMR (400 MHz, DMSO-d₆) δ 13.15 (s, 1H), 8.36 (dd, J=1.26, 7.83 Hz, 1H), 7.60-7.70 (m, 1H), 7.51-7.59 (m, 2H), 7.47 (d, J=8.08 Hz, 1H), 7.23-7.43 (m, 6H), 7.04-7.17 (m, 2H), 6.93-7.02 (m, 1H), 5.29 (s, 2H), 5.05 (s, 2H), 2.21 (s, 3H).

### Example 248

Step 1: Preparation of tent-butyl 3-bromo-5-fluoro-2-methyl-1H-indole-1-carboxylate, Intermediate 175. Prepared by following the procedure for intermediate 294 in 75% yield using as starting material 5-fluoro-2-methyl-1H-indole.

Step 2: Preparation of tert-butyl 5-fluoro-3-(isoquinolin-4-yl)-2-methyl-1H-indole-1-carboxylate, Intermediate 176. Synthesized by the method used for intermediate 295, in 30% yield using as starting material intermediate 297.

Step 3: Preparation of Methyl 2-(3-(2-benzyl-1-oxo-1,2-dihydroisoquinolin-4-yl)-5-fluoro-2-methyl-1H-indol-1-yl)acetic acid, Intermediate 177. Synthesized by the method used for intermediate 174, in 45% yield using as starting material intermediate 176.

Step 4: Preparation of 2-(3-(2-benzyl-1-oxo-1,2-dihydroisoquinolin-4-yl)-5-fluoro-2-methyl-1H-indol-1-yl)acetic acid (248). Synthesized by the method used for 247, using as starting material intermediate 177 in 25% yield. ¹H NMR (400 MHz, MeOD) δ 8.47 (dd, 1H), 7.52-7.68 (m, 2H), 7.25-7.44 (m, 8H), 6.90 (td, J=2.65, 9.16 Hz, 1H), 6.71 (dd, J=2.53, 9.60 Hz, 1H), 5.20-5.45 (m, 2H), 4.96 (s, 2H), 2.21 (s, 3H).

### Example 249

Step 1: Preparation of 2-isopropylisoquinolin-1(2H)-one, Intermediate 178. In a 1000 mL round bottom flask was isoquinolin-1(2H)-one (14.27 g, 98 mmol), 2-iodopropane (9.83 mL, 98 mmol), and cesium carbonate (32.0 g, 98 mmol) in DMF (328 mL) to give a pale yellow suspension. The reaction was heated to 50 degrees in an oil bath for 3 hours. The reaction mixture was diluted with ethyl acetate (600 mL). The organic layer was washed with water (4×250 mL) and dried (MgSO₄). The suspension was filtered and the solvent was removed under reduced pressure. A solution of the material in CH₂Cl₂ and MeOH was added to a biotage samplet. The samplet was then put in a chamber and evacuated to remove the excess solvent. Purification using a 65 sized Biotage column yielded 2-isopropylisoquinolin-1(2H)-one as a white solid (9.61 g; 52%) and 1-isopropoxyisoquinoline as a white solid (4.8 g; 26%). ¹H NMR (400 MHz, DMSO-d₆) δ 8.23 (d, J=8.08 Hz, 1H), 7.61-7.74 (m, 2H), 7.44-7.59 (m, 2H), 6.67 (d, J=7.58 Hz, 1H), 5.20 (dt, J=6.66, 13.71 Hz, 1H), 1.33 (d, 6H).

Step 2: Preparation of 4-iodo-2-isopropylisoquinolin-1(2H)-one, Intermediate 179. To a cooled 500 mL round-bottomed flask of Et₂O (103 mL) containing a suspension of silver trifluoromethanesulfonate (13.19 g, 51.3 mmol), potassium hydroxide (2.88 g, 51.3 mmol), and 2-isopropylisoquinolin-1(2H)-one (9.61 g, 51.3 mmol) was added iodine (13.03 g, 51.3 mmol) at 0 degrees to give a cloudy suspension. After 2 h, the suspension was diluted with diethylether (200 mL) and filtered to remove the silver. The organic layer was washed with 0.1 M sodium thiosulfate (200 mL) and brine (200 mL) and dried (MgSO₄). The solution was filtered and conc. The residue was purified via Biotage (15-25% Hex/EtOAc gradient; 65 column) to give a white solid (44%). ¹H NMR (400 MHz, DMSO-d₆) δ 8.17-8.30 (m, 1H), 7.93 (s, 1H), 7.83 (ddd, J=1.52, 7.14, 8.27 Hz, 1H), 7.62-7.66 (m, 1H), 7.56-7.61 (m, 1H), 5.13 (quin, J=6.82 Hz, 1H), 1.36 (d, J=6.82 Hz, 6H).

Step 3: Preparation of 2-isopropyl-4-(4,4,5,5-tetramethyl-1,3,2-dioxaborolan-2-yl)isoquinolin-1(2H)-one, Intermediate 180. In a 500 mL round-bottomed flask was bis(pinacolato)diboron (5.68 g, 22.35 mmol) and 4-iodo-2-isopropylisoquinolin-1(2H)-one (7.0 g, 22.35 mmol), acetic acid, potassium salt (6.58 g, 67.1 mmol) in DMSO (112 mL) to give a dark orange solution. The reaction was purged with nitrogen gas and then PdCl₂(dppf)-CH₂Cl₂ adduct (1.095 g, 1.341 mmol) was added. The reaction was heated at 80 degrees for 18 h and then cooled to room temperature. The reaction mixture was diluted with EtOAc (800 mL) and washed with water (3×300 mL) and saturated NaCl (1×300 mL). The organic layer was dried (MgSO₄), filtered and conc. The residue was purified via Biotage (20% EtOAc/Hexane; 40M column). Collected fractions: 2-isopropyl-4-(4,4,5,5-tetramethyl-1,3,2-dioxaborolan-2-yl)isoquinolin-1(2H)-one as a white solid (83%). ¹H NMR (400 MHz, DMSO-d₆) δ 8.32 (d, J=7.58 Hz, 1H), 8.26 (dq, J=0.71, 7.99 Hz, 1H), 7.70-7.78 (m, 2H), 7.47-7.54 (m, 1H), 5.13 (quin, J=6.82 Hz, 1H), 1.36 (d, J=7.07 Hz, 6H), 1.34 (s, 12H).

Step 4: Preparation of 2-(5-fluoro-3-(2-isopropyl-1-oxo-1,2-dihydroisoquinolin-4-yl)-2-methyl-1H-indol-1-yl)acetic acid (249). To a 75 mL sealed vessel was added 2-isopropyl-4-(4,4,5,5-tetramethyl-1,3,2-dioxaborolan-2-yl)isoquinolin-1(2H)-one (5.84 g, 18.65 mmol), methyl 2-(5-fluoro-3-iodo-2-methyl-1H-indol-1-yl)acetate (9.71 g, 28.0 mmol), potassium phosphate tribasic monohydrate (7.92 g, 37.3 mmol), and 2-dicyclohexylphosphino-2′,6′-dimethoxy-1,1′-biphenyl (0.765 g, 1.865 mmol) in butan-1-ol (133 mL) and water (53.3 mL) to give a suspension. The reaction was purged with nitrogen. The catalyst, Palladium (II) acetate (0.209 g, 0.932 mmol) was added and the vessel was sealed. The tube was heated at 100 degrees in an oil bath for 18 h. To the aqueous layer was added 1 N HCl until the solution was acidic by litmus test. The solution was stirred rigorously for 5 minutes and allowed to partition into two layers. The aqueous layer was decanted using a pipette. The organic layer was filtered through filter paper to remove the catalyst and then concentrated under reduced pressure to remove the nBuOH. The material was dissolved in MeOH (80 mL) and THF (80 mL) and treated with 50 mL of 1N aqueous NaOH. After stirring for 20 minutes, the organic layer was removed under reduced pressure and the aqueous layer was rendered acidic by addition of 1N HCl (50 mL). The product was extracted with EtOAc (3×200 mL). The organic layer was dried (MgSO₄), filtered and concentrated to give the crude material. The crude material was crystallized with acetonitrile and then tritrated with EtOAc to provide the desired material in >99% purity to give a white solid (5.46 g; 74%). ¹H NMR (400 MHz, DMSO-d₆) δ 13.11 (s, 1H), 8.32-8.40 (m, 1H), 7.63 (td, J=1.39, 7.64 Hz, 1H), 7.48-7.56 (m, 2H), 7.42 (s, 1H), 7.22 (s, 1H), 6.97 (td, J=2.65, 9.16 Hz, 1H), 6.81 (dd, J=2.53, 9.85 Hz, 1H), 5.22-5.36 (m, 1H), 5.10 (d, J=2.53 Hz, 2H), 2.22 (s, 3H), 1.39 (dd, J=3.79, 6.82 Hz, 6H).

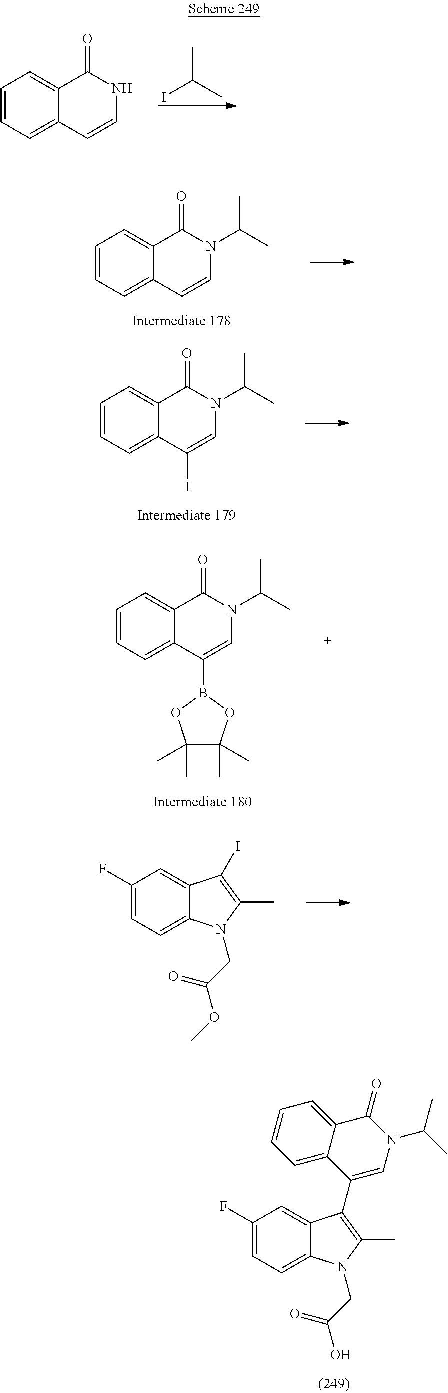

### Example 250

Step 1: Preparation of 2-(2,2,2-trifluoroethyl)isoquinolin-1(2H)-one, Intermediate 181. Synthesized by the method used for intermediate 178, using as starting material isoquinolin-1(2H)-one (1.66 g, 11.4 mmol). Gave the desired product as a white solid (2.52 g, 97%). ¹H NMR (400 MHz, DMSO-d₆) δ 8.21-8.30 (m, 1H), 7.73-7.80 (m, 1H), 7.67-7.72 (m, 1H), 7.56 (ddd, J=1.26, 7.01, 8.15 Hz, 1H), 7.47 (d, J=7.33 Hz, 1H), 6.72 (d, J=7.07 Hz, 1H), 4.94 (q, 2H).

Step 2: Preparation of 4-iodo-2-(2,2,2-trifluoroethyl)isoquinolin-1(2H)-one, Intermediate 182. Bis(pyridine)iodonium tetrafluoroborate (5.67 g, 15.25 mmol) was dissolved in dry dichloromethane (69.3 mL) and added slowly to 2-(2,2,2-trifluoroethyl)isoquinolin-1(2H)-one (3.15 g, 13.87 mmol) and trifluoromethanesulfonic acid (2.71 mL, 30.5 mmol) in CH₂Cl₂. After completion of the reaction by TLC, the reaction was quenched by the addition of 0.1 M sodium thiosulfate (˜100 mL) and washed with saturated NaCl (250 mL). The organic layer was isolated, dried (MgSO₄), filtered, and concentrated to give a dark orange solid. The solid was tritrated with diethylether (15 mL) and the solvent was decanted. The resulting solid was dried in the vacuum oven to constant weight to give 2.7 g of an orange solid. The ether layer was concentrated and purified via biotage CC using a 40S column to give 1.2 g of pure material which was combined with the tritrated solids to give an orange solid (3.9 g; 80%). ¹H NMR (400 MHz, DMSO-d₆) δ 8.23-8.28 (m, 1H), 8.03 (s, 1H), 7.90 (td, J=1.39, 7.64 Hz, 1H), 7.69 (d, J=7.58 Hz, 1H), 7.62-7.67 (m, 1H), 4.92 (q, J=9.35 Hz, 2H).

Step 3: Preparation of 4-(4,4,5,5-tetramethyl-1,3,2-dioxaborolan-2-yl)-2-(2,2,2-trifluoroethyl)-isoquinolin-1(2H)-one, Intermediate 183. Synthesized by the method used for intermediate 180, using as starting material 4-iodo-2-(2,2,2-trifluoroethyl)isoquinolin-1(2H)-one (3.31 g, 9.37 mmol) to give the desired product as a white solid (2.28 g, 69%). ¹H NMR (400 MHz, DMSO-d₆) δ 8.21-8.31 (m, 1H), 8.03 (s, 1H), 7.86-7.93 (m, 1H), 7.69 (d, J=7.58 Hz, 1H), 7.65 (ddd, 1H), 4.93 (q, 2H), 1.17 (s, 12H).

Step 4: Preparation of 2-(5-fluoro-2-methyl-3-(1-oxo-2-(2,2,2-trifluoroethyl)-1,2-dihydroiso-quinolin-4-yl)-1H-indol-1-yl)acetic acid (250). Synthesized by the method used for 2-(5-fluoro-3-(2-isopropyl-1-oxo-1,2-dihydro-isoquinolin-4-yl)-2-methyl-1H-indol-1-yl)acetic acid (249) using as starting material 4-(4,4,5,5-tetramethyl-1,3,2-dioxaborolan-2-yl)-2-(2,2,2-trifluoroethyl)isoquinolin-1(2H)-one (2.67 g, 7.57 mmol) to give the desired product as a white solid (1.12 g, 34%). ¹H NMR (400 MHz, DMSO-d₆) δ 13.13 (s, 1H), 8.34-8.41 (m, 1H), 7.71 (ddd, J=1.52, 7.14, 8.27 Hz, 1H), 7.60 (ddd, J=1.26, 7.07, 8.08 Hz, 1H), 7.53 (dd, J=4.29, 9.09 Hz, 1H), 7.48 (s, 1H), 7.26 (d, J=7.33 Hz, 1H), 6.98 (td, J=2.53, 9.22 Hz, 1H), 6.83 (dd, J=2.40, 9.73 Hz, 1H), 5.12 (s, 2H), 4.93-5.09 (m, 2H), 2.23 (s, 3H).

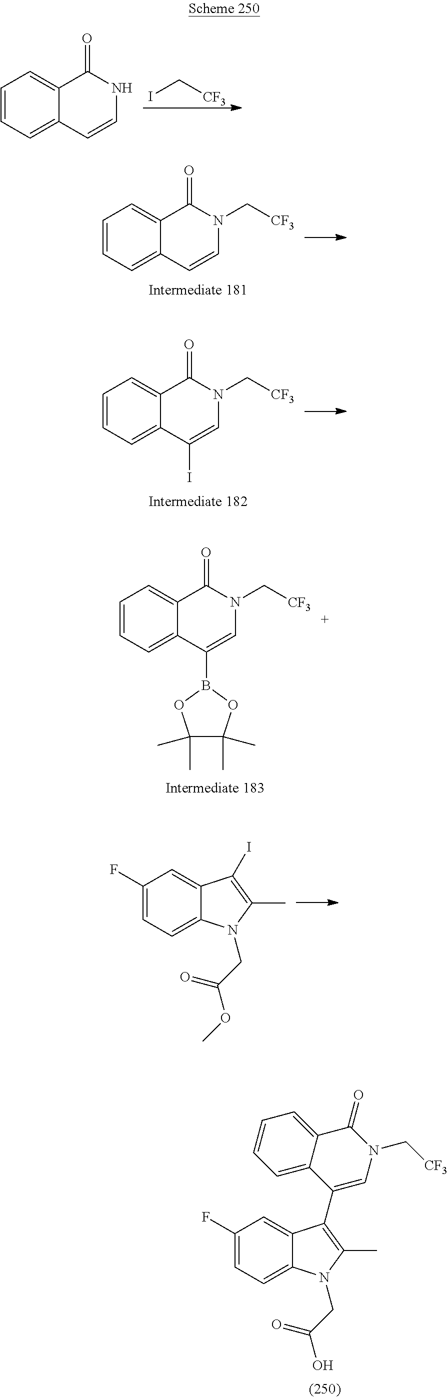

### Example 251

Preparation of 2-(2-methyl-3-(1-oxo-2-(2,2,2-trifluoroethyl)-1,2-dihydroisoquinolin-4-yl)-1H-indol-1-yl)acetic acid (251). Synthesized by the method used for 2-(5-fluoro-3-(2-isopropyl-1-oxo-1,2-dihydro-isoquinolin-4-yl)-2-methyl-1H-indol-1-yl)acetic acid (249). ¹H NMR (400 MHz, MeOD) δ 8.44 (dq, 1H), 7.61-7.67 (m, 1H), 7.54-7.61 (m, 1H), 7.43 (dt, J=0.82, 8.21 Hz, 1H), 7.33-7.39 (m, 2H), 7.10-7.19 (m, 2H), 6.95-7.04 (m, 1H), 4.88-5.06 (m, 4H), 2.30 (s, 3H).

### Example 252

Step 1: Preparation of 3-iodo-2-methyl-1-(4-(trifluoromethyl)phenylsulfonyl)-1H-indole, Intermediate 184. 3-iodo-2-methyl-1H-indole was prepared from 2-methyl-1H-indole according to Takahiro, K.; Yoshinori, K. Chem. Commun. 2006, 891-893 to afford a dark solid (3.91 g, 99%). The product was taken directly into the next step without further purification. To a round bottom flask containing 3-iodo-2-methyl-1H-indole (3 g, 11.7 mmol) in DMF (60 mL) at zero degrees was added NaH (116 mg, 2.9 mmol). The reaction was stirred for 10 min before adding a solution of 4-(trifluoromethyl)benzene-1-sulfonyl chloride (2.86 g, 11.7) in DMF (6 mL). The reaction was diluted with EtOAc (250 mL) and washed with H₂O (3×100 mL). The organic layer was dried (MgSO₄) and filtered. The solvent was removed under reduced pressure and the material was purified by Biotage to give the desired product (3.0 g, 56%).

Step 2: Preparation of 4-(2-methyl-1-(4-(trifluoromethyl)phenylsulfonyl)-1H-indol-3-yl)iso-quinoline, Intermediate 185. To a high pressure vessel was added 3-iodo-2-methyl-1-(4-(trifluoro-methyl)phenylsulfonyl)-1H-indole (279 mg, 0.6 mmol), isoquinolin-4-ylboronic acid (104 mg, 0.6 mmol), sodium carbonate (127 mg, 1.2 mmol) and Pd(PPh₃)₄ (46 mg, 0.04 mmol) in THF-H₂O (6 mL, 2:1). The reaction was sealed and heated in an oil bath at 100 degrees for 18 h. The reaction was filtered to remove the catalyst. The organic layer was concentrated and purified by to give a light orange solid (235 mg, 84%).

Step 3: Preparation of 2-isopropyl-4-(2-methyl-1-(4-(trifluoromethyl)phenyl-sulfonyl)-1H-indol-3-yl)isoquinolin-1(2H)-one, Intermediate 186. To a high pressure vessel containing 4-(2-methyl-1-(4-(trifluoromethyl)phenylsulfonyl)-1H-indol-3-yl)isoquinoline (234 mg, 0.5 mmol) in DMF (5 mL) was added 2-iodopropane (2 mL, excess). The vessel was sealed and heated for 3 h at 150 degrees. The reaction was cooled to room temperature and the screw cap was carefully removed (Use caution when opening the lid due to the release of propene generated during the reaction.). The material was partitioned in DCM (300 mL) and water (50 mL) in a separatory funnel. The organic layer was dried (MgSO₄), filtered and concentrated. To the crude material was added THF (5 mL), 1.8 M KOH (1.1 mL) followed immediately by addition of K₃Fe(CN)₆ (494 mg, 1.5 mmol) in water (4 mL). The reaction was stirred for 1 h and then diluted with EtOAc (300 mL). The organic layer was washed with water (3×100 mL) and dried (MgSO₄). The solvent was removed and the crude material was purified by silica gel column chromatography eluting with 25% EtOAc-Hexane to give the product as a grey solid (118 mg, 45%)

Step 4: Preparation of 2-isopropyl-4-(2-methyl-1H-indol-3-yl)isoquinolin-1(2H)-one, Intermediate 187. Literature procedure was followed for the preparation of 2-isopropyl-4-(2-methyl-1H-indol-3-yl)isoquinolin-1(2H)-one: Org. Process Res. Dev. 2008, 12, 778-780. The material was taken directly into the next step to prepare 2-(3-(2-isopropyl-1-oxo-1,2-dihydroisoquinolin-4-yl)-2-methyl-1H-indol-1-yl)acetic acid.

Step 5: Preparation of methyl 2-(3-(2-isopropyl-1-oxo-1,2-dihydroisoquinolin-4-yl)-2-methyl-1H-indol-1-yl)acetate, Intermediate 188. Synthesized by the method used for intermediate 136, using as starting material intermediate 187 to give the product as a white solid.

Step 6: Preparation of 2-(3-(2-isopropyl-1-oxo-1,2-dihydroisoquinolin-4-yl)-2-methyl-1H-indol-1-yl)acetic acid (252). Synthesized by the method used for 139, using as starting material intermediate 311 to give the product as a white solid (41 mg, 48%; 3 steps).

### Example 253

Step 1: Preparation of 4-(2-methyl-1-(4-(trifluoromethyl)phenylsulfonyl)-1H-indol-3-yl)-2-(2,2,2-trifluoroethyl)isoquinolin-1(2H)-one, Intermediate 189. Intermediate 189 was prepared according to the method described for 2-(5-fluoro-3-(2-isopropyl-1-oxo-1,2-dihydroisoquinolin-4-yl)-2-methyl-1H-indol-1-yl)acetic acid (249; Yield 67%).

Step 2: Preparation of 2-(2,2-difluoro-2-methoxyethyl)-4-(2-methyl-1H-indol-3-yl)isoquinolin-1(2H)-one, Intermediate 190. In a 20 mL microwave vessel was placed 4-(2-methyl-1-(4-(trifluoro-methyl)phenylsulfonyl)-1H-inden-3-yl)-2-(2,2,2-trifluoroethyl)isoquinolin-1(2H)-one (0.658 g, 1.168 mmol) and a solution of 1.8 M potassium hydroxide in THF (3.89 mL), MeOH (3.89 mL), to give a light yellow solution. The reaction was heated at 150 degrees in the microwave. The residue was purified by silica gel column chromatography eluting with an EtOAc-Hexane gradient to give the product as a white solid (113 mg, 26%).

Step 3: Preparation of methyl 2-(3-(2-(2,2-difluoro-2-methoxyethyl)-1-oxo-1,2-dihydroisoquinolin-4-yl)-2-methyl-1H-indol-1-yl)acetate, Intermediate 191. Intermediate 191 was prepared according to the method described for intermediate 188 (Yield 36%).

Step 4: Preparation of 2-(3-(2-(2,2-difluoro-2-methoxyethyl)-1-oxo-1,2-dihydroisoquinolin-4-yl)-2-methyl-1H-indol-1-yl)acetic acid (253). The title compound was prepared according to the method described for 2-(3-(2-isopropyl-1-oxo-1,2-dihydroisoquinolin-4-yl)-2-methyl-1H-indol-1-yl)acetic acid (252) (white solid; 97%). ¹H NMR (400 MHz, MeOD) δ 8.42 (d, J=7.83Hz, 1H), 7.59-7.66 (m, 1H), 7.52-7.59 (m, 1H), 7.43 (d, J=8.34 Hz, 1H), 7.34-7.39 (m, 1H), 7.32 (s, 1H), 7.11-7.20 (m, 2H), 6.95-7.03 (m, 1H), 5.00 (s, 2H), 4.68-4.82 (m, 1H), 4.57-4.68 (m, 1H), 3.62 (s, 3H), 2.31 (s, 3H).

### Example 254

Step 1: Preparation of 2-(2-hydroxy-2-methylpropyl)-4-iodoisoquinolin-1(2H)-one, Intermediate 192. Intermediate 192 was prepared according to the method described for example 131 to give the product as a white solid (510 mg, 81%)

Step 2: Preparation of 2-(2-hydroxy-2-methylpropyl)-4-(4,4,5,5-tetramethyl-1,3,2-dioxaborolan-2-yl)isoquinolin-1(2H)-one, Intermediate 193. Intermediate 193 was prepared according to the method for intermediate 180 (Yield 75%).

Step 3: Preparation of 2-(5-fluoro-3-(2-(2-hydroxy-2-methylpropyl)-1-oxo-1,2-dihydroisoquinolin-4-yl)-2-methyl-1H-indol-1-yl)acetic acid (254). The title compound was prepared according to the method described for 2-(5-fluoro-3-(2-isopropyl-1-oxo-1,2-dihydroisoquinolin-4-yl)-2-methyl-1H-indol-1-yl)acetic acid (249). The material was purified by reverse phase HPLC to give the product as a white solid (90 mg, 24%). ¹H NMR (400 MHz, DMSO-d₆) δ 8.35 (d, J=7.33 Hz, 1H), 7.64 (td, J=1.39, 7.64 Hz, 1H), 7.47-7.55 (m, 2H), 7.44 (s, 1H), 7.25 (d, J=8.08 Hz, 1H), 6.95 (td, J=2.53, 9.09 Hz, 1H), 6.89 (dd, J=2.53, 9.60 Hz, 1H), 5.05 (s, 2H), 3.17 (s, 3H), 2.23 (s, 3H), 1.15 (d, J=3.28 Hz, 6H).

### Example 255

Step 1: Preparation of 4-iodo-2-(3,3,3-trifluoro-2-hydroxy-2-(trifluoromethyl)-propyl)isoquinolin-1(2H)-one, Intermediate 194. Intermediate 194 was prepared according to the method for intermediate 192 (Yield 99%).

Step 2: Preparation of 4-(4,4,5,5-tetramethyl-1,3,2-dioxaborolan-2-yl)-2-(3,3,3-trifluoro-2-hydroxy-2-(trifluoromethyl)propyl)isoquinolin-1(2H)-one, Intermediate 195. Intermediate 195 was prepared according to the method for intermediate 180 (Yield 50%).

Step 3: Preparation of 2-(5-fluoro-2-methyl-3-(1-oxo-2-(3,3,3-trifluoro-2-hydroxy-2-(trifluoro-methyl)propyl)-1,2-dihydroisoquinolin-4-yl)-1H-indol-1-yl)acetic acid (255). The title compound was prepared according to the method described for 2-(5-fluoro-3-(2-isopropyl-1-oxo-1,2-dihydroisoquinolin-4-yl)-2-methyl-1H-indol-1-yl)acetic acid (249; Yield 11%).

### Example 256

Step 1: Preparation of 2-(4,4,4-trifluorobutyl)isoquinolin-1(2H)-one, Intermediate 196. Intermediate 196 was prepared according to the method for intermediate 178 (Yield 93%).

Step 2: Preparation of 4-iodo-2-(4,4,4-trifluorobutyl)isoquinolin-1(2H)-one, Intermediate 197. Intermediate 197 was prepared according to the method for example 88 (Yield 58%).

Step 3: Preparation of 4-(4,4,5,5-tetramethyl-1,3,2-dioxaborolan-2-yl)-2-(4,4,4-trifluorobutyl)iso-quinolin-1(2H)-one, Intermediate 198. Intermediate 198 was prepared according to the method for example 111 (Yield 71%).

Step 4: Preparation of (5-fluoro-2-methyl-3-(1-oxo-2-(4,4,4-trifluorobutyl)-1,2-dihydroisoquinolin-4-yl)-1H-indol-1-yl)acetic acid (256). The title compound was prepared according to the method described for 2-(5-fluoro-3-(2-isopropyl-1-oxo-1,2-dihydroisoquinolin-4-yl)-2-methyl-1H-indol-1-yl)acetic acid (249). The material was purified by HPLC to give a white solid (125 mg, 64%). ¹H NMR (400 MHz, DMSO-d₆) δ 13.09 (br. s., 1H), 8.31-8.39 (m, 1H), 7.61-7.68 (m, 1H), 7.50-7.58 (m, 2H), 7.50 (s, 1H), 7.21 (d, J=7.58 Hz, 1H), 6.96 (td, J=2.53, 9.22 Hz, 1H), 6.86 (dd, J=2.53, 9.60 Hz, 1H), 5.10 (s, 2H), 4.07-4.18 (m, 2H), 2.35 (d, J=11.37 Hz, 2H), 2.22 (s, 3H), 1.98 (d, J=7.07 Hz, 2H).

### Example 257

Step 1: Preparation of 2-neopentylisoquinolin-1(2H)-one, Intermediate 199. Intermediate 199 was prepared according to the method for intermediate 178 (Yield 57)

Step 2: Preparation of 4-iodo-2-neopentylisoquinolin-1(2H)-one, Intermediate 200. Intermediate 200 was prepared according to the method for intermediate 197 (Yield 73%).

Step 3: Preparation of 2-neopentyl-4-(4,4,5,5-tetramethyl-1,3,2-dioxaborolan-2-yl)isoquinolin-1(2H)-one, Intermediate 201. Intermediate 201 was prepared according to the method for intermediate 180 (Yield 50%).

Step 4: Preparation of 2-(5-fluoro-2-methyl-3-(2-neopentyl-1-oxo-1,2-dihydroisoquinolin-4-yl)-1H-indol-1-yl)acetic acid (257). The title compound was prepared according to the method described for 2-(5-fluoro-3-(2-isopropyl-1-oxo-1,2-dihydroisoquinolin-4-yl)-2-methyl-1H-indol-1-yl)acetic acid (249). The material was purified by HPLC to give a white solid (47%). ¹H NMR (400 MHz, DMSO-d₆) δ 13.11 (br. s., 1H), 8.35 (dd, J=1.01, 8.08 Hz, 1H), 7.63 (td, J=1.52, 7.58 Hz, 1H), 7.47-7.56 (m, 2H), 7.35 (s, 1H), 7.19 (d, J=7.58 Hz, 1H), 6.96 (td, J=2.65, 9.16 Hz, 1H), 6.80 (dd, J=2.53, 9.60 Hz, 1H), 5.10 (s, 2H), 3.85-4.07 (m, 2H), 2.22 (s, 3H), 0.99 (s, 9H).

### Example 258

Step 1: Preparation of tert-butyl 2-(3-(3-(2-amino-2-oxoethyl)-4-oxo-3,4-dihydro-phthalazin-1-yl)-5-fluoro-2-methyl-1H-indol-1-yl)acetate, Intermediate 202. The title compound was prepared according to the method described for 1, using as starting material intermediate 5, K₂CO₃ and 2-bromoacetamide to give a beige solid (99%).

Step 2: Preparation of 2-(3-(3-(2-amino-2-oxo ethyl)-4-oxo-3,4-dihydrophthalazin-1-yl)-5-fluoro-2-methyl-1H-indol-1-yl)acetic acid (258). The title compound was prepared according to the method described for 1, using as starting material intermediate 202 to give a beige solid (87%).

### Example 259

Step 1: Preparation of tent-butyl 2-(3-(3-(2-amino-2-oxoethyl)-4-oxo-3,4-dihydro-phthalazin-1-yl)-5-chloro-2-methyl-1H-indol-1-yl)acetate, Intermediate 203. Intermediate 203 was prepared according to the method for intermediate 202 (Yield 86%).

Step 2: Preparation of 2-(3-(3-(2-amino-2-oxoethyl)-4-oxo-3,4-dihydrophthalazin-1-yl)-5-chloro-2-methyl-1H-indol-1-yl)acetic acid (259c). The title compound was prepared according to the method described for 258 (Yield 26%).

Step 3: Preparation of 2-(3-(3-((4H-1,2,4-triazol-3-yl)methyl)-4-oxo-3,4-dihydrophthalazin-1-yl)-5-chloro-2-methyl-1H-indol-1-yl)acetic acid (259a). To a round bottom flask containing 2-(3-(3-(2-amino-2-oxoethyl)-4-oxo-3,4-dihydro-phthalazin-1-yl)-5-chloro-2-methyl-1H-indol-1-yl)acetic acid (259c, 351 mg, 0.83 mmol) in DME (20 mL) was added 1,1-dimethoxy-N,N-dimethylmethanamine (˜3 mL). The reaction was heated to 50 degrees for 2 h and cooled to room temperature. The material was treated with hydrazine hydrate (60 μL), 70% aqueous acetic acid (10 mL) and heated to 90 degrees for 5 h. The reaction was cooled to room temperature and diluted with H₂O (100 mL). The aqueous layer was extracted with EtOAc (3×50 mL) and dried (MgSO₄). The organic layer was concentrated and the crude material was purified by silica gel column chromatography. The purified material was treated with TFA (3 mL) and concentrated after 30 min. The residue was dissolved in CH₂Cl₂ (50 mL) and washed with water (50 mL). The organic layer was dried (MgSO₄), filtered and concentrated to give the product (Yield 34%). ¹H NMR (400 MHz, DMSO-d₆) δ 13.91 (br. s., 1H), 8.47-8.55 (m, 1H), 8.34-8.44 (m, 2H), 7.78-8.00 (m, 2H), 7.56 (d, J=8.84 Hz, 1H), 7.53 (br. s., 1H), 7.07-7.27 (m, 2H), 5.40-5.62 (m, 2H), 5.10 (s, 2H), 2.23 (s, 3H).

Step 4: Preparation of 2-(5-chloro-2-methyl-3-(3-((5-methyl-4H-1,2,4-triazol-3-yl)methyl)-4-oxo-3,4-dihydrophthalazin-1-yl)-1H-indol-1-yl)acetic acid (259b). The title compound was prepared according to the method described for 2-(3-(3-((4H-1,2,4-triazol-3-yl)methyl)-4-oxo-3,4-dihydrophthalazin-1-yl)-5-chloro-2-methyl-1H-indol-1-yl)acetic acid (259a; Yield 18%). ¹H NMR (400 MHz, DMSO-d₆) δ 13.45 (br. s., 1H), 13.24 (br. s., 1H), 8.31-8.48 (m, 1H), 7.85-7.95 (m, 2H), 7.51-7.61 (m, 2H), 7.22 (br. s., 1H), 7.15 (dd, J=2.02, 8.84 Hz, 1H), 5.41-5.53 (m, 1H), 5.25-5.39 (m, 1H), 5.11 (s, 2H), 2.31 (br. s., 3H), 2.24 (s, 3H).

### Example 260

Step 1: Preparation of methyl 2-(3-(1-(2-amino-2-oxoethyl)-6-oxo-1,6-dihydro-pyridazin-3-yl)-5-fluoro-2-methyl-1H-indol-1-yl)acetate, Intermediate 204. Intermediate 204 was prepared according to the method for intermediate 202; Yield 51%.

Step 2: Preparation of 2-(3-(1-((4H-1,2,4-triazol-3-yl)methyl)-6-oxo-1,6-dihydropyridazin-3-yl)-5-fluoro-2-methyl-1H-indol-1-yl)acetic acid (260a). The title compound was prepared according to the method described for 259a (Yield 20%). ¹H NMR (400 MHz, DMSO-d₆) δ 7.72-7.80 (m, 2H), 7.43-7.53 (m, 2H), 7.36 (dd, J=2.53, 10.36 Hz, 1H), 6.95-7.09 (m, 4H), 5.07 (s, 2H), 2.40 (s, 3H).

Step 3: 2-(5-fluoro-2-methyl-3-(1-((5-methyl-4H-1,2,4-triazol-3-yl)methyl)-6-oxo-1,6-dihydro-pyridazin-3-yl)-1H-indol-1-yl)acetic acid (260b). The title compound was prepared according to the method described for 259a to produce a yellow solid (Yield 24%). ¹H NMR (400 MHz, DMSO-d₆) δ 13.50 (br. s., 1H), 7.74 (d, J=9.60 Hz, 1H), 7.43-7.51 (m, 2H), 7.38 (dd, J=2.65, 10.23 Hz, 1H), 6.92-7.07 (m, 2H), 5.30 (s, 2H), 5.03 (s, 2H), 2.41 (s, 3H), 2.32 (s, 3H).

### Example 261

Step 1: Preparation of tert-butyl 2-(5-fluoro-3-(3-(2-methoxy-2-oxoethyl)-4-oxo-3,4-dihydro-phthalazin-1-yl)-2-methyl-1H-indol-1-yl)acetate, Intermediate 205. The title compound was prepared according to the method described for 1 Yield (91%).

Step 2: Preparation of 2-(5-fluoro-3-(3-(2-methoxy-2-oxo ethyl)-4-oxo-3,4-dihydrophthalazin-1-yl)-2-methyl-1H-indol-1-yl)acetic acid, Intermediate 206. Intermediate 206 was prepared according to the method for intermediate 202 (Yield 87%).

Step 3: Preparation of 2-(5-fluoro-3-(3-(2-hydrazinyl-2-oxo ethyl)-4-oxo-3,4-dihydrophthalazin-1-yl)-2-methyl-1H-indol-1-yl)acetic acid, Intermediate 207. To a round bottom flask equipped with reflux condenser containing 2-(5-fluoro-3-(3-(2-methoxy-2-oxo ethyl)-4-oxo-3,4-dihydrophthalazin-1-yl)-2-methyl-1H-indol-1-yl)acetic acid (883 mg, 2.1 mmol) in MeOH (30 mL) was added hydrazine (1.5 mL). The reaction was heated at reflux for 18 h and cooled to room temperature. The reaction was concentrated under reduced pressure to give a yellow solid (880 mg, 99%).

Step 4: Preparation of 2-(5-fluoro-2-methyl-3-(4-oxo-3-((1-phenyl-1H-1,2,4-triazol-5-yl)methyl)-3,4-dihydrophthalazin-1-yl)-1H-indol-1-yl)acetic acid (261). A round bottom flask containing 2-(5-fluoro-3-(3-(2-hydrazinyl-2-oxo ethyl)-4-oxo-3,4-dihydrophthalazin-1-yl)-2-methyl-1H-indol-1- yl)acetic acid (218 mg, 0.52 mmol) in DMF (4 mL) was treated with N,N-dimethylformamide dimethylacetal (337 μL, 2.6 mmol) and heated at 50 degrees for 30 min. The reaction was concentrated under reduced pressure and diluted with AcOH (4 mL). The solution was transferred to a microwave vial and aniline (70 μL, 0.77 mmol) was added. The vessel was sealed and heated at 150 degrees for 20 min. The material was purified by HPLC to give a solid (58 mg, 15%). ¹H NMR (400 MHz, DMSO-d₆) δ 13.26 (br. s., 1H), 8.77 (s, 1H), 8.24-8.30 (m, 1H), 7.83-7.90 (m, 2H), 7.49-7.58 (m, 4H), 7.41-7.49 (m, 3H), 7.00 (td, J=2.65, 9.16 Hz, 1H), 6.89 (dd, J=2.53, 9.60 Hz, 1H), 5.52-5.67 (m, 2H), 5.09 (s, 2H), 2.22 (s, 3H).

### Example 262

Step 1: Preparation of 6-(5-fluoro-2-methyl-1H-indol-3-yl)pyridazin-3 (2H)-one, Intermediate 208. 3-(6-Chloropyridazin-3-yl)-5-fluoro-2-methyl-1H-indole was prepared by the method described for intermediate 1 using as starting material 5-fluoro-2-methylindole. Next it was converted to the title compound using method described for intermediate 2. (Yield 30%; 2 steps).

Step 2: Preparation of 2-benzyl-6-(5-fluoro-2-methyl-1H-indol-3-yl)pyridazin-3 (2H)-one, Intermediate 209. Title compound was prepared by the method described for example 1 using as starting material intermediate 208 to give a beige solid (44%).

Step 3: Preparation of 2-benzyl-6-(5-fluoro-1-(4-methoxybenzoyl)-2-methyl-1H-indol-3-yl)-pyridazin-3(2H)-one (262a). To a round bottom flask containing 2-benzyl-6-(5-fluoro-2-methyl-1H-indol-3-yl)pyridazin-3(2H)-one (125 mg, 0.37 mmol) and t-BuOK (54 mg, 0.56 mmol) in THF-DMF (1:1, 4 mL) was added 4-methoxybenzoyl chloride (76 μL, 0.56 mmol). The reaction was stirred for 1 h and diluted with EtOAc (50 mL). The organic layer was washed with H₂O (25 mL) and dried (MgSO₄). The solution was filtered and concentrated. The crude material was purified by silica gel column chromatography eluting with an EtOAc-Hexane gradient to give a pale yellow solid (107 mg, 62%). ¹H NMR (400 MHz, DMSO-d₆) δ 7.79 (d, J=9.60 Hz, 1H), 7.71-7.77 (m, 2H), 7.27-7.43 (m, 6H), 7.09-7.17 (m, 3H), 6.94-7.08 (m, 2H), 5.39 (s, 2H), 3.89 (s, 3H), 2.38 (s, 3H).

Step 4: Preparation of 2-benzyl-6-(5-fluoro-2-methyl-1-nicotinoyl-1H-indol-3-yl)pyridazin-3(2H)-one (262b). The title compound was prepared according to the method described for 2-benzyl-6-(5-fluoro-1-(4-methoxybenzoyl)-2-methyl-1H-indol-3-yl)pyridazin-3 (2H)-one (262a; Yield 4%). ¹H NMR (400 MHz, DMSO-d₆) δ 7.30-7.40 (m, 1H), 6.91 (d, J=9.60 Hz, 1H), 6.82 (dd, J=4.93, 7.96 Hz, 1H), 6.60-6.67 (m, 2H), 6.49-6.58 (m, 3H), 6.41-6.48 (m, 1H), 6.25-6.35 (m, 2H), 6.08 (td, 1H), 4.62 (s, 2H), 4.08 (s, 2H), 1.55 (s, 3H).

Step 5: Preparation of 6-(1-benzyl-5-fluoro-2-methyl-1H-indol-3-yl)-2-benzylpyridazin-3(2H)-one (262c). The title compound was prepared according to the method described for 2-benzyl-6-(5-fluoro-1-(4-methoxybenzoyl)-2-methyl-1H-indol-3-yl)pyridazin-3(2H)-one (262a; Yield 20%). ¹H NMR (400 MHz, CHLOROFORM-d) δ 8.07-8.14 (m, 1H), 7.44-7.56 (m, 3H), 7.27-7.39 (m, 6H), 7.16 (dd, J=4.29, 9.09 Hz, 1H), 7.06 (d, J=9.60 Hz, 1H), 6.98 (dd, J=1.64, 7.96 Hz, 2H), 6.91 (td, J=2.53, 8.97 Hz, 1H), 5.34 (s, 2H), 2.42 (s, 3H).

### Example 263

Step 1: Preparation of 3-bromo-2-methyl-1H-indole, Intermediate 210. The title compound was prepared according to the method described for intermediate 172 using 2-methylindole as a starting material.

Step 2: Preparation of tert-butyl 2-(3-bromo-2-methyl-1H-indol-1-yl)acetate, Intermediate 211. The title compound was prepared according to the method described for intermediate 1A using 3-bromo-2-methyl-1H-indole, K₂CO₃ and t-butylbromoacetate to give a beige solid (86%). ¹H NMR (400 MHz, DMSO-d₆) δ 7.44 (d, 1H), 7.29-7.38 (m, 1H), 7.05-7.21 (m, 2H), 5.03 (s, 2H), 2.33 (s, 3H), 1.42 (s, 9H).

Step 3: Preparation of tert-butyl 2-(3-(6-methoxypyridin-3-yl)-2-methyl-1H-indol-1-yl)acetate, Intermediate 212. To a microwave vessel was added tert-butyl 2-(3-bromo-2-methyl-1H-indol-1-yl)acetate (333 mg, 1.3 mmol), 6-methoxypyridin-3-ylboronic acid (0.47 g, 3.1 mmol), PdCl₂(dppf)-CH₂Cl₂ (49 mg, 0.07 mmol) and Cs₂CO₃ (2.0 g, 6.1 mmol) in DME (10.3 mL). The contents were degassed with nitrogen and sealed. The reaction was heated at 150 degrees for 20 min in the microwave. The reaction was diluted with EtOAc (250 mL) and washed with H₂O (3×100 mL). The organic layer was dried (MgSO₄) and filtered. The solvent was removed under reduced pressure and purified by silica gel column chromatography (0.26 g, 71%)

Step 4: Preparation of tert-butyl 2-(3-(1-benzyl-6-oxo-1,6-dihydropyridin-3-yl)-2-methyl-1H-indol-1-yl)acetate, Intermediate 213. The title compound was prepared according to the method described for intermediate 37 using intermediate 212, sodium iodide and benzyl bromide to give a reddish solid (65%). ¹H NMR (400 MHz, DMSO-d₆) δ 7.81 (d, 1H), 7.56 (dd, J=2.53, 9.35 Hz, 1H), 7.34-7.43 (m, 6H), 7.12 (td, J=1.26, 7.58 Hz, 1H), 7.00-7.06 (m, 1H), 6.55 (d, J=9.35 Hz, 1H), 5.21 (s, 2H), 5.00 (s, 2H), 2.30 (s, 3H), 1.43 (s, 9H).

Step 5: Preparation of 2-(3-(1-benzyl-6-oxo-1,6-dihydropyridin-3-yl)-2-methyl-1H-indol-1-yl)acetic acid (263). The title compound was prepared according to the method described for 1 to afford a pale beige solid (56%). ¹H NMR (400 MHz, DMSO-d₆) δ 13.15 (s, 1H), 7.81 (d, J=2.53 Hz, 1H), 7.57 (dd, J=2.53, 9.35 Hz, 1H), 7.34-7.44 (m, 6H), 7.31 (dd, J=3.16, 5.43 Hz, 1H), 7.07-7.15 (m, 1H), 6.97-7.06 (m, 1H), 6.55 (d, J=9.35 Hz, 1H), 5.21 (s, 2H), 4.96 (,s 2H), 2.31 (s, 3H).

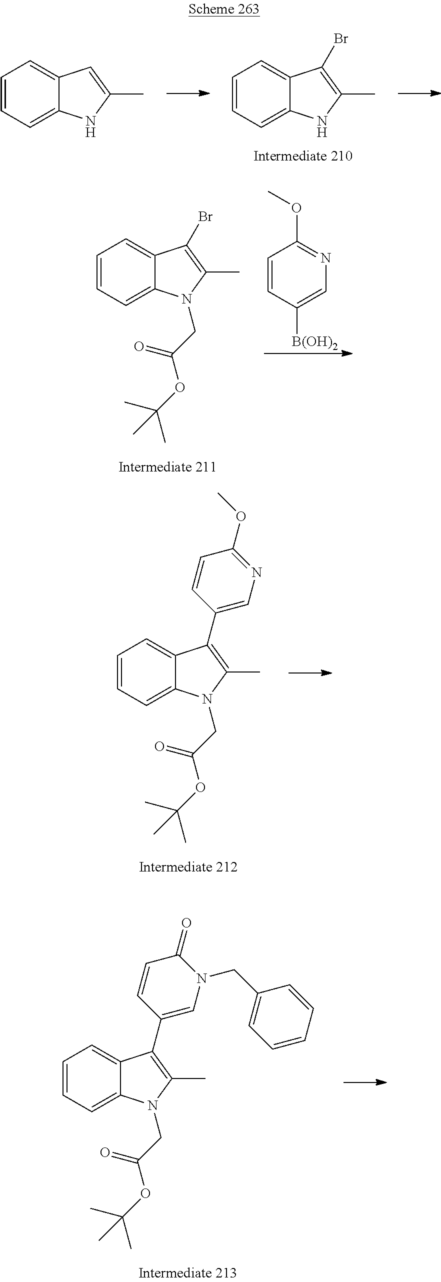

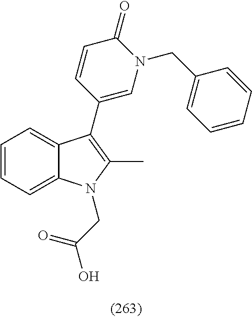

### Example 264

Step 1: Preparation of 3-bromo-5-chloro-2-methyl-1H-indole, Intermediate 214. Intermediate 214 was prepared according to the method described for intermediate 210.

Step 2: Preparation of tert-butyl 2-(3-bromo-5-chloro-2-methyl-1H-indol-1-yl)-acetate, Intermediate 215. Intermediate 215 was prepared according to the method for intermediate 211 (Yield 81%)

Step 3: Preparation of tert-butyl 2-(5-chloro-3-(6-methoxypyridin-3-yl)-2-methyl-1H-indol-1-yl)-acetate, Intermediate 216. Intermediate 216 was prepared according to the method for intermediate 212 (Yield 42%)

Step 4: Preparation of tert-butyl 2-(3-(1-benzyl-6-oxo-1,6-dihydropyridin-3-yl)-5-chloro-2-methyl-1H-indol-1-yl)acetate, Intermediate 217. Intermediate 217 was prepared according to the method for intermediate 213 (Yield 52%).

Step 5: 2-(3-(1-benzyl-6-oxo-1,6-dihydropyridin-3-yl)-5-chloro-2-methyl-1H-indol-1-yl)acetic acid (264). The title compound was prepared according to the method described for 2-(3-(1-benzyl-6-oxo-1,6-dihydropyridin-3-yl)-2-methyl-1H-indol-1-yl)acetic acid (263; Yield 99%). ¹H NMR (400 MHz, DMSO-d₆) δ 13.12 (br. s., 1H), 7.86 (d, J=2.02 Hz, 1H), 7.55 (dd, J=2.53, 9.35 Hz, 1H), 7.48 (d, J=8.59 Hz, 1H), 7.27-7.43 (m, 6H), 7.12 (dd, J=2.15, 8.72 Hz, 1H), 6.55 (d, J=9.35 Hz, 1H), 5.21 (s, 2H), 5.04 (s, 2H), 2.31 (s, 3H).

### Example 265

Step 1: Preparation of 3-bromo-5-fluoro-2-methyl-1H-indole, Intermediate 218. Intermediate 218 was prepared according to the method described for intermediate 210.

Step 2: Preparation of tert-butyl 2-(3-bromo-5-fluoro-2-methyl-1H-indol-1-yl)acetate, Intermediate 219. Intermediate 219 was prepared according to the method for intermediate 211 (Yield 39%).

Step 3: Preparation of tert-butyl 2-(5-fluoro-3-(6-methoxypyridin-3-yl)-2-methyl-1H-indol-1-yl)-acetate, Intermediate 220. Intermediate 220 was prepared according to the method for intermediate 212 (Yield 56%).

Step 4: Preparation of methyl 2-(3-(1-benzyl-6-oxo-1,6-dihydropyridin-3-yl)-5-fluoro-2-methyl-1H-indol-1-yl)acetate, Intermediate 221. Intermediate 221 was prepared according to the method for intermediate 213 (Yield 95%).

Step 5: Preparation of 2-(3-(1-benzyl-6-oxo-1,6-dihydropyridin-3-yl)-5-fluoro-2-methyl-1H-indol-1-yl)acetic acid (265). The title compound was prepared according to the method described for 252, Yield 48%. ¹H NMR (400MHz, DMSO-d₆) δ 13.10 (s, 1H), 7.84 (d, J=2.02 Hz, 1H), 7.56 (dd, J=2.65, 9.22 Hz, 1H), 7.45 (dd, J=4.29, 8.84 Hz, 1H), 7.27-7.41 (m, 5H), 7.10 (dd, J=2.53, 9.85 Hz, 1H), 6.95 (td, J=2.53, 9.09 Hz, 1H), 6.55 (d, J=9.09 Hz, 1H), 5.21 (s, 2H), 5.03 (s, 2H), 2.30 (s, 3H).

### Example 266

Preparation of 2-(5-fluoro-3-(1-(2-fluorobenzyl)-6-oxo-1,6-dihydropyridin-3-yl)-2-methyl-1H-indol-1-yl)acetic acid (266). The title compound was prepared according to the procedure of Example 265; Yield 81%. ¹H NMR (400 MHz, DMSO-d₆) δ 7.80 (d, J=2.53 Hz, 1H), 7.60 (dd, J=2.65, 9.22 Hz, 1H), 7.46 (dd, J=4.42, 8.97 Hz, 1H), 7.32-7.41 (m, 1H), 7.18-7.31 (m, 3H), 7.15 (dd, J=2.53, 9.85 Hz, 1H), 6.97 (td, J=2.53, 9.09 Hz, 1H), 6.55 (d, J=9.35 Hz, 1H), 5.26 (s, 2H), 5.18 (s, 2H), 3.70 (s, 3H), 2.32 (s, 3H).

### Example 267

Preparation of 2-(5-fluoro-3-(1-(4-fluorobenzyl)-6-oxo-1,6-dihydropyridin-3-yl)-2-methyl-1H-indol-1-yl)acetic acid (267). The title compound was prepared according to the procedure of Example 265; Yield 76¹H NMR (400 MHz, DMSO-d₆) δ 7.88 (d, J=2.02 Hz, 1H), 7.56 (dd, J=2.65, 9.22 Hz, 1H), 7.43-7.50 (m, 3H), 7.17-7.22 (m, 2H), 7.12 (dd, J=2.27, 9.85 Hz, 1H), 6.97 (td, J=2.65, 9.16 Hz, 1H), 6.55 (d, 1H), 5.19 (s, 2H), 5.17 (s, 2H), 2.31 (s, 3H).

### Example 268

Preparation of 2-(3-(1-(2,6-difluorobenzyl)-6-oxo-1,6-dihydropyridin-3-yl)-5-fluoro-2-methyl-1H-indol-1-yl)acetic acid (268). The title compound was prepared according to the procedure of Example 265; Yield 78%. ¹H NMR (400 MHz, DMSO-d₆) δ 13.16 (br. s., 1H), 7.81 (s, 1H), 7.55 (dd, J=2.53, 9.35 Hz, 1H), 7.37-7.51 (m, 2H), 7.07-7.19 (m, 3H), 6.97 (td, J=2.53, 9.09 Hz, 1H), 6.46 (d, J=9.35 Hz, 1H), 5.24 (s, 2H), 5.03 (s, 2H), 2.33 (s, 3H).

### Example 269

Preparation of 2-(3-(1-(2,3-difluorobenzyl)-6-oxo-1,6-dihydropyridin-3-yl)-5-fluoro-2-methyl-1H-indol-1-yl)acetic acid (269). The title compound was prepared according to the procedure of Example 265; Yield 86%. ¹H NMR (400 MHz, DMSO-d₆) δ 13.13 (br. s., 1H), 7.84 (d, J=2.53 Hz, 1H), 7.61 (dd, J=2.53, 9.35 Hz, 1H), 7.45 (dd, J=4.29, 8.84 Hz, 1H), 7.35-7.43 (m, 1H), 7.18-7.25 (m, 1H), 7.16 (dd, J=2.65, 9.73 Hz, 1H), 7.01-7.08 (m, 1H), 6.96 (td, J=2.53, 9.22 Hz, 1H), 6.55 (d, J=9.35 Hz, 1H), 5.30 (s, 2H), 5.02 (s, 2H), 2.33 (s, 3H).

### Example 270

2-(3-(3-((4H-1,2,4-triazol-3-yl)methyl)-4-oxo-3,4-dihydrophthalazin-1-yl)-5-fluoro-2-methyl-1H-indol-1-yl)acetic acid (270). The title compound was prepared according to the procedure of Example 259a; Yield 34%.%). ¹H NMR (400 MHz, DMSO-d₆) δ 13.92 (br. s., 1H), 8.40 (dt, J=2.31, 4.74 Hz, 1H), 7.84-7.99 (m, 2H), 7.50-7.63 (m, 2H), 7.00 (td, J=2.53, 9.22 Hz, 1H), 6.92 (br. s., 1H), 5.40-5.63 (m, 2H), 5.25 (s, 2H), 3.68-3.77 (m, 2H), 2.23 (s, 3H).

### Example 271

Preparation of 2-(5-fluoro-2-methyl-3-(6-oxo-1-(2,4,5-trifluorobenzyl)-1,6-dihydropyridin-3yl)-1H-indol-1-yl)acetic acid (271). The title compound was prepared according to the procedure of Example 265; ¹H NMR (400 MHz, DMSO-d₆) δ 7.78 (s, 1H), 7.52-7.68 (m, 2H), 7.39-7.50 (m, 1H), 7.35 (dd, J=4.67, 8.97 Hz, 1H), 7.13 (dd, J=2.53, 10.11 Hz, 1H), 6.91 (td, J=2.65, 9.16 Hz, 1H), 6.53 (d, J=9.35 Hz, 1H), 5.19 (s, 2H), 4.69 (br. s., 2H), 2.31 (s, 3H).

### Example 272

Step 1: Preparation of 4-bromo-2-methylbutan-2-ol, Intermediate 222. The procedure described by Fall and Vitale was used (Fall, Y.; Vitale, C.; Mourino, A. Tetrahedron Lett. 2000, 41, 7337).

Step 2: Preparation of 2-(3-hydroxy-3-methylbutyl)-4-iodoisoquinolin-1(2H)-one, Intermediate 223. Intermediate 223 was prepared according to the method for intermediate 178 (Yield 58%).

Step 3: Preparation of 2-(3-hydroxy-3-methylbutyl)-4-(4,4,5,5-tetramethyl-1,3,2-dioxaborolan-2-yl)isoquinolin-1(2H)-one, Intermediate 224. Intermediate 224 was prepared according to the method for intermediate 180 (Yield 66%).

Step 4: Preparation of 2-(5-fluoro-3-(2-(3-hydroxy-3-methylbutyl)-1-oxo-1,2-dihydroisoquinolin-4-yl)-2-methyl-1H-indol-1-yl)acetic acid (272). The title compound was prepared according to the method described for 2-(5-fluoro-3-(2-isopropyl-1-oxo-1,2-dihydroisoquinolin-4-yl)-2-methyl-1H-indol-1-yl)acetic acid (249; Yield 33%). ¹H NMR (400 MHz, DMSO-d₆) δ 13.50 (br. s., 1H), 8.34 (dd, J=1.14, 8.21 Hz, 1H), 7.58-7.66 (m, 1H), 7.46-7.57 (m, 2H), 7.42 (s, 1H), 7.21 (d, J=8.08 Hz, 1H), 6.95 (td, J=2.65, 9.16 Hz, 1H), 6.84 (dd, J=2.53, 9.85 Hz, 1H), 5.05 (s, 2H), 4.12 (dd, J=6.44, 10.99 Hz, 2H), 3.66 (d, 1H), 2.21 (s, 3H), 1.75-1.92 (m, 2H), 1.17 (s, 6H).

### Example 273

Preparation of 2-(3-(1-(2,4-difluorobenzyl)-6-oxo-1,6-dihydropyridin-3-yl)-5-fluoro-2-methyl-1H-indol-1-yl)acetic acid (273). The title compound was prepared according to the procedure of Example 265; Yield 64%.1H NMR (400 MHz, DMSO-d₆) δ ppm 2.29 (s, 3H), 4.34 (s, 2H), 5.21 (s, 2H), 6.53 (d, J=9.3 Hz, 1H), 6.86 (td, J=9.1, 2.5 Hz, 1H), 7.05-7.14 (m, 2H), 7.21-7.41 (m, 3H), 7.57 (dd, J=9.3, 2.5 Hz, 1H), 7.72 (d, J=2.3 Hz, 1H)

### Example 274

Step 1: Preparation of methyl 2-(3-bromo-5-chloro-2-methyl-1H-indol-1-yl)-acetate, Intermediate 225. Intermediate 225 was prepared according to the method for intermediate 219; Yield 63%

Step 2: Preparation of methyl 2-(5-chloro-3-(6-methoxypyridin-3-yl)-2-methyl-1H-indol-1-yl)-acetate, Intermediate 226. Intermediate 226 was prepared according to the method for intermediate 220; Yield (81%).

Step 3: Preparation of methyl 2-(5-chloro-3-(1-(2,4-difluorobenzyl)-6-oxo-1,6-dihydropyridin-3-yl)-2-methyl-1H-indol-1-yl)acetate, Intermediate 227. Intermediate 227 was prepared according to the method for Example 273; Yield (72%).

Step 4: Preparation of 2-(5-chloro-3-(1-(2,4-difluorobenzyl)-6-oxo-1,6-dihydropyridin-3-yl)-2-methyl-1H-indol-1-yl)acetic acid (274). The title compound was prepared according to the method described for 2-(3-(1-(2,4-difluorobenzyl)-6-oxo-1,6-dihydropyridin-3-yl)-5-fluoro-2-methyl-1H-indol-1-yl)acetic acid (273); Yield 60% 1H NMR (400 MHz, DMSO-d₆) δ ppm 2.23-2.33 (m, 3H), 4.47 (s, 2H), 5.21 (s, 2H), 6.53 (d, J=9.3 Hz, 1H), 7.04 (dd, J=8.6, 2.0 Hz, 1H), 7.10 (td, 1H), 7.21-7.43 (m, 4H), 7.57 (dd, J=9.3, 2.5 Hz, 1H), 7.74 (d, J=2.5 Hz, 1H)

### Example 275

Step 1: Preparation of methyl 2-(3-bromo-2-methyl-1H-indol-1-yl)acetate, Intermediate 228. Intermediate 228 was prepared according to the method for intermediate 219; Yield 80%

Step 2: Preparation of methyl 2-(3-(6-methoxypyridin-3-yl)-2-methyl-1H-indol-1-yl)acetate, Intermediate 229. Intermediate 229 was prepared according to the method for intermediate 220; Yield (88%).

Step 3: Preparation of methyl 2-(3-(1-(2,4-difluorobenzyl)-6-oxo-1,6-dihydro-pyridin-3-yl)-2-methyl-1H-indol-1-yl)acetate, Intermediate 230. Intermediate 230 was prepared according to the method for Example 158.; Yield (66%).

Step 4: Preparation of 2-(3-(1-(2,4-difluorobenzyl)-6-oxo-1,6-dihydropyridin-3-yl)-2-methyl-1H-indol-1-yl)acetic acid (275). The title compound was prepared according to the method described for 2-(3-(1-(2,4-difluorobenzyl)-6-oxo-1,6-dihydropyridin-3-yl)-5-fluoro-2-methyl-1H-indol-1-yl)acetic acid (273); Yield 73% 1H NMR (400 MHz, DMSO-d₆) δ ppm 2.33 (s, 3H), 5.02 (s, 2H), 5.21 (s, 2H), 6.54 (d, J=9.3 Hz, 1H), 6.98-7.17 (m, 3H), 7.24-7.48 (m, 4H), 7.60 (dd, J=9.3, 2.5 Hz, 1H), 7.78 (d, J=2.5 Hz, 1H), 13.07 (s, 1H)

### Example 276

Step 1: Preparation of methyl 2-(2-methyl-3-(6-oxo-1,6-dihydropyridin-3-yl)-1H-indol-1-yl)acetate, Intermediate 231. To a microwave vessel was added intermediate 229 (0.7 g, 2.25 mmol), MeOH (10 mL), and concentrated HCl (0.5 mL, 16.5 mmol). The reaction was heated at 125 degrees for 60 min in the microwave. The solvent was removed under reduced pressure and the crude material was purified by column chromatography to give a yellow-ish solid (0.5 g, 75%).

Step 2: Preparation of methyl 2-(3-(1-isopropyl-6-oxo-1,6-dihydropyridin-3-yl)-2-methyl-1H-indol-1-yl)acetate, Intermediate 232. To a microwave vessel was added methyl 2-(2-methyl-3-(6-oxo-1,6-dihydropyridin-3-yl)-1H-indol-1-yl)acetate (0.3 g, 1 mmol), potassium carbonate (0.7 g, 5 mmol), and DMF (12 mL). Then 2-bromopropane (0.19 mL, 2 mmol) was added and the vessel sealed. The reaction was heated at 65 degrees for 20 hours on an oil bath. The reaction was diluted with EtOAc (100 mL) and washed with brine (3×100 mL). The organic layer was dried (MgSO₄) and filtered. The crude product was purified by column chromatography to give a clear oil (0.21 g, 63%)

Step 3: Preparation of 2-(3-(1-isopropyl-6-oxo-1,6-dihydropyridin-3-yl)-2-methyl-1H-indol-1-yl)-acetic acid (276). The title compound was prepared according to the method described for 273; Yield 40% 1H NMR (400 MHz, DMSO-d₆) δ ppm 1.34 (d, J=6.3 Hz, 6H), 2.36 (s, 3H), 5.04 (s, 2H), 5.23-5.35 (m, 1H), 6.86 (d, J=8.3 Hz, 1H), 7.05 (t, J=7.5 Hz, 1H), 7.13 (td, J=7.6, 1.1 Hz, 1H), 7.45 (t, J=8.0 Hz, 1H), 7.76 (dd, J=8.6, 2.5 Hz, 1H), 8.20 (d, J=2.5 Hz, 1H), 13.08 (br. s., 1H)

### Example 277

Step 1: Preparation of methyl 2-(5-fluoro-2-methyl-3-(6-oxo-1,6-dihydropyridin-3-yl)-1H-indol-1-yl)acetate, Intermediate 233. Intermediate 233 was prepared according to the method for intermediate 231; Yield 68%.

Step 2: Preparation of methyl 2-(5-fluoro-3-(1-isopropyl-6-oxo-1,6-dihydro-pyridin-3-yl)-2-methyl-1H-indol-1-yl)acetate, Intermediate 234. Intermediate 234 was prepared according to the method for intermediate 232; Yield 64%

Step 3: Preparation of 2-(5-fluoro-3-(1-isopropyl-6-oxo-1,6-dihydropyridin-3-yl)-2-methyl-1H-indol-1-yl)acetic acid (277). The title compound was prepared according to the method described for 273; Yield 70% 1H NMR (400 MHz, DMSO-d₆) δ ppm 1.33 (d, J=6.3 Hz, 6H), 2.35 (s, 3H), 5.05 (s, 2H), 5.24-5.35 (m, 1H), 6.85 (d, J=8.3 Hz, 1H), 6.97 (td, J=9.1, 2.5 Hz, 1H), 7.16 (dd, J=9.9, 2.5 Hz, 1H), 7.47 (dd, J=8.8, 4.5 Hz, 1H), 7.75 (dd, J=8.5, 2.4 Hz, 1H), 8.19 (d, J=2.5 Hz, 1H), 13.11 (br. s., 1H)

### Example 278

Step 1: Preparation of methyl 2-(5-chloro-2-methyl-3-(6-oxo-1,6-dihydropyridin-3-yl)-1H-indol-1-yl)acetate, Intermediate 235. Intermediate 235 was prepared according to the method for intermediate 231; Yield 63%

Step 2: Preparation of methyl 2-(5-chloro-3-(1-isopropyl-6-oxo-1,6-dihydro-pyridin-3-yl)-2-methyl-1H-indol-1-yl)acetate, Intermediate 236. Intermediate 236 was prepared according to the method for intermediate 11; Yield g, 62%

Step 3: Preparation of 2-(5-chloro-3-(1-isopropyl-6-oxo-1,6-dihydropyridin-3-yl)-2-methyl-1H-indol-1-yl)acetic acid (278). The title compound was prepared according to the method described for 273; Yield 66% 1H NMR (400 MHz, DMSO-d₆) δ ppm 1.34 (d, J=6.1 Hz, 6H), 2.36 (s, 3H), 5.07 (s, 2H), 5.19-5.36 (m, 1H), 6.86 (d, J=8.3 Hz, 1H), 7.14 (dd, J=8.6, 2.0 Hz, 1H), 7.40 (d, J=2.0 Hz, 1H), 7.50 (d, J=8.8 Hz, 1H), 7.76 (dd, J=8.3, 2.5 Hz, 1H), 8.19 (d, J=2.5 Hz, 1H), 13.13 (s, 1H).

### Example 279

Step 1: Preparation of methyl 2-(2-methyl-3-(6-oxo-1-(2,2,2-trifluoroethyl)-1,6-dihydropyridin-3-yl)-1H-indol-1-yl)acetate, Intermediate 237. Intermediate 237 was prepared according to the method for intermediate 232; Yield 50%.

Step 2: Preparation of 2-(2-methyl-3-(6-oxo-1-(2,2,2-trifluoroethyl)-1,6-dihydropyridin-3-yl)-1H-indol-1-yl)acetic acid (279). The title compound was prepared according to the method described for 273; Yield 60% 1H NMR (400 MHz, DMSO-d₆) δ ppm 2.35 (s, 3H), 4.97 (q, J=9.3 Hz, 2H), 5.04 (s, 2H), 6.61 (d, J=9.3 Hz, 1H), 7.06 (t, J=7.6 Hz, 1H), 7.13 (t, J=8.0 Hz, 1H), 7.44 (d, J=8.6 Hz, 2H), 7.65 (dd, J=9.5, 2.7 Hz, 1H), 7.74 (d, J=2.3 Hz, 1H), 13.08 (br. s., 1H)

### Example 280

Step 1: Preparation of methyl 2-(5-fluoro-2-methyl-3-(6-oxo-1-(2,2,2-trifluoro-ethyl)-1,6-dihydropyridin-3-yl)-1H-indol-1-yl)acetate, Intermediate 238. Intermediate 238 was prepared according to the method \for intermediate 232; Yield (24%).

Step 2: Preparation of 2-(5-fluoro-2-methyl-3-(6-oxo-1-(2,2,2-trifluoro ethyl)-1,6-dihydropyridin-3-yl)-1H-indol-1-yl)acetic acid (280). The title compound was prepared according to the method 273; Yield 70% 1H NMR (400 MHz, DMSO-d₆) δ ppm 2.34 (s, 3H), 4.97 (q, J=9.3 Hz, 2H), 5.05 (s, 2H), 6.61 (d, J=9.3 Hz, 1H), 6.98 (td, J=9.2, 2.3 Hz, 1H), 7.18 (dd, J=9.9, 2.5 Hz, 1H), 7.47 (dd, J=9.0, 4.4 Hz, 1H), 7.64 (dd, J=9.6, 2.5 Hz, 1H), 7.75 (d, J=2.0 Hz, 1H), 13.12 (s, 1H)

### Example 281

Step 1: Preparation of methyl 2-(5-chloro-2-methyl-3-(6-oxo-1-(2,2,2-trifluoro-ethyl)-1,6-dihydropyridin-3-yl)-1H-indol-1-yl)acetate, Intermediate 239. Intermediate 239 was prepared according to the method for intermediate 232; Yield (33%)

Step 2: Preparation of 2-(5-chloro-2-methyl-3-(6-oxo-1-(2,2,2-trifluoroethyl)-1,6-dihydropyridin-3-yl)-1H-indol-1-yl)acetic acid (281). The title compound was prepared according to the method described for 273; Yield 58% 1H NMR (400 MHz, DMSO-d₆) δ ppm 2.34 (s, 3H), 4.97 (q, J=9.2 Hz, 2H), 5.06 (s, 2H), 6.61 (d, J=9.3 Hz, 1H), 7.14 (dd, J=8.6, 2.0 Hz, 1H), 7.43 (d, J=2.3 Hz, 1H), 7.50 (d, J=8.6 Hz, 1H), 7.64 (dd, J=9.3, 2.5 Hz, 1H), 7.77 (d, J=1.5 Hz, 1H), 13.14 (s, 1H)

### Example 282

Step 1: Preparation of methyl 2-(3-(1-(2-fluorobenzyl)-6-oxo-1,6-dihydropyridin-3-yl)-2-methyl-1H-indol-1-yl)acetate, Intermediate 240. Intermediate 240 was prepared according to the method for example 274; Yield (65%).

Step 2: Preparation of 2-(3-(1-(2-fluorobenzyl)-6-oxo-1,6-dihydropyridin-3-yl)-2-methyl-1H-indol-1-yl)acetic acid (282). The title compound was prepared according to the method described for 273; Yield 59% 1H NMR (400 MHz, DMSO-d₆) δ ppm 2.33 (s, 3H), 5.02 (s, 2H), 5.25 (s, 2H), 6.55 (d, J=9.3 Hz, 1H), 7.04 (t, J=7.5 Hz, 1H), 7.12 (td, J=7.6, 0.9 Hz, 1H), 7.17-7.31 (m, 3H), 7.31-7.46 (m, 3H), 7.60 (dd, J=9.3, 2.5 Hz, 1H), 7.77 (d, J=2.3 Hz, 1H), 13.06 (br. s., 1H)

### Example 283

Step 1: Preparation of methyl 2-(3-(1-(4-fluorobenzyl)-6-oxo-1,6-dihydropyridin-3-yl)-2-methyl-1H-indol-1-yl)acetate, Intermediate 241. Intermediate 241 was prepared according to the method for example 274; Yield (63%).

Step 2: Preparation of 2-(3-(1-(4-fluorobenzyl)-6-oxo-1,6-dihydropyridin-3-yl)-2-methyl-1H-indol-1-yl)acetic acid (283). The title compound was prepared according to the method described for 273; Yield 81% 1H NMR (400 MHz, DMSO-d₆) δ ppm 2.31 (s, 3H), 5.01 (s, 2H), 5.19 (s, 2H), 6.55 (d, J=9.1 Hz, 1H), 7.03 (t, J=7.1 Hz, 1H), 7.11 (td, J=7.5 , 0.9 Hz, 1H), 7.15-7.26 (m, 2H), 7.40 (dd, J=12.8, 8.0 Hz, 2H), 7.44-7.50 (m, 2H), 7.57 (dd, J=9.3, 2.5 Hz, 1H), 7.85 (d, J=2.5 Hz, 1H), 13.06 (s, 1H)

### Example 284

Step 1: Preparation of methyl 2-(3-(1-(2,3-difluorobenzyl)-6-oxo-1,6-dihydro-pyridin-3-yl)-2-methyl-1H-indol-1-yl)acetate, Intermediate 242. Intermediate 242 was prepared according to the method for example 274; Yield (55%).

Step 2: Preparation of 2-(3-(1-(2,3-difluorobenzyl)-6-oxo-1,6-dihydropyridin-3-yl)-2-methyl-1H-indol-1-yl)acetic acid (284). The title compound was prepared according to the method described for 273; Yield 74% 1H NMR (400 MHz, DMSO-d₆) δ ppm 2.34 (s, 3H), 5.02 (s, 2H), 5.30 (s, 2H), 6.56 (d, J=9.3 Hz, 1H), 7.04 (t, J=7.7 Hz, 2H), 7.09-7.16 (m, 1H), 7.18-7.28 (m, 1H), 7.32-7.48 (m, 3H), 7.62 (dd, J=9.2, 2.7 Hz, 1H), 7.82 (d, J=2.3 Hz, 1H), 13.07 (br. s., 1H)

### Example 285

Step 1: Preparation of methyl 2-(3-(1-(2,6-difluorobenzyl)-6-oxo-1,6-dihydro-pyridin-3-yl)-2-methyl-1H-indol-1-yl)acetate, Intermediate 243. Intermediate 243 was prepared according to the method for example 274; Yield (68%).

Step 2: Preparation of 2-(3-(1-(2,6-difluorobenzyl)-6-oxo-1,6-dihydropyridin-3-yl)-2-methyl-1H-indol-1-yl)acetic acid (285). The title compound was prepared according to the method described for 273; Yield 76% 1H NMR (400 MHz, DMSO-d₆) δ ppm 2.34 (s, 3H), 5.02 (s, 2H), 5.24 (s, 2H), 6.47 (d, J=9.3 Hz, 1H), 7.05 (t, J=7.5 Hz, 1H), 7.08-7.18 (m, 3H), 7.35-7.50 (m, 3H), 7.56 (dd, J=9.3, 2.5 Hz, 1H), 7.78 (s, 1H), 13.07 (br. s., 1H)

### Example 286

Step 1: Preparation of methyl 2-(2-methyl-3-(6-oxo-1-(2,4,5-trifluorobenzyl)-1,6-dihydropyridin-3-yl)-1H-indol-1-yl)acetate, Intermediate 244. Intermediate 244 was prepared according to the method for example 274; Yield (71%).

Step 2: Preparation of 2-(2-methyl-3-(6-oxo-1-(2,4,5-trifluorobenzyl)-1,6-dihydropyridin-3-yl)-1H-indol-1-yl)acetic acid (286). The title compound was prepared according to the method described for 273; Yield 63%

### Example 287

Step 1: Preparation of methyl 2-(3-(1-isobutyl-6-oxo-1,6-dihydropyridin-3-yl)-2-methyl-1H-indol-1-yl)acetate, Intermediate 245. Intermediate 245 was prepared according to the method for intermediate 362; Yield 42%.

Step 2: Preparation of 2-(3-(1-isobutyl-6-oxo-1,6-dihydropyridin-3-yl)-2-methyl-1H-indol-1-yl)-acetic acid (287). The title compound was prepared according to the method described for 232; Yield 37% 1H NMR (400 MHz, DMSO-d₆) δ ppm 0.91 (d, J=6.8 Hz, 6H), 2.05-2.24 (m, 1H), 2.34 (s, 3H), 3.80 (d, J=7.3 Hz, 2H), 5.02 (s, 2H), 6.50 (d, J=9.1 Hz, 1H), 7.05 (td, J=7.5, 0.9 Hz, (d, J=2.0 Hz, 1H), 13.09 (br. s., 1H)

### Example 288

Step 1: Preparation of methyl 2-(3-(1-cyclopentyl-6-oxo-1,6-dihydropyridin-3-yl)-2-methyl-1H-indol-1-yl)acetate, Intermediate 246. To a round bottom flask containing intermediate 231 (0.3 g, 1.0 mmol) in bromocyclopentane (4 mL, 37 mmol) was added 18-crown-6 (0.13 g, 0.5 mmol) and NaH (0.16 g, 4 mmol). The reaction was heated at 80 degrees for 24 hours until both TLC and LC/MS indicated the starting material had been consumed. The reaction was quenched by the addition of MeOH then the solvent was removed under reduced pressure and the crude material was purified by silica gel column chromatography to give a tan solid (0.07 g, 19%).

Step 2: Preparation of 2-(3-(1-cyclopentyl-6-oxo-1,6-dihydropyridin-3-yl)-2-methyl-1H-indol-1-yl)-acetic acid (288). The title compound was prepared according to the method described for 273; Yield 60% 1H NMR (400 MHz, DMSO-d₆) δ ppm 1.57-1.88 (m, 6H), 1.99-2.12 (m, 2H), 2.35 (s, 3H), 5.02 (s, 2H), 5.13-5.26 (m, 1H), 6.51 (d, J=9.1 Hz, 1H), 7.06 (td, J=7.5, 1.0 Hz, 1H), 7.12 (td, J=7.5, 1.1 Hz, 1H), 7.42 (dd, J=7.3, 5.3 Hz, 2H), 7.51 (dd, J=9.1, 2.5 Hz, 1H), 7.57 (d, J=2.3 Hz, 1H), 13.10 (br. s., 1H)

### Example 289

Step 1: Preparation of methyl 2-(5-chloro-3-(1-(4-fluorobenzyl)-6-oxo-1,6-dihydropyridin-3-yl)-2-methyl-1H-indol-1-yl)acetate, Intermediate 247. Intermediate 247 was prepared according to the method for Example 274.

Step 2: Preparation of 2-(5-chloro-3-(1-(4-fluorobenzyl)-6-oxo-1,6-dihydropyridin-3-yl)-2-methyl-1H-indol-1-yl)acetic acid (289). The title compound was prepared according to the method described for 273; Yield 66% for 2 steps. 1H NMR (400 MHz, DMSO-d₆) δ ppm 2.31 (s, 3H), 5.04 (s, 2H), 5.19 (s, 2H), 6.55 (d, J=9.3 Hz, 1H), 7.12 (dd, J=8.6, 2.0 Hz, 1H), 7.15-7.24 (m, 2H), 7.33 (d, J=1.8 Hz, 1H), 7.43-7.51 (m, 3H), 7.55 (dd, J=9.3, 2.8 Hz, 1H), 7.88 (d, J=2.0 Hz, 1H), 13.13 (br. s., 1H)

### Example 290

Step 1: Preparation of methyl 2-(5-chloro-3-(1-(2-fluorobenzyl)-6-oxo-1,6-dihydropyridin-3-yl)-2-methyl-1H-indol-1-yl)acetate, Intermediate 248. Intermediate 248 was prepared according to the method described for example 274.

Step 2: Preparation of 2-(5-chloro-3-(1-(2-fluorobenzyl)-6-oxo-1,6-dihydropyridin-3-yl)-2-methyl-1H-indol-1-yl)acetic acid (290). The title compound was prepared according to the method described for 273; Yield 50% for 2 steps. 1H NMR (400 MHz, DMSO-d₆) δ ppm 2.32 (s, 3H), 5.05 (s, 2H), 5.25 (s, 2H), 6.55 (d, J=9.3 Hz, 1H), 7.12 (dd, J=8.7, 2.1 Hz, 1H), 7.17-7.31 (m, 3H), 7.32-7.42 (m, 2H), 7.49 (d, J=8.3 Hz, 1H), 7.59 (dd, J=9.3, 2.8 Hz, 1H), 7.79 (d, J=2.5 Hz, 1H), 13.13 (s, 1H)

### Example 291

Step 1: Preparation of methyl 2-(5-chloro-3-(1-(2,6-difluorobenzyl)-6-oxo-1,6-dihydropyridin-3-yl)-2-methyl-1H-indol-1-yl)acetate, Intermediate 249. Intermediate 249 was prepared according to the method described for example 274.

Step 2: Preparation of 2-(5-chloro-3-(1-(2,6-difluorobenzyl)-6-oxo-1,6-dihydropyridin-3-yl)-2-methyl-1H-indol-1-yl)acetic acid (291). The title compound was prepared according to the method described for 273; Yield 63% for 2 steps. 1H NMR (400 MHz, DMSO-d₆) δ ppm 2.33 (s, 3H), 5.05 (s, 2H), 5.25 (s, 2H), 6.47 (d, J=9.3 Hz, 1H), 7.07-7.17 (m, 3H), 7.38 (d, J=2.0 Hz, 1H), 7.39-7.48 (m, 1H), 7.49 (d, J=8.3 Hz, 1H), 7.55 (dd, J=9.3, 2.5 Hz, 1H), 7.82 (s, 1H), 13.14 (br. s., 1H).

### Example 292

Step 1: Preparation of methyl 2-(5-chloro-3-(1-(2,3-difluorobenzyl)-6-oxo-1,6-dihydropyridin-3-yl)-2-methyl-1H-indol-1-yl)acetate, Intermediate 250. Intermediate 250 was prepared according to the method described for example 274

Step 2: Preparation of 2-(5-chloro-3-(142,3-difluorobenzyl)-6-oxo-1,6-dihydropyridin-3-yl)-2-methyl-1H-indol-1-yl)acetic acid (292). The title compound was prepared according to the method described for 273; Yield 60% for 2 steps. 1H NMR (400 MHz, DMSO-d₆) δ ppm 2.33 (s, 3H), 5.05 (s, 2H), 5.30 (s, 2H), 6.56 (d, J=9.3 Hz, 1H), 7.01-7.09 (m, 1H), 7.13 (dd, J=8.6, 2.0 Hz, 1H), 7.17-7.27 (m, 1H), 7.32-7.45 (m, 2H), 7.49 (d, J=8.8 Hz, 1H), 7.60 (dd, J=9.3, 2.5 Hz, 1H), 7.85 (d, J=2.5 Hz, 1H), 13.14 (br. s., 1H)

### Example 293

Step 1: Preparation of methyl 2-(5-chloro-2-methyl-3-(6-oxo-1-(2,4,5-trifluoro-benzyl)-1,6-dihydropyridin-3-yl)-1H-indol-1-yl)acetate, Intermediate 251. Intermediate 251 was prepared according to the method described for example 274.

Step 2: Preparation of 2-(5-chloro-2-methyl-3-(6-oxo-1-(2,4,5-trifluorobenzyl)-1,6-dihydropyridin-3-yl)-1H-indol-1-yl)acetic acid (293). The title compound was prepared according to the method for 273; Yield 66%. 1H NMR (400 MHz, DMSO-d₆) δ ppm 2.32 (s, 3H), 4.84 (s, 2H), 5.20 (s, 2H), 6.54 (d, J=9.3 Hz, 1H), 7.09 (dd, J=8.7, 2.1 Hz, 1H), 7.33-7.49 (m, 3H), 7.53-7.68 (m, 2H), 7.80 (d, J=2.5 Hz, 1H)

### Example 294

Step 1: Preparation of methyl 2-(5-chloro-3-(1-(3,5-difluorobenzyl)-6-oxo-1,6-dihydropyridin-3-yl)-2-methyl-1H-indol-1-yl)acetate, Intermediate 252. Intermediate 252 was prepared according to the method described for example 274

Step 2: Preparation of 2-(5-chloro-3-(1-(3 ,5-difluorobenzyl)-6-oxo-1,6-dihydropyridin-3-yl)-2-methyl-1H-indol-1-yl)acetic acid (294). The title compound was prepared according to the method described for 273; Yield 49% for 2 steps. 1H NMR (400 MHz, DMSO-d₆) δ ppm 2.33 (s, 3H), 5.05 (s, 2H), 5.21 (s, 2H), 6.57 (d, J=9.9 Hz, 1H), 7.12 (dd, J=8.6, 2.0 Hz, 3H), 7.20 (tt, J=9.4, 2.4 Hz, 1H), 7.38 (d, J=2.0 Hz, 1H), 7.49 (d, J=8.8 Hz, 1H), 7.58 (dd, J=9.2, 2.7 Hz, 1H), 7.93 (d, J=2.0 Hz, 1H), 13.13 (br. s., 1H)

### Example 295

Step 1: Preparation of methyl 2-(5-chloro-3-(1-(3-fluorobenzyl)-6-oxo-1,6-dihydropyridin-3-yl)-2-methyl-1H-indol-1-yl)acetate, Intermediate 253. Intermediate 253 was prepared according to the method described for example 274

Step 2: Preparation of 2-(5-chloro-3-(1-(3-fluorobenzyl)-6-oxo-1,6-dihydropyridin-3-yl)-2-methyl-1H-indol-1-yl)acetic acid (295). The title compound was prepared according to the method described for 273; Yield 36% for 2 steps. 1H NMR (400 MHz, DMSO-d₆) δ ppm 2.32 (s, 3H), 5.02 (s, 2H), 5.21 (s, 2H), 6.56 (d, J=9.3 Hz, 1H), 7.06-7.19 (m, 2H), 7.20-7.28 (m, 2H), 7.35 (d, J=1.8 Hz, 1H), 7.38-7.45 (m, 1H), 7.47 (d, J=8.8 Hz, 1H), 7.57 (dd, J=9.3, 2.8 Hz, 1H), 7.90 (d, J=2.0 Hz, 1H)

### Example 296

Step 1: Preparation of methyl 2-(2-methyl-3-(6-oxo-1-(4,4,4-trifluorobutyl)-1,6-dihydropyridin-3-yl)-1H-indol-1-yl)acetate, Intermediate 254. Intermediate 254 was prepared according to the method described for intermediate 232

Step 2: Preparation of 2-(2-methyl-3-(6-oxo-1-(4,4,4-trifluorobutyl)-1,6-dihydropyridin-3-yl)-1H-indol-1-yl)acetic acid (296). The title compound was prepared according to the method described for 273; Yield 28% 1H NMR (400 MHz, DMSO-d₆) δ ppm 1.86-2.00 (m, 2H), 2.23-2.42 (m, 5H), 4.05 (t, J=7.2 Hz, 2H), 5.02 (s, 2H), 6.52 (d, J=9.3 Hz, 1H), 7.05 (td, J=7.4 1.1 Hz, 1H), 7.12 (td, J=7.6, 1.1 Hz, 1H), 7.38-7.47 (m, 2H), 7.55 (dd, J=9.3, 2.5 Hz, 1H), 7.74 (d, J=2.0 Hz, 1H), 13.04 (br. s., 1H)

### Example 297

Step 1: Preparation of methyl 2-(5-fluoro-2-methyl-3-(6-oxo-1-(4,4,4-trifluoro-butyl)-1,6-dihydropyridin-3-yl)-1H-indol-1-yl)acetate, Intermediate 255. Intermediate 255 was prepared according to the method described for intermediate 232

Step 2: Preparation of 2-(5-fluoro-2-methyl-3-(6-oxo-1-(4,4,4-trifluorobutyl)-1,6-dihydropyridin-3-yl)-1H-indol-1-yl)acetic acid (297). The title compound was prepared according to the method for 273;Yield 70% 1H NMR (400 MHz, DMSO-d₆) δ ppm 1.87-2.01 (m, 2H), 2.23-2.42 (m, 5H), 4.05 (t, J=7.3 Hz, 2H), 5.02 (s, 2H), 6.51 (d, J=9.1 Hz, 1H), 6.95 (td, J=9.2, 2.5 Hz, 1H), 7.18 (dd, J=9.9, 2.5 Hz, 1H), 7.44 (dd, J=8.8, 4.3 Hz, 1H), 7.54 (dd, J=9.2, 2.7 Hz, 1H), 7.75 (d, J=2.3 Hz, 1H), 13.24 (br. s., 1H)

### Example 298

Step 1: Preparation of methyl 2-(3-bromo-5-methoxy-2-methyl-1H-indol-1-yl)-acetate, Intermediate 256. Intermediate 256 was prepared according to the method for intermediate 219; Yield 92%

Step 2: Preparation of methyl 2-(5-methoxy-3-(6-methoxypyridin-3-yl)-2-methyl-1H-indol-1-yl)-acetate, Intermediate 257. Intermediate 257 was prepared according to the method for intermediate 220; Yield 54%.

Step 3: Preparation of methyl 2-(5-methoxy-2-methyl-3-(6-oxo-1,6-dihydro-pyridin-3-yl)-1H-indol-1-yl)acetate, Intermediate 258. Intermediate 258 was prepared according to the method for intermediate 231; Yield 46%

Step 4: Preparation of methyl 2-(5-methoxy-2-methyl-3-(6-oxo-1-(2,2,2-trifluoro-ethyl)-1,6-dihydropyridin-3-yl)-1H-indol-1-yl)acetate, Intermediate 259. Intermediate 259 was prepared according to the method described for intermediate 232

Step 5: Preparation of 2-(5-methoxy-2-methyl-3-(6-oxo-1-(2,2,2-trifluoroethyl)-1,6-dihydropyridin-3-yl)-1H-indol-1-yl)acetic acid (298). The title compound was prepared according to the method described for 273; Yield 17% for 2 steps. 1H NMR (400 MHz, DMSO-d₆) δ ppm 2.32 (s, 3H), 3.74 (s, 3H), 4.89-5.02 (m, 4H), 6.61 (d, J=9.3Hz, 1H), 6.77 (dd, J=8.8, 2.3Hz, 1H), 6.91 (d, J=2.3 Hz, 1H), 7.34 (d, J=8.6 Hz, 1H), 7.64 (dd, J=9.5, 2.7 Hz, 1H), 7.73 (d, J=2.5 Hz, 1H), 13.01 (br. s., 1H).

### Example 299

Step 1: Preparation of methyl 2-(3-(1-(2,3-difluorobenzyl)-6-oxo-1,6-dihydro-pyridin-3-yl)-5-fluoro-2-methyl-1H-indol-1-yl)acetate, Intermediate 260. Intermediate 260 was prepared according to the method for example 274; Yield (78%).

Step 2: Preparation of 2-(3-(1-(2,3-difluorobenzyl)-6-oxo-1,6-dihydropyridin-3-yl)-5-fluoro-2-methyl-1H-indol-1-yl)acetic acid, Intermediate 261. The title compound was prepared according to the method for 273; Yield 82%

Step 3: Preparation of 1-(2,3-difluorobenzyl)-5-(5-fluoro-2-methyl-1-(2-oxo-2-(pyrrolidin-1-yl)-ethyl)-1H-indol-3-yl)pyridin-2(1H)-one (299). To a round bottom flask containing intermediate 261 (200 mg, 0.469 mmol), BOP (228 mg, 0.52 mmol), pyrrolidine (43 uL, 0.52 mmol) in DMF (5 ml) was added DIEA (122 uL, 0.7 mmol) to give a orange solution. This was stirred at room temp for 18 hrs aqueous workup followed by prep HPLC purification afforded the title compound as a white solid. Yield 56%. 1H NMR (400 MHz, DMSO-d₆) δ ppm 1.76-1.87 (m, 2H), 1.91-2.03 (m, 2H), 2.29 (s, 3H), 3.31-3.36 (m, 2H), 3.65 (t, J=6.8 Hz, 2H), 5.06 (s, 2H), 5.30 (s, 2H), 6.55 (d, J=9.3 Hz, 1H), 6.93 (td, J=9.2, 2.7 Hz, 1H), 7.05 (td, J=6.3, 1.5 Hz, 1H), 7.15 (dd, J=9.9, 2.5 Hz, 1H), 7.17-7.26 (m, 1H), 7.32-7.46 (m, 2H), 7.60 (dd, J=9.2, 2.7 Hz, 1H), 7.81 (d, J=2.5 Hz, 1H)

### Example 300

Preparation of 2-(3-(1-(2,3-difluorobenzyl)-6-oxo-1,6-dihydropyridin-3-yl)-5-fluoro-2-methyl-1H-indol-1-yl)-N,N-dimethylacetamide (300). The title compound was prepared according to the method described for 299; Yield 60%. 1H NMR (400 MHz, DMSO-d₆) δ ppm 2.27 (s, 3H), 2.86 (s, 3H), 3.16 (s, 3H), 5.15 (s, 2H), 5.30 (s, 2H), 6.55 (d, J=9.3 Hz, 1H), 6.92 )td, J=9.2, 2.5 Hz, 1H), 7.05 (td, J=6.3 1.5 Hz, 1H), 7.14 (dd, J=10.0, 2.4 Hz, 1H), 7.17-7.26 (m, 1H), 7.32-7.44 (m, 2H), 7.60 (dd, J=9.2, 2.7 Hz, 1H), 7.80 (d, J=2.5 Hz, 1H)

### Example 301

Preparation of 2-(3-(1-(2,3-difluorobenzyl)-6-oxo-1,6-dihydropyridin-3-yl)-5-fluoro-2-methyl-1H-indol-1-yl)acetamide (301). The title compound was prepared according to the method described for 299; Yield 75%. 1H NMR (400 MHz, DMSO-d₆) δ ppm 2.34 (s, 3H), 4.81 (s, 2H), 5.30 (s, 2H), 6.55 (d, J=9.3 Hz, 1H), 6.96 (td, J=9.2, 2.5 Hz, 1H), 7.02-7.09 (m, 1H), 7.15 (dd, J=10.0, 2.4 Hz, 1H), 7.18-7.25 (m, J=8.1, 8.1, 5.1, 1.5 Hz, 1H), 7.28 (s, 1H), 7.34-7.43 (m, 2H), 7.60 (dd, J=9.3, 2.5 Hz, 2H), 7.80 (d, J=2.5 Hz, 1H)

### Example 302

Preparation of 2-(3-(1-(2,3-difluorobenzyl)-6-oxo-1,6-dihydropyridin-3-yl)-5-fluoro-2-methyl-1H-indol-1-yl)-N-(methylsulfonypacetamide (302). The title compound was prepared according to the method described for 299. Yield 58%. 1H NMR (400 MHz, DMSO-d₆) δ ppm 2.33 (s, 3H), 3.27 (s, 3H), 5.05 (s, 2H), 5.30 (s, 2H), 6.55 (d, J=9.3 Hz, 1H), 6.92-7.09 (m, 2H), 7.11-7.27 (m, 2H), 7.33-7.49 (m, 2H), 7.60 (dd, J=9.3, 2.5 Hz, 1H), 7.83 (d, J=2.3 Hz, 1H)

### Example 303

Step 1: Preparation of methyl 3-(3-bromo-5-fluoro-2-methyl-1H-indol-1-yl)-propanoate, Intermediate 262. Intermediate 262 was prepared according to the method for intermediate 219; Yield 45%

Step 2: Preparation of methyl 3-(5-fluoro-3-(6-methoxypyridin-3-yl)-2-methyl-1H-indol-1-yl)- propanoate, Intermediate 263. Intermediate 263 was prepared according to the method for intermediate 220; Yield 53%. ¹H

Step 3: Preparation of methyl 3-(3-(1-(2,3-difluorobenzyl)-6-oxo-1,6-dihydro-pyridin-3-yl)-5-fluoro-2-methyl-1H-indol-1-yl)propanoate, Intermediate 264. Intermediate 264 was prepared according to the method described for intermediate 232.

Step 4: Preparation of 3-(3-(1-(2,3-difluorobenzyl)-6-oxo-1,6-dihydropyridin-3-yl)-5-fluoro-2-methyl-1H-indol-1-yl)propanoic acid (303). The title compound was prepared according to the method described for 273; Yield 12% for 2 steps. ¹H NMR (400 MHz, DMSO-d₆) δ ppm 2.42 (s, 3H), 2.67 (t, J=7.3 Hz, 2H), 4.41 (t, J=7.1 Hz, 2H), 5.28 (s, 2H), 6.54 (d, J=9.3 Hz, 1H), 6.97 (td, J=9.2, 2.7 Hz, 1H), 7.02-7.08 (m, 1H), 7.14 (dd, J=9.9, 2.5 Hz, 1H), 7.17 -7.26 (m, 1H), 7.34-7.45 (m, 1H), 7.51 (dd, J=9.0, 4.4 Hz, 1H), 7.58 (dd, J=9.3, 2.5 Hz, 1H), 7.80 (d, J=2.5 Hz, 1H), 12.48 (br. s., 1H)

### Example 304

Step 1: Preparation of methyl 2-(3-bromo-2,5-dimethyl-1H-indol-1-yl)acetate, Intermediate 265. Intermediate 265 was prepared according to the method for intermediate 219. Yield 76%

Step 2: Preparation of methyl 2-(3-(6-methoxypyridin-3-yl)-2,5-dimethyl-1H-indol-1-yl)acetate, Intermediate 266. Intermediate 266 was prepared according to the method for intermediate 220. Yield 66%.

Step 3: Preparation of methyl 2-(2,5-dimethyl-3-(6-oxo-1,6-dihydropyridin-3-yl)-1H-indol-1-yl)-acetate, Intermediate 267. Intermediate 267 was prepared according to the method for intermediate 231Yield 78%

Step 4: Preparation of methyl 2-(2,5-dimethyl-3-(6-oxo-1-(2,2,2-trifluoroethyl)-1,6-dihydropyridin-3-yl)-1H-indol-1-yl)acetate, Intermediate 268. Intermediate 268 was prepared according to the method for intermediate 232. Yield 38%.

Step 5: Preparation of 2-(2,5-dimethyl-3-(6-oxo-1-(2,2,2-trifluoroethyl)-1,6-dihydropyridin-3-yl)-1H-indol-1-yl)acetic acid (304). The title compound was prepared according to the method described for 273. Yield 72%. ¹H NMR (400 MHz, DMSO-d₆) δ ppm 2.32 (s, 3H), 2.36 (s, 3H), 4.87-5.05 (m, 4H), 6.61 (d, J=9.3 Hz, 1H), 6.95 (dd, J=8.5, 1.4 Hz, 1H), 7.22 (s, 1H), 7.31 (d, J=8.3 Hz, 1H), 7.64 (dd, J=9.5, 2.7 Hz, 1H), 7.71 (d, J=2.3 Hz, 1H), 13.02 (br. s., 1H)

### Example 305

Preparation of 1-(2,3-difluorobenzyl)-5-(5-fluoro-1-(2-hydroxyethyl)-2-methyl-1H-indol-3-yl)-pyridin-2(1H)-one (305). To intermediate 261 (100 mg, 0.235 mmol) in THF (5 ml) in a 25 ml RBF was added borane-tetrahydrofuran complex (0.704 ml, 0.704 mmol) dropwise. This was stirred at RT for 3 hrs, aqueous workup followed by prep HPLC afforded the title compound as a yellow solid. Yield 52%. ¹H NMR (400 MHz, DMSO-d₆) δ ppm 2.42 (s, 3H), 3.68 (q, J=6.0 Hz, 2H), 4.23 (t, J=5.8 Hz, 2H), 4.89 (t, 1H), 5.29 (s, 2H), 6.54 (d, J=9.3 Hz, 1H), 6.95 (td, J=9.1, 2.5 Hz, 1H), 7.01-7.09 (m, J=1.5 Hz, 1H), 7.13 (dd, J=9.9, 2.5 Hz, 1H), 7.17-7.26 (m, 1H), 7.33-7.44 (m, 1H), 7.46 (dd, J=8.8, 4.5 Hz, 1H), 7.58 (dd, J=9.2, 2.7 Hz, 1H), 7.78 (d, J=2.5 Hz, 1H).

### Example 306

Step 1: Preparation of methyl 2-(3-bromo-5-fluoro-2-methyl-1H-indol-1-yl)-propanoate, Intermediate 269. Intermediate 269 was prepared according to the method for intermediate 219. Yield 36%

Step 2: Preparation of methyl 2-(5-fluoro-3-(6-methoxypyridin-3-yl)-2-methyl-1H-indol-1-yl)-propanoate, Intermediate 270. Intermediate 270 was prepared according to the method for intermediate 220. Yield 41%.

Step 3: Preparation of methyl 2-(3-(1-(2,3-difluorobenzyl)-6-oxo-1,6-dihydropyridin-3-yl)-5-fluoro-2-methyl-1H-indol-1-yl)propanoate, Intermediate 271. Intermediate 271 was prepared according to the method for intermediate 232. 75% yield

Step 4: Preparation of (S)-2-(3-(1-(2,3-difluorobenzyl)-6-oxo-1,6-dihydropyridin-3-yl)-5-fluoro-2-methyl-1H-indol-1-yl)propanoic acid (306a) and (R)-2-(3-(1-(2,3-difluorobenzyl)-6-oxo-1,6-dihydropyridin-3-yl)-5-fluoro-2-methyl-1H-indol-1-yl)propanoic acid (306b). The title compounds were prepared according to the method described for 273. The enantiomers were separated by chiral HPLC. Yield 15% for 306a 1H NMR (400 MHz, DMSO-d₆) δ ppm 1.62 (d, J=7.3 Hz, 3H), 2.38 (s, 3H), 5.28 (s, 2H), 5.46 (q, J=7.2 Hz, 1H), 6.54 (d, J=9.1 Hz, 1H), 6.96 (td, J=9.2, 2.7 Hz, 1H), 7.02-7.09 (m, 1H), 7.14 (dd, J=9.9, 2.5 Hz, 1H), 7.21 (qd, J=8.1, 5.1, 1.5 Hz, 1H), 7.30-7.44 (m, 2H), 7.60 (dd, J=9.2, 2.7 Hz, 1H), 7.83 (d, J=2.5 Hz, 1H), 13.13 (br. s., 1H). Yield 16% for 306b 1H NMR (400 MHz, DMSO-d₆) δ ppm 1.62 (d, J=7.1 Hz, 3H), 2.38 (s, 3H), 5.29 (s, 2H), 5.44 (q, J=6.3 Hz, 1H), 6.54 (d, J=9.3 Hz, 1H), 6.95 (td, J=9.0, 2.3 Hz, 1H), 7.05 (t, J=6.9 Hz, 1H), 7.14 (dd, J=9.9, 2.3 Hz, 1H), 7.17-7.27 (m, 1H), 7.29-7.46 (m, 2H), 7.60 (dd, J=9.3, 2.3 Hz, 1H), 7.84 (d, J=1.8 Hz, 1H), 13.20 (br. s., 1H).

### Example 307

Step 1: Preparation of methyl 2-(5-fluoro-2-methyl-3-(6-oxo-1-(3,3,3-trifluoro-propyl)-1,6-dihydropyridin-3-yl)-1H-indol-1-yl)acetate, Intermediate 272. Intermediate 272 was prepared according to the method for intermediate 232.

Step 2: Preparation of 2-(5-fluoro-2-methyl-3-(6-oxo-1-(3,3,3-trifluoropropyl)-1,6-dihydropyridin-3-yl)-1H-indol-1-yl)acetic acid (307). The title compound was prepared according to the method described for 273. Yield 25% (2 steps). 1H NMR (400 MHz, DMSO-d₆) δ ppm 2.33 (s, 3H), 2.81 (dq, J=18.4, 11.6, 11.5, 7.2 Hz, 2H), 4.24 (t, J=6.9 Hz, 2H), 5.04 (s, 2H), 6.53 (d, J=9.3 Hz, 1H), 6.96 (td, J=9.2, 2.7 Hz, 1H), 7.20 (dd, J=9.9, 2.5 Hz, 1H), 7.46 (dd, J=9.0, 4.4 Hz, 1H), 7.56 (dd, J=9.3, 2.5 Hz, 1H), 7.79 (d, J=2.3 Hz, 1H), 13.09 (br. s., 1H)

### Example 308

Preparation of 2-(5-fluoro-2-methyl-3-(6-oxo-1-(pyridin-4-ylmethyl)-1,6-dihydropyridin-3-yl)-1H-indol-1-yl)acetic acid (308). To intermediate 220 (300 mg, 0.954 mmol) in DMF (8 ml) at 0° C. was added sodium hydride (115 mg, 2.86 mmol). After 10 mins, lithium bromide (166 mg, 1.909 mmol) was added and the reaction was stirred for 1 hour. A solution of 4-(bromomethyl)pyridine hydrobromide (266 mg, 1.050 mmol) in DMF (2 ml) was added and the reaction allowed to warm to RT and stirred for 18 hrs. Upon quenching with MeOH, the ester was hydrolyzed to the acid. Purification via prep HPLC gave a brown solid. Yield 12% (2 steps). 1H NMR (400 MHz, DMSO-d₆) δ ppm 2.32 (s, 3H), 4.43 (s, 2H), 5.30 (s, 2H), 6.52 (d, J=9.3 Hz, 1H), 6.87 (td, J=9.2, 2.5 Hz, 1H), 7.18 (dd, J=10.1, 2.5 Hz, 1H), 7.24-7.38 (m, 3H), 7.58 (dd, J=9.3, 2.5 Hz, 1H), 7.76-7.85 (m, 2H), 8.55 (dt, J=2.9, 1.8 Hz, 1H)

### Example 309

Preparation of 2-(5-fluoro-2-methyl-3-(6-oxo-1-(pyridin-2-ylmethyl)-1,6-dihydropyridin-3-yl)-1H-indol-1-yl)acetic acid (309). The title compound was prepared according to the method described for 308. Yield 7% (2 steps). 1H NMR (400 MHz, DMSO-d₆) δ ppm 2.32 (s, 3H), 4.43 (s, 2H), 5.30 (s, 2H), 6.52 (d, J=9.3 Hz, 1H), 6.87 (td, J=9.2, 2.5 Hz, 1H), 7.18 (dd, J=10.1, 2.5 Hz, 1H), 7.24-7.38 (m, 3H), 7.58 (dd, J=9.3, 2.5 Hz, 1H), 7.76-7.85 (m, 2H), 8.55 (dt, J=2.9, 1.8 Hz, 1H)

### Example 310

Step 1: Preparation of 2-(3-bromo -5-fluoro-2-methyl-1H-indol-1-yl)acetonitrile, Intermediate 273. Intermediate 273 was prepared according to the method for intermediate 219. Yield 21%

Step 2: Preparation of 2-(5-fluoro-3-(6-methoxypyridin-3-yl)-2-methyl-1H-indol-1-yl)acetonitrile, Intermediate 274. Intermediate 274 was prepared according to the method for intermediate 220. Yield 54%.

Step 3: Preparation of 2-(3-(1-(2,3-difluorobenzyl)-6-oxo-1,6-dihydropyridin-3-yl)-5-fluoro-2-methyl-1H-indol-1-yl)acetonitrile (310). 310 was prepared according to the method described for intermediate 232. Purified via prep HPLC afforded the title compound as a orange solid. Yield 35%. 1H NMR (400 MHz, DMSO-d₆) δ ppm 2.46 (s, 3H), 5.28 (s, 2H), 5.59 (s, 2H), 6.55 (d, J=9.3 Hz, 1H), 7.00-7.14 (m, 2H), 7.18-7.25 (m, 2H), 7.25-7.45 (m, 1H), 7.59-7.68 (m, 2H), 7.89 (d, J=2.5 Hz, 1H)

### Example 311

Preparation of 5-(1-((2H-tetrazol-5-yl)methyl)-5-fluoro-2-methyl-1H-indol-3-yl)-1-(2,3-difluoro-benzyl)pyridin-2(1H)-one (311). To 310 (300 mg, 0.736 mmol) is added sodium azide (96 mg, 1.473 mmol) and ammonium chloride (79 mg, 1.473 mmol) followed by DMF (5 ml). The reaction is heated at 95° C. overnight. Aqueous workup followed by prep-HPLC purification afforded the title compound as a white solid. Yield 18%. 1H NMR (400 MHz, DMSO-d₆) δ ppm 2.46 (s, 3H), 5.29 (s, 2H), 5.79 (s, 2H), 6.55 (d, J=9.3 Hz, 1H), 6.94-7.08 (m, 2H), 7.13-7.26 (m, 2H), 7.32-7.45 (m, 1H), 7.54 (dd, J=9.0, 4.4 Hz, 1H), 7.60 (dd, J=9.3, 2.8 Hz, 1H), 7.83 (d, J=2.5 Hz, 1H)

### Example 312

Preparation of 2-(3-(1-benzyl-6-oxo-1,6-dihydropyridin-3-yl)-5-fluoro-2-methyl-1H-indol-1-yl)-N-(phenylsulfonyl)acetamide (312). The title compound was prepared from 265 according to the method described for 299. Yield 48%. 1H NMR (400 MHz, DMSO-d₆) δ ppm 2.12 (s, 3H), 4.98 (s, 2H), 5.18 (s, 2H), 6.52 (d, J=9.3 Hz, 1H), 6.90 (td, J=9.2, 2.5 Hz, 1H), 7.06 (dd, J=9.7, 2.4 Hz, 1H), 7.25 (dd, J=8.8, 4.3 Hz, 1H), 7.27-7.39 (m, 5H), 7.49 (dd, J=9.2, 2.7 Hz, 1H), 7.56-7.64 (m, 2H), 7.65-7.72 (m, 1H), 7.78 (d, J=2.0 Hz, 1H), 7.87-7.94 (m, 2H)

### Example 313

Preparation of 2-(3-(1-benzyl-6-oxo-1,6-dihydropyridin-3-yl)-5-fluoro-2-methyl-1H-indol-1-yl)-N- (methylsulfonyl)acetamide (313). The title compound was prepared from 265 according to the method described for 299. Yield 18%. 1H NMR (400 MHz, DMSO-d₆) δ ppm 2.30 (s, 3H), 3.21 (s, 3H), 4.99 (s, 2H), 5.21 (s, 2H), 6.55 (d, J=8.6 Hz, 1H), 6.97 (td, J=9.1, 2.5 Hz, 1H), 7.11 (dd, J=9.9, 2.5 Hz, 1H), 7.25-7.33 (m, 1H), 7.33-7.44 (m, 5H), 7.55 (dd, J=9.2, 2.7 Hz, 1H), 7.83 (d, J=2.3 Hz, 1H), 12.29 (br. s., 1H)

### Example 314

Preparation of 2-(3-(1-benzyl-6-oxo-1,6-dihydropyridin-3-yl)-5-fluoro-2-methyl-1H-indol-1-yl)-N- (o-tolyl-sulfonyl)acetamide (314). The title compound was prepared from 265 according to the method described for 299. Yield 18%. 1H NMR (400 MHz, DMSO-d₆) δ ppm 2.15 (s, 3H), 2.64 (s, 3H), 5.00 (s, 2H), 5.18 (s, 2H), 6.52 (d, J=9.3 Hz, 1H), 6.93 (td, J=9.1, 2.3 Hz, 1H), 7.06 (dd, J=9.7, 2.4 Hz, 1H), 7.22-7.44 (m, 8H), 7.45-7.59 (m, 2H), 7.77 (d, J=2.3 Hz, 1H), 7.89 (d, J=7.6 Hz, 1H), 12.82 (br. s., 1H)

### Example 315

Preparation of 2-(5-fluoro-2-methyl-3-(6-oxo-1-(pyridin-3-ylmethyl)-1,6-dihydropyridin-3-yl)-1H-indol-1-yl)acetic acid (315). The title compound was prepared according to the method described for 308. Yield 25% (2 steps). 1H NMR (400 MHz, DMSO-d₆) δ ppm 2.32 (s, 3H), 5.01 (s, 2H), 5.23 (s, 2H), 6.55 (d, J=9.3 Hz, 1H), 6.95 (td, J=9.2, 2.7 Hz, 1H), 7.14 (dd, J=9.9, 2.5 Hz, 1H), 7.35-7.49 (m, 2H), 7.57 (dd, J=9.3, 2.5 Hz, 1H), 7.80 (d, J=7.8 Hz, 1H), 7.96 (d, J=2.3 Hz, 1H), 8.50 (dd, J=4.8, 1.3 Hz, 1H), 8.65 (d, J=2.0 Hz, 1H)

### Example 316

Step 1: Preparation of methyl 2-(5-fluoro-2-methyl-3-(6-oxo-1-(4,4,4-trifluoro-3-(trifluoro-methyl)butyl)-1,6-dihydropyridin-3-yl)-1H-indol-1-yl)acetate, Intermediate 275. Intermediate 275 was prepared according to the method described for intermediate 232.

Step 2: Preparation of 2-(5-fluoro-2-methyl-3-(6-oxo-1-(4,4,4-trifluoro-3-(trifluoromethyl)butyl)-1,6-dihydropyridin-3-yl)-1H-indol-1-yl)acetic acid (316). The title compound was prepared according to the method described for 273. Yield 16% 1H NMR (400 MHz, DMSO-d₆) δ ppm 2.17-2.29 (m, 2H), 2.34 (s, 3H), 2.52-2.56 (m, 1H), 4.17 (t, J=7.7 Hz, 2H), 5.05 (s, 2H), 6.54 (d, J=9.1 Hz, 1H), 6.97 (td, J=9.2, 2.5 Hz, 1H), 7.22 (dd, J=10.0, 2.4 Hz, 1H), 7.46 (dd, J=8.8, 4.3 Hz, 1H), 7.56 (dd, J=9.3, 2.5 Hz, 1H), 7.83 (d, J=2.5 Hz, 1H), 13.10 (br. s., 1H)

### Example 317

Preparation of N-(cyclopropylsulfonyl)-2-(5-fluoro-2-methyl-3-(6-oxo-1-(4,4,4-trifluorobutyl)-1,6-dihydropyridin-3-yl)-1H-indol-1-yl)acetamide (317). The title compound was prepared from 297 according to the method described for 299. Yield 16%. 1H NMR (400 MHz, DMSO-d₆) δ ppm 0.97-1.12 (m, 4H), 1.87-2.03 (m, 2H), 2.23-2.43 (m, 5H), 2.85-2.98 (m, 1H), 4.06 (t, J=7.2 Hz, 2H), 5.02 (s, 2H), 6.53 (d, J=9.3 Hz, 1H), 6.99 (td, J=9.2, 2.5 Hz, 1H), 7.20 (dd, J=9.9, 2.3 Hz, 1H), 7.42 (dd, J=8.8, 4.5 Hz, 1H), 7.55 (dd, J=9.3, 2.5 Hz, 1H), 7.77 (d, J=2.5 Hz, 1H), 12.31 (br. s., 1H)

### Example 318

Preparation of 2-(5-fluoro-2-methyl-3-(6-oxo-1-(4,4,4-trifluorobutyl)-1,6-dihydropyridin-3-yl)-1H-indol-1-yl)-N-(methylsulfonyl)acetamide (318). The title compound was prepared from 297 according to the method described for 299. Yield 34%. 1H NMR (400 MHz, DMSO-d₆) δ ppm 1.88-2.01 (m, 2H), 2.26-2.42 (m, 5H), 3.28 (s, 3H), 4.06 (t, J=7.2 Hz, 2H), 5.06 (s, 2H), 6.53 (d, J=9.3 Hz, 1H), 7.00 (td, J=9.1, 2.5 Hz, 1H), 7.21 (dd, J=9.7, 2.4 Hz, 1H), 7.43 (dd, J=8.8, 4.3 Hz, 1H), 7.55 (dd, J=9.3, 2.5 Hz, 1H), 7.78 (d, J=2.0 Hz, 1H), 12.31 (br. s., 1H)

### Example 319

Preparation of 2-(3-(1-benzyl-6-oxo-1,6-dihydropyridin-3-yl)-5-fluoro-2-methyl-1H-indol-1-yl)-N- (cyclopropyl-sulfonyl)acetamide (319). The title compound was prepared from 265 according to the method described for 299. Yield 14%. 1H NMR (400 MHz, DMSO-d₆) δ ppm 1.00-1.14 (m, 4H), 2.30 (s, 3H), 2.94 (tt, J=7.7, 5.1 Hz, 1H), 5.08 (s, 2H), 5.21 (s, 2H), 6.55 (d, J=9.3 Hz, 1H), 6.99 (td, J=9.2, 2.5 Hz, 1H), 7.12 (dd, J=9.9, 2.3 Hz, 1H), 7.23-7.48 (m, 6H), 7.56 (dd, J=9.3, 2.5 Hz, 1H), 7.84 (d, J=2.3 Hz, 1H), 12.28 (br. s., 1H)

### Example 320

Preparation of 2-(3-((1-(2,4-difluorobenzyl)-6-oxo-1,6-dihydropyridazin-3-yl)methyl)-5-fluoro-2-methyl-1H-indol-1-yl)-N-(methylsulfonyl)acetamide (320). The title compound was prepared from 83 according to the method described 299. Yield 64%. 1H NMR (400 MHz, DMSO-d₆) δ ppm 2.28 (s, 3H), 3.24 (s, 3H), 3.92 (s, 2H), 4.95 (s, 2H), 5.25 (s, 2H), 6.83-6.93 (m, 2H), 7.03 (qd, J=8.5, 2.6, 1.1 Hz, 1H), 7.09 (dd, J=9.9, 2.5 Hz, 1H), 7.18 (d, J=9.6 Hz, 1H), 7.20-7.28 (m, 1H), 7.28-7.37 (m, 2H), 12.24 (br. s., 1H)

### Example 321

Preparation of 2-(3-((1-(3,4-difluorobenzyl)-6-oxo-1,6-dihydropyridin-3-yl)methyl)-5-fluoro-2-methyl-1H-indol-1-yl)-N-(methylsulfonyl)acetamide (321). The title compound was prepared from 33 according to the method described for 299. Yield 60%. 1H NMR (400 MHz, DMSO-d₆) δ ppm 2.31 (s, 3H), 3.25 (s, 3H), 3.73 (s, 2H), 4.96 (s, 2H), 5.03 (s, 2H), 6.33 (d, J=9.3 Hz, 1H), 6.89 (td, J=9.2, 2.7 Hz, 1H), 7.09-7.24 (m, 3H), 7.31 (dd, J=8.8, 4.3 Hz, 1H), 7.33-7.42 (m, 2H), 7.77 (d, J=2.0 Hz, 1H), 12.25 (br. s., 1H)

### Example 322

Preparation of 2-(5-chloro-2-methyl-3-(4-oxo-3-(2-phenoxyethyl)-3,4-dihydrophthalazin-1-yl)-1H-indol-1-yl)-N-(methylsulfonypacetamide (322). The title compound was prepared from 221 according to the method described for 299.Yield 76%. 1H NMR (400 MHz, DMSO-d₆) δ ppm 2.26 (s, 3H), 3.29 (s, 3H), 4.41-4.49 (m, 2H), 4.53-4.68 (m, 2H), 5.14 (s, 2H), 6.87-6.98 (m, 3H), 7.17-7.30 (m, 4H), 7.47-7.58 (m, 2H), 7.85-7.94 (m, 2H), 8.36-8.43 (m, 1H), 12.37 (s, 1H),

### Example 323

Preparation of 2-(3-(2-benzyl-1-oxo-1,2-dihydroisoquinolin-4-yl)-5-fluoro-2-methyl-1H-indol-1-yl)-N-(methylsulfonyl)acetamide (323). The title compound was prepared from 248 according to the method described for 299. Yield 58%. 1H NMR (400 MHz, DMSO-d₆) δ ppm 2.18 (s, 3H), 3.28 (s, 3H), 5.10 (s, 2H), 5.28 (d, J=1.8 Hz, 2H), 6.79 (dd, J=9.7, 2.4 Hz, 1H), 6.98 (td, J=9.1, 2.5 Hz, 1H), 7.20-7.32 (m, 2H), 7.33-7.42 (m, 4H), 7.47 (dd, J=8.8, 4.3 Hz, 1H), 7.51-7.59 (m, 2H), 7.66 (ddd, J=8.2, 6.9, 1.5 Hz, 1H), 8.36 (d, J=8.1 Hz, 1H), 12.32 (br. s., 1H)

### Example 324

Preparation of 2-(3-(1-benzyl-6-oxo-1,6-dihydropyridazin-3-yl)-5-fluoro-2-methyl-1H-indol-1-yl)-acetic acid (324). The title compound was prepared according to the procedure of Example 12; Yield: 68%.

### Example 325

Preparation of 2-(3-(1-(2,4-dichlorobenzyl)-6-oxo-1,6-dihydropyridazin-3-yl)-5-fluoro-2-methyl-1H-indol-1-yl)acetic acid (325). The title compound was prepared according to the procedure of Example 12; Yield: 55%.

### Example 326

Preparation of [3-(3-isopropyl-4-oxo-3,4-dihydrophthalazin-1-yl)-2-methyl-1H-indol-1-yl]acetic acid (326). The title compound was prepared according to the procedure of Example 1; Yield: 30%.

### Example 327

Preparation of {5-fluoro-2-methyl-3-[3-(2-methylpropyl)-4-oxo-3,4-dihydrophthalazin-1-yl]-1H-indol-1-yl}acetic acid (327). The title compound was prepared according to the procedure of Example 1; Yield: 52.6%.

### Example 328

Preparation of [3-(3-benzyl-4-oxo-3,4-dihydrophthalazin-1-yl)-5-fluoro-2-methyl-1H-indol-1-yl]-acetic acid (328). The title compound was prepared according to the procedure of Example 1; Yield: 27%.

### Example 329

Preparation of {5-fluoro-3-[3-(3-fluorobenzyl)-4-oxo-3,4-dihydrophthalazin-1-yl]-2-methyl-1H-indol-1-yl}acetic acid (329). The title compound was prepared according to the procedure of Example 1; Yield: 48.6%.

### Example 330

Preparation of {5-fluoro-2-methyl-3-[3-(1-methylethyl)-4-oxo-3,4-dihydrophthalazin-1-yl]-1H-indol-1-yl}acetic acid (330). The title compound was prepared according to the procedure of Example 1; Yield: 41.8%.

### Example 331

Preparation of {5-chloro-3-[3-(2,4-dichlorobenzyl)-4-oxo-3,4-dihydrophthalazin-1-yl]-2-methyl-1H-indol-1-yl}acetic acid (331). The title compound was prepared according to the procedure of Example 1; Yield: 56.5%.

### Example 332

Preparation of (5-chloro-2-methyl-3-{3-[4-(methylsulfonyl)benzyl]-4-oxo-3,4-dihydro-phthalazin-1-yl}-1H-indol-1-yl)acetic acid (332). The title compound was prepared according to the procedure of Example 1; Yield: 17.6%.

### Example 333

Preparation of 2-(5-fluoro-3-(1-isobutyl-6-oxo-1,6-dihydropyridazin-3-yl)-2-methyl-1H-indol-1-yl)acetic acid (333). The title compound was prepared according to the procedure of Example 12; Yield: 52%.

### Example 334

Preparation of [5-fluoro-3-(1-isopropyl-6-oxo-1,6-dihydropyridazin-3-yl)-2-methyl-1H-indol-1-yl]-acetic acid (334). The title compound was prepared according to the procedure of Example 12; Yield: 67.8%.

### Example 335

Step 1: Preparation of 2,4-difluorobenzylhydrazine, Intermediate 276. The procedure described above for example 81 was followed, reacting hydrazine with 2,4-difluorobenzylbromide and purifying by distillation. Intermediate 410 ditilled at 95° C. at 8 ton pressure (71.8% yield)

Step 2: Preparation of methyl 1-(2,4-difluorobenzyl)-6-oxo-1,4,5,6-tetrahydro-pyridazine-3-carboxylate, Intermediate 277. The procedure described above for example 81 was followed, reacting 2,4-difluorobenzylhydrazine with dimethyl 2-oxoglutarate (85.7% yield).

Step 3: Preparation of 6-(hydroxymethyl)-2-(2,4-difluorobenzyl)-4,5-dihydropyridazin-3(2H)-one, intermediate 278. The procedure described above for example 81 was followed, reacting intermediate 277 with sodium borohydride (33% yield) ¹H NMR (400 MHz, DMSO-d₆) δ 7.17-7.33 (m, 2H), 7.00-7.07 (m, 1H), 5.21 (t, J=5.94 Hz, 1H), 4.82 (s, 2H), 4.01 (d, J=6.06 Hz, 2H), 2.54-2.60 (m, 2H), 2.41-2.47 (m, 2H).

Step 4: Preparation of 6-oxo-1-(2,4-difluoro benzyl)-1,6-dihydropyridazine-3-carbaldehyde, Intermediate 279. The procedure described above for example 81 was followed, reacting of intermediate 278 with manganese dioxide (38% yield)

Step 5: Preparation of (3-{[1-(2,4-difluorobenzyl)-6-oxo-1,6-dihydropyridazin-3-yl]methyl}-2-methyl-1H- indol-1-yl)acetic acid (335). The procedure described above for example 81 was followed, reacting intermediate 279 with methyl 2-(2-methyl-1H-indol-1-yl)acetate then deprotecting with LiOH following procedure for 81 and purifying (43% yield) ¹H NMR (400 MHz, DMSO-d₆) δ 12.99 (br. s., 1H), 7.21-7.46 (m, 4H), 7.14 (d, J=9.60 Hz, 1H), 6.96-7.10 (m, 2H), 6.78-6.94 (m, 2H), 5.26 (s, 2H), 4.92 (s, 2H), 3.93 (s, 2H), 2.30 (s, 3H)

### Example 336

Step 1: Preparation of methyl 2-(3-((1-benzyl-6-oxo-1,6-dihydropyridazin-3-yl)-methyl)-2-methyl-1H-indol-1-yl)acetate, Intermediate 280. The procedure described above for example 74 was followed, reacting intermediate 36 with intermediate 95 (43% yield)

Step 2: Preparation of methyl 2-(3-((1-benzyl-6-oxo-1,6-dihydropyridazin-3-yl)-methyl)-2-methyl-1H-indol-1-yl)acetate, Intermediate 281. The procedure described above for example 74 was followed, reacting intermediate 280 with aluminium chloride. Crude compound used in the next step.

Step 3: Preparation of (3-{[1-(2,5-difluorobenzyl)-6-oxo-1,6-dihydropyridazin-3-yl]methyl}-5-fluoro-2-methyl-1H-indol-1-yl)acetic acid (336). The procedure described above for example 74 was followed, reacting intermediate 281 with 2,5-difluorobenzylbromide then deprotecting with LiOH following procedure for 81 and purifying (21.8% yield over 3 steps) ¹H NMR (400 MHz, DMSO-d₆) δ 7.16-7.38 (m, 4H), 7.12 (dd, J=2.53, 9.85 Hz, 1H), 7.02 (ddd, J=3.28, 5.62, 8.78 Hz, 1H), 6.83-6.91 (m, 2H), 5.28 (s, 2H), 4.92 (s, 2H), 3.94 (s, 2H), 2.30 (s, 3H)

### Example 337

Preparation of {3-[(1-benzyl-6-oxo-1,6-dihydropyridazin-3-yl)methyl]-5-fluoro-2-methyl-1H-indol-1-yl}acetic acid (337). Intermediate 280 was deprotecting with LiOH following procedure for 81 and purifying (31% yield) ¹H NMR (400 MHz, DMSO-d₆) δ 13.02 (br. s., 1H), 7.27-7.39 (m, 6H), 7.14-7.21 (m, 2H), 6.82-6.91 (m, 2H), 5.22 (s, 2H), 4.94 (s, 2H), 3.96 (s, 2H), 2.30 (s, 3H)

### Example 338

Preparation of (3-{[1-(2,6-difluorobenzyl)-6-oxo-1,6-dihydropyridazin-3-yl]methyl}-5-fluoro-2-methyl-1H-indol-1-yl)acetic acid (338). The procedure described above for intermediate 213 was followed, reacting intermediate 281 with 2,6-difluorobenzyl bromide then deprotecting with LiOH following procedure for 79 and purifying (28% yield over 3 steps). ¹H NMR (400 MHz, DMSO-d₆) δ 13.13 (br. s., 1H), 7.52 (tt, J=6.66, 8.37 Hz, 1H), 7.38 (dd, J=4.42, 8.97 Hz, 1H), 7.12-7.22 (m, 3H), 7.09 (dd, J=2.53, 9.85 Hz, 1H), 6.86-6.95 (m, 2H), 5.36 (s, 2H), 4.95 (s, 2H), 3.89 (s, 2H), 2.26 (s, 3H)

### Example 339

Preparation of (3-{[1-(2,3-difluorobenzyl)-6-oxo-1,6-dihydropyridazin-3-yl]methyl}-5-fluoro-2-methyl-1H-indol-1-yl)acetic acid (339). The procedure described above for intermediate 213 was followed, reacting intermediate 281 with 2,3-difluorobenzyl bromide then deprotecting with LiOH following procedure for 79 and purifying (30.3% yield over 3 steps). ¹H NMR (400 MHz, DMSO-d₆) δ 13.13 (br. s., 1H), 7.52 (tt, J=6.66, 8.37 Hz, 1H), 7.38 (dd, J=4.42, 8.97 Hz, 1H), 7.12-7.22 (m, 3H), 7.09 (dd, J=2.53, 9.85 Hz, 1H), 6.86-6.95 (m, 2H), 5.36 (s, 2H), 4.95 (s, 2H), 3.89 (s, 2H), 2.26 (s, 3H)

### Example 340

Preparation of (3-{[1-(2-fluorobenzyl)-6-oxo-1,6-dihydropyridazin-3-yl]methyl}-5-fluoro-2-methyl-1H-indol-1-yl)acetic acid (340). The procedure described above for intermediate 213 was followed, reacting intermediate 281 with 2-fluorobenzyl bromide then deprotecting with LiOH following procedure for 79 and purifying (33% yield over 3 steps). ¹H NMR (400 MHz, DMSO-d₆) δ 13.03 (br. s., 1H), 7.32-7.40 (m, 2H), 7.11-7.25 (m, 5H), 6.84-6.90 (m, 2H), 5.28 (s, 2H), 4.93 (s, 2H), 3.93 (s, 2H), 2.28 (s, 3H)

### Example 341

Preparation of (3-{[1-(3-fluorobenzyl)-6-oxo-1,6-dihydropyridazin-3-yl]methyl}-5-fluoro-2-methyl-1H-indol-1-yl)acetic acid (341). The procedure described above for intermediate 213 was followed, reacting intermediate 281 with 3-fluorobenzyl bromide then deprotecting with LiOH following procedure for 79 and purifying (23.1% yield over 3 steps). ¹H NMR (400 MHz, DMSO-d₆) δ 7.31-7.40 (m, 2H), 7.06-7.19 (m, 5H), 6.82-6.89 (m, 2H), 5.24 (s, 2H), 4.87 (s, 2H), 3.96 (s, 2H), 2.30 (s, 3H)

### Example 342

Preparation of (3-{[1-(4-fluorobenzyl)-6-oxo-1,6-dihydropyridazin-3-yl]methyl}-5-fluoro-2-methyl-1H-indol-1-yl)acetic acid (342). The procedure described above for intermediate 213 was followed, reacting intermediate 281 with 4-fluorobenzyl bromide then deprotecting with LiOH following procedure for 79 and purifying (21.2% yield over 3 steps). ¹H NMR (400 MHz, DMSO-d₆) δ 7.23-7.33 (m, 3H), 7.02-7.12 (m, 4H), 6.74-6.84 (m, 2H), 5.14 (s, 2H), 4.85 (s, 2H), 3.88 (s, 2H), 2.23 (s, 3H)

### Example 343

Preparation of (3-{[1-(2,2-dimethylpropyl)-6-oxo-1,6-dihydropyridazin-3-yl]methyl}-5-fluoro-2-methyl-1H-indol-1-yl)acetic acid (343). The procedure described above for example 83 was followed, reacting methyl 2-(5-fluoro-2-methyl-3-((6-oxo-1,6-dihydropyridazin-3-yl)methyl)-1H-indol-1-yl)acetate with 1-bromo-2,2-dimethylpropane then deprotecting with LiOH following procedure for 83 and purifying (19.6% yield). ¹H NMR (400 MHz, DMSO-d₆) δ 7.35 (dd, J=4.29, 8.84 Hz, 1H), 7.23 (dd, J=2.53, 9.85 Hz, 1H), 7.14 (d, J=9.60 Hz, 1H), 6.88 (td, J=2.40, 9.16 Hz, 1H), 6.81 (d, J=9.35 Hz, 1H), 4.90 (s, 2H), 3.95 (s, 2H), 3.91 (s, 2H), 2.34 (s, 3H), 0.95 (s, 9H)

### Example 344

Preparation of 2-(5-fluoro-2-methyl-3-((6-oxo-1-(4,4,4-trifluorobutyl)-1,6-dihydropyridazin-3-yl)-methyl)-1H-indol-1-yl)acetic acid (344). The procedure described above for example 83 was followed, reacting methyl 2-(5-fluoro-2-methyl-3-((6-oxo-1,6-dihydropyridazin-3-yl)methyl)-1H-indol-1-yl)acetate with 4-bromo-1,1,1-trifluorobutane then deprotecting with LiOH following procedure for 83 and purifying (63.2% yield). ¹H NMR (400 MHz, DMSO-d₆) δ 7.35 (dd, J=4.29, 8.84 Hz, 1H), 7.23 (dd, J=2.53, 9.85 Hz, 1H), 7.14 (d, J=9.60 Hz, 1H), 6.88 (td, J=2.40, 9.16 Hz, 1H), 6.81 (d, J=9.35 Hz, 1H), 4.90 (s, 2H), 3.95 (s, 2H), 3.91 (s, 2H), 2.34 (s, 3H), 0.95 (s, 9H)

### Example 345

Preparation of (5-fluoro-2-methyl-3-{[6-oxo-1-(2,2,2-trifluoroethyl)-1,6-dihydropyridazin-3-yl]-methyl}-1H-indol-1-yl)acetic acid (345). The procedure described above for Example 83 was followed, reacting methyl 2-(5-fluoro-2-methyl-3-((6-oxo-1,6-dihydropyridazin-3-yl)methyl)-1H-indol-1-yl)acetate with 4-bromo-1,1,1-trifluorobutane, deprotecting with LiOH following procedure for 83 and purifying (36.5% yield). ¹H NMR (400 MHz, DMSO-d₆) δ 7.27 (dd, J=4.55, 8.84 Hz, 1H), 7.10-7.18 (m, 2H), 6.76-6.87 (m, 2H), 4.77-4.90 (m, 4H), 3.89 (s, 2H), 2.26 (s, 3H)

### Example 346

Preparation of {3-[(1-benzyl-6-oxo-1,6-dihydropyridin-3-yl)methyl]-5-fluoro-2-methyl-1H-indol-1-yl}acetic acid (346). The procedure described above for example 81 was followed, reacting intermediate 282, 1-benzyl-6-oxo-1,6-dihydropyridine-3-carbaldehyde (prepared by ref: Tetrahedron 1998, 54, 243) with methyl 2-(5-fluoro-2-methyl-1H-indol-1-yl)acetate then deprotecting with LiOH following procedure for 81 and purifying (51.2% yield) ¹H NMR (400 MHz, DMSO-d₆) δ 7.74 (d, J=2.02 Hz, 1H), 7.22-7.37 (m, 6H), 7.19 (td, J=2.53, 9.35 Hz, 2H), 6.86 (td, J=2.65, 9.28 Hz, 1H), 6.31 (d, J=9.35 Hz, 1H), 5.05 (s, 2H), 4.92 (s, 2H), 3.72 (s, 2H), 2.30 (s, 3H)

### Example 347

Preparation of {3-[(1-benzyl-6-oxo-1,6-dihydropyridin-3-yl)methyl]-5-chloro-2-methyl-1H-indol-1-yl}acetic acid (347). The procedure described above for example 81 was followed, reacting intermediate 282, with methyl 2-(5-chloro-2-methyl-1H-indol-1-yl)acetate then deprotecting with LiOH following procedure for 81 and purifying (79% yield) ¹H NMR (400 MHz, DMSO-d₆) δ 13.04 (br. s., 1H), 7.77 (s, 1H), 7.47 (d, J=2.02 Hz, 1H), 7.21-7.42 (m, 6H), 7.16 (dd, J=2.53, 9.35 Hz, 1H), 7.03 (dd, J=2.02, 8.84 Hz, 1H), 6.32 (d, J=9.35 Hz, 1H), 5.04 (s, 2H), 4.96 (s, 2H), 3.75 (s, 2H), 2.31 (s, 3H)

### Example 348

Preparation of {3-[(1-benzyl-6-oxo-1,6-dihydropyridin-3-yl)methyl]-2-methyl-1H-indol-1-yl}acetic acid (348). The procedure described above for example 81 was followed, reacting intermediate 282 (prepared by ref: Tetrahedron 1998, 54, 243) with methyl 2-(2-methyl-1H-indol-1-yl)acetate then deprotecting with LiOH following procedure for 81 and purifying (83.1% yield) ¹H NMR (400 MHz, DMSO-d₆) δ 13.01 (br. s., 1H), 7.71 (d, J=2.27 Hz, 1H), 7.39 (d, J=7.58 Hz, 1H), 7.23-7.36 (m, 6H), 7.17 (dd, J=2.65, 9.22 Hz, 1H), 7.00-7.05 (m, 1H), 6.90-6.95 (m, 1H), 6.31 (d, J=9.35 Hz, 1H), 5.05 (s, 2H), 4.91 (s, 2H), 3.75 (s, 2H), 2.30 (s, 3H).

FRET Assay: To evaluate potency compounds were tested in a FRET based competitive immunoassay in which CRTH2 expressing CHO cells were incubated in the presence of forskolin and PGD2 along with Eu3+ cryptate-labeled anti-cAMP and d2-labeled cAMP.

Summary of Biological Data:

| Example # | IC50 (FRET) μM |
|---|---|
| 1 | 0.015 |
| 2 | 0.018 |
| 3 | 0.058 |
| 4 | 0.009 |
| 5 | 0.01 |
| 6 | 0.023 |
| 7 | 0.004 |
| 8 | 0.005 |
| 9 | 0.021 |
| 10 | 0.106 |
| 11 | 0.039 |
| 12 | 0.006 |
| 13 | 0.005 |
| 14 | 0.169 |
| 15 | 0.214 |
| 16 | 0.031 |
| 17 | 2.782 |
| 18 | 0.192 |
| 19 | 0.109 |
| 20 | 0.005 |
| 21 | 0.018 |
| 22 | 0.765 |
| 23 | 0.017 |
| 24 | 0.028 |
| 25 | 0.007 |
| 26 | 0.008 |
| 27 | 0.137 |
| 28 | 0.017 |
| 29 | 0.072 |
| 30 | 0.153 |
| 31 | 0.041 |
| 32 | 0.008 |
| 33 | 0.011 |
| 34 | 0.054 |
| 35 | 0.065 |
| 36 | 0.002 |
| 37 | 0.035 |
| 38 | 0.007 |
| 39 | 0.004 |
| 40 | 0.03 |
| 41 | 0.003 |
| 42 | 0.007 |
| 43 | 0.009 |
| 44 | 0.02 |
| 45 | 0.005 |
| 46 | 0.055 |
| 47 | >2.000 |
| 48 | >2.000 |
| 49 | >2.000 |
| 50 | >1.500 |
| 51 | >2.000 |
| 52 | >2.000 |
| 53 | 0.055 |
| 54 | 0.329 |
| 55 | >2.000 |
| 56 | 0.005 |
| 57 | 0.005 |
| 58 | 0.018 |
| 59 | 0.012 |
| 60 | 0.011 |
| 61 | 0.011 |
| 62 | 0.009 |
| 63 | 0.015 |
| 64 | 0.009 |
| 65 | 0.018 |
| 66 | 0.143 |
| 67 | 0.023 |
| 68 | 0.01 |
| 69 | 0.007 |
| 70 | 0.016 |
| 71 | 0.017 |
| 72 | 0.043 |
| 73 | 0.076 |
| 74 | 0.002 |
| 75 | 0.101 |
| 76 | 0.016 |
| 77 | 0.032 |
| 78 | 0.002 |
| 79 | 0.022 |
| 80 | 0.016 |
| 81 | 0.023 |
| 82 | 0.016 |
| 83 | 0.003 |
| 84 | 0.028 |
| 85 | 0.062 |
| 86 | 0.197 |
| 87 | 0.21 |
| 88 | 0.197 |
| 89 | 0.123 |
| 90 | >1.000 |
| 91 | 0.107 |
| 92 | >1.000 |
| 93 | 0.021 |
| 94 | 0.119 |
| 95 | 0.009 |
| 96 | 0.296 |
| 97 | 0.007 |
| 98 | 0.017 |
| 99 | 0.01 |
| 100 | 0.081 |
| 101 | 0.01 |
| 102 | 0.027 |
| 103 | 0.011 |
| 104 | 0.01 |
| 105 | 0.121 |
| 106 | 0.023 |
| 107 | 0.02 |
| 108 | 0.022 |
| 109 | 0.042 |
| 110 | 0.007 |
| 111 | >0.020 |
| 112 | 0.019 |
| 113 | 0.014 |
| 114 | 0.013 |
| 115 | 0.064 |
| 116 | 0.007 |
| 117 | 0.022 |
| 118 | 0.009 |
| 119 | 0.017 |
| 120 | 0.201 |
| 121 | 0.055 |
| 122 | 0.027 |
| 123 | 0.022 |
| 124 | 0.008 |
| 125 | 0.004 |
| 126 | 0.008 |
| 127 | 0.004 |
| 128 | 0.006 |
| 129 | 0.136 |
| 130 | >1.000 |
| 131 | 0.054 |
| 132 | 0.075 |
| 133 | 0.008 |
| 134 | 0.003 |
| 135 | 0.005 |
| 136 | 0.004 |
| 137 | 0.021 |
| 138 | 0.005 |
| 139 | 0.024 |
| 140 | 0.016 |
| 141 | 0.046 |
| 142 | 0.019 |
| 143 | 0.055 |
| 144 | 0.011 |
| 145 | 0.144 |
| 146 | 0.074 |
| 147 | 0.334 |
| 148 | 0.042 |
| 149 | 2.155 |
| 150 | 0.559 |
| 151 | 0.02 |
| 152 | 0.012 |
| 153 | 0.025 |
| 154 | 0.03 |
| 155 | 0.02 |
| 156 | 0.025 |
| 157 | 0.057 |
| 158 | 0.073 |
| 159 | 2.491 |
| 160 | 0.375 |
| 161 | 0.023 |
| 162 | 0.462 |
| 163 | 0.865 |
| 164 | 0.409 |
| 165 | >1.000 |
| 166 | 0.02 |
| 167 | 0.014 |
| 168 | 0.062 |
| 169 | 0.014 |
| 170 | 0.025 |
| 171 | 0.296 |
| 172 | 0.04 |
| 173 | 0.076 |
| 174 | 0.98 |
| 175 | 0.175 |
| 176 | 0.123 |
| 177 | 0.215 |
| 178 | 0.215 |
| 179 | >1.000 |
| 180 | 0.096 |
| 181 | 0.92 |
| 182 | 1.72 |
| 183 | 4.992 |
| 184 | 0.062 |
| 185 | >1.000 |
| 186 | 0.15 |
| 187 | 0.075 |
| 188 | 0.227 |
| 189 | 0.045 |
| 190 | 0.867 |
| 191 | 0.064 |
| 192 | 0.004 |
| 193 | 0.008 |
| 194 | 0.098 |
| 195 | 0.012 |
| 196 | 0.018 |
| 197 | 0.27 |
| 198 | 0.046 |
| 199 | 0.019 |
| 200 | 0.045 |
| 201 | 0.022 |
| 202 | 0.009 |
| 203 | 0.01 |
| 204 | 0.01 |
| 205 | 0.06 |
| 206 | 0.32 |
| 207 | 0.588 |
| 208 | 0.003 |
| 209 | 0.014 |
| 210 | 0.002 |
| 211 | 0.022 |
| 212 | 0.033 |
| 213 | 0.019 |
| 214 | 0.351 |
| 215 | 0.027 |
| 216 | 0.014 |
| 217 | 0.034 |
| 218 | 0.024 |
| 219 | 0.03 |
| 220 | 0.018 |
| 221 | 0.006 |
| 222 | 0.001 |
| 223 | 0.006 |
| 224 | 0.093 |
| 225 | 0.027 |
| 226 | 0.002 |
| 227 | 0.003 |
| 228 | 0.147 |
| 229 | 0.136 |
| 230 | 0.232 |
| 231 | 0.401 |
| 232 | 1.08 |
| 233 | 0.147 |
| 234 | 1.001 |
| 235 | 1.583 |
| 236 | >1.000 |
| 237 | >1.000 |
| 238 | 0.013 |
| 239 | 0.007 |
| 240 | 0.004 |
| 241 | 0.006 |
| 242 | 0.028 |
| 243 | 0.033 |
| 244 | 0.06 |
| 245 | 0.587 |
| 246 | >2.000 |
| 247 | 0.012 |
| 248 | 0.015 |
| 249 | 0.005 |
| 250 | 0.004 |
| 251 | 0.205 |
| 252 | 0.012 |
| 253 | 0.009 |
| 254 | 0.015 |
| 255 | 0.055 |
| 256 | 0.002 |
| 257 | 0.003 |
| 258 | 0.011 |
| 259a | 0.061 |
| 259b | 0.026 |
| 259c | 0.023 |
| 260a | 3.384 |
| 260b | 1.652 |
| 261 | 0.011 |
| 262a | >10.000 |
| 262b | >10.000 |
| 262c | >10.000 |
| 263 | 0.042 |
| 264 | 0.01 |
| 265 | 0.005 |
| 266 | 0.009 |
| 267 | 0.006 |
| 268 | 0.007 |
| 269 | 0.001 |
| 270 | >5.500 |
| 271 | 0.012 |
| 272 | 0.023 |
| 273 | 0.028 |
| 274 | 0.003 |
| 275 | 0.03 |
| 276 | 0.577 |
| 277 | 0.071 |
| 278 | 0.198 |
| 279 | 0.715 |
| 280 | 0.117 |
| 281 | 0.24 |
| 282 | 0.011 |
| 283 | 0.016 |
| 284 | 0.015 |
| 285 | 0.023 |
| 286 | 0.011 |
| 287 | 0.305 |
| 288 | 0.117 |
| 289 | 0.006 |
| 290 | 0.007 |
| 291 | 0.008 |
| 292 | 0.011 |
| 293 | 0.02 |
| 294 | 0.002 |
| 295 | 0.011 |
| 296 | 0.397 |
| 297 | 0.038 |
| 298 | >2.000 |
| 299 | >2.000 |
| 300 | >2.000 |
| 301 | >2.000 |
| 302 | 0.159 |
| 303 | >2.000 |
| 304 | 1.18 |
| 305 | >2.000 |
| 306a | >2.000 |
| 306b | >2.000 |
| 307 | 0.028 |
| 308 | 0.035 |
| 309 | 0.105 |
| 310 | >1.000 |
| 311 | >1.000 |
| 312 | >1.000 |
| 313 | 0.032 |
| 314 | >1.000 |
| 315 | 0.032 |
| 316 | 0.039 |
| 317 | >1.000 |
| 318 | >1.000 |
| 319 | >1.000 |
| 320 | >1.000 |
| 321 | >1.000 |
| 322 | >1.000 |
| 323 | 0.518 |
| 324 | 0.006 |
| 325 | 0.02 |
| 326 | 0.061 |
| 327 | 0.013 |
| 328 | 0.012 |
| 329 | 0.003 |
| 330 | 0.015 |
| 331 | 0.003 |
| 332 | 0.026 |
| 333 | 0.111 |
| 334 | 0.21 |
| 335 | 0.02 |
| 336 | 0.005 |
| 337 | 0.01 |
| 338 | 0.005 |
| 339 | 0.004 |
| 340 | 0.003 |
| 341 | 0.033 |
| 342 | 0.013 |
| 343 | 0.142 |
| 344 | 0.017 |
| 345 | 0.031 |
| 346 | 0.011 |
| 347 | 0.011 |
| 348 | 0.082 |

Variations, modifications, and other implementations of what is described herein will occur to those skilled in the art without departing from the spirit and the essential characteristics of the present teachings. Accordingly, the scope of the present teachings is to be defined not by the preceding illustrative description but instead by the following claims, and all changes that come within the meaning and range of equivalency of the claims are intended to be embraced therein.

Each of the printed publications, including but not limited to patents, patent applications, books, technical papers, trade publications and journal articles described or referenced in this specification are herein incorporated by reference in their entirety and for all purposes.

1. A compound of Formula (I): 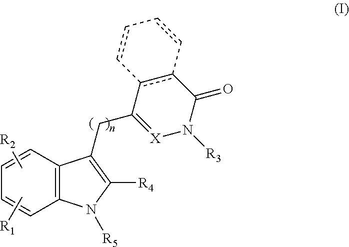
or a pharmaceutically acceptable salt thereof; wherein:
is a single bond or a double bond or is absent;
R₁ and R₂ are each independently H, halogen, OR₆, SO₂R₇, NR₈R₉, or alkyl; wherein
R₆ is H or alkyl;
R₇ is alkyl;
R₈ and R₉ are each independently H, COCH₃ or alkyl;
R₃ is hydroxy, alkyl, alkenyl, alkynyl, aryl, heteroaryl, cycloalkyl or heterocycloalkyl, wherein each alkyl, alkenyl, aryl, heteroaryl, cycloalkyl, heterocycloalkyl is optionally substituted with Rₐ; wherein
Rₐ is alkyl, aryl, heteroaryl, cycloalkyl, alkoxy, phenoxy, halogen, hydroxy, amino, mono- or di-alkylamino, nitro, haloalkyl, haloalkoxy, halophenoxy, CO, carboxamide, sulfonamide or SO₂Me, wherein each alkyl, aryl, heteroaryl is further optionally substituted with H, alkyl, aryl, alkoxy, phenoxy, halogen, hydroxy, haloalkyl, haloalkoxy, halophenoxy or SO₂Me;
R₄ is H or alkyl;
R₅ is CR₁₀R₁₁COOR₁₂, CR₁₀R₁₁CR₁₃NR₁₄R₁₅, COR₁₇, CR₁₀R₁₁CN, CR₁₀R₁₁CR₁₉; wherein
R₁₀ and R₁₁ are each independently H or alkyl;
R₁₂ is H or alkyl;
R₁₃ is O;
R₁₄ and R₁₅ are each independently H, COCH₃, SO₂R₁₆, alkyl, aryl, heteroaryl, cycloalkyl or heterocycloalkyl; wherein
R₁₆ is H, alkyl, aryl, heteroaryl, cycloalkyl or heterocycloalkyl;
R₁₇ is alkyl, aryl, heteroaryl, wherein each of which is optionally substituted with —OH or OR₁₈; wherein
R₁₈ is alkyl;
R₁₉ is alkyl, aryl, heteroaryl, or alkyl optionally substituted with —OH;
X is CH or N; and
n is 0 or 1.

2. The compound of claim 1, wherein R₁ is halogen.

3. The compound of claim 1, wherein R₁ is alkyl.

4. The compound of claim 1, wherein R₁ is SO₂Me.

5. The compound of claim 1, wherein R₂ is halogen.

6. The compound of claim 1, wherein R₂ is alkyl.

7. The compound of claim 1, wherein R₂ is SO₂Me.

8. The compound of claim 1, wherein R₃ is alkyl optionally substituted with alkyl, aryl, heteroaryl, cycloalkyl, alkoxy, halogen, hydroxy, amino, mono- or di-alkylamino, nitro, haloalkyl, haloalkoxy, carboxamide, sulfonamide or SO₂Me.

9. The compound of claim 1, wherein R₃ is aryl optionally substituted with alkyl, aryl, heteroaryl, cycloalkyl, alkoxy, halogen, hydroxy, amino, mono- or di-alkylamino, nitro, haloalkyl, haloalkoxy, carboxamide, sulfonamide or SO₂Me.

10. The compound of claim 1, wherein R₃ is heteroaryl optionally substituted with alkyl, aryl, heteroaryl, cycloalkyl, alkoxy, halogen, hydroxy, amino, mono- or di-alkylamino, nitro, haloalkyl, haloalkoxy, carboxamide, sulfonamide or SO₂Me.

11. The compound of claim 1, wherein R₃ is cycloalkyl optionally substituted with alkyl, aryl, heteroaryl, cycloalkyl, alkoxy, halogen, hydroxy, amino, mono- or di-alkylamino, nitro, haloalkyl, haloalkoxy, carboxamide, sulfonamide or SO₂Me.

12. The compound of claim 1, wherein R₄ is alkyl.

13. The compound of claim 1, wherein R₅ is CH₂COOH.

14. The compound of claim 1, wherein R₅ is CH₂CONHSO₂Me.

15. The compound of claim 1, wherein X is CH.

16. The compound of claim 1, wherein X is N.

17. The compound of claim 1, wherein n is 0.

18. The compound of claim 1, wherein n is 1.

19. The compound of claim 1, wherein the compound is selected from the group consisting of:
2-(5-Chloro-3-(3-(4-chlorobenzyl)-4-oxo-3,4-dihydrophthalazin-1-yl)-2-methyl-1H-indol-1-yl)acetic acid;
2-(5-Chloro-3-(3-(4-chloro-3-fluorobenzyl)-4-oxo-3,4-dihydrophthalazin-1-yl)-2-methyl-1H-indol-1-yl)acetic acid;
2-(5-Chloro-3-(3-(3-fluoro-4-(trifluoromethyl)benzyl)-4-oxo-3,4-dihydrophthalazin-1-yl)-2-methyl-1H-indol-1-yl)acetic acid;
2-(3-(3-(4-Chlorobenzyl)-4-oxo-3,4-dihydropthalazin-1-yl)-5-fluoro-2-methyl-1H-indol-1-yl)acetic acid;
2-(3-(3-(4-Chloro-3-fluorobenzyl)-4-oxo-3,4-dihydrophthalazin-1-yl)-5-fluoro-2-methyl-1H-indol-1-yl)acetic acid;
2-(5-Fluoro-3-(3-(3-fluoro-4-(trifluoromethyl)benzyl)-4-oxo-3,4-dihydrophthalazin-1-yl)-2-methyl-1H-indol-1-yl)acetic acid;
2-(3-(3-(2,4-dichlorobenzyl)-4-oxo-3,4-dihydrophthalazin-1-yl)-5-fluoro-2-methyl-1H-indol-1-yl)acetic acid;
2-(5-Chloro-3-(1-(4-chloro-3-fluorobenzyl)-6-oxo-1,6-dihydropyridazin-3-yl)-2-methyl-1H-indol-1-yl)acetic acid;
2-(5-Chloro-3-(1-(4-chlorobenzyl)-6-oxo-1,6-dihydropyridazin-3-yl)-2-methyl-1H-indol-1-yl)acetic acid;
2-(3-(1-Benzyl-6-oxo-1,6-dihydropyridazin-3-yl)-5-chloro-7-fluoro-2-methyl-1H-indol-1-yl)acetic acid;
2-(3-(3-Benzyl-4-oxo-3,4-dihydrophthalazin-1-yl)-5-chloro-7-fluoro-2-methyl-1H-indol-1-yl)acetic acid;
2-(3-(1-(4-Chloro-3-fluorobenzyl)-6-oxo-1,6-dihydropyridazin-3-yl)-5-fluoro-2-methyl-1H-indol-1-yl)acetic acid;
2-(3-(1-(4-Chlorobenzyl)-6-oxo-1,6-dihydropyridazin-3-yl)-5-fluoro-2-methyl-1H-indol-1-yl)acetic acid;
2-(3-(3-Benzyl-4-oxo-3,4-dihydrophthalazin-1-yl)-7-chloro-2-methyl-5-(methylsulfonyl)-1H-indol-1-yl)acetic acid;
2-(3-(3-Benzyl-4-oxo-3,4-dihydrophthalazin-1-yl)-7-fluoro-2-methyl-5-(methylsulfonyl)-1H-indol-1-yl)acetic acid;
2-(3-(3-Benzyl-4-oxo-3,4-dihydrophthalazin-1-yl)-5-methoxy-2-methyl-1H-indol-1-yl)acetic acid;
2-(3-(1-Benzyl-6-oxo-1,6-dihydropyridazin-3-yl)-7-fluoro-2-methyl-5-(methylsulfonyl)-1H-indol-1-yl)acetic acid;
2-(3-(1-Benzyl-6-oxo-1,6-dihydropyridazin-3-yl)-5-methoxy-2-methyl-1H-indol-1-yl)acetic acid;
2-(3-(3-Isopropyl-4-oxo-3,4-dihydrophthalazin-1-yl)-5-methoxy-2-methyl-1H-indol-1-yl)-acetic acid;
2-(3-((1-(2,4-Difluorobenzyl)-6-oxo-1,6-dihydropyridin-3-yl)methyl)-5-fluoro-2-methyl-1H-indol-1-yl)acetic acid;
2-(3-((1-(2,4-Difluorobenzyl)-6-oxo-1,6-dihydropyridin-3-yl)methyl)-2-methyl-1H-indol-1-yl)acetic acid;
2-(3-((1-Isopropyl-6-oxo-1,6-dihydropyridin-3-yl)methyl-2-methyl-1H-indol-1-yl)acetic acid;
2-(5-Fluoro-2-methyl-3-((6-oxo-1-(2,4,5-trifluorobenzyl)-1,6-dihydropyridin-3-yl)methyl)-1H-indol-1-yl)acetic acid;
2-(2-Methyl-3-((6-oxo-1-(2,4,5-trifluorobenzyl)-1,6-dihydropyridin-3-yl)methyl-1H-indol-1-yl)acetic acid;
2-(3-((1-(2,5-Difluorobenzyl)-6-oxo-1,6-dihydropyridin-3-yl)methyl)-2-methyl-1H-indol-1-yl)acetic acid;
2-(3-((1-(2,5-Difluorobenzyl)-6-oxo-1,6-dihydropyridin-3-yl)methyl)-5-fluoro-2-methyl-1H-indol-1-yl)acetic acid;
2-(5-fluoro-3-((1-isopropyl-6-oxo-1,6-dihydropyridin-3-yl)methyl)-2-methyl-1H-indol-1-yl)acetic acid;
2-(3-((1-(2,3-Difluorobenzyl)-6-oxo-1,6-dihydropyridin-3-yl)methyl)-5-fluoro-2-methyl-1H-indol-1-yl)acetic acid;
2-(3-(1-(2,3-Difluorobenzyl)-6-oxo-1,6-dihydropyridin-3-yl)methyl)-2-methyl-1H-indol-1-yl)acetic acid;
2-(3-((1-(3,4-Difluorobenzyl)-6-oxo-1,6-dihydropyridin-3-yl)methyl)-2-methyl-1H-indol-1-yl)acetic acid;
2-(3-((1-(2-Fluorobenzyl)-6-oxo-1,6-dihydropyridin-3-yl)methyl)-2-methyl-1H-indol-1-yl)-acetic acid;
2-(5-Fluoro-3-((1-(2-fluorobenzyl)-6-oxo-1,6-dihydropyridin-3-yl)methyl)-2-methyl-1H-indol-1-yl)acetic acid;
2-(3-((1-(3,4-Difluorobenzyl)-6-oxo-1,6-dihydropyridin-3-yl)methyl)-5-fluoro-2-methyl-1H-indol-1-yl)acetic acid;
2-(3-((1-(3-Fluorobenzyl)-6-oxo-1,6-dihydropyridin-3-yl)methyl)-2-methyl-1H-indol-1-yl)-acetic acid;
2-(3-((1-(3,5-Difluorobenzyl)-6-oxo-1,6-dihydropyridin-3-yl)methyl)-2-methyl-1H-indol-1-yl)acetic acid;
2-(3-((1-(3,5-Difluorobenzyl)-6-oxo-1,6-dihydropyridin-3-yl)methyl)-5-fluoro-2-methyl-1H-indol-1-yl)acetic acid;
2-(3-((1-(2,6-Difluorobenzyl)-6-oxo-1,6-dihydropyridin-3-yl)methyl)-2-methyl-1H-indol-1-yl)acetic acid;
2-(3-((1-(2,6-Difluorobenzyl)-6-oxo-1,6-dihydropyridin-3-yl)methyl)-5-fluoro-2-methyl-1H-indol-1-yl)acetic acid;
2-(5-Fluoro-3-((1-(3-fluorobenzyl)-6-oxo-1,6-dihydropyridin-3-yl)methyl)-2-methyl-1H-indol-1-yl)acetic acid;
2-(3-((1-(4-Fluorobenzyl)-6-oxo-1,6-dihydropyridin-3-yl)methyl)-2-methyl-1H-indol-1-yl)-acetic acid;
2-(5-Fluoro-3-((1-(4-fluorobenzyl)-6-oxo-1,6-dihydropyridin-3-yl)methyl)-2-methyl-1H-indol-1-yl)acetic acid;
2-(5-Chloro-3-((1-(2,4-difluorobenzyl)-6-oxo-1,6-dihydropyridin-3-yl)methyl)-2-methyl-1H-indol-1-yl)acetic acid;
2-(5-Chloro-3-((1-(2,3-difluorobenzyl)-6-oxo-1,6-dihydropyridin-3-yl)methyl)-2-methyl-1H-indol-1-yl)acetic acid;
2-(5-Chloro-2-methyl-3-((6-oxo-1-(2,4,5-trifluorobenzyl)-1,6-dihydropyridin-3-yl)methyl)-1H-indol-1-yl)acetic acid;
2-(5-Chloro-3-((1-(3-fluorobenzyl)-6-oxo-1,6-dihydropyridin-3-yl)methyl)-2-methyl-1H-indol-1-yl)acetic acid;
2-(5-Fluoro-2-methyl-3-((6-oxo-1-(2,2,2-trifluoroethyl)-1,6-dihydropyridin-3-yl)methyl-1H-indol-1-yl)acetic acid;
2-(3-(3-Benzyl-4-oxo-3,4-dihydrophthalazin-1-yl)-2-methyl-1H-indol-1-yl)acetamide;
2-(3-(3-Benzyl-4-oxo-3,4-dihydrophthalazin-1-yl)-2-methyl-1H-indol-1-yl)-N,N-dimethylacetamide;
2-Benzyl-4-(2-methyl-1-(2-oxo-2-(pyrrolidin-1-yl)ethyl)-1H-indol-3-yl)phthalazin-1(2H)-one;
2-(3-(3-Benzyl-4-oxo-3,4-dihydrophthalazin-1-yl)-2-methyl-1H-indol-1-yl)-N-(methyl-sulfonyl)acetamide;
4-(1-((2H-Tetrazol-5-yl)methyl)-2-methyl-1H-indol-3-yl)-2-benzylphthalazin-1(2H)-one;
2-Benzyl-4-(1-(2-hydroxyethyl)-2-methyl-1H-indol-3-yl)phthalazin-1(2H)-one;
2-(5-Chloro-2-methyl-3-((6-oxo-1-(2,2,2-trifluoroethyl)-1,6-dihydropyridin-3-yl)methyl)-1H-indol-1-yl)acetic acid;
2-(5-Fluoro-2-methyl-3-((6-oxo-1-(4,4,4-trifluorobutyl)-1,6-dihydropyridin-3-yl)methyl)-1H-indol-1-yl)acetic acid;
2-(4-Acetamido-3-((1-(2,4-difluorobenzyl)-6-oxo-1,6-dihydropyridazin-3-yl)methyl)-2-methyl-1H-indol-1-yl)acetic acid;
2-(3-((1-(2,4-Difluorobenzyl)-6-oxo-1,4,5,6-tetrahydropyridazin-3-yl)methyl)-5-fluoro-2-methyl-1H-indol-1-yl)acetic acid;
2-(5-Fluoro-2-methyl-3-((6-oxo-1-(2,2,2-trifluoroethyl)-1,4,5,6-tetrahydropyridazin-3-yl)-methyl)-1H-indol-1-yl)acetic acid;
2-(5-Fluoro-2-methyl-3-((6-oxo-1-(4,4,4-trifluorobutyl)-1,4,5,6-tetrahydropyridazin-3-yl)-methyl)-1H-indol-1-yl)acetic acid;
2-(5-Chloro-3-(3-(2,5-difluorobenzyl)-4-oxo-3,4-dihydrophthalazin-1-yl)-2-methyl-1H-indol-1-yl)acetic acid;
2-(5-Chloro-2-methyl-3-(4-oxo-3-(2,4,5-trifluorobenzyl)-3,4-dihydrophthalazin-1-yl)-1H-indol-1-yl)acetic acid;
2-(5-Chloro-3-(3-(2,4-difluorobenzyl)-4-oxo-3,4-dihydrophthalazin-1-yl)-2-methyl-1H-indol-1-yl)acetic acid;
2-(3-(3-(2,5-Difluorobenzyl)-4-oxo-3,4-dihydrophthalazin-1-yl)-5-fluoro-2-methyl-1H-indol-1-yl)acetic acid;
2-(5-Fluoro-2-methyl-3-(4-oxo-3-(2,4,5-trifluorobenzyl)-3,4-dihydrophthalazin-1-yl)-1H-indol-1-yl)acetic acid;
2-(3-(3-(2,4-Difluorobenzyl)-4-oxo-3,4-dihydrophthalazin-1-yl)-5-fluoro-2-methyl-1H-indol-1-yl)acetic acid;
2-(5-Fluoro-2-methyl-3-(3-(4-(methylsulfonyl)benzyl)-4-oxo-3,4-dihydrophthalazin-1-yl)-1H-indol-1-yl)acetic acid;
2-(5-Chloro-2-methyl-3-(1-(4-(methylsulfonyl)benzyl)-6-oxo-1,6-dihydropyridazin-3-yl)-1H-indol-1-yl)acetic acid;
2-(5-Chloro-3-(1-(2,5-difluorobenzyl)-6-oxo-1,6-dihydropyridazin-3-yl)-2-methyl-1H-indol-1-yl)acetic acid;
2-(5-Chloro-3-(1-(2,4-difluorobenzyl)-6-oxo-1,6-dihydropyridazin-3-yl)-2-methyl-1H-indol-1-yl)acetic acid;
2-(5-Chloro-2-methyl-3-(6-oxo-1-(2,4,5-trifluorobenzyl)-1,6-dihydropyridazin-3-yl)-1H-indol-1-yl)acetic acid;
2-(5-Fluoro-2-methyl-3-(6-oxo-1-(2,4,5-trifluorobenzyl)-1,6-dihydropyridazin-3-yl)-1H-indol-1-yl)acetic acid;
2-(3-(1-(2,4-Difluorobenzyl)-6-oxo-1,6-dihydropyridazin-3-yl)-5-fluoro-2-methyl-1H-indol-1-yl)acetic acid;
2-(3-(1-(2,5-Difluorobenzyl)-6-oxo-1,6-dihydropyridazin-3-yl)-5-fluoro-2-methyl-1H-indol-1-yl)acetic acid;
2-(5-Fluoro-2-methyl-3-(1-(4-(methylsulfonyl)benzyl)-6-oxo-1,6-dihydropyridazin-3-yl)-1H-indol-1-yl)acetic acid;
2-(3-((1-(2-Fluorobenzyl)-6-oxo-1,6-dihydropyridazin-3-yl)methyl)-2-methyl-1H-indol-1-yl)acetic acid;
2-(3-((1-(3-Fluorobenzyl)-6-oxo-1,6-dihydropyridazin-3-yl)methyl)-2-methyl-1H-indol-1- yl)acetic acid;
2-(3-((1-(4-Fluorobenzyl)-6-oxo-1,6-dihydropyridazin-3-yl)methyl)-2-methyl-1H-indol-1-yl)acetic acid;
2-(3-((1-Benzyl-6-oxo-1,6-dihydropyridazin-3-yl)methyl)-2-methyl-1H-indol-1-yl)acetic acid;
2-(3-((1-Benzyl-6-oxo-1,4,5,6-tetrahydropyridazin-3-yl)methyl)-5-fluoro-2-methyl-1H-indol-1-yl)acetic acid;
2-(3-((1-(2,3-Difluorobenzyl)-6-oxo-1,6-dihydropyridazin-3-yl)methyl)-2-methyl-1H-indol-1-yl)acetic acid;
2-(3-((1-Benzyl-6-oxo-1,6-dihydropyridazin-3-yl)methyl)-5-chloro-2-methyl-1H-indol-1-yl)acetic acid;
2-(5-Chloro-2-methyl-3-((6-oxo-1-(4-(trifluoromethyl)benzyl)-1,6-dihydropyridazin-3-yl)-methyl)-1H-indol-1-yl)acetic acid;
2-(5-Fluoro-2-methyl-3-((6-oxo-1-(4-(trifluoromethyl)benzyl)-1,6-dihydropyridazin-3-yl)-methyl)-1H-indol-1-yl)acetic acid;
2-(3-((1-(2,4-Difluorobenzyl)-6-oxo-1,6-dihydropyridazin-3-yl)methyl)-5-fluoro-2-methyl-1H-indol-1-yl)acetic acid;
2-(3-((3-Benzyl-4-oxo-3,4-dihydrophthalazin-1-yl)methyl)-5-chloro-2-methyl-1H-indol-1-yl)acetic acid;
2-(3-((2-Benzyl-1-oxo-1,2-dihydroisoquinolin-4-yl)methyl)-5-fluoro-2-methyl-1H-indol-1-yl)acetic acid;
2-(3-((2-Benzyl-1-oxo-1,2-dihydroisoquinolin-4-yl)methyl)-2-methyl-1H-indol-1-yl)acetic acid;
2-(3-((2-Benzyl-1-oxo-1,2-dihydroisoquinolin-4-yl)methyl)-5-chloro-2-methyl-1H-indol-1-yl)acetic acid;
2-(3-(2-Benzyl-1-oxo-1,2,5,6,7,8-hexahydroisoquinolin-4-yl)-5-fluoro-2-methyl-1H-indol-1-yl)acetic acid;
2-(5-Fluoro-2-methyl-3-(1-oxo-2-(4,4,4-trifluorobutyl)-1,2,5,6,7,8-hexahydroisoquinolin-4-yl)-1H-indol-1-yl)acetic acid;
2-(5-Fluoro-2-methyl-3-(1-oxo-2-(2,2,2-trifluoroethyl)-1,2,5,6,7,8-hexahydroisoquinolin-4-yl)-1H-indol-1-yl)acetic acid;
2-(5-Fluoro-3-(2-isopropyl-1-oxo-1,2,5,6,7,8-hexahydroisoquinolin-4-yl)-2-methyl-1H-indol-1-yl)acetic acid;
2-(5-Fluoro-3-(2-(2-hydroxy-2-methylpropyl)-1-oxo-1,2,5,6,7,8-hexahydroisoquinolin-4-yl)-2-methyl-1H-indol-1-yl)acetic acid;
2-(5-Fluoro-2-methyl-3-(1-oxo-2-phenethyl-1,2,5,6,7,8-hexahydroisoquinolin-4-yl)-1H-indol-1-yl)acetic acid;
2-(3-(2-(2,4-Difluorobenzyl)-1-oxo-1,2,5,6,7,8-hexahydroisoquinolin-4-yl)-5-fluoro-2-methyl-1H-indol-1-yl)acetic acid;
2-(5-Fluoro-2-methyl-3-(1-oxo-2-(pyridin-2-ylmethyl)-1,2-dihydroisoquinolin-4-yl)-1H-indol-1-yl)acetic acid;
2-(5-Fluoro-2-methyl-3-(1-oxo-2-(4,4,4-trifluoro-3-(trifluoromethyl)butyl)-1,2-dihydro-isoquinolin-4-yl)-1H-indol-1-yl)acetic acid;
2-(5-Chloro-3-(3-(2,3-difluorobenzyl)-4-oxo-3,4-dihydrophthalazin-1-yl)-2-methyl-1H-indol-1-yl)acetic acid;
2-(5-Chloro-3-(3-(2-fluorobenzyl)-4-oxo-3,4-dihydrophthalazin-1-yl)-2-methyl-1H-indol-1-yl)acetic acid;
2-(5-Chloro-3-(3-((5-fluorobenzo[d]thiazol-2-yl)methyl)-4-oxo-3,4-dihydrophthalazin-1-yl)-2-methyl-1H-indol-1-yl)acetic acid;
2-(5-Chloro-2-methyl-3-(4-oxo-3-((5-(trifluoromethyl)benzo[d]thiazol-2-yl)methyl)-3,4-dihydrophthalazin-1-yl)-1H-indol-1-yl)acetic acid;
2-(5-Chloro-3-(3-(2,6-difluorobenzyl)-4-oxo-3,4-dihydrophthalazin-1-yl)-2-methyl-1H-indol-1-yl)acetic acid;
2-(5-Chloro-2-methyl-3-(4-oxo-3-(4-(trifluoromethoxy)benzyl)-3,4-dihydrophthalazin-1-yl)-1H-indol-1-yl)acetic acid;
2-(5-Chloro-2-methyl-3-(4-oxo-3-(quinolin-2-ylmethyl)-3,4-dihydrophthalazin-1-yl)-1H-indol-1-yl)acetic acid;
2-(5-Chloro-2-methyl-3-(3-((2-methylquinolin-4-yl)methyl)-4-oxo-3,4-dihydrophthalazin-1-yl)-1H-indol-1-yl)acetic acid;
2-(5-Chloro-2-methyl-3-(3-methyl-4-oxo-3,4-dihydrophthalazin-1-yl)-1H-indol-1-yl)-acetic acid;
2-(5-Chloro-3-(3-ethyl-4-oxo-3,4-dihydrophthalazin-1-yl)-2-methyl-1H-indol-1-yl)acetic acid;
2-(5-Chloro-3-(3-isopropyl-4-oxo-3,4-dihydrophthalazin-1-yl)-2-methyl-1H-indol-1-yl)acetic acid;
2-(5-Chloro-3-(3-(cyclopropylmethyl)-4-oxo-3,4-dihydrophthalazin-1-yl)-2-methyl-1H-indol-1-yl)acetic acid;
2-(5-Chloro-2-methyl-3-(4-oxo-3-(2,2,2-trifluoroethyl)-3,4-dihydrophthalazin-1-yl)-1H-indol-1-yl)acetic acid;
2-(3-(3-(Benzo[d]thiazol-2-ylmethyl)-4-oxo-3,4-dihydrophthalazin-1-yl)-5-fluoro-2-methyl-1H-indol-1-yl)acetic acid;
2-(5-Fluoro-3-(3-(4-fluorobenzyl)-4-oxo-3,4-dihydrophthalazin-1-yl)-2-methyl-1H-indol-1-yl)acetic acid;
2-(3-(3-(2,3-Difluorobenzyl)-4-oxo-3,4-dihydrophthalazin-1-yl)-5-fluoro-2-methyl-1H-indol-1-yl)acetic acid;
2-(5-Fluoro-3-(3-(2-fluorobenzyl)-4-oxo-3,4-dihydrophthalazin-1-yl)-2-methyl-1H-indol-1-yl)acetic acid;
2-(5-Fluoro-3-(3-((5-fluorobenzo[d]thiazol-2-yl)methyl)-4-oxo-3,4-dihydrophthalazin-1-yl)-2-methyl-1H-indol-1-yl)acetic acid;
2-(5-Fluoro-2-methyl-3-(4-oxo-3-((5-(trifluoromethyl)benzo[d]thiazol-2-yl)methyl)-3,4-dihydrophthalazin-1-yl)-1H-indol-1-yl)acetic acid;
2-(3-(3-(2,6-Difluorobenzyl)-4-oxo-3,4-dihydrophthalazin-1-yl)-5-fluoro-2-methyl-1H-indol-1-yl)acetic acid;
2-(5-Fluoro-2-methyl-3-(4-oxo-3-(4-(trifluoromethoxy)benzyl)-3,4-dihydrophthalazin-1-yl)-1H-indol-1-yl)acetic acid;
2-(5-Fluoro-2-methyl-3-(4-oxo-3-(quinolin-2-ylmethyl)-3,4-dihydrophthalazin-1-yl)-1H- indol-1-yl)acetic acid;
2-(5-Fluoro-2-methyl-3-(3-((2-methylquinolin-4-yl)methyl)-4-oxo-3,4-dihydrophthalazin-1-yl)-1H-indol-1-yl)acetic acid;
2-(5-Fluoro-2-methyl-3-(3-methyl-4-oxo-3,4-dihydrophthalazin-1-yl)-1H-indol-1-yl)acetic acid;
2-(3-(3-Ethyl-4-oxo-3,4-dihydrophthalazin-1-yl)-5-fluoro-2-methyl-1H-indol-1-yl)acetic acid;
2-(3-(3-(Cyclopropylmethyl)-4-oxo-3,4-dihydrophthalazin-1-yl)-5-fluoro-2-methyl-1H-indol-1-yl)acetic acid;
2-(3-(3-Cyclopropyl-4-oxo-3,4-dihydrophthalazin-1-yl)-5-fluoro-2-methyl-1H-indol-1-yl)acetic acid;
2-(5-Fluoro-2-methyl-3-(4-oxo-3-(2,2,2-trifluoroethyl)-3,4-dihydrophthalazin-1-yl)-1H-indol-1-yl)acetic acid;
2-(5-Fluoro-2-methyl-3-(4-oxo-3-(4,4,4-trifluorobutyl)-3,4-dihydrophthalazin-1-yl)-1H-indol-1-yl)acetic acid;
2-(5-Fluoro-2-methyl-3-(3-neopentyl-4-oxo-3,4-dihydrophthalazin-1-yl)-1H-indol-1-yl)-acetic acid;
2-(5-Fluoro-2-methyl-3-(4-oxo-3-(3,3,3-trifluoropropyl)-3,4-dihydrophthalazin-1-yl)-1H-indol-1-yl)acetic acid;
2-(3-(3-(2-Ethyl-2-hydroxybutyl)-4-oxo-3,4-dihydrophthalazin-1-yl)-5-fluoro-2-methyl-1H-indol-1-yl)acetic acid;
2-(5-Fluoro-3-(3-(2-hydroxy-2-methylpropyl)-4-oxo-3,4-dihydrophthalazin-1-yl)-2-methyl-1H-indol-1-yl)acetic acid;
2-(5-Fluoro-2-methyl-3-(3-(3-methylbut-2-enyl)-4-oxo-3,4-dihydrophthalazin-1-yl)-1H-indol-1-yl)acetic acid;
2-(5-Fluoro-2-methyl-3-(4-oxo-3-(3,3,3-trifluoro-2-hydroxy-2-(trifluoromethyl)propyl)-3,4-dihydrophthalazin-1-yl)-1H-indol-1-yl)acetic acid;
2-(5-Fluoro-3-(3-(3-hydroxy-3-methylbutyl)-4-oxo-3,4-dihydrophthalazin-1-yl)-2-methyl-1H-indol-1-yl)acetic acid;
2-(5-Fluoro-2-methyl-3-(4-oxo-3-(2-oxobutyl)-3,4-dihydrophthalazin-1-yl)-1H-indol-1-yl)-acetic acid;
2-(5-Fluoro-2-methyl-3-(4-oxo-3-(pyridin-4-ylmethyl)-3,4-dihydrophthalazin-1-yl)-1H-indol-1-yl)acetic acid;
2-(5-Fluoro-2-methyl-3-(4-oxo-3-(pyridin-3-ylmethyl)-3,4-dihydrophthalazin-1-yl)-1H-indol-1-yl)acetic acid;
2-(5-Fluoro-2-methyl-3-(4-oxo-3-(pyridin-2-ylmethyl)-3,4-dihydrophthalazin-1-yl)-1H-indol-1-yl)acetic acid;
2-(5-Fluoro-2-methyl-3-(4-oxo-3-(4,4,4-trifluoro-3-(trifluoromethyl)butyl)-3,4-dihydrophthalazin-1-yl)-1H-indol-1-yl)acetic acid;
2-(5-Fluoro-3-(3-((3-fluoropyridin-4-yl)methyl)-4-oxo-3,4-dihydrophthalazin-1-yl)-2-methyl-1H-indol-1-yl)acetic acid;
2-(5-Chloro-3-(1-(4-fluorobenzyl)-6-oxo-1,6-dihydropyridazin-3-yl)-2-methyl-1H-indol-1-yl)acetic acid;
2-(5-Chloro-3-(1-(2-(4-chlorophenoxy)ethyl)-6-oxo-1,6-dihydropyridazin-3-yl)-2-methyl-1H-indol-1-yl)acetic acid;
2-(3-(1-(Benzo[d]thiazol-2-ylmethyl)-6-oxo-1,6-dihydropyridazin-3-yl)-5-chloro-2-methyl-1H-indol-1-yl)acetic acid;
2-(5-Chloro-3-(1-(4-fluoro-2-(trifluoromethyl)benzyl)-6-oxo-1,6-dihydropyridazin-3-yl)-2-methyl-1H-indol-1-yl)acetic acid;
2-(5-Chloro-3-(1-(3-fluoro-2-(trifluoromethyl)benzyl)-6-oxo-1,6-dihydropyridazin-3-yl)-2-methyl-1H-indol-1-yl)acetic acid;
2-(5-Chloro-2-methyl-3-(6-oxo-1-(quinolin-2-ylmethyl)-1,6-dihydropyridazin-3-yl)-1H-indol-1-yl)acetic acid;
2-(5-chloro-2-methyl-3-(1-((2-methylquinolin-4-yl)methyl)-6-oxo-1,6-dihydropyridazin-3-yl)-1H-indol-1-yl)acetic acid;
2-(5-Chloro-2-methyl-3-(1-(4-methylbenzyl)-6-oxo-1,6-dihydropyridazin-3-yl)-1H-indol-1-yl)acetic acid;
2-(5-Chloro-3-(1-(4-isopropylbenzyl)-6-oxo-1,6-dihydropyridazin-3-yl)-2-methyl-1H-indol-1-yl)acetic acid;
2-(5-chloro-3-(1-(4-methoxybenzyl)-6-oxo-1,6-dihydropyridazin-3-yl)-2-methyl-1H-indol-1-yl)acetic acid;
2-(5-Chloro-2-methyl-3-(1-methyl-6-oxo-1,6-dihydropyridazin-3-yl)-1H-indol-1-yl)-acetic acid;
2-(5-Chloro-3-(1-ethyl-6-oxo-1,6-dihydropyridazin-3-yl)-2-methyl-1H-indol-1-yl)acetic acid;
2-(5-Fluoro-3-(1-(4-fluorobenzyl)-6-oxo-1,6-dihydropyridazin-3-yl)-2-methyl-1H-indol-1-yl)acetic acid;
2-(3-(1-(2-(4-Chlorophenoxy)ethyl)-6-oxo-1,6-dihydropyridazin-3-yl)-5-fluoro-2-methyl-1H-indol-1-yl)acetic acid;
2-(3-(1-(Benzo[d]thiazol-2-ylmethyl)-6-oxo-1,6-dihydropyridazin-3-yl)-5-fluoro-2-methyl-1H-indol-1-yl)acetic acid;
2-(5-Fluoro-3-(1-(3-fluorobenzyl)-6-oxo-1,6-dihydropyridazin-3-yl)-2-methyl-1H-indol-1-yl)acetic acid;
2-(5-Fluoro-3-(1-(4-fluoro-2-(trifluoromethyl)benzyl)-6-oxo-1,6-dihydropyridazin-3-yl)-2-methyl-1H-indol-1-yl)acetic acid;
2-(5-Fluoro-3-(1-(3-fluoro-2-(trifluoromethyl)benzyl)-6-oxo-1,6-dihydropyridazin-3-yl)-2-methyl-1H-indol-1-yl)acetic acid;
2-(5-fluoro-2-methyl-3-(6-oxo-1-(quinolin-2-ylmethyl)-1,6-dihydropyridazin-3-yl)-1H-indol-1-yl)acetic acid;
2-(5-Fluoro-2-methyl-3-(1-((2-methylquinolin-4-yl)methyl)-6-oxo-1,6-dihydropyridazin-3-yl)-1H-indol-1-yl)acetic acid;
2-(5-Fluoro-2-methyl-3-(1-methyl-6-oxo-1,6-dihydropyridazin-3-yl)-1H-indol-1-yl)acetic acid;
2-(3-(1-Ethyl-6-oxo-1,6-dihydropyridazin-3-yl)-5-fluoro-2-methyl-1H-indol-1-yl)acetic acid;
2-(3-(1-(Cyclopropylmethyl)-6-oxo-1,6-dihydropyridazin-3-yl)-5-fluoro-2-methyl-1H-indol-1-yl)acetic acid;
2-(5-Fluoro-2-methyl-3-(6-oxo-1-(2,2,2-trifluoroethyl)-1,6-dihydropyridazin-3-yl)-1H-indol-1-yl)acetic acid;
2-(5-Fluoro-2-methyl-3-(6-oxo-1-(4,4,4-trifluorobutyl)-1,6-dihydropyridazin-3-yl)-1H-indol-1-yl)acetic acid;
2-(5-Fluoro-2-methyl-3-(6-oxo-1-(3,3,3-trifluoropropyl)-1,6-dihydropyridazin-3-yl)-1H-indol-1-yl)acetic acid;
2-(5-Fluoro-2-methyl-3-(1-neopentyl-6-oxo-1,6-dihydropyridazin-3-yl)-1H-indol-1-yl)acetic acid;
2-(3-(3-(4-Fluorobenzyl)-4-oxo-3,4-dihydrophthalazin-1-yl)-2-methyl-1H-indol-1-yl)acetic acid;
2-(3-(3-(Benzo[d]thiazol-2-ylmethyl)-4-oxo-3,4-dihydrophthalazin-1-yl)-2-methyl-1H-indol-1-yl)acetic acid;
2-(2-Methyl-3-(3-(4-(methylsulfonyl)benzyl)-4-oxo-3,4-dihydrophthalazin-1-yl)-1H-indol-1-yl)acetic acid;
2-(2-Methyl-3-(4-oxo-3-(quinolin-2-ylmethyl)-3,4-dihydrophthalazin-1-yl)-1H-indol-1-acetic acid;
2-(2-methyl-3-(4-oxo-3-(4-(trifluoromethoxy)benzyl)-3,4-dihydrophthalazin-1-yl)-1H-indol-1-yl)-1-yl)acetic acid;
2-(2-Methyl-3-(4-oxo-3-(4-(trifluoromethyl)benzyl)-3,4-dihydrophthalazin-1-yl)-1H-indol-1-yl)acetic acid;
2-(3-(3-(2,6-Difluorobenzyl)-4-oxo-3,4-dihydrophthalazin-1-yl)-2-methyl-1H-indol-1-yl)acetic acid;
2-(2-Methyl-3-(3-(4-methylbenzyl)-4-oxo-3,4-dihydrophthalazin-1-yl)-1H-indol-1-yl)-acetic acid;
2-(2-Methyl-3-(3-methyl-4-oxo-3,4-dihydrophthalazin-1-yl)-1H-indol-1-yl)acetic acid;
2-(3-(3-Ethyl-4-oxo-3,4-dihydrophthalazin-1-yl)-2-methyl-1H-indol-1-yl)acetic acid;
2-(3-(3-(Cyclopropylmethyl)-4-oxo-3,4-dihydrophthalazin-1-yl)-2-methyl-1H-indol-1-yl)acetic acid;
2-(2-Methyl-3-(4-oxo-3-(2,2,2-trifluoroethyl)-3,4-dihydrophthalazin-1-yl)-1H-indol-1-yl)-acetic acid;
2-(3-(3-cyclopropyl-4-oxo-3,4-dihydrophthalazin-1-yl)-2-methyl-1H-indol-1-yl)acetic acid;
2-(3-(3-Cyclopentyl-4-oxo-3,4-dihydrophthalazin-1-yl)-2-methyl-1H-indol-1-yl)acetic acid;
2-(3-(1-Benzyl-6-oxo-1,6-dihydropyridazin-3-yl)-2-methyl-1H-indol-1-yl)acetic acid;
2-(3-(1-Isopropyl-6-oxo-1,6-dihydropyridazin-3-yl)-2-methyl-1H-indol-1-yl)acetic acid;
2-(3-(1-Ethyl-6-oxo-1,6-dihydropyridazin-3-yl)-2-methyl-1H-indol-1-yl)acetic acid;
2-(2-Methyl-3-(1-methyl-6-oxo-1,6-dihydropyridazin-3-yl)-1H-indol-1-yl)acetic acid;
2-(3-(1-(Cyclopropylmethyl)-6-oxo-1,6-dihydropyridazin-3-yl)-2-methyl-1H-indol-1-yl)-acetic acid;
2-(2-Methyl-3-(6-oxo-1-(2,2,2-trifluoroethyl)-1,6-dihydropyridazin-3-yl)-1H-indol-1-yl)-acetic acid;
Methyl 2-(3-(1-(2,4-difluorobenzyl)-6-oxo-1,6-dihydropyridazin-3-yl)-2-methyl-1H-indol-1-yl)acetate;
2-(2-Methyl-3-(6-oxo-1-(2,4,5-trifluorobenzyl)-1,6-dihydropyridazin-3-yl)-1H-indol-1-yl)-acetic acid;
2-(3-(1-(2,3-Difluorobenzyl)-6-oxo-1,6-dihydropyridazin-3-yl)-2-methyl-1H-indol-1-yl)-acetic acid;
2-(3-(1-(4-Fluorobenzyl)-6-oxo-1,6-dihydropyridazin-3-yl)-2-methyl-1H-indol-1-yl)acetic acid;
2-(3-(1-(2-Fluorobenzyl)-6-oxo-1,6-dihydropyridazin-3-yl)-2-methyl-1H-indol-1-yl)acetic acid;
2-(3-(1-Benzyl-6-oxo-1,6-dihydropyridazin-3-yl)-2,5-dimethyl-1H-indol-1-yl)acetic acid;
2-(3-(3-Benzyl-4-oxo-3,4-dihydrophthalazin-1-yl)-2,5-dimethyl-1H-indol-1-yl)acetic acid;
2-(5-Fluoro-2-methyl-3-((6-oxo-1-(2,4,5-trifluorobenzyl)-1,6-dihydropyridazin-3-yl)-methyl)-1H-indol-1-yl)acetic acid;
2-(5-Fluoro-2-methyl-3-((6-oxo-1-(pyridin-4-ylmethyl)-1,6-dihydropyridazin-3-yl)methyl)-1H-indol-1-yl)acetic acid;
2-(5-Fluoro-2-methyl-3-((6-oxo-1-(pyridin-3-ylmethyl)-1,6-dihydropyridazin-3-yl)methyl)-1H-indol-1-yl)acetic acid;
2-(5-Fluoro-2-methyl-3-((6-oxo-1-(pyridin-2-ylmethyl)-1,6-dihydropyridazin-3-yl)methyl)-1H-indol-1-yl)acetic acid;
(5-Fluoro-3-{[1-(2-hydroxy-2-methylpropyl)-6-oxo-1,6-dihydropyridazin-3-yl]methyl}-2-methyl-1H-indol-1-yl)acetic acid;
2-(5-Chloro-2-methyl-3-(4-oxo-3-(4-(trifluoromethyl)benzyl)-3,4-dihydrophthalazin-1-yl)-1H-indol-1-yl)acetic acid;
2-(5-Chloro-2-methyl-3-(4-oxo-3-(2-(trifluoromethyl)benzyl)-3,4-dihydrophthalazin-1-yl)-1H-indol-1-yl)acetic acid;
2-(5-Chloro-2-methyl-3-(4-oxo-3-(3-(trifluoromethyl)benzyl)-3,4-dihydrophthalazin-1-yl)-1H-indol-1-yl)acetic acid;
[5-fluoro-2-methyl-3-({6-oxo-1-[3,3,3-trifluoro-2-hydroxy-2-(trifluoromethyl)propyl]-1,6-dihydro-pyridazin-3-yl}methyl)-1H-indol-1-yl]acetic acid;
2-(5-Fluoro-2-methyl-3-(4-oxo-3-(2-(trifluoromethyl)benzyl)-3,4-dihydrophthalazin-1-yl)-1H-indol-1-yl)acetic acid;
2-(5-Fluoro-2-methyl-3-(4-oxo-3-(3-(trifluoromethyl)benzyl)-3,4-dihydrophthalazin-1-yl)-1H-indol-1-yl)acetic acid;
2-(5-Fluoro-2-methyl-3-(4-oxo-3-(4-(trifluoromethyl)benzyl)-3,4-dihydrophthalazin-1-yl)-1H-indol-1-yl)acetic acid;
2-(5-Fluoro-2-methyl-3-(4-oxo-3-phenyl-3,4-dihydrophthalazin-1-yl)-1H-indol-1-yl)acetic acid;
2-(5-Fluoro-2-methyl-3-(4-oxo-3-(4-(trifluoromethyl)phenyl)-3,4-dihydrophthalazin-1-yl)-1H-indol-1-yl)acetic acid;
2-(5-Fluoro-2-methyl-3-(4-oxo-3-(3-(trifluoromethyl)phenyl)-3,4-dihydrophthalazin-1-yl)-1H-indol-1-yl)acetic acid;
2-(3-(3-((5-Chlorobenzo[d]thiazol-2-yl)methyl)-4-oxo-3,4-dihydrophthalazin-1-yl)-5-fluoro-2-methyl-1H-indol-1-yl)acetic acid;
2-(5-Chloro-3-(3-((5-chlorobenzo[d]thiazol-2-yl)methyl)-4-oxo-3,4-dihydrophthalazin-1-yl)-2-methyl-1H-indol-1-yl)acetic acid;
2-(3-(3-(2-(4-Chlorophenoxy)ethyl)-4-oxo-3,4-dihydrophthalazin-1-yl)-5-fluoro-2-methyl-1H-indol-1-yl)acetic acid;
2-(5-Chloro-2-methyl-3-(6-oxo-1-(2-(trifluoromethyl)benzyl)-1,6-dihydropyridazin-3-yl)-1H-indol-1-yl)acetic acid;
2-(5-Chloro-2-methyl-3-(6-oxo-1-(3-(trifluoromethyl)benzyl)-1,6-dihydropyridazin-3-yl)-1H-indol-1-yl)acetic acid;
2-(5-Chloro-2-methyl-3-(6-oxo-1-(4-(trifluoromethyl)benzyl)-1,6-dihydropyridazin-3-yl)-1H-indol-1-yl)acetic acid;
2-(3-(1-Benzyl-6-oxo-1,6-dihydropyridazin-3-yl)-2-methyl-5-(methylsulfonyl)-1H-indol-1-yl)acetic acid;
2-(3-(3-Benzyl-4-oxo-3,4-dihydrophthalazin-1-yl)-2-methyl-5-(methylsulfonyl)-1H-indol-1-yl)acetic acid;
2-(5-Fluoro-2-methyl-3-(6-oxo-1-(2-(trifluoromethyl)benzyl)-1,6-dihydropyridazin-3-yl)-1H-indol-1-yl)acetic acid;
2-(5-Fluoro-2-methyl-3-(6-oxo-1-(3-(trifluoromethyl)benzyl)-1,6-dihydropyridazin-3-yl)-1H-indol-1-yl)acetic acid;
2-(5-Fluoro-2-methyl-3-(6-oxo-1-(3-(trifluoromethyl)benzyl)-1,6-dihydropyridazin-3-yl)-1H-indol-1-yl)acetic acid;
2-(3-(1-Benzyl-6-oxo-1,6-dihydropyridazin-3-yl)-5-bromo-2-methyl-1H-indol-1-yl)acetic acid;
2-(5-Fluoro-2-methyl-3-(3-(2-methyl-2-phenoxypropyl)-4-oxo-3,4-dihydrophthalazin-1-yl)-1H-indol-1-yl)acetic acid;
2-(5-Chloro-2-methyl-3-(4-oxo-3-(2-phenoxyethyl)-3,4-dihydrophthalazin-1-yl)-1H-indol-1-yl)acetic acid;
2-(5-Fluoro-2-methyl-3-(4-oxo-3-(2-phenoxyethyl)-3,4-dihydrophthalazin-1-yl)-1H-indol-1-yl)acetic acid;
2-(2-Methyl-3-(4-oxo-3-(2-phenoxyethyl)-3,4-dihydrophthalazin-1-yl)-1H-indol-1-yl)acetic acid;
2-(3-(3-(4-Fluorophenethyl)-4-oxo-3,4-dihydrophthalazin-1-yl)-2-methyl-1H-indol-1-yl)-acetic acid;
2-(2-Methyl-3-(4-oxo-3-phenethyl-3,4-dihydrophthalazin-1-yl)-1H-indol-1-yl)acetic acid;
2-(5-Fluoro-2-methyl-3-(4-oxo-3-phenethyl-3,4-dihydrophthalazin-1-yl)-1H-indol-1-yl)-acetic acid;
2-(5-Fluoro-3-(3-(4-fluorophenethyl)-4-oxo-3,4-dihydrophthalazin-1-yl)-2-methyl-1H-indol-1-yl)acetic acid;
2-(2-Methyl-3-(6-oxo-1-phenethyl-1,6-dihydropyridazin-3-yl)-1H-indol-1-yl)acetic acid;
2-(3-(1-(4-Fluorophenethyl)-6-oxo-1,6-dihydropyridazin-3-yl)-2-methyl-1H-indol-1-yl)-acetic acid;
2-(5-Fluoro-2-methyl-3-(1-(2-methyl-2-phenoxypropyl)-6-oxo-1,6-dihydropyridazin-3-yl)-1H-indol-1-yl)acetic acid;
2-(3-(1-Benzyl-6-oxo-1,6-dihydropyridazin-3-yl)-7-chloro-5-fluoro-2-methyl-1H-indol-1-yl)acetic acid;
2-(7-Chloro-5-fluoro-3-(1-isopropyl-6-oxo-1,6-dihydropyridazin-3-yl)-2-methyl-1H-indol-1-yl)acetic acid;
2-(3-(1-Benzyl-6-oxo-1,6-dihydropyridazin-3-yl)-5,7-difluoro-2-methyl-1H-indol-1-yl)acetic acid;
2-(3-(1-Benzyl-6-oxo-1,6-dihydropyridazin-3-yl)-5,7-dichloro-2-methyl-1H-indol-1-yl)-acetic acid;
2-(5,7-Dichloro-3-(1-isopropyl-6-oxo-1,6-dihydropyridazin-3-yl)-2-methyl-1H-indol-1-yl)-acetic acid;
2-(3-(1-Benzyl-6-oxo-1,6-dihydropyridazin-3-yl)-5-fluoro-2-methyl-7-(methylsulfonyl)-1H-indol-1-yl)acetic acid;
2-(3-(3-Benzyl-4-oxo-3,4-dihydrophthalazin-1-yl)-5-fluoro-2-methyl-7-(methylsulfonyl)-1H-indol-1-yl)acetic acid;
2-(5-Fluoro-3-((1-(4-(2-hydroxypropan-2-yl)benzyl)-6-oxo-1,6-dihydropyridazin-3-yl)-methyl)-2-methyl-1H-indol-1-yl)acetic acid;
2-(5-Fluoro-3-((1-(4-(1,1,1,3,3,3-hexafluoro-2-hydroxypropan-2-yl)benzyl)-6-oxo-1,6-dihydropyridazin-3-yl)methyl)-2-methyl-1H-indol-1-yl)acetic acid;
2-(5-Fluoro-3-((1-(3-(2-hydroxypropan-2-yl)benzyl)-6-oxo-1,6-dihydropyridazin-3-yl)-methyl)-2-methyl-1H-indol-1-yl)acetic acid;
2-(5-Fluoro-3-((1-((3-fluoropyridin-4-yl)methyl)-6-oxo-1,6-dihydropyridazin-3-yl)methyl)-2-methyl-1H-indol-1-yl)acetic acid;
2-(3-((1-(2,4-Difluorobenzyl)-6-oxo-1,6-dihydropyridazin-3-yl)methyl)-5,7-difluoro-2-methyl-1H-indol-1-yl)acetic acid;
2-(3-(3-Benzyl-4-oxo-3,4-dihydrophthalazin-1-yl)-7-chloro-5-fluoro-2-methyl-1H-indol-1-yl)acetic acid;
2-(3-(3-Benzyl-4-oxo-3,4-dihydrophthalazin-1-yl)-5,7-difluoro-2-methyl-1H-indol-1-yl)-acetic acid;
2-(3-(3-Benzyl-4-oxo-3,4-dihydrophthalazin-1-yl)-5,7-dichloro-2-methyl-1H-indol-1-yl)-acetic acid;
2-(3-(1-Benzyl-6-oxo-1,6-dihydropyridazin-3-yl)-7-bromo-2-methyl-1H-indol-1-yl)acetic acid;
2-(3-(2-Benzyl-1-oxo-1,2-dihydroisoquinolin-4-yl)-2-methyl-1H-indol-1-yl)acetic acid;
2-(3-(2-Benzyl-1-oxo-1,2-dihydroisoquinolin-4-yl)-5-fluoro-2-methyl-1H-indol-1-yl)-acetic acid;
2-(5-Fluoro-3-(2-isopropyl-1-oxo-1,2-dihydroisoquinolin-4-yl)-2-methyl-1H-indol-1-yl)-acetic acid;
2-(5-Fluoro-2-methyl-3-(1-oxo-2-(2,2,2-trifluoroethyl)-1,2-dihydroisoquinolin-4-yl)-1H-indol-1-yl)acetic acid;
2-(2-Methyl-3-(1-oxo-2-(2,2,2-trifluoroethyl)-1,2-dihydroisoquinolin-4-yl)-1H-indol-1-yl)-acetic acid;
2-(3-(2-Isopropyl-1-oxo-1,2-dihydroisoquinolin-4-yl)-2-methyl-1H-indol-1-yl)acetic acid;
2-(3-(2-(2,2-Difluoro-2-methoxyethyl)-1-oxo-1,2-dihydroisoquinolin-4-yl)-2-methyl-1H-indol-1-yl)acetic acid;
2-(5-Fluoro-3-(2-(2-hydroxy-2-methylpropyl)-1-oxo-1,2-dihydroisoquinolin-4-yl)-2-methyl-1H-indol-1-yl)acetic acid;
2-(5-Fluoro-2-methyl-3-(1-oxo-2-(3,3,3-trifluoro-2-hydroxy-2-(trifluoromethyl)propyl)-1,2-dihydroisoquinolin-4-yl)-1H-indol-1-yl)acetic acid;
(5-Fluoro-2-methyl-3-(1-oxo-2-(4,4,4-trifluorobutyl)-1,2-dihydroisoquinolin-4-yl)-1H-indol-1-yl)acetic acid;
2-(5-Fluoro-2-methyl-3-(2-neopentyl-1-oxo-1,2-dihydroisoquinolin-4-yl)-1H-indol-1-yl)acetic acid;
2-(3-(3-((4H-1,2,4-Triazol-3-yl)methyl)-4-oxo-3,4-dihydrophthalazin-1-yl)-5-fluoro-2-methyl-1H-indol-1-yl)acetic acid;
2-(3-(3-(2-Amino-2-oxoethyl)-4-oxo-3,4-dihydrophthalazin-1-yl)-5-chloro-2-methyl-1H-indol-1-yl)acetic acid;
2-(5-Chloro-2-methyl-3-(3-((5-methyl-4H-1,2,4-triazol-3-yl)methyl)-4-oxo-3,4-dihydrophthalazin-1-yl)-1H-indol-1-yl)acetic acid;
2-(3-(3-((4H-1,2,4-Triazol-3-yl)methyl)-4-oxo-3,4-dihydrophthalazin-1-yl)-5-chloro-2-methyl-1H-indol-1-yl)acetic acid;
2-(3-(1-((4H-1,2,4-Triazol-3-yl)methyl)-6-oxo-1,6-dihydropyridazin-3-yl)-5-fluoro-2-methyl-1H-indol-1-yl)acetic acid;
2-(5-Fluoro-2-methyl-3-(1-((5-methyl-4H-1,2,4-triazol-3-yl)methyl)-6-oxo-1,6-dihydropyridazin-3-yl)-1H-indol-1-yl)acetic acid;
2-(5-Fluoro-2-methyl-3-(4-oxo-3-((1-phenyl-1H-1,2,4-triazol-5-yl)methyl)-3,4-dihydrophthalazin-1-yl)-1H-indol-1-yl)acetic acid;
2-Benzyl-6-(5-fluoro-1-(4-methoxybenzoyl)-2-methyl-1H-indol-3-yl)pyridazin-3(2H)-one;
2-Benzyl-6-(5-fluoro-2-methyl-1-nicotinoyl-1H-indol-3-yl)pyridazin-3(2H)-one;
6-(1-Benzyl-5-fluoro-2-methyl-1H-indol-3-yl)-2-benzylpyridazin-3(2H)-one;
2-(3-(1-Benzyl-6-oxo-1,6-dihydropyridin-3-yl)-2-methyl-1H-indol-1-yl)acetic acid;
2-(3-(1-Benzyl-6-oxo-1,6-dihydropyridin-3-yl)-5-chloro-2-methyl-1H-indol-1-yl)acetic acid;
2-(3-(1-Benzyl-6-oxo-1,6-dihydropyridin-3-yl)-5-fluoro-2-methyl-1H-indol-1-yl)acetic acid;
2-(5-Fluoro-3-(1-(2-fluorobenzyl)-6-oxo-1,6-dihydropyridin-3-yl)-2-methyl-1H-indol-1-yl)-acetic acid;
2-(5-Fluoro-3-(1-(4-fluorobenzyl)-6-oxo-1,6-dihydropyridin-3-yl)-2-methyl-1H-indol-1-yl)-acetic acid;
2-(3-(1-(2,6-Difluorobenzyl)-6-oxo-1,6-dihydropyridin-3-yl)-5-fluoro-2-methyl-1H-indol-1-yl)acetic acid;
2-(3-(1-(2,3-Difluorobenzyl)-6-oxo-1,6-dihydropyridin-3-yl)-5-fluoro-2-methyl-1H-indol-1-yl)acetic acid;
2-(3-(1-(2,4-Difluorobenzyl)-6-oxo-1,6-dihydropyridin-3-yl)-5-fluoro-2-methyl-1H-indol-1-yl)acetic acid;
2-(5-Fluoro-2-methyl-3-(6-oxo-1-(2,4,5-trifluorobenzyl)-1,6-dihydropyridin-3-yl)-1H-indol-1-yl)acetic acid;
2-(5-Fluoro-3-(2-(3-hydroxy-3-methylbutyl)-1-oxo-1,2-dihydroisoquinolin-4-yl)-2-methyl-1H-indol-1-yl)acetic acid;
2-(5-Chloro-3-(1-(2,4-difluorobenzyl)-6-oxo-1,6-dihydropyridin-3-yl)-2-methyl-1H-indol-1-yl)acetic acid;
2-(3-(1-(2,4-Difluorobenzyl)-6-oxo-1,6-dihydropyridin-3-yl)-2-methyl-1H-indol-1-yl)acetic acid;
2-(3-(1-Isopropyl-6-oxo-1,6-dihydropyridin-3-yl)-2-methyl-1H-indol-1-yl)acetic acid;
2-(5-Fluoro-3-(1-isopropyl-6-oxo-1,6-dihydropyridin-3-yl)-2-methyl-1H-indol-1-yl)acetic acid;
2-(5-Chloro-3-(1-isopropyl-6-oxo-1,6-dihydropyridin-3-yl)-2-methyl-1H-indol-1yl)acetic acid;
2-(2-Methyl-3-(6-oxo-1-(2,2,2-trifluoroethyl)-1,6-dihydropyridin-3-yl)-1H-indol-1-yl)acetic acid;
2-(5-Fluoro-2-methyl-3-(6-oxo-1-(2,2,2-trifluoroethyl)-1,6-dihydropyridin-3-yl)-1H-indol-1-yl)acetic acid;
2-(5-Chloro-2-methyl-3-(6-oxo-1-(2,2,2-trifluoroethyl)-1,6-dihydropyridin-3-yl)-1H-indol-1-yl)acetic acid;
2-(3-(1-(2-Fluorobenzyl)-6-oxo-1,6-dihydropyridin-3-yl)-2-methyl-1H-indol-1-yl)acetic acid;
2-(3-(1-(4-Fluorobenzyl)-6-oxo-1,6-dihydropyridin-3-yl)-2-methyl-1H-indol-1-yl)acetic acid;
2-(3-(1-(2,3-Difluorobenzyl)-6-oxo-1,6-dihydropyridin-3-yl)-2-methyl-1H-indol-1-yl)acetic acid;
2-(3-(1-(2,6-Difluorobenzyl)-6-oxo-1,6-dihydropyridin-3-yl)-2-methyl-1H-indol-1-yl)acetic acid;
2-(2-Methyl-3-(6-oxo-1-(2,4,5-trifluorobenzyl)-1,6-dihydropyridin-3-yl)-1H-indol-1-yl)-acetic acid;
2-(3-(1-Isobutyl-6-oxo-1,6-dihydropyridin-3-yl)-2-methyl-1H-indol-1-yl)acetic acid;
2-(3-(1-Cyclopentyl-6-oxo-1,6-dihydropyridin-3-yl)-2-methyl-1H-indol-1-yl)acetic acid;
2-(5-Chloro-3-(1-(4-fluorobenzyl)-6-oxo-1,6-dihydropyridin-3-yl)-2-methyl-1H-indol-1-yl)-acetic acid;
2-(5-Chloro-3-(1-(2-fluorobenzyl)-6-oxo-1,6-dihydropyridin-3-yl)-2-methyl-1H-indol-1-yl)-acetic acid;
2-(5-Chloro-3-(1-(2,6-difluorobenzyl)-6-oxo-1,6-dihydropyridin-3-yl)-2-methyl-1H-indol-1-yl)acetic acid;
2-(5-Chloro-3-(1-(2,3-difluorobenzyl)-6-oxo-1,6-dihydropyridin-3-yl)-2-methyl-1H-indol-1-yl)acetic acid;
2-(5-Chloro-2-methyl-3-(6-oxo-1-(2,4,5-trifluorobenzyl)-1,6-dihydropyridin-3-yl)-1H-indol-1-yl)acetic acid;
2-(5-Chloro-3-(1-(3,5-difluorobenzyl)-6-oxo-1,6-dihydropyridin-3-yl)-2-methyl-1H-indol-1-yl)acetic acid;
2-(5-Chloro-3-(1-(3-fluorobenzyl)-6-oxo-1,6-dihydropyridin-3-yl)-2-methyl-1H-indol-1-yl)-acetic acid;
2-(2-Methyl-3-(6-oxo-1-(4,4,4-trifluorobutyl)-1,6-dihydropyridin-3-yl)-1H-indol-1-yl)acetic acid;
2-(5-Fluoro-2-methyl-3-(6-oxo-1-(4,4,4-trifluorobutyl)-1,6-dihydropyridin-3-yl)-1H-indol-1-yl)acetic acid;
2-(5-Methoxy-2-methyl-3-(6-oxo-1-(2,2,2-trifluoroethyl)-1,6-dihydropyridin-3-yl)-1H-indol-1-yl)acetic acid;
1-(2,3-Difluorobenzyl)-5-(5-fluoro-2-methyl-1-(2-oxo-2-(pyrrolidin-1-yl)ethyl)-1H-indol-3-yl)pyridin-2(1H)-one;
2-(3-(1-(2,3-Difluorobenzyl)-6-oxo-1,6-dihydropyridin-3-yl)-5-fluoro-2-methyl-1H-indol-1-yl)-N,N-dimethylacetamide;
2-(3-(1-(2,3-Difluorobenzyl)-6-oxo-1,6-dihydropyridin-3-yl)-5-fluoro-2-methyl-1H-indol-1-yl)acetamide;
2-(3-(1-(2,3-Difluorobenzyl)-6-oxo-1,6-dihydropyridin-3-yl)-5-fluoro-2-methyl-1H-indol-1-yl)-N-(methylsulfonyl)acetamide;
3-(3-(1-(2,3-Difluorobenzyl)-6-oxo-1,6-dihydropyridin-3-yl)-5-fluoro-2-methyl-1H-indol-1-yl)propanoic acid;
2-(2,5-Dimethyl-3-(6-oxo-1-(2,2,2-trifluoroethyl)-1,6-dihydropyridin-3-yl)-1H-indol-1-yl)-acetic acid;
1-(2,3-Difluorobenzyl)-5-(5-fluoro-1-(2-hydroxyethyl)-2-methyl-1H-indol-3-yl)pyridin-2(1H)-one;
(S)-2-(3-(1-(2,3-Difluorobenzyl)-6-oxo-1,6-dihydropyridin-3-yl)-5-fluoro-2-methyl-1H-indol-1-yl)propanoic acid;
(R)-2-(3-(1-(2,3-Difluorobenzyl)-6-oxo-1,6-dihydropyridin-3-yl)-5-fluoro-2-methyl-1H-indol-1-yl)propanoic acid;
2-(5-Fluoro-2-methyl-3-(6-oxo-1-(3,3,3-trifluoropropyl)-1,6-dihydropyridin-3-yl)-1H-indol-1-yl)acetic acid;
2-(5-Fluoro-2-methyl-3-(6-oxo-1-(pyridin-4-ylmethyl)-1,6-dihydropyridin-3-yl)-1H-indol-1-yl)acetic acid;
2-(5-Fluoro-2-methyl-3-(6-oxo-1-(pyridin-2-ylmethyl)-1,6-dihydropyridin-3-yl)-1H-indol-1-yl)acetic acid;
2-(3-(1-(2,3-Difluorobenzyl)-6-oxo-1,6-dihydropyridin-3-yl)-5-fluoro-2-methyl-1H-indol-1-yl)acetonitrile;
5-(1-((2H-Tetrazol-5-yl)methyl)-5-fluoro-2-methyl-1H-indol-3-yl)-1-(2,3-difluorobenzyl)-pyridin-2(1H)-one;
2-(3-(1-Benzyl-6-oxo-1,6-dihydropyridin-3-yl)-5-fluoro-2-methyl-1H-indol-1-yl)-N-(phenylsulfonyl)acetamide;
2-(3-(1-Benzyl-6-oxo-1,6-dihydropyridin-3-yl)-5-fluoro-2-methyl-1H-indol-1-yl)-N-(methyl-sulfonyl)acetamide;
2-(3-(1-Benzyl-6-oxo-1,6-dihydropyridin-3-yl)-5-fluoro-2-methyl-1H-indol-1-yl)-N-(o-tolyl-sulfonyl)acetamide;
2-(5-Fluoro-2-methyl-3-(6-oxo-1-(pyridin-3-ylmethyl)-1,6-dihydropyridin-3-yl)-1H-indol-1-yl)acetic acid;
2-(5-Fluoro-2-methyl-3-(6-oxo-1-(4,4,4-trifluoro-3-(trifluoromethyl)butyl)-1,6-dihydro-pyridin-3-yl)-1H-indol-1-yl)acetic acid;
N-(Cyclopropylsulfonyl)-2-(5-fluoro-2-methyl-3-(6-oxo-1-(4,4,4-trifluorobutyl)-1,6-dihydropyridin-3-yl)-1H-indol-1-yl)acetamide;
2-(5-Fluoro-2-methyl-3-(6-oxo-1-(4,4,4-trifluorobutyl)-1,6-dihydropyridin-3-yl)-1-H-indol-1-yl)-N-(methylsulfonyl)acetamide;
2-(3-(1-Benzyl-6-oxo-1,6-dihydropyridin-3-yl)-5-fluoro-2-methyl-1H-indol-1-yl)-N-(cyclo-propyl-sulfonyl)acetamide;
2-(3-((1-(2,4-Difluorobenzyl)-6-oxo-1,6-dihydropyridazin-3-yl)methyl)-5-fluoro-2-methyl-1H-indol-1-yl)-N-(methylsulfonyl)acetamide;
2-(3-((1-(3,4-Difluorobenzyl)-6-oxo-1,6-dihydropyridin-3-yl)methyl)-5-fluoro-2-methyl-1H-indol-1-yl)-N-(methylsulfonyl)acetamide;
2-(5-Chloro-2-methyl-3-(4-oxo-3-(2-phenoxyethyl)-3,4-dihydrophthalazin-1-yl)-1H-indol-1-yl)-N-(methylsulfonyl)acetamide;
2-(3-(2-Benzyl-1-oxo-1,2-dihydroisoquinolin-4-yl)-5-fluoro-2-methyl-1H-indol-1-yl)-N-(methylsulfonyl)acetamide;
2-(3-(1-Benzyl-6-oxo-1,6-dihydropyridazin-3-yl)-5-fluoro-2-methyl-1H-indol-1yl)acetic acid;
2-(3-(1-(2,4-Dichlorobenzyl)-6-oxo-1,6-dihydropyridazin-3-yl)-5-fluoro-2-methyl-1H-indol-1-yl)acetic acid;
[3-(3-Isopropyl-4-oxo-3,4-dihydrophthalazin-1-yl)-2-methyl-1H-indol-1-yl]acetic acid;
{5-Fluoro-2-methyl-3-[3-(2-methylpropyl)-4-oxo-3,4-dihydrophthalazin-1-yl]-1H-indol-1-yl}acetic acid;
[3-(3-Benzyl-4-oxo-3,4-dihydrophthalazin-1-yl)-5-fluoro-2-methyl-1H-indol-1-yl]acetic acid;
{5-Fluoro-3-[3-(3-fluorobenzyl)-4-oxo-3,4-dihydrophthalazin-1-yl]-2-methyl-1H-indol-1-yl}acetic acid;
{5-Fluoro-2-methyl-3-[3-(1-methylethyl)-4-oxo-3,4-dihydrophthalazin-1-yl]-1H-indol-1-yl}-acetic acid;
{5-Chloro-3-[3-(2,4-dichlorobenzyl)-4-oxo-3,4-dihydrophthalazin-1-yl]-2-methyl-1H-indol-1-yl}acetic acid;
(5-Chloro-2-methyl-3-{3-[4-(methylsulfonyl)benzyl]-4-oxo-3,4-dihydrophthalazin-1-yl}-1H-indol-1-yl)acetic acid;
2-(5-Fluoro-3-(1-isobutyl-6-oxo-1,6-dihydropyridazin-3-yl)-2-methyl-1H-indol-1-yl)acetic acid;
[5-Fluoro-3-(1-isopropyl-6-oxo-1,6-dihydropyridazin-3-yl)-2-methyl-1H-indol-1-yl]acetic acid;
(3-{[1-(2,4-Difluorobenzyl)-6-oxo-1,6-dihydropyridazin-3-yl]methyl}-2-methyl-1H-indol-1-yl)acetic acid;
(3-{[1(2,5-Difluorobenzyl)-6-oxo-1,6-dihydropyridazin-3-yl]methyl}-5-fluoro-2-methyl-1H-indol-1-yl)acetic acid;
{3-[(1-Benzyl-6-oxo-1,6-dihydropyridazin-3-yl)methyl]-5-fluoro-2-methyl-1H-indol-1-yl}-acetic acid;
(3-{[1-(2,6-Difluorobenzyl)-6-oxo-1,6-dihydropyridazin-3-yl]methyl}-5-fluoro-2-methyl-1H-indol-1-yl)acetic acid;
(3-{[1(2,3-Difluorobenzyl)-6-oxo-1,6-dihydropyridazin-3-yl]methyl}-5-fluoro-2-methyl-1H-indol-1-yl)acetic acid;
(3-{[1-(2-Fluorobenzyl)-6-oxo-1,6-dihydropyridazin-3-yl]methyl}-5-fluoro-2-methyl-1H-indol-1-yl)acetic acid;
(3-{[1(3-fluorobenzyl)-6-oxo-1,6-dihydropyridazin-3-yl]methyl}-5-fluoro-2-methyl-1H-indol-1-yl)acetic acid;
(3-{[1-(4-Fluorobenzyl)-6-oxo-1,6-dihydropyridazin-3-yl]methyl}-5-fluoro-2-methyl-1-H-indol-1-yl)acetic acid;
(3-{[1-(2,2-Dimethylpropyl)-6-oxo-1,6-dihydropyridazin-3-yl]methyl}-5-fluoro-2-methyl-1H-indol-1-yl)acetic acid;
2-(5-Fluoro-2-methyl-3-((6-oxo-1-(4,4,4-trifluorobutyl)-1,6-dihydropyridazin-3-yl)methyl)-1H-indol-1-yl)acetic acid;
(5-Fluoro-2-methyl-3-{[6-oxo-1-(2,2,2-trifluoroethyl)-1,6-dihydropyridazin-3-yl]methyl}-1H-indol-1-yl)acetic acid;
{3-[(1-Benzyl-6-oxo-1,6-dihydropyridin-3-yl)methyl]-5-fluoro-2-methyl-1H-indol-1-yl}acetic acid;
{3-[(1-Benzyl-6-oxo-1,6-dihydropyridin-3-yl)methyl]-5-chloro-2-methyl-1H-indol-1-yl}acetic acid;
{3-[(1-Benzyl-6-oxo-1,6-dihydropyridin-3-yl)methyl]-2-methyl-1H-indol-1-yl}acetic acid; and
{3-[3-(2-amino-2-oxoethyl)-4-oxo-3,4-dihydrophthalazin-1-yl]-5-fluoro-2-methyl-1H-indol-1-yl}acetic acid or
a pharmaceutically acceptable salt thereof.

20. A pharmaceutical composition comprising a compound of claim 1 or a pharmaceutically acceptable salt thereof and a pharmaceutically acceptable carrier.

21. A method of treating a disease or a disorder in a patient, comprising administering to a patient in need thereof a compound of claim 1 or a pharmaceutically acceptable salt thereof.

22. The method of claim 21, wherein the disease or disorder is selected from the group consisting of asthma, chronic obstructive pulmonary disease (COPD), bronchitis, rhinitis, nasal polyposis, sarcoidosis, farmer's lung, fibroid lung, idiopathic intestinal pneumonia, cystic fibrosis, cough, psoriasis, dermatitis, urticaria, cutaneous eosinophilias, chronic sinusitis, eosinophilic esophagitis, eosinophilic gastroenteritis, eosinophilic colitis, eosinophilic fasciitis, lupus, rheumatoid arthritis, inflammatory Bowel disease, Celiac disease, scleroderma, ankylosing spondylitis, autoimmune diseases, allergic diseases and hyper IgE syndrome.

23. The method of claim 21, wherein the treatment of a disease or a disorder further comprises administering an additional therapeutic agent.

24. The method of claim 21, wherein the disease or disorder is characterized by elevated levels of prostaglandin D₂ (PGD₂) or a metabolite thereof.

25. The method of claim 21, wherein the disease or disorder is characterized by elevated levels of a thromboxane metabolite.

26. A method of inhibiting the binding of endogenous ligands to the CRTH-2 receptor in a cell, comprising contacting the cell with a therapeutically effective amount of a compound of claim 1 or a pharmaceutically acceptable salt thereof.

27. The method of claim 26, wherein the endogenous ligand is prostaglandin D₂ (PGD₂) or a metabolite thereof.

28. The method of claim 26, wherein the endogenous ligand is a thromboxane metabolite.
